(19) United States
(12) Patent Application Publication
(10) Pub. No.: US 2025/0066456 A1
(43) Pub. Date: Feb. 27, 2025

(54) ANTI-SARS-CoV-2 SPIKE (S) ANTIBODIES AND THEIR USE IN TREATING COVID-19

(71) Applicant: NOVAVAX, INC., Gaithersburg, MD (US)

(72) Inventors: Gale SMITH, Gaithersburg, MD (US); Nita PATEL, Gaithersburg, MD (US)

(21) Appl. No.: 18/723,454

(22) PCT Filed: Dec. 23, 2022
(86) PCT No.: PCT/US2022/082331

§ 371 Date: Jun. 23, 2024

Related U.S. Application Data
(60) Provisional application No. 63/293,451, filed on Dec. 23, 2021.

(51) Int. Cl. C07K 16/10

(52) U.S. Cl. CPC C07K 16/1003; C07K 2317/33; C07K 2317/565; C07K 2317/92

(57) ABSTRACT

Infectious diseases remain a problem throughout the world. The outbreak of sudden acute respiratory syndrome coronavirus 2 (SARS-Co V-2) has infected more than 640 million people worldwide. SARS-Co V-2 causes the disease COVID-19. Mutations in the SARS-CoV-2 S spike protein enable SARS-CoV-2 variants to escape neutralizing monoclonal antibodies produced from previous infection with SARS-CoV2 or by vaccination. The present invention provides antibodies that bind to the SARS-Co V-2 Spike (S) protein. The invention further relates to pharmaceutical compositions, immunotherapeutic compositions, and methods using the aforementioned antibodies that bind to the SARS-CoV-2 Spike (S) protein.

### CROSS-REFERENCE TO RELATED APPLICATIONS

This application claims priority to U.S. Provisional Application No. 63/293,451, filed on Dec. 23, 2021. The aforementioned application is incorporated by reference herein in its entirety.

### REFERENCE TO AN ELECTRONIC SEQUENCE LISTING

The contents of the electronic sequence listing (NOVV_097_01WO_SeqList_ST26.xml; Size: 221,021 bytes; and Date of Creation: Dec. 22, 2022) are herein incorporated by reference in its entirety.

### FIELD

The present disclosure is generally related to anti-sudden acute respiratory syndrome coronavirus 2 (SARS-CoV-2) Spike (S) antibodies and fragments thereof, which are useful for treating viral infections. In particular, the anti-SARS-CoV-2 Spike (S) antibodies and fragments thereof are used to treat coronavirus 19 disease (COVID-19).

### BACKGROUND OF THE INVENTION

Infectious diseases remain a problem throughout the world. The outbreak of sudden acute respiratory syndrome coronavirus 2 (SARS-CoV-2) has infected more than 640 million people worldwide. Worldwide, the death toll has surpassed 6.6 million. The SARS-CoV-2 coronavirus belongs to the same family of viruses as severe acute respiratory syndrome coronavirus (SARS-CoV) and Middle East respiratory syndrome coronavirus (MERS-CoV), which have killed hundreds of people in the past 17 years. SARS-CoV-2 causes the disease COVID-19. Mutations in the SARS-CoV-2 S spike protein enable SARS-CoV-2 variants to escape neutralizing monoclonal antibodies produced from previous infection with SARS-CoV-2 or by vaccination.

Thus, the development of broadly neutralizing antibodies that treat COVID-19 is desirable.

### SUMMARY

Provided herein are antibodies or fragments thereof that bind to a sudden acute respiratory syndrome coronavirus 2 (SARS-CoV-2) Spike (S) protein. In embodiments, the light chain complementarity-determining region 1 (VL CDR1) is selected from the group consisting of SEQ ID NOS: 11-14 and 76; the light chain complementarity-determining region 2 (VL CDR2) is selected from the group consisting of SEQ ID NOS: 15-18 and 77; the light chain complementarity-determining region 3 (VL CDR3) is selected from the group consisting of SEQ ID NOS: 19-22 and 78; the heavy chain complementarity-determining region 1 (VH CDR1) is selected from the group consisting of SEQ ID NOS: 23-26 and 79; the heavy chain complementarity-determining region 2 (VH CDR2) is selected from the group consisting of SEQ ID NOS: 27-30 and 80; and the heavy chain complementarity-determining region 3 (VH CDR3) is selected from the group consisting of SEQ ID NOS: 31-34 and 81. In embodiments, the antibody or fragment thereof comprises (i) a VH CDR1 according to SEQ ID NO: 23, a VH CDR2 according to SEQ ID NO: 27, and a VH CDR3 according to SEQ ID NO: 31; a VL CDR1 according to SEQ ID NO: 11, a VL CDR2 according to SEQ ID NO: 15; and a VL CDR3 according to SEQ ID NO: 19; (ii) a VH CDR1 according to SEQ ID NO: 24; a VH CDR2 according to SEQ ID NO: 28; a VH CDR3 according to SEQ ID NO: 32; a VL CDR1 according to SEQ ID NO: 12; a VL CDR2 according to SEQ ID NO: 16; and a VL CDR3 according to SEQ ID NO: 20; (iii) a VH CDR1 according to SEQ ID NO: 25; a VH CDR2 according to SEQ ID NO: 29; a VH CDR3 according to SEQ ID NO: 33; a VL CDR1 according to SEQ ID NO: 13; a VL CDR2 according to SEQ ID NO: 17; and a VL CDR3 according to SEQ ID NO: 21; (iv) a VH CDR1 according to SEQ ID NO: 26; a VH CDR2 according to SEQ ID NO: 30; a VH CDR3 according to SEQ ID NO: 34; a VL CDR1 according to SEQ ID NO: 14; a VL CDR2 according to SEQ ID NO: 18; and a VL CDR3 according to SEQ ID NO: 22; or (v) a VH CDR1 according to SEQ ID NO: 79; a VH CDR2 according to SEQ ID NO: 80; a VH CDR3 according to SEQ ID NO: 81; a VL CDR1 according to SEQ ID NO: 76; a VL CDR2 according to SEQ ID NO: 77; and a VL CDR3 according to SEQ ID NO: 78. In embodiments, the amino acid sequence of the variable heavy (VH) domain comprises or consists of an amino acid sequence with at least 90%, at least 95%, at least 96%, at least 97%, at least 98%, at least 99%, or 100% identity to a polypeptide having the amino acid sequence of any one of SEQ ID NOS: 5-8 and 75. In embodiments, the amino acid sequence of the variable light (VL) domain comprises or consists of an amino acid sequence with at least 90%, at least 95%, at least 96%, at least 97%, at least 98%, at least 99%, or 100% identity to a polypeptide having the amino acid sequence of any one of SEQ ID NOS: 1-4 and 74. In embodiments, provided herein is an antibody is selected from the group consisting of: an antibody comprising (i) a VH comprising an amino acid sequence with at least 90%, at least 95%, at least 96%, at least 97%, at least 98%, at least 99%, or 100% identity to SEQ ID NO:5; and (ii) a VL comprising an amino acid sequence with at least 90%, at least 95%, at least 96%, at least 97%, at least 98%, at least 99%, or 100% identity to SEQ ID NO: 1; an antibody comprising a (i) a VH comprising an amino acid sequence with at least 90%, at least 95%, at least 96%, at least 97%, at least 98%, at least 99%, or 100% identity to SEQ ID NO:6; and (ii) a VL comprising an amino acid sequence with at least 90%, at least 95%, at least 96%, at least 97%, at least 98%, at least 99%, or 100% identity to SEQ ID NO: 2; an antibody comprising (i) a VH comprising an amino acid sequence with at least 90%, at least 95%, at least 96%, at least 97%, at least 98%, at least 99%, or 100% identity to SEQ ID NO:7; and (ii) a VL comprising an amino acid sequence with at least 90%, at least 95%, at least 96%, at least 97%, at least 98%, at least 99%, or 100% identity to SEQ ID NO: 3; an antibody comprising (i) a VH comprising an amino acid sequence with at least 90%, at least 95%, at least 96%, at least 97%, at least 98%, at least 99%, or 100% identity to SEQ ID NO: 4; and (ii) a VL comprising an amino acid sequence with at least 90%, at least 95%, at least 96%, at least 97%, at least 98%, at least 99%, or 100% identity to SEQ ID NO: 8; and an antibody comprising (i) a VH comprising an amino acid sequence with at least 90%, at least 95%, at least 96%, at least 97%, at least 98%, at least 99%, or 100% identity to SEQ ID NO: 75; and (ii) a VL comprising an amino acid sequence with at least 90%, at least 95%, at least 96%, at least 97%, at least 98%, at least 99%, or 100% identity to SEQ ID NO: 74. In embodiments, the antibody or fragment thereof is a monoclonal antibody, a Fab, F(ab′)₂, Fab′, a scFv, or a single domain antibody (sdAb). In embodiments, the antibody comprises a human IgG1 or IgG4 domain. In embodiments, the antibody or fragment thereof has a dissociation constant (K_{D}) for a SARS-CoV-2 S polypeptide or variant thereof of 50 nM or less, 10 nM or less, 1 nM or less, 0.5 nM or less, 0.1 nM or less, 0.05 nM or less, 0.01 nM or less, or 0.001 nM or less. In embodiments, the antibody or fragment thereof binds to one or more CoV S polypeptides with at least 90%, at least 91%, at least 92%, at least 93%, at least 94%, at least 95%, at least 96%, at least 97%, at least 98%, at least 99%, or 100% identity to a polypeptide according to any one of SEQ ID NOS: 9, 10, 35-43, 72-73; and 90-139. In embodiments, provided herein is an isolated nucleic acid molecule encoding any one of the aforementioned antibodies or fragments. In embodiments, provided herein is an expression vector comprising a nucleic acid molecule encoding any one of the aforementioned antibodies or fragments. In embodiments, provided herein is a host cell comprising the aforementioned expression vector. In embodiments, provided herein is a pharmaceutical composition comprising an antibody or fragment provided herein and a pharmaceutically-acceptable carrier. In embodiments, provided herein is a method of treating a subject in need thereof infected with a SARS-CoV-2 virus or variant thereof comprising administering to the subject an antibody or fragment thereof described herein. In embodiments, the subject is aged 65 or older. In embodiments, the subject is immunocompromised. In embodiments, the subject is a pregnant female. In embodiments, the SARS-CoV-2 variant has a PANGO lineage selected from the group consisting of B.1.1.529; BA. 1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1.

Provided herein is an antibody or fragment thereof that binds to a sudden acute respiratory syndrome coronavirus 2 (CoV) Spike (S) glycoprotein, wherein the antibody or fragment thereof comprises: (i) a light chain complementarity-determining region 1 (VL CDR1) with at least 80%, at least 85%, at least 90%, at least 95%, or 100% identity to a sequence selected from the group consisting of SEQ ID NOS: 11-14 and 76; (ii) a light chain complementarity-determining region 2 (VL CDR2) with at least 80%, at least 85%, at least 90%, at least 95%, or 100% identity to a sequence selected from the group consisting of SEQ ID NOS: 15-18 and 77; (iii) a light chain complementarity-determining region 3 (VL CDR3) with at least 80%, at least 85%, at least 90%, at least 95%, or 100% identity to a sequence selected from the group consisting of SEQ ID NOS: 19-22 and 78; (iv) a heavy chain complementarity-determining region 1 (VH CDR1) with at least 80%, at least 85%, at least 90%, at least 95%, or 100% identity to a sequence selected from the group consisting of SEQ ID NOS: 23-26 and 79; (v) a heavy chain complementarity-determining region 2 (VH CDR2) with at least 80%, at least 85%, at least 90%, at least 95%, or 100% identity to a sequence selected from the group consisting of SEQ ID NOS: 27-30 and 80; and (vi) a heavy chain complementarity-determining region 3 (VH CDR3) with at least 80%, at least 85%, at least 90%, at least 95%, or 100% identity to a sequence selected from the group consisting of SEQ ID NOS: 31-34 and 81.Provided herein is an antibody or fragment thereof that binds to a sudden acute respiratory syndrome coronavirus 2 (SARS-CoV-2) Spike (S) protein, wherein the antibody or fragment thereof comprises: (i) a variable heavy (VH) domain comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NOS: 5-8 and 75; and (ii) a variable light (VL) domain comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1-4 and 74. Provided herein is an antibody or fragment thereof comprising: a VH CDR1 according to SEQ ID NO: 23, a VH CDR2 according to SEQ ID NO: 27, and a VH CDR3 according to SEQ ID NO: 31; a VL CDR1 according to SEQ ID NO: 11, a VL CDR2 according to SEQ ID NO: 15; and a VL CDR3 according to SEQ ID NO: 19. Provided herein is an antibody or fragment thereof comprising Provided herein is an antibody or fragment thereof comprising a VH CDR1 according to SEQ ID NO: 24; a VH CDR2 according to SEQ ID NO: 28; a VH CDR3 according to SEQ ID NO: 32; a VL CDR1 according to SEQ ID NO: 12; a VL CDR2 according to SEQ ID NO: 16; and a VL CDR3 according to SEQ ID NO: 20. Provided herein is an antibody or fragment thereof comprising a VH CDR1 according to SEQ ID NO: 25; a VH CDR2 according to SEQ ID NO: 29; a VH CDR3 according to SEQ ID NO: 33; a VL CDR1 according to SEQ ID NO: 13; a VL CDR2 according to SEQ ID NO: 17; and a VL CDR3 according to SEQ ID NO: 21. Provided herein is an antibody or fragment thereof comprising a VH CDR1 according to SEQ ID NO: 26; a VH CDR2 according to SEQ ID NO: 30; a VH CDR3 according to SEQ ID NO: 34; a VL CDR1 according to SEQ ID NO: 14; a VL CDR2 according to SEQ ID NO: 18; and a VL CDR3 according to SEQ ID NO: 22. Provided herein is an antibody or fragment thereof comprising a VH CDR1 according to SEQ ID NO: 79; a VH CDR2 according to SEQ ID NO: 80; a VH CDR3 according to SEQ ID NO: 81; a VL CDR1 according to SEQ ID NO: 76; a VL CDR2 according to SEQ ID NO: 77; and a VL CDR3 according to SEQ ID NO: 78. Provided herein is an antibody or fragment thereof comprising (i) a VH comprising the amino acid sequence of SEQ ID NO:5; and (ii) a VL comprising the amino acid sequence of SEQ ID NO: 1. Provided herein is an antibody or fragment thereof comprising (i) a VH comprising the amino acid sequence of SEQ ID NO:6; and (ii) a VL comprising the amino acid sequence of SEQ ID NO: 2. Provided herein is an antibody or fragment thereof comprising (i) a VH comprising the amino acid sequence of SEQ ID NO:7; and (ii) a VL comprising the amino acid sequence of SEQ ID NO: 3. Provided herein is an antibody or fragment thereof comprising (i) a VH comprising the amino acid sequence of SEQ ID NO:8; and (ii) a VL comprising the amino acid sequence of SEQ ID NO: 4. Provided herein is an antibody or fragment thereof comprising (i) a VH comprising the amino acid sequence of SEQ ID NO:75; and (ii) a VL comprising the amino acid sequence of SEQ ID NO: 74. In embodiments, the antibody or fragment thereof is a monoclonal antibody, a Fab, F(ab′)₂, Fab′, a scFv, or a single domain antibody (sdAb). In embodiments, the antibody comprises a human IgG1 or IgG4 domain. In embodiments, the antibody or fragment thereof has an equilibrium dissociation constant (K_{D}) for a CoV S glycoprotein or variant thereof of 50 nM or less, 10 nM or less, 1 nM or less, 0.5 nM or less, 0.1 nM or less, 0.05 nM or less, 0.01 nM or less, or 0.001 nM or less. In embodiments, the antibody or fragment thereof binds to a CoV S glycoprotein or variant thereof with an equilibrium dissociation constant (Kd) of less than 1.0×10⁻⁹ moles per liter (M), less than 1.0×10⁻¹⁰ M, less than 1.0×10⁻¹¹ M, or less than 1.0×10⁻¹² M. In embodiments, the antibody or fragment thereof binds to one or more CoV S polypeptides with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide according to any one of SEQ ID NOS: 9, 10, 35-43, 72-73, 90-139, and 145-147. In embodiments, the antibody or fragment thereof binds to from about 2 to about 20 CoV S glycoproteins. In embodiments, the antibody or fragment thereof is a broadly neutralizing antibody. In embodiments, the antibody or fragment thereof binds to an epitope on a CoV S glycoprotein, wherein the epitope comprises amino acids 476, 485, 486, 487, and 489 of the CoV S glycoprotein of SEQ ID NO: 10. In embodiments, the antibody or fragment thereof binds to an epitope on a CoV S glycoprotein, wherein the epitope comprises amino acids 485, 486, 487, and 489 of the CoV S glycoprotein of SEQ ID NO: 10. In embodiments, the antibody or fragment thereof binds to an epitope on a CoV S glycoprotein, wherein the epitope comprises amino acids 378 and 385 of the CoV S glycoprotein of SEQ ID NO: 10. In embodiments, the antibody or fragment thereof binds to an epitope on a CoV S glycoprotein, wherein the epitope comprises amino acids 444, 445, 446, and 448 of the CoV S glycoprotein of SEQ ID NO: 10. Provided herein is an isolated nucleic acid molecule encoding an antibody or fragment thereof provided herein. Provided herein is an isolated nucleic acid molecule encoding an antibody or fragment thereof provided herein. Provided herein is an expression vector comprising a nucleic acid described herein. Provided herein is a host cell comprising an expression vector provided herein. Provided herein is a pharmaceutical composition comprising an antibody or fragment thereof provided herein and a pharmaceutically-acceptable carrier. In embodiments, the pharmaceutical composition comprises up to two, up to three, up to four, up to five, up to six, up to seven, up to eight, up to nine, or up to ten antibodies or fragments thereof provided herein. Provided herein is a method of treating a subject in need thereof infected with a SARS-CoV-2 virus or variant thereof comprising administering to the subject an antibody or fragment thereof or pharmaceutical composition provided herein. In embodiments, the subject is aged 65 or older. In embodiments, the subject is immunocompromised. In embodiments, the subject is under 2 years old. In embodiments, the subject is a pregnant female. In embodiments, the SARS-CoV-2 variant has a PANGO lineage selected from the group consisting of B.1.1.529; BA.1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1. In embodiments, the SARS-CoV-2 variant has a World Health Organization Label of alpha, beta, gamma, delta, epsilon, iota, kappa, zeta, mu, or omicron.

### BRIEF DESCRIPTION OF THE DRAWINGS

FIGS. 1A-1D shows binding curves of 239.12 (FIG. 1A), 322.3 (FIG. 1B), 425.6 (FIG. 1C), and 35.13 (FIG. 1D) to the SARS-CoV-2 S proteins to SARS-CoV-2 S polypeptides related to the SARS-CoV-2 parent strain (SEQ ID NO: 35), the SARS-CoV-2 gamma strain (SEQ ID NO: 38), the SARS-CoV-2 beta strain (SEQ ID NO: 36), the SARS-CoV-2 delta strain (SEQ ID NO: 37), the SARS-CoV-2 alpha strain (SEQ ID NO: 39), and the SARS-CoV-2 omicron strain (SEQ ID NO: 42).

FIGS. 1E-1I shows binding curves of 239.12 (FIG. 1E), 322.3 (FIG. 1F), 425.6 (FIG. 1G), 35.13 (FIG. 1H), and 199.9 (FIG. 1I) to various SARS-CoV-2 S proteins related to the SARS-CoV-2 S omicron strain.

FIGS. 2A-2E show the EC50 of binding of 239.12 (FIG. 2A), 322.3 (FIG. 2B), 425.6 (FIG. 2C), 35.13 (FIG. 2D) to various recombinant SARS-CoV-2 S proteins. FIGS. 2E-2I show the EC50 of binding of 239.12 (FIG. 2E), 322.3 (FIG. 2F), 425.6 (FIG. 2G), 35.13 (FIG. 2I), and 199.9 (FIG. 2I) to various SARS-CoV-2 S proteins related to the SARS-CoV-2 S omicron strain. FIG. 2I further shows the EC50 of binding of 199.9 to a SARS-CoV-2 S protein derived from the SARS-CoV-2 parent strain (SEQ ID NO: 35).

FIGS. 3A-3D show a crystal structure of a SARS-CoV-2 S glycoprotein (Protein Databank ID: 6XCN. Critical residues for binding the 35.13 (FIG. 3A), 425.6 (FIG. 3B), 239.12 (FIG. 3C), and 322.3 (FIG. 3D) Fabs are shown as spheres. The right structure in each figure shows the critical residues of the SARS-CoV-2 S receptor binding domain (RBD) for binding each Fab (Protein Databank ID: 6Z2M).

FIGS. 4A-4C shows the minimum sample dilution of 35.13 (FIG. 4A), 425.6 (FIG. 4B), and 322.3 (FIG. 4C) required to neutralize greater than 99% of the concentration of SARS-CoV-2 tested (Neut99).

FIGS. 5A-5C show hACE2 receptor inhibition of the antibodies 35.13 (FIG. 5A), 425.6 (FIG. 5B), and 322.3 (FIG. 5C).

FIGS. 6A-6B show pseudovirus neutralization by antibodies 35.13 (FIG. 6A) and 425.6 (FIG. 6B).

FIG. 7 shows an alignment of the SARS-CoV-2 S glycoproteins from the ancestral, beta, delta, gamma, BA.1, BA.2, BA.5, and BQ.1.1 SARS-CoV-2 viruses. Critical amino acids (K378 and T385) for binding of the 322.3 to the SARS-CoV-2 S glycoproteins are boxed. Critical amino acids (K444, V445, G446, and N448) for binding of the 425.6 to the SARS-CoV-2 S glycoproteins are boxed. Critical amino acids (K444, V445, G446, and N448) for binding of the 425.6 to the SARS-CoV-2 S glycoproteins are boxed. The numbering of the critical amino acids is relative to a SARS-CoV-2 S glycoprotein of SEQ ID NO: 10.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses. As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an antibody sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In aspects, the adults are seniors about 65 years or older, or about 60 years or older. In aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

In aspects, the subject is immunocompromised. In embodiments, the immunocompromised subject is administered a medication that causes immunosuppression. Non-limiting examples of medications that cause immunosuppression include corticosteroids (e.g., prednisone), alkylating agents (e.g., cyclophosphamide), antimetabolites (e.g., azathioprine or 6-mercaptopurine), transplant-related immunosuppressive drugs (e.g., cyclosporine, tacrolimus, sirolimus, or mycophenolate mofetil), mitoxantrone, chemotherapeutic agents, methotrexate, tumor necrosis factor (TNF)-blocking agents (e.g., etanercept, adalimumab, infliximab). In embodiments, the immunocompromised subject is infected with a virus (e.g., human immunodeficiency virus or Epstein-Barr virus). In embodiments, the virus is a respiratory virus, such as respiratory syncytial virus, influenza, parainfluenza, adenovirus, or a picornavirus. In embodiments, the immunocompromised subject has acquired immunodeficiency syndrome (AIDS). In embodiments, the immunocompromised subject is a person living with human immunodeficiency virus (HIV). In embodiments, the immunocompromised subject is immunocompromised due to a treatment regiment designed to prevent inflammation or prevent rejection of a transplant. In embodiments, the immunocompromised subject is a subject who has received a transplant. In embodiments, the immunocompromised subject has undergone radiation therapy or a splenectomy. In embodiments, the immunocompromised subject has been diagnosed with cancer, an autoimmune disease, tuberculosis, a substance use disorder (e.g., an alcohol, opioid, or cocaine use disorder), stroke or cerebrovascular disease, a solid organ or blood stem cell transplant, sickle cell disease, thalassemia, autoimmune lymphoproliferative syndrome (ALPS), autoimmune polyglandular syndrome type 1 (APS-1), B-cell expansion with NF-κB and T-cell anergy (BENTA) disease, capsase eight deficiency state (CEDS), chronic granulomatous disease (CGD), common variable immunodeficiency (CVID), congenital neutropenia syndromes, a deficiency in the cytotoxic T-lymphocyte-associated antigen 4 (CTLA-4), a DOCK8 deficiency, a GATA2 deficiency, a glycosylation disorder with immunodeficiency, a hyper-immunoglobulin E syndrome (HIES), hyper-immunoglobulin M syndrome, diabetes, type 1 diabetes, type 2 diabetes, interferon gamma deficiency, interleukin 12 deficiency, interleukin 23 deficiency, leukocyte adhesion deficiency, lipopolysaccharide-responsive beige-like anchor (LRBA) deficiency, PI3 kinase disease, PLCG2-associated antibody deficiency and immune dysregulation (PLAID), severe combined immunodeficiency (SCID), STAT3 dominant-negative disease, STAT3 gain-of-function disease, warts, hypogammaglobulinemia, infections, and myelokathexis (WHIM) syndrome, Wisckott-Aldrich syndrome (WAS), X-linked agammaglobulinemia (XLA), X-linked lymphoproliferative disease (XLP), uremia, malnutrition, or XMEN disease. In embodiments, the immunocompromised subject is a current or former cigarette smoker. In embodiments, the immunocompromised subject has a B-cell defect, T-cell defect, macrophage defect, cytokine defect, phagocyte deficiency, phagocyte dysfunction, complement deficiency or a combination thereof.

In embodiments, the subject is overweight or obese. In embodiments, an overweight subject has a body mass index (BMI) ≥25 kg/m² and <30 kg/m². In embodiments, an obese subject has a BMI that is ≥30 kg/m². In embodiments, the subject has a mental health condition. In embodiments, the mental health condition is depression, schizophrenia, or anxiety.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

As used herein, the term “modification” as it refers to a SARS-CoV-2 spike (S) polypeptide refers to mutation, deletion, or addition of one or more amino acids of the CoV S polypeptide. The location of a modification within a CoV S polypeptide can be determined based on aligning the sequence of the polypeptide to SEQ ID NO: 10 (CoV S polypeptide containing signal peptide) or SEQ ID NO: 9 (mature CoV S polypeptide lacking a signal peptide).

The term SARS-CoV-2 “variant”, used interchangeably herein with a “heterogeneous SARS-CoV-2 strain,” refers to a SARS-CoV-2 virus comprising a CoV S polypeptide having one or more modifications as compared to a SARS-CoV S polypeptide having the amino acid sequence of SEQ ID NO: 9. For example, a SARS-CoV-2 variant may have at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 modifications, as compared to a CoV S polypeptide having the amino acid sequence of SEQ ID NO: 9. For example, a SARS-CoV-2 variant may have at least one and up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35 modifications, up to 40 modifications, up to 45 modifications, up to 50 modifications, up to 55 modifications, up to 60 modifications, up to 65 modifications, up to 70 modifications, up to 75 modifications, up to 80 modifications, up to 85 modifications, up to 90 modifications, up to 95 modifications, or up to 100 modifications as compared to a CoV S polypeptide having the amino acid sequence of SEQ ID NO: 9. In aspects, a SARS-CoV-2 variant may have between about 2 and about 35 modifications, between about 5 and about 10 modifications, between about 5 and about 20 modifications, between about 10 and about 20 modifications, between about 15 and about 25 modifications, between about 20 and 30 modifications, between about 20 and about 40 modifications, between about 25 and about 45 modifications, between about 25 and about 100 modifications, between about 25 and about 45 modifications, between about 35 and about 100 modifications, as compared to a CoV S polypeptide having the amino acid sequence of SEQ ID NO: 9.

In embodiments, the heterogeneous SARS-CoV-2 strain is a SARS-CoV-2 virus comprising a CoV S polypeptide with at least about 70%, at least about 75%, at least about 80%, at least about 85%, at least about 90%, at least about 95%, at least about 96%, at least about 97%, at least about 98%, or at least about 99% identity to a CoV S polypeptide having the amino acid sequence of SEQ ID NO: 9. In embodiments, the heterogeneous SARS-CoV-2 strain is a SARS-CoV-2 virus comprising a CoV S polypeptide with between about 70% and about 99.9% identity to a CoV S polypeptide having the amino acid sequence of SEQ ID NO: 9. In embodiments, the heterogeneous SARS-CoV-2 strain is a SARS-CoV-2 virus comprising a CoV S polypeptide with between about 70% and about 99.5% identity to a CoV S polypeptide having the amino acid sequence of SEQ ID NO: 9. In embodiments, the heterogeneous SARS-CoV-2 strain is a SARS-CoV-2 virus comprising a CoV S polypeptide with between about 90% and about 99.9% identity to a CoV S polypeptide having the amino acid sequence of SEQ ID NO: 9 or SEQ ID NO: 10. In embodiments, the heterogeneous SARS-CoV-2 strain is a SARS-CoV-2 virus comprising a CoV S polypeptide with between about 90% and about 99.8% identity to a CoV S polypeptide having the amino acid sequence of SEQ ID NO: 9. In embodiments, the heterogeneous SARS-CoV-2 strain is a SARS-CoV-2 virus comprising a CoV S polypeptide with between about 95% and about 99.9% identity to a CoV S polypeptide having the amino acid sequence of SEQ ID NO: 9. In embodiments, the heterogeneous SARS-CoV-2 strain is a SARS-CoV-2 virus comprising a CoV S polypeptide with between about 95% and about 99.8% identity to a CoV S polypeptide having the amino acid sequence of SEQ ID NO: 9. In embodiments, the heterogeneous SARS-CoV-2 strain is a SARS-CoV-2 virus comprising a CoV S polypeptide with between about 95% and about 99% identity to a CoV S polypeptide having the amino acid sequence of SEQ ID NO: 9. In embodiments, the heterogeneous SARS-CoV-2 strain has a World Health Organization Label of alpha, beta, gamma, delta, epsilon, eta, iota, kappa, zeta, mu, or omicron. In embodiments, the heterogeneous SARS-CoV-2 strain has a PANGO lineage selected from the group consisting of B.1.1.529; BA.1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1. The following document describes the Pango lineage designation and is incorporated by reference herein in its entirety: O'Toole et al. BMC Genomics, 23, 121 (2022).

In embodiments, the heterogeneous SARS-CoV-2 strain has a World Health Organization Label of omicron. In embodiments, the heterogeneous SARS-CoV-2 strain with a World Health Organization Label of omicron has at least 35 modifications compared to the wild-type SARS-CoV-2 S polypeptide of SEQ ID NO: 9. In embodiments, the heterogeneous SARS-CoV-2 strain with a World Health Organization Label of omicron has from 35 to 55, from 35 to 65, from 35 to 75, from 35 to 85, from 35 to 95, or from 35 to 105 modifications compared to the wild-type SARS-CoV-2 S polypeptide of SEQ ID NO: 9. In embodiments, the modifications are selected from the group consisting of T6I, T6R, A14S, A54V, V70A, T82I, G129D, H133Q, K134E, W139R, E143G, F144L, Q170E, I197V, L199I, V200E, V200G, G239V, G244S, G326D, G326H, R333T, L355I, S358F, S358L, S360P, S362F, T363A, D392N, R395S, K404N, N427K, K431T, V432P, G433S, L439R, L439Q, N447K, S464N, T465K, E471A, F473V, F473S, F477S, Q480R, G483S, Q485R, N488Y, Y492H, T534K, T591I, D601G, G626V, H642Y, N645S, N666K, P668H, S691L, N751K, D783Y, N843K, Q941H, N956K, L968F, D1186N, deletion of amino acid 11, deletion of amino acid 12, deletion of amino acid 13, deletion of amino acid 56, deletion of amino acid 57, deletion of amino acid 130, deletion of amino acid 131, deletion of amino acid 132, deletion of amino acid 144, deletion of amino acid 145, deletion of amino acid 198, and insertion of a tripeptide having the amino acid sequence of EPE between amino acids 214 and 215, and combinations thereof.

In embodiments, the CoV S polypeptide of the variant comprises a combination of modifications selected from the group consisting of:
(i) A54V, T82I, G129D, L199I, G326D, S358L, S360P, S362F, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 56, deletion of amino acid 57, deletion of amino acid 130, deletion of amino acid 131, deletion of amino acid 132, deletion of amino acid 198, and insertion of a tripeptide having the amino acid sequence of EPE between amino acids 214 and 215;
(ii) T6I, A14S, G129D, V200G, G326D, S358F, S360P, S362F, T363A, D392N, R395S, K404N, N427K, S464N, T465K, E471A, Q480R, Q485R, N488Y, Y492H, D601G, H642Y, N666K, P668H, N751K, D783Y, Q941H, N956K, deletion of amino acid 11, deletion of amino acid 12, and deletion of amino acid 13;
(iii) T6R, A14S, T82I, G129D, E143G, L199I, G326D, S358L, S360P, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 144, deletion of amino acid 145, deletion of amino acid 198, and insertion of a tripeptide having the amino acid sequence of EPE between amino acids 214 and 215;
(iv) T6I, A14S, G129D, V200G, G326D, S358F, S360P, S362F, T363A, D392N, K404N, N427K, L439Q, S464N, T465K, E471A, Q480R, Q485R, N488Y, Y492H, D601G, H642Y, N666K, P668H, S691L, N751K, D783Y, Q941H, N956K, deletion of amino acid 11, deletion of amino acid 12, and deletion of amino acid 13;
(v) T6I, A14S, G129D, V200G, G326D, S358F, S360P, S362F, T363A, D392N, S464N, T465K, E471A, Q480R, Q485R, N488Y, Y492H, D601G, H642Y, N666K, P668H, D783Y, Q941H, N956K, deletion of amino acid 11, deletion of amino acid 12, and deletion of amino acid 13;
(vi) T6I, A14S, G129D, V200G, G326D, S358F, S360P, S362F, T363A, D392N, R395S, K404N, D601G, H642Y, N645S, N666K, N751K, D783Y, Q941H, N956K, deletion of amino acid 11, deletion of amino acid 12, deletion of amino acid 13, deletion of amino acid 56, and deletion of amino acid 57;
(vii) V3G, T6I, A14S, G129D, V200G, G326D, S358F, S360P, S362F, T363A, D392N, R395S, K404N, L439R, S464N, T465K, E471A, F473V, Q485R, N488Y, Y492H, D601G, G626V, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(viii) V3G, T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(ix) T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(x) T6I, A14S, G129D, K134E, W139R, F144L, I197V, V200G, G244S, G326H, S358F, S360P, S362F, T363A, D392N, R395S, K404N, N427K, G433S, N447K, S464N, T465K, E471A, Q485R, N488Y, Y492H, D601G, H642Y, N666K, P668H, N751K, D783Y, Q941H, N956K, deletion of amino acid 11, deletion of amino acid 12, and deletion of amino acid 13;
(xi) T6I, A14S, G129D, K134E, W139R, F144L, I197V, V200G, G244S, G326H, R333T, S358F, S360P, S362F, T363A, D392N, R395S, K404N, N427K, G433S, L439R, N447K, S464N, T465K, E471A, F473S, Q485R, N488Y, Y492H, T5911, D601G, H642Y, N666K, P668H, N751K, D783Y, Q941H, N956K, D1186N, deletion of amino acid 11, deletion of amino acid 12, and deletion of amino acid 13;
(xii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N645S, N666K, P668H, N751K, D783Y, Q941H, N956K, deletion of amino acid 11, deletion of amino acid 12, deletion of amino acid 13, deletion of amino acid 56, and deletion of amino acid 57;
(xiii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xiv) T6I, A14S, V70A, G129D, H133Q, Q170E, V200E, G239V, G326H, R333T, L3551, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, and deletion of amino acid 131;
(xv) T6I, A14S, G129D, H133Q, Q170E, V200E, G326H, R333T, L355I, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xvi) T6I, A14S, G129D, V200G, G326D, R333T, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xvii) T6I, A14S, G129D, V200G, G326D, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57; and
(xviii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xix) deletion of amino acid 56, deletion of amino acid 57, and deletion of amino acid 131, N488Y, A557D, D601G, P668H or P668R, T7031, S969A, and D1105H;
(xx) D67A, K404N, E471K, N488Y, D601G, and A688V;
(xxi) D67A, D202G, L229H, K404N, E471K, N488Y, D601G, and A688V;
(xxii) D67A, D202G, deletion of 1, 2, or 3 amino acids of amino acids 228-230, K404N, E471K, N488Y, D601G, and A688V;
(xxiii) D67A, L229H, R233I, N488Y, K404N, E471K, D601G, and A688V;
(xxiv) L5F, T7N, P13S, D125Y, R177S, K404T, E471K, N488Y, D601G, H642Y, T1014I, and V1163F;
(xxv) W139C and L439;
(xxvi) deletion of amino acid 144, deletion of amino acid 145, T6R, E143G, L439R, T465K, D601G, P668R, and D937N;
(xxvii) deletion of amino acid 144, deletion of amino acid 145, T6R, G129D, E143G, L439R, T465K, D601G, P668R, and D937N;
(xxviii) deletion of amino acid 144, deletion of amino acid 145, T6R, T82I, G129D, Y132H, E143G, A209V, K404N L439R, T465K, D601G, P668R, and D937N;
(xxix) deletion of amino acid 144, deletion of amino acid 145, T6R, G129D, E143G, W245I, K404N, N426K, L439R, T465K, E471K, N488Y, D601G, P668R, and D937N;
(xxx) deletion of amino acid 144, deletion of amino acid 145, T6R, W51H, H53W, G129D, E143G, D200V, L201R, W2451, K404N, N426K, L439R, T465K, E471K, N488Y, D601G, P668R, and D937N;
(xxxi) deletion of amino acid 144, deletion of amino acid 145, T6R, G129D, E143G, K404N, L439R, T465K, E471Q, D601G, P668R, and D937N;
(xxxii) Q39R, A54V, E471K; D601G, Q664H, F875L, and deletion of 1, 2, 3, or 4 of amino acids 56, 57, 131, 132;
(xxxiii) T82I, D240G, E471K, D601G, and A688V;
(xxxiv) L439R, E471Q, D601G, P668R, and Q1058H;
(xxxv) G62V, T63I, R233N, L439Q, F477S, D601G, T846N, and deletion of 1, 2, 3, 4, 5, or 6 of amino acids 234-240;
(xxxvi) T82I, Y131S, Y132N, R333K, E471K, N488Y, D601G, P668H, and D937N; and
(xxxvii) G129D, G326D, S360P, S362F, K404N, N427K, T465K, E471A or E471K, Q480K or Q480R, Q485R, N488Y, Y492H, D601G, H642Y, N666K, P668H, N751K, D783Y, Q941H, and N953K; wherein the amino acids of the CoV S glycoprotein are numbered with respect to a polypeptide having the sequence of SEQ ID NO: 9.

As used herein, the terms “antibody” and “antibodies” (immunoglobulins) encompass monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. Such antibodies may be derived from any mammal, including, but not limited to, humans, monkeys, pigs, horses, rabbits, dogs, cats, mice, etc.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FW). The variable domains of native heavy and light chains each comprise four FW regions, largely adopting a j-sheet configuration, connected by three CDRs, which form loops connecting, and in some cases forming part of, the j-sheet structure. The CDRs in each chain are held together in close proximity by the FW regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, MD (1991)). The constant domains are generally not involved directly in antigen binding, but may influence antigen binding affinity and may exhibit various effector functions, such as participation of the antibody in ADCC, CDC, and/or apoptosis.

The term “hypervariable region” when used herein refers to the amino acid residues of an antibody which are associated with its binding to antigen. The hypervariable regions encompass the amino acid residues of the “complementarity determining regions” or “CDRs” (e.g., residues 24-34 (VL CDR1), 50-56 (VL CDR2) and 89-97 (VL CDR3) of the light chain variable domain and residues 31-35 (VH CDR1), 50-65 (VH CDR2) and 95-102 (VH CDR3) of the heavy chain variable domain; Kabat et al., Sequences of Proteins of Immunological Interest, 5th Ed. Public Health Service, National Institutes of Health, Bethesda, MD (1991)) and/or those residues from a “hypervariable loop” (e.g., residues 26-32 (VL CDR1), 50-52 (VL CDR2) and 91-96 (VL CDR3) in the light chain variable domain and 26-32 (VH CDR1), 53-55 (VH CDR2) and 96-101 (VH CDR3) in the heavy chain variable domain; Chothia and Lesk, J. Mol. Biol., 196:901-917 (1987)). “Framework” or “FW” residues are those variable domain residues flanking the CDRs. FW residues are present in chimeric, humanized, human, domain antibodies, diabodies, vaccibodies, linear antibodies, and bispecific antibodies.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, monoclonal antibodies are advantageous in that they can be synthesized by hybridoma cells that are uncontaminated by other immunoglobulin producing cells. Alternative production methods are known to those trained in the art, for example, a monoclonal antibody may be produced by cells stably or transiently transfected with the heavy and light chain genes encoding the monoclonal antibody.

The modifier “monoclonal” indicates the character of the antibody as being obtained from a substantially homogeneous population of antibodies, and is not to be construed as requiring engineering of the antibody by any particular method. The term “monoclonal” is used herein to refer to an antibody that is derived from a clonal population of cells, including any eukaryotic, prokaryotic, or phage clone, and not the method by which the antibody was engineered. For example, the monoclonal antibodies to be used in accordance with the present invention may be made by the hybridoma method first described by Kohler et al., Nature, 256:495 (1975), or may be made by any recombinant DNA method (see, e.g., U.S. Pat. No. 4,816,567), including isolation from phage antibody libraries using the techniques described in Clackson et al., Nature, 352:624-628 (1991) and Marks et al., J. Mol. Biol., 222:581-597 (1991), for example. These methods can be used to produce monoclonal mammalian, chimeric, humanized, human, domain antibodies, diabodies, vaccibodies, linear antibodies, and bispecific antibodies.

The term “chimeric” antibodies includes antibodies in which at least one portion of the heavy and/or light chain is identical with or homologous to corresponding sequences in antibodies derived from a particular species or belonging to a particular antibody class or subclass, and at least one other portion of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

“Humanized” forms of nonhuman (e.g., murine) antibodies are chimeric antibodies that contain minimal sequence derived from nonhuman immunoglobulin. For the most part, humanized antibodies are human immunoglobulins (recipient antibody) in which the native CDR residues are replaced by residues from the corresponding CDR of a nonhuman species (donor antibody) such as mouse, rat, rabbit or nonhuman primate having the desired specificity, affinity, and capacity. In some instances, FW region residues of the human immunoglobulin are replaced by corresponding nonhuman residues. Furthermore, humanized antibodies may comprise residues that are not found in the recipient antibody or in the donor antibody. These modifications are made to further refine antibody performance. In general, a humanized antibody heavy or light chain will comprise substantially all of at least one or more variable domains, in which all or substantially all of the CDRs correspond to those of a nonhuman immunoglobulin and all or substantially all of the FWs are those of a human immunoglobulin sequence. In certain embodiments, the humanized antibody will comprise at least a portion of an immunoglobulin constant region (Fe), typically that of a human immunoglobulin. For further details, see, Jones et al., Nature, 321:522-525 (1986); Riechmann et al., Nature, 332:323-329 (1988); and Presta, Curr. Op. Struct. Biol., 2:593-596 (1992).

A “human antibody” can be an antibody derived from a human or an antibody obtained from a transgenic organism that has been “engineered” to produce specific human antibodies in response to antigenic challenge and can be produced by any method known in the art. In certain techniques, elements of the human heavy and light chain loci are introduced into strains of the organism derived from embryonic stem cell lines that contain targeted disruptions of the endogenous heavy chain and light chain loci. The transgenic organism can synthesize human antibodies specific for human antigens, and the organism can be used to produce human antibody-secreting hybridomas. A human antibody can also be an antibody wherein the heavy and light chains are encoded by a nucleotide sequence derived from one or more sources of human DNA. A fully human antibody also can be constructed by genetic or chromosomal transfection methods, as well as phage display technology, or in vitro activated B cells, all of which are known in the art.

“Antibody-dependent cell-mediated cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. In one embodiment, such cells are human cells. While not wishing to be limited to any particular mechanism of action, these cytotoxic cells that mediate ADCC generally express Fc receptors (FcRs). The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

“Complement dependent cytotoxicity” or “CDC” refers to the ability of a molecule to initiate complement activation and lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santaro et al., J. Immunol. Methods, 202:163 (1996), may be performed.

“Effector cells” are leukocytes which express one or more FcRs and perform effector functions. The cells express at least FcγRI, FCγRII, FcγRII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

The terms “Fc receptor” or “FcR” are used to describe a receptor that binds to the Fc region of an antibody. In one embodiment, the FcR is a native sequence human FcR. Moreover, in certain embodiments, the FcR is one which binds an IgG antibody (a gamma receptor) and includes receptors of the FcγRI, FcγRII, FcγRII, and FcγRIV subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (IT1M) in its cytoplasmic domain. (See, Daëron, Annu. Rev. Immunol., 15:203-234 (1997)). FcRs are reviewed in Ravetch and Kinet, Annu. Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., Immunol., 117:587 (1976) and Kim et al., J. Immunol., 24:249 (1994)).

“Fv” is the minimum antibody fragment which contains a complete antigen-recognition and binding site. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Affinity” of an antibody for an epitope to be used in the treatment(s) described herein is a term well understood in the art and means the extent, or strength, of binding of antibody to epitope. Affinity may be measured and/or expressed in a number of ways known in the art, including, but not limited to, equilibrium dissociation constant (KD or Kd), apparent equilibrium dissociation constant (KD′ or Kd′), and IC50 (amount needed to effect 50% inhibition in a competition assay). It is understood that, for purposes of this invention, an affinity is an average affinity for a given population of antibodies which bind to an epitope. Values of KD′ reported herein in terms of mg IgG per mL or mg/mL indicate mg Ig per mL of serum, although plasma can be used. When antibody affinity is used as a basis for administration of the treatment methods described herein, or selection for the treatment methods described herein, antibody affinity can be measured before and/or during treatment, and the values obtained can be used by a clinician in assessing whether a human patient is an appropriate candidate for treatment.

As used herein, the term “avidity” is a measure of the overall binding strength (i.e., both antibody arms) with which an antibody binds an antigen. Avidity depends on three factors: (i) affinity of the antibody for the epitope on the antigen; (ii) valency of both the antibody and antigen; and (iii) structural arrangement of the parts that interact. Antibody avidity can be determined by measuring the dissociation of the antigen-antibody bond in antigen excess using any means known in the art, such as, but not limited to, by the modification of indirect fluorescent antibody as described by Gray et al., J. Virol. Meth., 44:11-24. (1993)

As used herein, the term “neutralizing antibody” refers to an antibody that reduces the ability of a pathogen to initiate or sustain infection in a host. A neutralizing anti-CoV S glycoprotein antibody is an antibody that reduces the ability of a SARS-CoV-2 virus or variant thereof to initiate or sustain infection in a host.

An “epitope” is a term well understood in the art and means any chemical moiety that exhibits specific binding to an antibody. An “antigen” is a moiety or molecule that contains an epitope, and, as such, also specifically binds to antibody.

The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The term “isotype” refers to the classification of an antibody's heavy or light chain constant region. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called α, δ, ε, γ, and μ, respectively. The structures and three-dimensional configurations of different classes of immunoglobulins are well-known. Of the various human immunoglobulin classes, only human IgG1, IgG2, IgG3, IgG4, and IgM are known to activate complement. Human IgG1 and IgG3 are known to mediate ADCC in humans. Human light chain constant regions may be classified into two major classes, kappa and lambda.

As used herein, the term “immunogenicity” means that a compound is capable of provoking an immune response (stimulating production of specific antibodies and/or proliferation of specific T cells).

As used herein, the term “broadly neutralizing antibody” refers to an antibody or fragment thereof that binds to the SARS-CoV-2 S glycoprotein of more than one heterogeneous SARS-CoV-2 strain. In embodiments, the broadly neutralizing antibody binds the SARS-CoV-2 S glycoprotein of at least two, at least three, at least four, at least five, at least six, at least seven, at least eight, at least nine, at least ten, at least 11, at least 12, at least 13, at least 14, at least 15, at least 16, at least 17, at least 18, at least 19, or at least 20 heterogeneous SARS-CoV-2 strains. In embodiments, the broadly neutralizing antibody binds the SARS-CoV-2 S glycoprotein of at least two and up to three, up to four, up to five, up to six, up to seven, up to eight, up to nine, up to ten, up to 11, up to 12, up to 13, up to 14, up to 15, up to 16, up to 17, up to 18, up to 19, or up to 20 heterogeneous SARS-CoV-2 strains. In embodiments, the broadly neutralizing antibody binds the SARS-CoV-2 S glycoprotein of between 2 and 10 heterogeneous SARS-CoV-2 strains.

Antibodies that Bind to the SARS-CoV-2 Spike Polypeptide

The present invention relates to antibodies that bind to the SARS-CoV-2 Spike polypeptides and variants thereof (anti-CoV S glycoprotein antibodies), as well as to compositions comprising those antibodies. A SARS-CoV-2 Spike polypeptide (“CoV S glycoprotein”) may comprise the amino acid sequence of:

(SEQ ID NO: 9)
QCVNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNFKNLREF
VFKNIDGYFKIYSKHTPINLVRDLPQGFSALEPLVDLPIGINITRFQTLLALHRSYLTPGDS
SSGWTAGAAAYYVGYLQPRTELLKYNENGTITDAVDCALDPLSETKCTLKSFTVEKGIYQTS
NFRVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYFPLQSYGFQPTNG
VGYQPYRVVVLSFELLHAPATVCGPKKSTNLVKNKCVNFNFNGLTGTGVLTESNKKFLPFQQ
FGRDIADTTDAVRDPQTLEILDITPCSFGGVSVITPGTNTSNQVAVLYQDVNCTEVPVAIHA
DQLTPTWRVYSTGSNVFQTRAGCLIGAEHVNNSYECDIPIGAGICASYQTQTNSPRRARSVA
SQSIIAYTMSLGAENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGFNFSQILPDPSKP
SKRSFIEDLLENKVTLADAGFIKQYGDCLGDIAARDLICAQKFNGLTVLPPLLTDEMIAQYT
SALLAGTITSGWTFGAGAALQIPFAMQMAYRFNGIGVTQNVLYENQKLIANQFNSAIGKIQD
SLSSTASALGKLQDVVNQNAQALNTLVKQLSSNFGAISSVLNDILSRLDKVEAEVQIDRLIT
GRLQSLQTYVTQQLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFYEPQIITTDNTFV
SGNCDVVIGIVNNTVYDPLQPELDSFKEELDKYFKNHTSPDVDLGDISGINASVVNIQKEID
RLNEVAKNLNESLIDLQELGKYEQYIKWPWYIWLGFIAGLIAIVMVTIMLCCMTSCCSCLKG
CCSCGSCCKFDEDDSEPVLKGVKLHYT

In embodiments, the CoV S glycoprotein comprises an N-terminal signal peptide; this protein has the amino acid sequence of SEQ ID NO 10 The signal peptide is underlined

(SEQ ID NO: 10)
MFVFLVLLPLVSSQCVNLTTRTQLPPAYTNSFTRGVYYPDKVFRSSVLHSTQDLF
LPFFSNVTWFHAIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTFEYVSQPFLMDL
EGKQGNFKNLREFVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPCNGVEGFNCY
FPLQSYGFQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECDIPIGAGICASYQ
TQTNSPRRARSVASQSIIAYTMSLGAENSVAYSNNSIAIPTNFTISVTTEILPVSMTKTSVD
CTMYICGDSTECSNLLLQYGSFCTQLNRALTGIAVEQDKNTQEVFAQVKQIYKTPPIKDFGG
FNFSQILPDPSKPSKRSFIEDLLFNKVTLADAGFIKQYGDCLGDIAARDLICAQKFNGLTVL
PPLLTDEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDILSRLD
KVEAEVQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWFVTQRNF
YEPQIITTDNTFVSGNCDVVIGIVNNTVYDPLQPELDSFKEELDKYFKNHTSPDVDLGDISG
INASVVNIQKEIDRLNEVAKNLNESLIDLQELGKYEQYIKWPWYIWLGFIAGLIAIVMVTIM
LCCMTSCCSCLKGCCSCGSCCKFDEDDSEPVLKGVKLHYT

The CoV S glycoprotein (SEQ ID NO: 9) is divided into a S1 subunit (amino acids 1-672 of SEQ ID NO: 9) and a S2 subunit (amino acids 673-1260 of SEQ ID NO: 9). The S1 subunit is further divided into an N-terminal domain (NTD, amino acids 1-318 of SEQ ID NO: 9), a receptor binding domain (RBD, amino acids 318-514 of SEQ ID NO: 9), subdomains 1 and 2 (SD1/2, amino acids 529-668 of SEQ ID NO: 9), and a furin cleavage site (amino acids 669-672 of SEQ ID NO: 2). The S2 subunit comprises an HR1 domain (amino acids 889-971 of SEQ ID NO: 9), an HR2 domain (amino acids 1150-1200 of SEQ ID NO: 2), a transmembrane domain (TM, amino acids 1201-1224 of SEQ ID NO: 2), and a cytoplasmic domain (CD, amino acids 1225-1260 of SEQ ID NO: 9). In embodiments, an anti-CoV S glycoprotein antibody binds to the S1 subunit, the S2 subunit, the NTD, the RBD, a furin cleavage site, an HR1 domain, a TM domain, a CD, or a combination thereof of a SARS-CoV 2 S glycoprotein.

In embodiments, a CoV S glycoprotein has up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 modifications compared to the CoV S glycoprotein of SEQ TD NO: 9.

Exemplary modifications to the CoV S glycoprotein are shown in the table below.

| Position | Position | |
|---|---|---|
| within | within | |
| SEQ ID | SEQ ID | |
| NO: 10 | NO: 9 | Potential Modifications |
| 14-305 | 1-292 | deletion of up to about 1, 2, 3, 4, 5, 10, 20, 30, |
| | | 40, 50, 60, 70, 80, 90, 100, 110, 120, 130, 140, |
| | | 150, 160, 170, 180, 190, 200, 210, 220, 230, |
| | | 240, 250, 260, 270, 280, 290, or 292 |
| | | amino acids |
| 18 | 5 | mutation to phenylalanine |
| | | mutation to tyrosine |
| | | mutation to tryptophan |
| 19 | 6 | mutation to arginine |
| | | mutation to lysine |
| | | mutation to histidine |
| | | mutation to isoleucine |
| 20 | 7 | mutation to asparagine |
| | | mutation to glutamine |
| | | mutation to isoleucine |
| | | mutation to valine |
| 24 | 11 | mutation to serine |
| | | mutation to threonine |
| | | deletion |
| 25 | 12 | insertion of amino acids proline-proline-alanine |
| | | (PPA) after amino acid 25 |
| | | deletion |
| 26 | 13 | mutation to serine |
| | | mutation to threonine |
| | | deletion |
| 27 | 14 | mutation to serine |
| | | mutation to threonine |
| 52 | 39 | mutation to arginine |
| | | mutation to lysine |
| | | mutation to histidine |
| 64 | 51 | mutation to histidine |
| | | mutation to lysine |
| | | mutation to arginine |
| 66 | 53 | mutation to tryptophan |
| | | mutation to tyrosine |
| | | mutation to phenylalanine |
| 67 | 54 | mutation to valine |
| | | mutation to isoleucine |
| | | mutation to leucine |
| 69 | 56 | Deletion of amino acid |
| 70 | 57 | Deletion of amino acid |
| | | Mutation to phenylalanine |
| | | Mutation to tyrosine |
| | | Mutation to tryptophan |
| 75 | 62 | Mutation to valine |
| | | Mutation to leucine |
| | | Mutation to isoleucine |
| 76 | 63 | Mutation to isoleucine |
| | | Mutation to valine |
| | | Mutation to leucine |
| 80 | 67 | mutation to alanine |
| | | mutation to glycine |
| 83 | 70 | mutation to alanine |
| 95 | 82 | mutation to beta branched amino acid |
| | | mutation to isoleucine |
| | | mutation to valine |
| 138 | 125 | mutation to tyrosine |
| | | mutation to phenylalanine |
| | | mutation to tryptophan |
| 142 | 129 | mutation to aspartic acid |
| | | mutation to glutamic acid |
| 143 | 130 | deletion of amino acid |
| 144 | 131 | Deletion of amino acid |
| | | Mutation to serine |
| 145 | 132 | Deletion of amino acid |
| | | Mutation to histidine |
| | | Mutation to asparagine |
| | | Mutation to glutamine |
| 146 | 133 | mutation to aromatic amino acid |
| | | mutation to tyrosine |
| | | mutation to phenylalanine |
| | | mutation to tryptophan |
| | | mutation to glutamine |
| | | mutation to asparagine |
| 147 | 134 | mutation to glutamic acid |
| | | mutation to aspartic acid |
| 152 | 139 | mutation to cysteine |
| | | mutation to methionine |
| | | mutation to serine |
| | | mutation to threonine |
| | | mutation to arginine |
| | | mutation to lysine |
| 156 | 143 | mutation to glycine |
| | | mutation to alanine |
| 157 | 144 | deletion of amino acid |
| | | mutation to leucine |
| 158 | 144 | deletion of amino acid |
| 183 | 170 | mutation to glutamic acid |
| | | mutation to aspartic acid |
| 190 | 177 | mutation to serine |
| | | mutation to threonine |
| | | mutation to cysteine |
| 210 | 197 | mutation to valine |
| | | mutation to isoleucine |
| | | mutation to leucine |
| | | mutation to beta branched amino acid |
| 211 | 198 | mutation to isoleucine |
| | | mutation to valine |
| | | mutation to leucine |
| | | mutation to beta branched amino acid |
| | | deletion of amino acid |
| 212 | 199 | mutation to isoleucine |
| | | mutation to valine |
| | | mutation to leucine |
| | | mutation to beta branched amino acid |
| | | deletion of amino acid |
| 213 | 200 | mutation to valine |
| | | mutation to leucine |
| | | mutation to isoleucine |
| | | mutation to beta branched amino acid |
| | | mutation to proline |
| | | mutation to glycine |
| | | mutation to glutamic acid |
| | | mutation to aspartic acid |
| 214 | 201 | mutation to arginine |
| | | mutation to lysine |
| | | mutation to histidine |
| | | mutation to aspartic acid |
| | | mutation to glutamic acid |
| | | insertion of amino acids glutamic |
| | | acid-proline-glutamic acid (EPE) after 214 |
| 215 | 202 | mutation to glycine |
| | | mutation to alanine |
| | | insertion of amino acids glutamic |
| | | acid-proline-glutamic acid (EPE) after 215 |
| 222 | 209 | mutation to valine |
| | | mutation to leucine |
| | | mutation to isoleucine |
| 241-244 | 228-231 | deletion of 1, 2, 3, or 4 amino acids |
| 242 | 229 | mutation to histidine |
| | | mutation to lysine |
| | | mutation to arginine |
| 246 | 233 | mutation to beta-branched amino acid |
| | | mutation to isoleucine |
| | | mutation to valine |
| | | mutation to threonine |
| | | mutation to asparagine |
| 247 | 234 | deletion of amino acid |
| 248 | 235 | deletion of amino acid |
| 249 | 236 | deletion of amino acid |
| 250 | 237 | deletion of amino acid |
| 251 | 238 | deletion of amino acid |
| 252 | 239 | deletion of amino acid |
| | | mutation to valine |
| | | mutation to isoleucine |
| | | mutation to leucine |
| | | mutation to beta branched amino acid |
| 253 | 240 | mutation to glycine |
| | | deletion of amino acid |
| 257 | 244 | mutation to serine |
| | | mutation to threonine |
| 258 | 245 | mutation to isoleucine |
| | | mutation to valine |
| | | mutation to leucine |
| | | mutation to beta branched amino acid |
| 339 | 326 | mutation to aspartic acid |
| | | mutation to glutamic acid |
| | | mutation to histidine |
| 346 | 333 | mutation to lysine |
| | | mutation to arginine |
| | | mutation to histidine |
| | | mutation to threonine |
| | | mutation to serine |
| 368 | 355 | mutation to isoleucine |
| | | mutation to leucine |
| | | mutation to valine |
| | | mutation to beta-branched amino acid |
| 371 | 358 | mutation to leucine |
| | | mutation to isoleucine |
| | | mutation to valine |
| | | mutation to phenylalanine |
| | | mutation to tyrosine |
| | | mutation to tryptophan |
| 373 | 360 | mutation to proline |
| 375 | 362 | mutation to phenylalanine |
| | | mutation to tyrosine |
| | | mutation to tryptophan |
| 376 | 363 | mutation to alanine |
| | | mutation to glycine |
| 405 | 392 | mutation to asparagine |
| | | mutation to glutamine |
| 408 | 395 | mutation to serine |
| | | mutation to threonine |
| 417 | 404 | mutation to asparagine |
| | | mutation to threonine |
| | | mutation to isoleucine |
| | | mutation to valine |
| | | mutation to serine |
| | | mutation to glutamine |
| | | mutation to beta-branched amino acid |
| 432 | 419 | mutation to lysine |
| | | mutation to arginine |
| | | mutation to histidine |
| 439 | 426 | mutation to lysine |
| | | mutation to arginine |
| | | mutation to histidine |
| 440 | 427 | Mutation to lysine |
| | | Mutation to arginine |
| | | Mutation to histidine |
| 444 | 431 | Mutation to threonine |
| | | Mutation to serine |
| 445 | 432 | Mutation to proline |
| 446 | 433 | Mutation to serine |
| | | Mutation to threonine |
| 452 | 439 | mutation to arginine |
| | | mutation to lysine |
| | | mutation to histidine |
| | | mutation to glutamine |
| | | mutation to asparagine |
| 453 | 440 | mutation to phenylalanine |
| | | mutation to tryptophan |
| 460 | 447 | mutation to lysine |
| | | mutation to arginine |
| 477 | 464 | mutation to asparagine |
| | | mutation to glutamine |
| 478 | 465 | mutation to lysine |
| | | mutation to arginine |
| | | mutation to histidine |
| 484 | 471 | mutation to alanine |
| | | mutation to lysine |
| | | mutation to arginine |
| | | mutation to histidine |
| | | mutation to glutamine |
| | | mutation to asparagine |
| 486 | 473 | mutation to valine |
| | | mutation to leucine |
| | | mutation to isoleucine |
| | | mutation to serine |
| | | mutation to threonine |
| 490 | 477 | mutation to serine |
| | | mutation to threonine |
| 493 | 480 | mutation to lysine |
| | | mutation to arginine |
| | | mutation to histidine |
| 494 | 481 | mutation to proline |
| 496 | 483 | mutation to serine |
| | | mutation to threonine |
| 498 | 485 | mutation to lysine |
| | | mutation to arginine |
| | | mutation to histidine |
| 501 | 488 | mutation to tyrosine |
| | | mutation to phenylalanine |
| | | mutation to tryptophan |
| 505 | 492 | mutation to histidine |
| 547 | 534 | mutation to lysine |
| | | mutation to arginine |
| | | mutation to histidine |
| 570 | 557 | Mutation to aspartic acid |
| | | Mutation to glutamic acid |
| 604 | 591 | Mutation to isoleucine |
| | | Mutation to leucine |
| | | Mutation to valine |
| | | Mutation to beta-branched amino acid |
| 613 | 600 | Mutation to histidine |
| | | Mutation to lysine |
| | | Mutation to arginine |
| 614 | 601 | Mutation to glycine |
| | | Mutation to alanine |
| 639 | 626 | Mutation to valine |
| | | Mutation to leucine |
| | | Mutation to isoleucine |
| 655 | 642 | Mutation to tyrosine |
| | | Mutation to phenylalanine |
| | | Mutation to tryptophan |
| 658 | 645 | Mutation to serine |
| 677 | 664 | Mutation to histidine |
| 679 | 666 | Mutation to lysine |
| | | Mutation to arginine |
| | | Mutation to histidine |
| 681 | 668 | Mutation to histidine |
| | | Mutation to lysine |
| | | Mutation to arginine |
| 682-685 | 669-672 | Mutation of 1, 2, 3, or 4 amino acids |
| 689-698 | 676-685 | Deletion of up to about 1, up to about 2, |
| | | up to about 3, up to about 4, up to about 5, |
| | | up to about 6, up to about 7, up to about 8, |
| | | up to about 9, or up to about 10 amino acids |
| 701 | 688 | Mutation to beta-branched amino acid |
| | | Mutation to valine |
| | | Mutation to isoleucine |
| | | Mutation to threonine |
| 704 | 691 | Mutation to leucine |
| | | Mutation to isoleucine |
| | | Mutation to valine |
| 715-724 | 702-711 | Deletion of up to about 1, up to about 2, |
| | | up to about 3, up to about 4, up to about 5, |
| | | up to about 6, up to about 7, up to about 8, |
| | | up to about 9, or up to about 10 amino acids |
| 716 | 703 | Mutation to beta-branched amino acid |
| | | Mutation to valine |
| | | Mutation to isoleucine |
| 764 | 751 | Mutation to lysine |
| | | Mutation to arginine |
| | | Mutation to histidine |
| 788-806 | 775-793 | Deletion of up to about 1, up to about 2, |
| | | up to about 3, up to about 4, up to about 5, |
| | | up to about 6, up to about 7, up to about 8, |
| | | up to about 9, up to about 10, up to about 11, |
| | | up to about 12, up to about 13, up to about 14, |
| | | up to about 15, up to about 16, up to about 17, |
| | | up to about 18, or up to about 19 amino acids |
| 796 | 783 | Mutation to tyrosine |
| | | Mutation to phenylalanine |
| | | Mutation to tryptophan |
| 819-828 | 806-815 | Deletion of up to about 1, up to about 2, |
| | | up to about 3, up to about 4, up to about 5, |
| | | up to about 6, up to about 7, up to about 8, |
| | | up to about 9, or up to about 10 amino acids |
| 856 | 843 | Mutation to lysine |
| | | Mutation to arginine |
| | | Mutation to histidine |
| 859 | 846 | Mutation to asparagine |
| | | Mutation to glutamine |
| 888 | 875 | Mutation to leucine |
| | | Mutation to isoleucine |
| | | Mutation to valine |
| 950 | 937 | Mutation to asparagine |
| | | Mutation to glutamine |
| 954 | 941 | Mutation to histidine |
| | | Mutation to lysine |
| | | Mutation to arginine |
| 969 | 956 | Mutation to lysine |
| | | Mutation to arginine |
| | | Mutation to histidine |
| 981 | 968 | Mutation to phenylalanine |
| | | Mutation to tyrosine |
| | | Mutation to tryptophan |
| 982 | 969 | Mutation to alanine |
| | | Mutation to glycine |
| | | Mutation to threonine |
| 986 | 973 | Mutation to proline |
| | | Mutation to glycine |
| 987 | 974 | Mutation to proline |
| | | Mutation to glycine |
| 1027 | 1014 | Mutation to isoleucine |
| | | Mutation to valine |
| | | Mutation to serine |
| 1071 | 1058 | Mutation to histidine |
| | | Mutation to arginine |
| | | Mutation to lysine |
| 1118 | 1105 | Mutation to histidine |
| | | Mutation to lysine |
| | | Mutation to arginine |
| | | Mutation to asparagine |
| | | Mutation to glutamine |
| 1176 | 1163 | Mutation to phenylalanine |
| | | Mutation to tyrosine |
| | | Mutation to tryptophan |
| 1199 | 1186 | Mutation to asparagine |
| | | Mutation to glutamine |
| 1214-1237 | 1201-1224 | Deletion of one or more amino acids of TM |
| 1238-1273 | 1225-1260 | Deletion of one or more amino acids of CD |

In embodiments, the CoV S glycoprotein has a sequence that is at least 80% at least 81% at least 82% at least 83% at least 84% at least 85% at least 86% at least 87% at least 88% at least 89% at least 90% at least 91% at least 92% at least 93% at least 94%, at least 95%, at least 96%, at least 97%, at least 98%, at least 99%, or 100% identical to any one of SEQ ID NOS: 9, 10, 35-43, 72, 73, 90-139, and 145-147.

In embodiments an anti-CoV S glycoprotein antibody may mediate antigen-dependent-cell-mediated-cytotoxicity (ADCC). In embodiments, the present invention is directed toward anti-CoV S glycoprotein antibodies of the IgG1, IgG2, IgG3, IgG4, or IgG5 isotypes. In embodiments, the antibodies mediate human ADCC, CDC, and/or apoptosis.

In one embodiment, anti-CoV S glycoprotein antibodies comprise a variable heavy chain (VH) and a variable light chain (VL). In embodiments, the anti-CoV S glycoprotein antibody comprises a VL having the amino acid sequence of any one of SEQ ID NOS: 1-4 and 74 or an amino acid sequence that is at least 90%, at least 95%, at least 96%, at least 97%, at least 98%, at least 99%, or 100% identity to any one of SEQ ID NOS: 1-4 and 74. In embodiments, the anti-CoV S glycoprotein antibody comprises a VH having the amino acid sequence of any one of SEQ ID NOS: 5-8 and 75 or an amino acid sequence that is at least 90%, at least 95%, at least 96%, at least 97%, at least 98%, at least 99%, or 100% identity to any one of SEQ ID NOS: 5-8 and 75. In embodiments, an anti-CoV S glycoprotein antibody comprises a VL of SEQ ID NO: 1 or a VL that is at least 90%, at least 95%, at least 96%, at least 97%, at least 98%, at least 99%, or 100% identical to SEQ ID NO: 1 and a VH of SEQ ID NO: 5 or a VH that is at least 90%, at least 95%, at least 96%, at least 97%, at least 98%, at least 99%, or 100% identical to SEQ ID NO: 5. In embodiments, an anti-CoV S glycoprotein antibody comprises a VL of SEQ ID NO: 2 or a VL that is at least 90%, at least 95%, at least 96%, at least 97%, at least 98%, at least 99%, or 100% identical to SEQ ID NO: 2 and a VH of SEQ ID NO: 6 or a VH that is at least 90%, at least 95%, at least 96%, at least 97%, at least 98%, at least 99%, or 100% identical to SEQ ID NO: 6. In embodiments, an anti-CoV S glycoprotein antibody comprises a VL of SEQ ID NO: 3 or a VL that is at least 90%, at least 95%, at least 96%, at least 97%, at least 98%, at least 99%, or 100% identical to SEQ ID NO: 3 and a VH of SEQ ID NO: 7 or a VH that is at least 90%, at least 95%, at least 96%, at least 97%, at least 98%, at least 99%, or 100% identical to SEQ ID NO: 7. In embodiments, an anti-CoV S glycoprotein antibody comprises a VL of SEQ ID NO: 4 or a VL that is at least 90%, at least 95%, at least 96%, at least 97%, at least 98%, at least 99%, or 100% identical to SEQ ID NO: 4 and a VH of SEQ ID NO: 8 or a VH that is at least 90%, at least 95%, at least 96%, at least 97%, at least 98%, at least 99%, or 100% identical to SEQ ID NO: 8. In embodiments, an anti-CoV S glycoprotein antibody comprises a VL of SEQ ID NO: 75 or a VL that is at least 90%, at least 95%, at least 96%, at least 97%, at least 98%, at least 99%, or 100% identical to SEQ ID NO: 75 and a VH of SEQ ID NO: 74 or a VH that is at least 90%, at least 95%, at least 96%, at least 97%, at least 98%, at least 99%, or 100% identical to SEQ ID NO: 74.

In embodiments, a VL of SEQ ID NOS: 1-4 comprises a N-terminal leader sequence. Up to 1, 2, 3, 4, 5, 6, 7, 8, 9, 10, 11, 12, 13, 14, or 15 amino acids of the N-terminal leader sequence of any one of SEQ ID NOS: 1-4 and 74 may be removed. In embodiments, provided herein are antibodies comprising a VL without an N-terminal leader sequence. In embodiments, a VH of any one of SEQ ID NOS: 5-8 and 75 comprises a N-terminal leader sequence. Up to 1, 2, 3, 4, 5, 6, 7, 8, 9, 10, 11, 12, 13, 14, or 15 amino acids of the N-terminal leader sequence of any one of SEQ ID NOS: 5-8 and 75 may be removed. In embodiments, provided herein are antibodies comprising a VH without an N-terminal leader sequence. In embodiments, the antibodies described herein comprise up to 1, 2, 3, 4, 5, 6, 7, 8, 9, 10, 11, 12, 13, 14, or 15 amino acids of the N-terminal leader sequence of VH or VL.

In embodiments, the VL and VH are selected from Table 1 below. The amino acids underlined with a solid line are the N-terminal leader sequences of VL and VH. The bolded amino acids are the CDRs of each VL and VH. The framework regions are underlined with a dotted line.

TABLE 1
VL and VH Sequences of anti-CoV S
glycoprotein antibodies

| Antibody | | |
|---|---|---|
| Name | VL | VH |
| 425.6 | 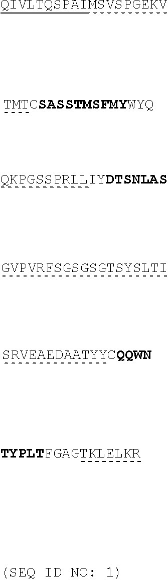 | 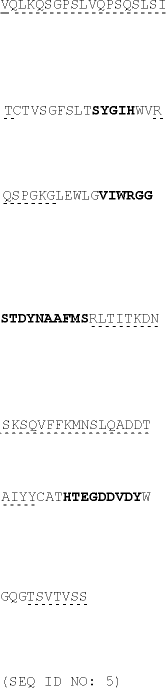 |
| 35.13 | 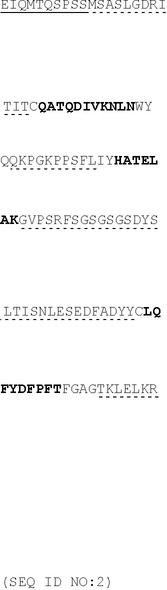 | 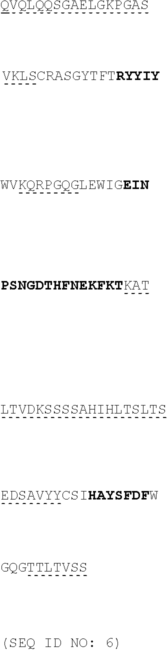 |
| 239.12 | 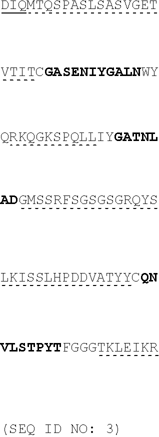 | 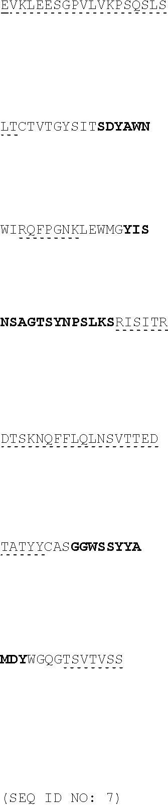 |
| 322.3 | 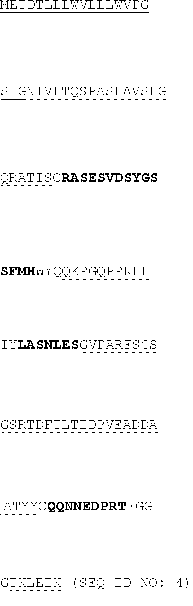 | 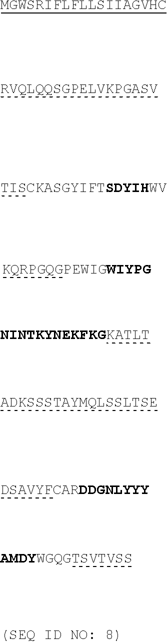 |
| 199.9 | 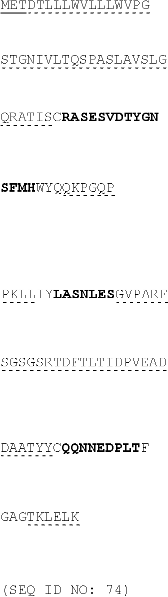 | 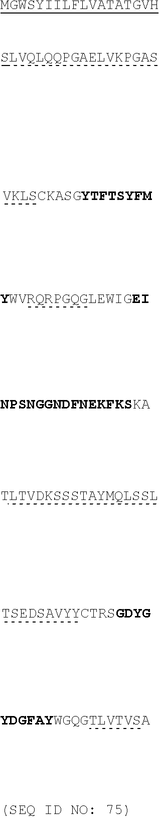 |

In embodiments, an anti-CoV S glycoprotein antibody comprises a variable heavy chain complementarity-determining region 1 (VH CDR1) having an amino acid sequence of any one of SEQ TD NOS: 23-26 and 79. In embodiments, an anti-CoV S glycoprotein antibody comprises a a variable heavy chain complementarity-determining region 2 (VH CDR2) having an amino acid sequence of any one of SEQ ID NOS: 27-30 and 80. In embodiments, an anti-CoV S glycoprotein antibody comprises a a variable heavy chain complementarity-determining region 3 (VH CDR3) having an amino acid sequence of any one of SEQ ID NOS: 31-34 and 81. In embodiments, an anti-CoV S glycoprotein antibody comprises a variable light chain complementarity-determining region 1 (VL CDR1) having an amino acid sequence of any one of SEQ ID NOS: 11-14 and 76. In embodiments, an anti-CoV S glycoprotein antibody comprises a variable light chain complementarity-determining region 2 (VL CDR2) having an amino acid sequence of any one of SEQ ID NOS: 15-18 and 77. In embodiments, an anti-CoV S glycoprotein antibody comprises a variable light chain complementarity-determining region 3 (VL CDR3) having an amino acid sequence of any one of SEQ ID NOS: 19-22 and 78.

In embodiments, provided herein is an anti-CoV S glycoprotein antibody comprising a VL CDR 1 selected from the group consisting of SEQ ID NOS: 11-14 and 76; a VL CDR 2 selected from the group consisting of SEQ ID NOS: 15-18 and 77; a VL CDR 3 selected from the group consisting of SEQ ID NOS: 19-22 and 78; a VH CDR 1 selected from the group consisting of SEQ ID NOS: 23-26 and 79; a VH CDR 2 selected from the group consisting of SEQ ID NOS: 27-30 and 80; and a VH CDR 3 selected from the group consisting of SEQ ID NOS: 31-34 and 81.

In embodiments, the VH CDR1, VH CDR2, VH CDR3, VL CDR1, VL CDR2, and VL CDR3 are independently selected from Table 2.

TABLE 2
CDR Sequences of anti-CoV S glycoprotein antibodies

| Antibody | | | | | | |
|---|---|---|---|---|---|---|
| Name | VL CDR1 | VL CDR2 | VL CDR3 | VH CDR1 | VH CDR2 | VH CDR3 |
| 425.6 | SASSTMS | DTSNLAS | QQWNTY | SYGIH | VIWRGGS | HTEGDD |
| | FMY (SEQ | (SEQ ID | PLT (SEQ | (SEQ ID | TDYNAA | VDY (SEQ |
| | ID NO: 11) | NO: 15) | ID NO: 19) | NO: 23) | FMS (SEQ | ID NO: 31) |
| | | | | | ID NO: 27) | |
| 35.13 | QATQDIV | HATELA | LQFYDFP | RYYIY | EINPSNG | HAYSFDF |
| | KNLN | K (SEQ ID | FT (SEQ | (SEQ ID | DTHFNEK | (SEQ ID |
| | (SEQ ID | NO: 16) | ID NO: 20) | NO: 24) | FKT (SEQ | NO: 32) |
| | NO: 12) | | | | ID NO: 28) | |
| 239.12 | GASENIY | GATNLA | QNVLSTP | SDYAWN | YISNSAG | GGWSSY |
| | GALN | D (SEQ ID | YT (SEQ | (SEQ ID | TSYNPSL | YAMDY |
| | (SEQ ID | NO: 17) | ID NO: 21) | NO: 25) | KS (SEQ | (SEQ ID |
| | NO: 13) | | | | ID NO: 29) | NO: 33) |
| 322.3 | RASESVD | LASNLES | QQNNED | SDYIH | WIYPGNI | DDGNLY |
| | SYGSSFM | (SEQ ID | PRT (SEQ | (SEQ ID | NTKYNE | YYAMDY |
| | H (SEQ ID | NO: 18) | ID NO: 22) | NO: 26) | KFKG | (SEQ ID |
| | NO: 14) | | | | (SEQ ID | NO: 34) |
| | | | | | NO: 30) | |
| 199.9 | RASESVD | LASNLES | QQNNED | YTFTSYF | EINPSNG | GDYGYD |
| | TYGNSF | (SEQ ID | PLT (SEQ | MY (SEQ | GNDFNE | GFAY |
| | MH (SEQ | NO: 77) | ID NO: 78) | ID NO: 79) | KFKS | (SEQ ID |
| | ID NO: 76) | | | | (SEQ ID | NO: 81) |
| | | | | | NO: 80) | |

In embodiments, an anti-CoV S glycoprotein antibody comprises a VH CDR1 of SEQ TD NO: 23, a VH CDR2 of SEQ ID NO: 27, and a VH CDR3 of SEQ TD NO: 31. In embodiments, an anti-CoV S glycoprotein antibody comprises a VL CDR1 of SEQ TD NO: 11, a VL CDR2 of SEQ TD NO: 15, and a VL CDR3 of SEQ TD NO: 19. In embodiments, an anti-CoV S glycoprotein antibody comprises comprises a VH CDR1 of SEQ TD NO: 23, a VH CDR2 of SEQ ID NO: 27, and a VH CDR3 of SEQ ID NO: 31 and a VL CDR1 of SEQ ID NO: 11, a VL CDR2 of SEQ ID NO: 15, and a VL CDR3 of SEQ ID NO: 19.

In embodiments, an anti-CoV S glycoprotein antibody comprises a VH CDR1 of SEQ ID NO: 24, a VH CDR2 of SEQ ID NO: 28, and a VH CDR3 of SEQ ID NO: 32. In embodiments, an anti-CoV S glycoprotein antibody comprises a VL CDR1 of SEQ ID NO: 12, a VL CDR2 of SEQ ID NO: 16, and a VL CDR3 of SEQ ID NO: 20. In embodiments, an anti-CoV S glycoprotein antibody comprises comprises a VH CDR1 of SEQ ID NO: 24, a VH CDR2 of SEQ ID NO: 28, and a VH CDR3 of SEQ ID NO: 32 and a VL CDR1 of SEQ ID NO: 12, a VL CDR2 of SEQ ID NO: 16, and a VL CDR3 of SEQ ID NO: 20.

In embodiments, an anti-CoV S glycoprotein antibody comprises a VH CDR1 of SEQ ID NO: 25, a VH CDR2 of SEQ ID NO: 29, and a VH CDR3 of SEQ ID NO: 33. In embodiments, an anti-CoV S glycoprotein antibody comprises a VL CDR1 of SEQ ID NO: 13, a VL CDR2 of SEQ ID NO: 17, and a VL CDR3 of SEQ ID NO: 21. In embodiments, an anti-CoV S glycoprotein antibody comprises comprises a VH CDR1 of SEQ ID NO: 25, a VH CDR2 of SEQ ID NO: 29, and a VH CDR3 of SEQ ID NO: 33, and a VL CDR1 of SEQ ID NO: 13, a VL CDR2 of SEQ ID NO: 17, and a VL CDR3 of SEQ ID NO: 21.

In embodiments, an anti-CoV S glycoprotein antibody comprises a VH CDR1 of SEQ ID NO: 26, a VH CDR2 of SEQ ID NO: 30, and a VH CDR3 of SEQ ID NO: 34. In embodiments, an anti-CoV S glycoprotein antibody comprises a VL CDR1 of SEQ ID NO: 14, a VL CDR2 of SEQ ID NO: 18, and a VL CDR3 of SEQ ID NO: 22. In embodiments, an anti-CoV S glycoprotein antibody comprises comprises a VH CDR1 of SEQ ID NO: 26, a VH CDR2 of SEQ ID NO: 30, and a VH CDR3 of SEQ ID NO: 34 and a VL CDR1 of SEQ ID NO: 14, a VL CDR2 of SEQ ID NO: 18, and a VL CDR3 of SEQ ID NO: 22.

In embodiments, an anti-CoV S glycoprotein antibody comprises a VH CDR1 of SEQ ID NO: 79, a VH CDR2 of SEQ ID NO: 80, and a VH CDR3 of SEQ ID NO: 81. In embodiments, an anti-CoV S glycoprotein antibody comprises a VL CDR1 of SEQ ID NO: 76, a VL CDR2 of SEQ ID NO: 77, and a VL CDR3 of SEQ ID NO: 78. In embodiments, an anti-CoV S glycoprotein antibody comprises comprises a VH CDR1 of SEQ ID NO: 79, a VH CDR2 of SEQ ID NO: 80, and a VH CDR3 of SEQ ID NO: 81 and a VL CDR1 of SEQ ID NO: 76, a VL CDR2 of SEQ ID NO: 77, and a VL CDR3 of SEQ ID NO: 78.

The present invention encompasses antibodies that bind to CoV S glycoproteins, comprising derivatives of the VH domains, VH CDR1s, VH CDR2s, VH CDR3s, VK domains, VK CDR1s, VK CDR2s, or VK CDR3s described herein that may bind to a SARS-CoV 2 S glycoprotein or a variant thereof. In embodiments, the anti-CoV S glycoprotein antibodies bind to a CoV S glycoprotein of a SARS-CoV-2 strain having a PANGO lineage selected from the group consisting of B.1.1.529; BA.1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1.

Standard techniques known to those of skill in the art can be used to introduce modifications (e.g., additions, deletions, and/or substitutions) in the nucleotide sequence encoding an antibody, including, for example, site-directed mutagenesis and PCR-mediated mutagenesis that are routinely used to generate amino acid substitutions. In another embodiment, the VH and/or VK CDRs derivatives may have conservative amino acid substitutions (e.g. supra) made at one or more predicted non-essential amino acid residues (i.e., amino acid residues which are not critical for the antibody to specifically bind to SARS-CoV-2 S glycoprotein). Mutations can also be introduced randomly along all or part of the VH and/or VL CDR coding sequences, such as by saturation mutagenesis, and the resultant mutants can be screened for biological activity to identify mutants that retain activity. Following mutagenesis, the encoded antibody can be expressed and the activity of the antibody can be determined.

The present invention further encompasses antibodies that bind to SARS-CoV-2 S glycoproteins, wherein said antibodies or antibody fragments comprising one or more CDRs wherein said CDRs comprise an amino acid sequence that is at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of one or more CDRs described herein. The percent identity of two amino acid sequences can be determined by any method known to one skilled in the art, including, but not limited to, BLAST protein searches.

### Framework Regions of Anti-CoV S Glycoprotein Antibody

In embodiments, the anti-CoV S glycoprotein antibodies comprise a VL and VH that each contain four framework regions (FW1, FW2, FW3, and FW4). In embodiments, FW1, FW2, FW3, and FW4 of VL are independently selected from Table 4. In embodiments, FW1, FW2, FW3, and FW4 of VH are independently selected from Table 5. Table 4. FW 1, FW2, FW3, FW4 of VL of anti-CoV S glycoprotein antibodies

TABLE 4
FW1, FW2, FW3, FW4 of VL of anti-CoV S glycoprotein antibodies

| Anti- | | | | |
|---|---|---|---|---|
| body | | | | |
| Name | FW1 | FW2 | FW3 | FW4 |
| 425.6 | MSVSPGEKVTMT (SEQ ID | QKPGS | GVPVRFSGSGSGTSYSL | TKL |
| | NO: 44) | SPRLL | TISRVEAEDAATYY | ELKR |
| | | (SEQ ID | (SEQ ID NO: 52) | (SEQ |
| | | NO: 48) | | ID NO: |
| | | | | 56) |
| 35.13 | MSASLGDRITIT (SEQ ID | QKPGK | GVPSRFSGSGSGSDYSL | TKL |
| | NO: 45) | PPSFL | TISNLESEDFADYY | ELKR |
| | | (SEQ ID | (SEQ ID NO: 53) | (SEQ |
| | | NO: 49) | | ID NO: |
| | | | | 57) |
| 239.12 | MTQSPASLSASVGETVTIT | RKQGK | GMSSRFSGSGSGRQYS | TKL |
| | (SEQ ID NO: 46) | SPQLL | LKISSLHPDDVATYY | EIKR |
| | | (SEQ ID | (SEQ ID NO: 54) | (SEQ |
| | | NO: 50) | | ID NO: |
| | | | | 58) |
| 322.3 | NIVLTQSPASLAVSLGQRATIS | QKPGQ | GVPARFSGSGSRTDFTL | TKL |
| | (SEQ ID NO: 47) | PPKLL | TIDPVEADDAATYY | EIK |
| | | (SEQ ID | (SEQ ID NO: 55) | (SEQ |
| | | NO: 51) | | ID NO: |
| | | | | 59) |
| 199.9 | DTLLLWVLLLWVPGSTGNIV | QKPGQ | GVPARFSGSGSRTDFTL | TKL |
| | LTQSPASLAVSLGQRATIS | PPKLL | TIDPVEADDAATYY | ELK |
| | (SEQ ID NO: 82) | (SEQ ID | (SEQ ID NO: 84) | (SEQ |
| | | NO: 83) | | ID NO: |
| | | | | 85) |

TABLE 5
FW1, FW2, FW3, FW4 of VH of anti-CoV S glycoprotein antibodies

| Antib | | | | |
|---|---|---|---|---|
| ody | | | | |
| Name | FW1 | FW2 | FW3 | FW4 |
| 425.6 | QLKQSGPSLVQPSQS | RQSPG | RLTITKDNSKSQVFFKMNSLQ | TSVT |
| | LSIT (SEQ ID NO: 60) | KG | ADDTAIYY (SEQ ID NO: 140) | VSS |
| | | (SEQ | | (SEQ |
| | | ID NO: | | ID NO: |
| | | 64) | | 68) |
| 35.13 | VQLQQSGAELGKPG | KQRPG | KATLTVDKSSSSAHIHLTSLT | TTLT |
| | ASVKLS (SEQ ID NO: | QG | SEDSAVYY (SEQ ID NO: 141) | VSS |
| | 61) | (SEQ | | (SEQ |
| | | ID NO: | | ID NO: |
| | | 65) | | 69) |
| 239.12 | VKLEESGPVLVKPSQ | RQFPG | RISITRDTSKNQFFLQLNSVTT | TSVT |
| | SLSLT (SEQ ID NO: | NK | EDTATYY (SEQ ID NO: 142) | VSS |
| | 62) | (SEQ | | (SEQ |
| | | ID NO: | | ID NO: |
| | | 66) | | 70) |
| 322.3 | RVQLQQSGPELVKPG | KQRPG | KATLTADKSSSTAYMQLSSL | TSVT |
| | ASVTIS (SEQ ID NO: | QG | TSEDSAVYF (SEQ ID NO: 143) | VSS |
| | 63) | (SEQ | | (SEQ |
| | | ID NO: | | ID NO: |
| | | 67) | | 71) |
| 199.9 | LVQLQQPGAEL VKPG | RQRPG | LTVDKSSSTAYMQLSSLTSED | TLVT |
| | ASVKLS (SEQ ID NO: | QG | SAVYY (SEQ ID NO: 88) | VS |
| | 86) | (SEQ | | (SEQ |
| | | ID NO: | | ID NO: |
| | | 87) | | 89) |

Kabat numbering is based on the seminal work of Kabat et al. (1991) Sequences of Proteins of Immunological Interest, Publication No. 91-3242, published as a three volume set by the National Institutes of Health, National Technical Information Service (hereinafter “Kabat”). Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (latest downloadable version 1997). Any immunoglobulin sequence can be numbered according to Kabat by performing an alignment with the Kabat reference sequence. Accordingly, the Kabat numbering system provides a uniform system for numbering immunoglobulin chains. Unless indicated otherwise, all immunoglobulin amino acid sequences described herein are numbered according to the Kabat numbering system. Similarly, all single amino acid positions referred to herein are numbered according to the Kabat numbering system.

In another embodiment, an anti-CoV S glycoprotein antibody of the invention may have an affinity constant or Kₐ (kₒₙ/k_{off}) of at least 10² M⁻¹, at least 5×10² M⁻¹, at least 10³ M ⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹ at least 5×10¹¹ M⁻¹, at least 10¹² M⁻¹, at least 5×10¹² M⁻¹, at least 1013 M⁻¹ at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In embodiments, an anti-CoV S glycoprotein antibody of the invention may have a dissociation constant or Kₐ (k_{off}/kₒₙ) of less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M as assessed using a method described herein or known to one of skill in the art (e.g., a BIAcore assay, ELISA).

The invention further provides polynucleotides comprising a nucleotide sequence encoding an anti-CoV S glycoprotein antibody described herein or fragments thereof. The invention also encompasses polynucleotides that hybridize under stringent or lower stringency hybridization conditions, e.g., as defined herein, to polynucleotides that encode an anti-CoV S glycoprotein antibody.

Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

A polynucleotide encoding an antibody may also be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably polyA+RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

The present invention also provides polynucleotide sequences encoding VH and VL framework regions and CDRs of antibodies described herein as well as expression vectors for their efficient expression in mammalian cells.

In one embodiment, an anti-CoV S glycoprotein antibody described herein mediates antibody-dependent cellular cytotoxicity (ADCC), complement-dependent cell-mediated cytotoxicity (CDC), and/or apoptosis. In one embodiment, an anti-CoV S glycoprotein antibody of the invention mediates antibody-dependent cellular cytotoxicity (ADCC) and/or apoptosis. In one embodiment, an anti-CoV S glycoprotein antibody of the invention has enhanced antibody-dependent cellular cytotoxicity (ADCC). In one embodiment, an anti-CoV S glycoprotein antibody of the invention comprises a variant Fc region that mediates enhanced antibody-dependent cellular cytotoxicity (ADCC). In one embodiment, an anti-CoV S glycoprotein antibody of the invention comprises an Fc region having complex N-glycoside-linked sugar chains linked to Asn297 in which fucose is not bound to N-acetylglucosamine in the reducing end, wherein said Fc region mediates enhanced antibody-dependent cellular cytotoxicity (ADCC).

### Production of Humanized Anti-CoV S Glycoprotein Antibodies

Humanized antibodies described herein can be produced using a variety of techniques known in the art, including, but not limited to, CDR-grafting (see e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology 28(4/5):489-498; Studnicka et al., 1994, Protein Engineering, 7(6):805-814; and Roguska et al., 1994, Proc. Natl. Acad. Sci; 91:969,973, each of which is incorporated herein by its entirety by reference), chain shuffling (see, e.g., U.S. Pat. No. 5,565,332, which is incorporated herein in its entirety by reference), and techniques disclosed in, e.g., U.S. Pat. Nos. 6,407,213, 5,766,886, International Publication No. WO 9317105, Tan et al., J Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, FW residues in the FW regions will be substituted with the corresponding residue from the CDR donor antibody to alter, preferably improve, antigen binding. These FW substitutions are identified by methods well known in the art, e.g., by modeling of the interactions of the CDR and FW residues to identify FW residues important for antigen binding and sequence comparison to identify unusual FW residues at particular positions. (See, e.g., Queen et al., U.S. Pat. No. 5,585,089; and Riechmann et al., 1988, Nature, 332:323, which are incorporated herein by reference in their entireties.)

A humanized anti-CoV S glycoprotein antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FW residues are substituted by residues from analogous sites in rodent antibodies. Humanization of an anti-CoV S glycoprotein antibody can also be achieved by veneering or resurfacing (EP 592,106; EP 519,596; Padlan, 1991, Molecular Immunology 28(4/5):489-498; Studnicka et al., Protein Engineering, 7(6):805-814 (1994); and Roguska et al., Proc. Natl. Acad. Sci., 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequences which are most closely related to that of the rodent are then screened for the presences of specific residues that may be critical for antigen binding, appropriate structural formation and/or stability of the intended humanized mAb (Sims et al., J. Immunol., 151:2296 (1993); Chothia et al., J. Mol. Biol., 196:901 (1987), the contents of which are incorporated herein by reference in their entirety). The resulting FW sequences matching the desired criteria are then be used as the human donor FW regions for the humanized antibody.

Another method uses a particular FW derived from the consensus sequence of all human antibodies of a particular subgroup of light or heavy chains. The same FW may be used for several different humanized anti-CoV S glycoprotein antibodies (Carter et al., Proc. Natl. Acad. Sci. USA, 89:4285 (1992); Presta et al., J. Immunol., 151:2623 (1993), the contents of which are incorporated herein by reference in their entirety).

Anti-CoV S glycoprotein antibodies can be humanized with retention of high affinity for SARS-CoV-2 S glycoprotein and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind SARS-CoV-2 S glycoprotein. In this way, FW residues can be selected and combined from the recipient and import sequences so that the desired antibody characteristic, for example affinity for SARS-CoV-2 S glycoprotein, is achieved. In general, the CDR residues are directly and most substantially involved in influencing antigen binding.

A “humanized” antibody may retain a similar antigenic specificity as the original antibody, i.e., in the present invention, the ability to bind the SARS-CoV-2 S glycoprotein. However, using certain methods of humanization, the affinity and/or specificity of binding of the antibody for the SARS-CoV-2 S glycoprotein may be altered using methods of “directed evolution,” as described by Wu et al., J. Mol. Biol, 294:151 (1999), the contents of which are incorporated herein by reference herein in their entirety.

Humanized anti-CoV S glycoprotein antibodies described herein can be constructed by the selection of distinct human framework regions for grafting of the 239.12, 322.3, 425.6, and 35.13 CDRs as described herein.

### Monoclonal Anti-CoV S Glycoprotein Antibodies

A monoclonal anti-CoV S glycoprotein antibody exhibits binding specificity to SARS-CoV-2 antigen and may mediate human ADCC, CDC and/or apoptotic mechanisms. Such an antibody can be generated using a wide variety of techniques known in the art including the use of hybridoma, recombinant, and phage display technologies, or a combination thereof. Antibodies are highly specific, being directed against a single antigenic site. An engineered anti-CoV S glycoprotein antibody can be produced by any means known in the art, including, but not limited to, those techniques described below and improvements to those techniques. Large-scale high-yield production typically involves culturing a host cell that produces the engineered anti-CoV S glycoprotein antibody and recovering the anti-CoV S glycoprotein antibody from the host cell culture.

### Hybridoma Technique

Monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in Monoclonal Antibodies and T Cell Hybridomas, 563-681 (Elsevier, N.Y., 1981) (said references incorporated herein by reference in their entireties). For example, in the hybridoma method, a mouse or other appropriate host animal, such as a hamster or macaque monkey, is immunized to elicit lymphocytes that produce or are capable of producing antibodies that will specifically bind to the protein used for immunization. Lymphocytes may also be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that contains one or more substances that inhibit the growth or survival of the unfused, parental mycloma cells. For example, if the parental mycloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the SARS-CoV-2 S glycoprotein. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, or serum by conventional immunoglobulin purification procedures such as, for example, protein A-Sepharose, hydroxylapatite chromatography, gel electrophoresis, dialysis, or affinity chromatography.

### Recombinant DNA Techniques

DNA encoding an anti-CoV S glycoprotein antibody described herein is readily isolated and sequenced using conventional procedures (e.g., by using oligonucleotide probes that are capable of binding specifically to genes encoding the heavy and light chains of anti-CoV S glycoprotein antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of anti-CoV S glycoprotein antibodies in the recombinant host cells.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods is typically filamentous phage including fd and M13 and the V_{H} and V_{L} domains are usually recombinantly fused to either the phage gene III or gene VIII. Phage expressing an antigen-binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods, 182:41-50; Ames et al., 1995, J. Immunol. Methods, 184:177-186; Kettleborough et al., 1994, Eur. J. Immunol., 24:952-958; Persic et al., 1997, Gene, 187:9-18; Burton et al., 1994, Advances in Immunology, 57:191-280; International Application No. PCT/GB91/O1 134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108; each of which is incorporated herein by reference in its entirety.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT Publication No. WO 92/22324; Mullinax et al., 1992, BioTechniques, 12(6):864-869; Sawai et al., 1995, AJRI, 34:26-34; and Better et al., 1988, Science, 240:1041-1043 (said references incorporated by reference in their entireties).

Antibodies may be isolated from antibody phage libraries generated using the techniques described in McCafferty et al., Nature, 348:552-554 (1990). Clackson et al., Nature, 352:624-628 (1991). Marks et al., J. Mol. Biol., 222:581-597 (1991) describe the isolation of murine and human antibodies, respectively, using phage libraries. Chain shuffling can be used in the production of high affinity (nM range) human antibodies (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of anti-CoV S glycoprotein antibodies.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. The vectors for expressing the VH or VL domains may comprise an EF-la promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also may be modified, for example, by substituting the coding sequence for human heavy and light chain constant domains in place of the homologous murine sequences (U.S. Pat. No. 4,816,567; Morrison et al., Proc. Natl. Acad. Sci. USA, 81:6851 (1984)), or by covalently joining to the immunoglobulin coding sequence all or part of the coding sequence for a non-immunoglobulin polypeptide.

### Chimeric Antibodies

The anti-CoV S glycoprotein antibodies herein specifically include chimeric antibodies (immunoglobulins) in which a portion of the heavy and/or light chain is identical with or homologous to corresponding sequences in antibodies derived from a particular species or belonging to a particular antibody class or subclass, while another portion of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

In embodiments the K_{D} of anti-CoV S glycoprotein antibodies described herein, or an for a SARS-CoV-2 S glycoprotein may be 50 nM or less, 10 nM or less, 1 nM or less, 0.5 nM or less, 0.1 nM or less, 0.05 nM or less, 0.01 nM or less, or 0.001 nM or less. Methods and reagents suitable for determination of such binding characteristics of an antibody of the present invention, or an altered/mutant derivative thereof, are known in the art and/or are commercially available (se above and, e.g., U.S. Pat. Nos. 6,849,425, 6,632,926, 6,294,391, and 6,143,574, each of which is hereby incorporated by reference in its entirety). Moreover, equipment and software designed for such kinetic analyses are commercially available (e.g. Biacore® A100, and Biacore® 2000 instruments; Biacore International AB, Uppsala, Sweden).

Identity or similarity with respect to a sequence is defined herein as the percentage of amino acid residues in the candidate sequence that are identical (i.e., same residue) or similar (i.e., amino acid residue from the same group based on common side-chain properties, see below) with anti-CoV S glycoprotein antibodies, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Methods for comparing the identity of two or more sequences are well known in the art. Percentage identity is calculated using the tool CLUSTALW2, which is available online. The following default parameters may be used for CLUSTALW2 Pairwise alignment: Protein Weight Matrix=Gonnet; Gap Open=10; Gap Extension=0.1. Unless described otherwise, the CLUSTALW2 tool is utilized to calculate percent identity herein.

To generate an altered antibody, one or more amino acid alterations (e.g., substitutions) are introduced in one or more of the hypervariable regions of the species-dependent antibody. One or more alterations (e.g., substitutions) of framework region residues may also be introduced in anti-CoV S glycoprotein antibodies where these result in an improvement in the binding affinity of the antibody mutant for the antigen from the second mammalian species. Examples of framework region residues to modify include those which non-covalently bind antigen directly (Amit et al., Science, 233:747-753 (1986)); interact with/effect the conformation of a CDR (Chothia et al., J. Mol. Biol., 196:901-917 (1987)); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hypervariable region residues have been altered. Normally, however, an altered antibody will comprise additional hypervariable region alteration(s).

The hypervariable region residues which are altered may be changed randomly, especially where the starting binding affinity of anti-CoV S glycoprotein antibodies for the antigen from the second mammalian species is such that such randomly produced altered antibody can be readily screened.

One useful procedure for generating such an altered antibody is called “alanine scanning mutagenesis” (Cunningham and Wells, Science, 244:1081-1085 (1989)). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing additional or other mutations at or for the sites of substitution. Thus, while the site for introducing an amino acid sequence variation is predetermined, the nature of the mutation per se need not be predetermined. The Ala-mutants produced this way are screened for their biological activity as described herein.

Another procedure for generating such an altered antibody involves affinity maturation using phage display (Hawkins et al., J. Mol. Biol., 254:889-896 (1992) and Lowman et al., Biochemistry, 30(45):10832-10837 (1991)). Briefly, several hypervariable region sites (e.g., 6-7 sites) are mutated to generate all possible amino acid substitutions at each site. The antibody mutants thus generated are displayed in a monovalent fashion from filamentous phage particles as fusions to the gene 111 product of M13 packaged within each particle. The phage-displayed mutants are then screened for their biological activity (e.g., binding affinity) as herein disclosed.

Mutations in antibody sequences may include substitutions, deletions, including internal deletions, additions, including additions yielding fusion proteins, or conservative substitutions of amino acid residues within and/or adjacent to the amino acid sequence, but that result in a “silent” change, in that the change produces a functionally equivalent anti-CoV S glycoprotein antibodies. Conservative amino acid substitutions may be made on the basis of similarity in polarity, charge, solubility, hydrophobicity, hydrophilicity, and/or the amphipathic nature of the residues involved. For example, non-polar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues that can influence chain orientation. Non-conservative substitutions will entail exchanging a member of one of these classes for a member of another class. Furthermore, if desired, non-classical amino acids or chemical amino acid analogs can be introduced as a substitution or addition into the antibody sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

In another embodiment, the sites selected for modification are affinity matured using phage display (see above).

Any technique for mutagenesis known in the art can be used to modify individual nucleotides in a DNA sequence, for purposes of making amino acid substitution(s) in the antibody sequence, or for creating/deleting restriction sites to facilitate further manipulations. Such techniques include, but are not limited to, chemical mutagenesis, in vitro site-directed mutagenesis (Kunkel, Proc. Natl. Acad. Sci. USA, 82:488 (1985); Hutchinson, C. et al., J. Biol. Chem., 253:6551 (1978)), oligonucleotide-directed mutagenesis (Smith, Ann. Rev. Genet., 19:423-463 (1985); Hill et al., Methods Enzymol., 155:558-568 (1987)), PCR-based overlap extension (Ho et al., Gene, 77:51-59 (1989)), PCR-based megaprimer mutagenesis (Sarkar et al., Biotechniques, 8:404-407 (1990)), etc. Modifications can be confirmed by double-stranded dideoxy DNA sequencing.

In certain embodiments of the invention, anti-CoV S glycoprotein antibodies can be modified to produce fusion proteins; i.e., the antibody, or a fragment thereof, fused to a heterologous protein, polypeptide or peptide.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of the anti-CoV S glycoprotein antibody (e.g., an antibody or a fragment thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol. 16(2):76-82; Hansson et al., 1999, J. Mol. Biol., 287:265-76; and Lorenzo and Blasco, 1998, Biotechniques 24(2):308-313 (each of these patents and publications are hereby incorporated by reference in its entirety). The antibody can further be a binding-domain immunoglobulin fusion protein as described in U.S. Publication 20030118592, U.S. Publication 200330133939, and PCT Publication WO 02/056910, all to Ledbetter et al., which are incorporated herein by reference in their entireties.

### Domain Antibodies

Anti-CoV S glycoprotein antibodies of compositions and methods of the invention can be domain antibodies, e.g., antibodies containing the small functional binding units of antibodies, corresponding to the variable regions of the heavy (V_{H}) or light (V_{L}) chains of human antibodies. Examples of domain antibodies include, but are not limited to, those available from Domantis Limited (Cambridge, UK) and Domantis Inc. (Cambridge, MA, USA) that are specific to therapeutic targets (see, for example, W004/058821; W004/003019; U.S. Pat. Nos. 6,291,158; 6,582,915; 6,696,245; and 6,593,081.

### Diabodies

In certain embodiments of the invention, anti-CoV S glycoprotein antibodies are “diabodies”. The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

### Linear Antibodies

In certain embodiments of the invention, anti-CoV S glycoprotein antibodies are linear antibodies. Linear antibodies comprise a pair of tandem Fd segments (V_{H}-C_{H1}-V_{H}-C_{H1}) which form a pair of antigen-binding regions. Linear antibodies can be bispecific or monospecific. See, Zapata et al., Protein Eng., 8(10):1057-1062 (1995).

### Antibody Fragments

“Antibody fragments” comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

Traditionally, these fragments were derived via proteolytic digestion of intact antibodies (see, e.g., Morimoto et al., Journal of Biochemical and Biophysical Methods, 24:107-117 (1992) and Brennan et al., Science, 229:81 (1985)). However, these fragments can now be produced directly by recombinant host cells. For example, the antibody fragments can be isolated from the antibody phage libraries discussed above. Fab′-SH fragments can also be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See, for example, WO 93/16185. In certain embodiments, the antibody is not a Fab fragment.

### Bispecific Antibodies

Bispecific antibodies are antibodies that have binding specificities for at least two different epitopes.

Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089.)

In one embodiment, where an anti-CoV S glycoprotein antibody of compositions and methods of the invention is bispecific, the anti-CoV S glycoprotein antibody may be human or humanized and may have specificity for SARS-CoV-2 S glycoprotein and an epitope on a T cell or may be capable of binding to a human effector cell such as, for example, a monocyte/macrophage and/or a natural killer cell to effect cell death.

In one embodiment, an anti-CoV S glycoprotein antibody of the invention is a bispecific antibody capable of specifically binding to a first and second antigen, wherein said first antigen is a SARS-CoV-2 S glycoprotein and said second antigen is an Fc gamma receptor selected from the group consisting of FcγRI, FcγRIIA, FcγRIIB, FcγRIIIA and/or FcγRIV. In a further embodiment, an anti-CoV S glycoprotein antibody of the invention is a bispecific antibody capable of specifically binding to SARS-CoV-2 and FcγRIIB. In another embodiment, an anti-CoV S glycoprotein antibody of the invention is a bispecific antibody capable of specifically binding to SARS-CoV-2 S glycoprotein and human FcγRIIB.

### Variant Fc Regions

The present invention provides an anti-CoV S glycoprotein antibody with a variant Fc domain. That is, a non naturally occurring Fc region, for example an Fc region comprising one or more non naturally occurring amino acid residues. Also encompassed by the variant Fc regions of present invention are Fc regions which comprise amino acid deletions, additions and/or modifications.

It will be understood that Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, VA). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region including, but not limited to, proteins comprising variant Fc regions, which are non naturally occurring variants of an Fc. Note: Polymorphisms have been observed at a number of Fc positions, including but not limited to Kabat 270, 272, 312, 315, 356, and 358, and thus slight differences between the presented sequence and sequences in the prior art may exist.

The present invention encompasses anti-CoV S glycoprotein antibody with variant Fc domains. The variant Fc domains may have altered binding properties for an Fc ligand (e.g., an Fc receptor, Clq) relative to a comparable molecule (e.g., a protein having the same amino acid sequence except having a wild type Fc region). Examples of binding properties include but are not limited to, binding specificity, equilibrium dissociation constant (K_{D}), dissociation and association rates (k_{off} and kₒₙ respectively), binding affinity and/or avidity. It is generally understood that a binding molecule (e.g., a Fc variant protein such as an antibody) with a low K_{D} may be preferable to a binding molecule with a high K_{D}. However, in some instances the value of the icon or k_{off} may be more relevant than the value of the K_{D}. One skilled in the art can determine which kinetic parameter is most important for a given antibody application.

The affinities and binding properties of an Fc domain for its ligand may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immunoabsorbent assay (ELISA), or radioimmunoassay (RIA)), or kinetics (e.g., BIACORE© analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In one embodiment, an anti-CoV S glycoprotein antibody with a variant Fc domain has enhanced binding to one or more Fc ligand relative to a comparable molecule. In another embodiment, an anti-CoV S glycoprotein antibody with a variant Fc domain has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. In a specific embodiment, an anti-CoV S glycoprotein antibody with a variant Fc domain has enhanced binding to an Fc receptor. In another specific embodiment, an anti-CoV S glycoprotein antibody with a variant Fc domain has enhanced binding to the Fc receptor FcγRIIIA. In a further specific embodiment, an anti-CoV S glycoprotein antibody with a variant Fc domain has enhanced biding to the Fc receptor FcγRIIB. In still another specific embodiment, an anti-CoV S glycoprotein antibody with a variant Fc domain has enhanced binding to the Fc receptor FcRn. In yet another specific embodiment, an anti-CoV S glycoprotein antibody with a variant Fc domain has enhanced binding to Clq relative to a comparable molecule.

In one embodiment, an anti-CoV S glycoprotein antibody of the invention comprises a variant Fc domain wherein said variant Fc domain has enhanced binding affinity to Fc gamma receptor IIB relative to a comparable non-variant Fc domain. In a further embodiment, an anti-CoV S glycoprotein antibody of the invention comprises a variant Fc domain wherein said variant Fc domain has an affinity for Fc gamma receptor IIB that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable non-variant Fc domain.

The serum half-life of proteins comprising Fc regions may be increased by increasing the binding affinity of the Fc region for FcRn. In one embodiment, the antibody comprising a variant Fc domain has enhanced serum half life relative to comparable molecule.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement

The ability of an antibody comprising a variant Fc domain to mediate lysis of the target cell by ADCC can be assayed. To assess ADCC activity an Fc variant protein of interest is added to target cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Tmmunol Methods 184:29-38. ADCC activity of the Fc variant protein of interest may also be assessed in vivo, e.g., in a animal model such as that disclosed in Clynes et al., 1998, Proc. Natl. Acad. Sci. USA 95:652-656.

In one embodiment, an antibody having a variant Fc domain has enhanced ADCC activity relative to a comparable molecule. In a specific embodiment an antibody having a variant Fc domain has ADCC activity that is at least 2 fold, or at least 3 fold, or at least 5 fold or at least 10 fold or at least 50 fold or at least 100 fold greater than that of a comparable molecule. In another specific embodiment, an antibody having a variant Fc domain has enhanced binding to the Fc receptor FcγRIIIA and has enhanced ADCC activity relative to a comparable molecule. In other embodiments, an antibody having a variant Fc domain has both enhanced ADCC activity and enhanced serum half life relative to a comparable molecule.

In one embodiment, an antibody having a variant Fc domain has reduced ADCC activity relative to a comparable molecule. In a specific embodiment, an Fc variant protein has ADCC activity that is at least 2 fold, or at least 3 fold, or at least 5 fold or at least 10 fold or at least 50 fold or at least 100 fold lower than that of a comparable molecule. In another specific embodiment, an antibody having a variant Fc domain has reduced binding to the Fc receptor FcγRIIIA and has reduced ADCC activity relative to a comparable molecule. In other embodiments, an antibody having a variant Fc domain has both reduced ADCC activity and enhanced serum half life relative to a comparable molecule.

“Complement dependent cytotoxicity” and “CDC” refer to the lysing of a target cell in the presence of complement. The complement activation pathway is initiated by the binding of the first component of the complement system (Clq) to a molecule, an antibody for example, complexed with a cognate antigen. To assess complement activation, a CDC assay, e.g. as described in Gazzano-Santoro et al., 1996, J. Immunol. Methods, 202:163, may be performed. In one embodiment, an antibody having a variant Fc domain has enhanced CDC activity relative to a comparable molecule. In a specific embodiment, an Fc variant protein has CDC activity that is at least 2 fold, or at least 3 fold, or at least 5 fold or at least 10 fold or at least 50 fold or at least 100 fold greater than that of a comparable molecule. In other embodiments, an antibody having a variant Fc domain has both enhanced CDC activity and enhanced serum half life relative to a comparable molecule.

In one embodiment, an antibody having a variant Fc domain has reduced binding to one or more Fc ligand relative to a comparable molecule. In another embodiment, an antibody having a variant Fc domain has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold lower than that of a comparable molecule. In a specific embodiment, an antibody having a variant Fc domain has reduced binding to an Fc receptor. In another specific embodiment, an antibody having a variant Fc domain has reduced binding to the Fc receptor FcγRIIIA. In a further specific embodiment, an antibody having a variant Fc domain described herein has an affinity for the Fc receptor FcγRIIIA that is at least about 5 fold lower than that of a comparable molecule, wherein said an antibody having a variant Fc domain has an affinity for the Fc receptor FcγRIIB that is within about 2 fold of that of a comparable molecule. In still another specific embodiment, the Fc variant protein has reduced binding to the Fc receptor FcRn. In yet another specific embodiment, an antibody having a variant Fc domain has reduced binding to Clq relative to a comparable molecule.

In one embodiment, the present invention provides an antibody having a variant Fc domain, wherein the Fc region comprises a non naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In one embodiment, the present invention provides formulations, wherein the Fc region comprises a non naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In a specific embodiment, the present invention provides an antibody having a variant Fc domain, wherein the Fc region comprises at least one non naturally occurring amino acid residue selected from the group consisting of 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 3051, 313F, 316D, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In a specific embodiment, the present invention provides an antibody having a variant Fc domain, wherein the Fc region comprises at least one non naturally occurring amino acid residue selected from the group consisting of 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 3051, 313F, 316D, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331I, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In another embodiment, the present invention provides an antibody having a variant Fc domain, wherein the Fc region comprises at least one non naturally occurring amino acid at one or more positions selected from the group consisting of 239, 330 and 332, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat and at least one non naturally occurring amino acid at one or more positions selected from the group consisting of 252Y, 254T and 256E, as numbered by the EU index as set forth in Kabat.

In another embodiment, the present invention provides an antibody having a variant Fc domain, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat. In a further specific embodiment, an Fc variant of the invention comprises the 234F, 235F, and 331S non naturally occurring amino acid residues, as numbered by the EU index as set forth in Kabat. In another specific embodiment, the Fc domain of the invention comprises the 234F, 235Y, and 331S non naturally occurring amino acid residues, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

In another embodiment, the present invention provides an antibody having a variant Fc domain, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat and at least one non naturally occurring amino acid at one or more positions are selected from the group consisting of 252Y, 254T and 256E, as numbered by the EU index as set forth in Kabat.

In another embodiment, the present invention provides an antibody having a variant Fc domain, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

In one embodiment, the Fc variants of the present invention may be combined with other known Fc variants such as those disclosed in Ghetie et al., 1997, Nat Biotech. 15:637-40; Duncan et al, 1988, Nature 332:563-564; Lund et al., 1991, J. Immunol 147:2657,2662; Lund et al, 1992, Mol Immunol 29:53-59; Alegre et al, 1994, Transplantation 57:1537,1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351. Also encompassed by the present invention are Fc regions which comprise deletions, additions and/or modifications. Still other modifications/substitutions/additions/deletions of the Fc domain will be readily apparent to one skilled in the art.

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492 (1985)), PCR mutagenesis (Higuchi, in “PCR Protocols: A Guide to Methods and Applications”, Academic Press, San Diego, pp. 177-183 (1990)), and cassette mutagenesis (Wells et al., Gene 34:315-323 (1985)). Preferably, site-directed mutagenesis is performed by the overlap-extension PCR method (Higuchi, in “PCR Technology: Principles and Applications for DNA Amplification”, Stockton Press, New York, pp. 61-70 (1989)). The technique of overlap-extension PCR (Higuchi, ibid.) can also be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351).

In some embodiments, an antibody having a variant Fc domain comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

### Glycosylation of Antibodies

In still another embodiment, the glycosylation of antibodies utilized in accordance with the invention is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861. One or more amino acid substitutions can also be made that result in elimination of a glycosylation site present in the Fc region (e.g., Asparagine 297 of IgG). Furthermore, aglycosylated antibodies may be produced in bacterial cells which lack the necessary glycosylation machinery.

An antibody can also be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, U.S. Pat. No. 6,946,292; European Patent No: EP 1,176,195; PCT Publications WO 03/035835; WO 99/54342 each of which is incorporated herein by reference in its entirety.

### Engineering Effector Function

It may be desirable to modify an anti-CoV S glycoprotein antibody of the invention with respect to effector function. For example, cysteine residue(s) may be introduced in the Fc region, thereby allowing interchain disulfide bond formation in this region. The homodimeric antibody thus generated may have improved internalization capability and/or increased complement-mediated cell killing and/or antibody-dependent cellular cytotoxicity (ADCC). See, Caron et al., J. Exp Med., 176:1191-1195 (1992) and Shopes, B., J. Immunol., 148:2918,2922 (1992). Homodimeric antibodies with enhanced anti-tumor activity may also be prepared using heterobifunctional cross-linkers as described in Wolff et al., Cancer Research, 53:2560-2565 (1993). An antibody can also be engineered which has dual Fc regions and may thereby have enhanced complement lysis and ADCC capabilities. See, Stevenson et al., Anti-Cancer Drug Design, 3:219-230 (1989).

Other methods of engineering Fc regions of antibodies so as to alter effector functions are known in the art (e.g., U.S. Patent Publication No. 20040185045 and PCT Publication No. WO 2004/016750, both to Koenig et al., which describe altering the Fc region to enhance the binding affinity for FcγRIIB as compared with the binding affinity for FcγRIIA; see, also, PCT Publication Nos. WO 99/58572 to Armour et al., WO 99/51642 to ldusogie et al., and U.S. Pat. No. 6,395,272 to Deo et al.; the disclosures of which are incorporated herein in their entireties). Methods of modifying the Fc region to decrease binding affinity to FcγRIIB are also known in the art (e.g., U.S. Patent Publication No. 20010036459 and PCT Publication No. WO 01/79299, both to Ravetch et al., the disclosures of which are incorporated herein in their entireties). Modified antibodies having variant Fc regions with enhanced binding affinity for FcγRIIIA and/or FcγRIIA as compared with a wildtype Fc region have also been described (e.g., PCT Publication Nos. WO 2004/063351, to Stavenhagen et al., the disclosure of which is incorporated herein in its entirety).

In vitro assays known in the art can be used to determine whether anti-CoV S glycoprotein antibody used in compositions and methods of the invention are capable of mediating ADCC, such as those described herein.

### Manufacture/Production of Anti-CoV S Glycoprotein Antibodies

Once a desired anti-CoV S glycoprotein antibody is engineered, the anti-CoV S glycoprotein antibody can be produced on a commercial scale using methods that are well-known in the art for large scale manufacturing of antibodies. For example, this can be accomplished using recombinant expressing systems such as, but not limited to, those described below.

### Recombinant Expression Systems

Recombinant expression of an antibody or variant thereof, generally requires construction of an expression vector containing a polynucleotide that encodes the antibody. Once a polynucleotide encoding an antibody molecule or a heavy or light chain of an antibody, or portion thereof, has been obtained, the vector for the production of the antibody molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well-known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

In another embodiment, anti-CoV S glycoprotein antibodies can be made using targeted homologous recombination to produce all or portions of the anti-CoV S glycoprotein antibodies (see, U.S. Pat. Nos. 6,063,630, 6,187,305, and 6,692,737). In certain embodiments, anti-CoV S glycoprotein antibody can be made using random recombination techniques to produce all or portions of the anti-CoV S glycoprotein antibody (see, U.S. Pat. Nos. 6,361,972, 6,524,818, 6,541,221, and 6,623,958). Anti-CoV S glycoprotein antibody can also be produced in cells expressing an antibody from a genomic sequence of the cell comprising a modified immunoglobulin locus using Cre-mediated site-specific homologous recombination (see, U.S. Pat. No. 6,091,001). The host cell line may be derived from human or nonhuman species including but not limited to mouse, and Chinese hamster. Where human or humanized antibody production is desired, the host cell line should be a human cell line. These methods may advantageously be used to engineer stable cell lines which permanently express the antibody molecule.

Once the expression vector is transferred to a host cell by conventional techniques, the transfected cells are then cultured by conventional techniques to produce an antibody. Thus, the invention includes host cells containing a polynucleotide encoding an antibody of the invention or fragments thereof, or a heavy or light chain thereof, or portion thereof, or a single-chain antibody of the invention, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express an anti-CoV S glycoprotein antibody or portions thereof that can be used in the engineering and generation of anti-CoV S glycoprotein antibodies (see, e.g., U.S. Pat. No. 5,807,715). For example, mammalian cells such as Chinese hamster ovary cells (CHO), in conjunction with a vector such as the major intermediate early gene promoter element from human cytomegalovirus is an effective expression system for antibodies (Foecking et al., Gene, 45:101 (1986); and Cockett et al., Bio/Technology, 8:2 (1990)). In addition, a host cell strain may be chosen which modulates the expression of inserted antibody sequences, or modifies and processes the antibody gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any functional immunoglobulin chains), CRL7030 and HsS78Bst cells.

In bacterial systems, a number of expression vectors may be advantageously selected depending upon the use intended for the antibody molecule being expressed. For example, when a large quantity of such an antibody is to be produced, for the generation of pharmaceutical compositions comprising an anti-CoV S glycoprotein antibody, vectors which direct the expression of high levels of fusion protein products that are readily purified may be desirable. Such vectors include, but are not limited to, the E. coli expression vector pUR278 (Ruther et al., EMBO, 12:1791 (1983)), in which the antibody coding sequence may be ligated individually into the vector in frame with the lac Z coding region so that a fusion protein is produced; pIN vectors (Inouye & Inouye, 1985, Nucleic Acids Res. 13:3101-3109 (1985); Van Heeke & Schuster, 1989, J. Biol. Chem., 24:5503-5509 (1989)); and the like. pGEX vectors may also be used to express foreign polypeptides as fusion proteins with glutathione-S-transferase (GST). In general, such fusion proteins are soluble and can easily be purified from lysed cells by adsorption and binding to glutathione-agarose affinity matrix followed by elution in the presence of free glutathione. The pGEX vectors are designed to introduce athrombin and/or factor Xa protease cleavage sites into the expressed polypeptide so that the cloned target gene product can be released from the GST moiety.

In an insect system, Autographa californica nuclear polyhedrosis virus (AcNPV) is used as a vector to express foreign genes. The virus grows in Spocloptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of virus based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion into a non-essential region of the viral genome (e.g., region E1 or E3) will result in a recombinant virus that is viable and capable of expressing the antibody molecule in infected hosts (e.g., see, Logan & Shenk, Proc. Natd. Acad. Sci. USA, 81:355-359 (1984)). Specific initiation signals may also be required for efficient translation of inserted antibody coding sequences. These signals include the ATG initiation codon and adjacent sequences. Furthermore, the initiation codon should generally be in frame with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol., 153:51-544(1987)).

Stable expression can be used for long-term, high-yield production of recombinant proteins. For example, cell lines which stably express the antibody molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci which in turn can be cloned and expanded into cell lines. Plasmids that encode an anti-CoV S glycoprotein antibody can be used to introduce the gene/cDNA into any cell line suitable for production in culture.

A number of selection systems may be used, including, but not limited to, the herpes simplex virus thymidine kinase (Wigler et al., Cell, 11:223 (1977)), hypoxanthineguanine phosphoribosyltransferase (Szybalska & Szybalski, Proc. Natl. Acad Sci. USA, 48:202 (1992)), and adenine phosphoribosyltransferase (Lowy et al., Cell, 22:8-17 (1980)) genes can be employed in tk⁻, hgprt⁻ or aprrcells, respectively. Also, antimetabolite resistance can be used as the basis of selection for the following genes: dhfr, which confers resistance to methotrexate (Wigler et al., Natl. Acad Sci. USA, 77:357 (1980); O'Hare et al., Proc. Natl. Acad Sci. USA, 78:1527 (1981)); gpt, which confers resistance to mycophenolic acid (Mulligan & Berg, Proc. Natl. Acad Sci. USA, 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene, 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kricgler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol., 150:1, which are incorporated by reference herein in their entireties.

The expression levels of an antibody molecule can be increased by vector amplification (for a review, see, Bebbington and Hentschel, The use of vectors based on gene amplification for the expression of cloned genes in mammalian cells in DNA cloning, Vol. 3. Academic Press, New York (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol., 3:257 (1983)). Antibody expression levels may be amplified through the use recombinant methods and tools known to those skilled in the art of recombinant protein production, including technologies that remodel surrounding chromatin and enhance transgene expression in the form of an active artificial transcriptional domain.

The host cell may be co-transfected with two expression vectors, the first vector encoding a heavy chain derived polypeptide and the second vector encoding a light chain derived polypeptide. The two vectors may contain identical or different selectable markers. A single vector which encodes, and is capable of expressing, both heavy and light chain polypeptides may also be used. In such situations, the light chain should be placed 5′ to the heavy chain to avoid an excess of toxic free heavy chain (Proudfoot, Nature 322:562-65 (1986); and Kohler, 1980, Proc. Natl. Acad. Sci. USA, 77:2197 (1980)). The coding sequences for the heavy and light chains may comprise cDNA or genomic DNA.

Once an antibody molecule has been produced by recombinant expression, it may be purified by any method known in the art for purification of an immunoglobulin molecule, for example, by chromatography (e.g., ion exchange, affinity, particularly by affinity for the specific antigens Protein A or Protein G, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### Antibody Purification and Isolation

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio Technology, 10:163-167 (1992) describe a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody mutant is secreted into the medium, supernatants from such expression systems are generally first concentrated using a commercially available protein concentration filter, for example, an Amicon or Millipore Pcllicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain that is present in the antibody mutant. Protein A can be used to purify antibodies that are based on human γ1, γ2, or γ⁴ heavy chains (Lindmark et al., J. Immunol. Methods, 62:1-13 (1983)). Protein G is recommended for all mouse isotypes and for human γ3 (Guss et al., EMBO J., 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the Bakerbond ABX resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, and performed at low salt concentrations (e.g., from about 0-0.25 M salt).

### Therapeutic Anti-CoV S Glycoprotein Antibodies

An anti-CoV S glycoprotein antibody used in compositions and methods of the invention may be a human antibody or a humanized antibody that may treat COVID-19 or neutralize a SARS-CoV-2 virus or a variant thereof. In certain embodiments, anti-CoV S glycoprotein antibodies can be chimeric antibodies or mouse antibodies. In certain embodiments, anti-CoV S glycoprotein antibodies can be a monoclonal human, humanized, or chimeric antibodies. An anti-CoV S glycoprotein antibody used in compositions and methods of the invention may be a human antibody or a humanized antibody of the IgG1 or IgG3 human isotype or any IgG1 or IgG3 allele found in the human population. In other embodiments, an anti-CoV S glycoprotein antibody used in compositions and methods of the invention can be a human antibody or a humanized antibody of the IgG2 or IgG4 human isotype or any IgG2 or IgG4 allele found in the human population.

In certain embodiments, the antibody is an isotype switched variant of a known antibody (e.g., to an IgG1 or IgG3 human isotype) such as those described above.

Anti-CoV S glycoprotein antibody used in compositions and methods of the disclosure can be naked antibodies, immunoconjugates or fusion proteins.

### Screening of Antibodies for SARS-CoV-2 S Glycoprotein Binding

Binding assays can be used to identify antibodies that bind the SARS-CoV-2 S glycoprotein. Binding assays may be performed either as direct binding assays or as competition-binding assays. Binding can be detected using standard ELISA or standard Flow Cytometry assays. In a direct binding assay, a candidate antibody is tested for binding to a SARS-CoV-2 S glycoprotein. Competition-binding assays, on the other hand, assess the ability of a candidate antibody to compete with a known anti-CoV S glycoprotein antibody or other compound that binds SARS-CoV-2 S glycoprotein.

In a direct binding assay, the SARS-CoV-2 S glycoprotein is contacted with a candidate antibody under conditions that allow binding of the candidate antibody to the SARS-CoV-2 S glycoprotein. The binding may take place in solution or on a solid surface. The candidate antibody may have been previously labeled for detection. Any detectable compound can be used for labeling, such as, but not limited to, a luminescent, fluorescent, or radioactive isotope or group containing same, or a nonisotopic label, such as an enzyme or dye. After a period of incubation sufficient for binding to take place, the reaction is exposed to conditions and manipulations that remove excess or non-specifically bound antibody. Typically, it involves washing with an appropriate buffer. Finally, the presence of a complex between the candidate antibody and SARS-CoV-2 S glycoprotein is detected.

In a competition-binding assay, a candidate antibody is evaluated for its ability to inhibit or displace the binding of a known anti-CoV S glycoprotein antibody (or other compound) to the SARS-CoV-2 S glycoprotein. A labeled known binder of SARS-CoV-2 S glycoprotein may be mixed with the candidate antibody, and placed under conditions in which the interaction between them would normally occur, with and without the addition of the candidate antibody. The amount of labeled known binder of SARS-CoV-2 glycoprotein that binds the SARS-CoV-2 glycoprotein may be compared to the amount bound in the presence or absence of the candidate antibody.

In one embodiment, the binding assay is carried out with one or more components immobilized on a solid surface to facilitate antibody antigen complex formation and detection. In various embodiments, the solid support could be, but is not restricted to, polyvinylidene fluoride, polycarbonate, polystyrene, polypropylene, polyethylene, glass, nitrocellulose, dextran, nylon, polyacrylamide and agarose. The support configuration can include beads, membranes, microparticles, the interior surface of a reaction vessel such as a microtiter plate, test tube or other reaction vessel. The immobilization of SARS-CoV-2 S glycoprotein or a fragment thereof, or other component, can be achieved through covalent or non-covalent attachments. In one embodiment, the attachment may be indirect, i.e., through an attached antibody. In another embodiment, the SARS-CoV-2 S glycoprotein and negative controls are tagged with an epitope, such as glutathione S-transferase (GST) so that the attachment to the solid surface can be mediated by a commercially available antibody such as anti-GST (Santa Cruz Biotechnology).

For example, such an affinity binding assay may be performed using the SARS-CoV-2 S glycoprotein which is immobilized to a solid support. Typically, the non-mobilized component of the binding reaction, in this case the candidate anti-CoV S glycoprotein antibody, is labeled to enable detection. A variety of labeling methods are available and may be used, such as luminescent, chromophore, fluorescent, or radioactive isotope or group containing same, and nonisotopic labels, such as enzymes or dyes. In one embodiment, the candidate anti-CoV S glycoprotein antibody antibody is labeled with a fluorophore such as fluorescein isothiocyanate (FITC, available from Sigma Chemicals, St. Louis). Such an affinity binding assay may be performed using the SARS-CoV-2 S glycoprotein immobilized on a solid surface. anti-CoV S glycoprotein antibody are then incubated with the antigen and the specific binding of antibodies is detected by methods known in the art including, but not limited to, BiaCore Analyses, ELISA, FMET and R1A methods.

Finally, the label remaining on the solid surface may be detected by any detection method known in the art. For example, if the candidate anti-CoV S glycoprotein antibody is labeled with a fluorophore, a fluorimeter may be used to detect complexes.

The SARS-CoV-2 S glycoprotein can be added to binding assays in the form of intact cells that express the SARS-CoV-2 S glycoprotein, or isolated membranes containing human the SARS-CoV-2 S glycoprotein. Thus, direct binding to SARS-CoV-2 glycoprotein may be assayed in intact cells in culture or in animal models in the presence and absence of the candidate anti-CoV S glycoprotein antibody. A labeled candidate anti-CoV S glycoprotein antibody may be mixed with cells that express the SARS-CoV-2 S glycoprotein, and the candidate anti-CoV S glycoprotein antibody may be added. Isolated membranes may be used to identify candidate anti-CoV S glycoprotein antibody that interact with SARS-CoV-2 S glycoprotein. For example, in a typical experiment using isolated membranes, cells may be genetically engineered to express a SARS-CoV-2 S glycoprotein. Membranes can be harvested by standard techniques and used in an in vitro binding assay. Labeled candidate anti-CoV S glycoprotein antibody (e.g., fluorescent labeled antibody) is bound to the membranes and assayed for specific activity; specific binding is determined by comparison with binding assays performed in the presence of excess unlabeled (cold) candidate anti-CoV S glycoprotein antibody. Polypeptides corresponding to one or more regions of the SARS-CoV-2 S glycoprotein (e.g., the RBD), or fusion proteins containing one or more regions of the SARS-CoV-2 S glycoprotein can also be used in non-cell based assay systems to identify antibodies that bind to portions of SARS-CoV-2 S glycoproteins. In non-cell based assays the recombinantly expressed human SARS-CoV-2 S glycoproteins are attached to a solid substrate such as a test tube, microliter well or a column, by means well-known to those in the art (see, Ausubel et al., supra). The test antibodies are then assayed for their ability to bind to SARS-CoV-2 S glycoprotein.

The binding reaction may also be carried out in solution. In this assay, the labeled component is allowed to interact with its binding partner(s) in solution. If the size differences between the labeled component and its binding partner(s) permit such a separation, the separation can be achieved by passing the products of the binding reaction through an ultrafilter whose pores allow passage of unbound labeled component but not of its binding partner(s) or of labeled component bound to its partner(s). Separation can also be achieved using any reagent capable of capturing a binding partner of the labeled component from solution, such as an antibody against the binding partner and so on.

In one embodiment, for example, a phage library can be screened by passing phage from a continuous phage display library through a column containing a SARS-CoV-2 S glycoprotein or portion thereof (e.g., the RBD of SARS-CoV-2 S glycoprotein), or derivative, analog, fragment, or domain, thereof, linked to a solid phase, such as plastic beads. By altering the stringency of the washing buffer, it is possible to enrich for phage that express peptides with high affinity for the SARS-CoV-2 S glycoprotein. Phage isolated from the column can be cloned and affinities can be measured directly. Knowing which antibodies and their amino acid sequences confer the strongest binding to the SARS-CoV-2 S glycoprotein, computer models can be used to identify the molecular contacts between SARS-CoV-2 S glycoprotein and the candidate antibody.

In another specific embodiment, the solid support is membrane containing a SARS-CoV-2 S glycoprotein is attached to a microtiter dish. Candidate antibodies, for example, can bind cells that express library antibodies cultivated under conditions that allow expression of the library members in the microliter dish. Library members that bind to the SARS-CoV-2 are harvested. Such methods, are generally described by way of example in Parmley and Smith, 1988, Gene, 73:305-318; Fowlkes et al., 1992, BioTechniques, 13:422-427; PCT Publication No. WO94/18318; and in references cited hereinabove. Antibodies identified as binding to SARS-CoV-2 S glycoprotein can be of any of the types or modifications of antibodies described above.

### Screening of Antibodies for Human ADCC Effector Function

Antibodies of the human IgG class, which have functional characteristics such a long half-life in serum and the ability to mediate various effector functions are used in certain embodiments of the invention (Monoclonal Antibodies: Principles and Applications, Wiley-Liss, Inc., Chapter 1 (1995)). The human IgG class antibody is further classified into the following 4 subclasses: IgG1, IgG2, IgG3 and IgG4. A large number of studies have so far been conducted for ADCC and CDC as effector functions of the IgG class antibody, and it has been reported that among antibodies of the human IgG class, the IgG1 subclass has the highest ADCC activity and CDC activity in humans (Chemical Immunology, 65, 88 (1997)).

Expression of ADCC activity and CDC activity of the human IgG1 subclass antibodies generally involves binding of the Fc region of the antibody to a receptor for an antibody (hereinafter referred to as “FcγR”) existing on the surface of effector cells such as killer cells, natural killer cells or activated macrophages. Various complement components can be bound. Regarding the binding, it has been suggested that several amino acid residues in the hinge region and the second domain of C region (hereinafter referred to as “Cγ2 domain”) of the antibody are important (Eur. J. Immunol., 23, 1098 (1993), Immunology, 86, 319 (1995), Chemical Immunology, 65, 88 (1997)) and that a sugar chain in the Cγ2 domain (Chemical Immunology, 65, 88 (1997)) is also important.

Anti-CoV S glycoprotein antibodies can be modified with respect to effector function, e.g., so as to enhance ADCC and/or complement dependent cytotoxicity (CDC) of the antibody. This may be achieved by introducing one or more amino acid substitutions in the Fc region of an antibody. Cysteine residue(s) may also be introduced in the Fc region, allowing for interchain disulfide bond formation in this region. In this way a homodimeric antibody can be generated that may have improved internalization capability and or increased complement-mediated cell killing and ADCC (Caron et al., J. Exp. Med., 176:1191-1195 (1992) and Shopes, J. Immunol., 148:2918-2922 (1992)). Heterobifunctional cross-linkers can also be used to generate homodimeric antibodies with enhanced anti-tumor activity (Wolff et al., Cancer Research, 53:2560-2565 (1993)). Antibodies can also be engineered to have two or more Fc regions resulting in enhanced complement lysis and ADCC capabilities (Stevenson et al., Anti-Cancer Drug Design, (3)219-230 (1989)).

Other methods of engineering Fc regions of antibodies so as to alter effector functions are known in the art (e.g., U.S. Patent Publication No. 20040185045 and PCT Publication No. WO 2004/016750, both to Koenig et al., which describe altering the Fc region to enhance the binding affinity for FcγRIIB as compared with the binding affinity for FCγRIIA; see also PCT Publication Nos. WO 99/58572 to Armour et al., WO 99/51642 to Idusogic et al., and U.S. Pat. No. 6,395,272 to Dco et al.; the disclosures of which are incorporated herein in their entireties). Methods of modifying the Fc region to decrease binding affinity to FcγRII1B are also known in the art (e.g., U.S. Patent Publication No. 20010036459 and PCT Publication No. WO 01/79299, both to Ravetch et al., the disclosures of which are incorporated herein in their entireties). Modified antibodies having variant Fc regions with enhanced binding affinity for FcγRIIIA and/or FcγRIIA as compared with a wildtype Fc region have also been described (e.g., PCT Publication No. WO 2004/063351, to Stavenhagen et al.; the disclosure of which is incorporated herein in its entirety).

At least four different types of FcγR have been found, which are respectively called FcγRI (CD64), FcγRII (CD32), FcγRIII (CD16), and FcγRIV. In human, FcγRII and FcγRIII are further classified into FcγRIIa and FcγRIIb, and FcγRIIIa and FcγRIIIb, respectively. FcγR is a membrane protein belonging to the immunoglobulin superfamily, FcγRII, FcγRIII, and FcγRIV have an a chain having an extracellular region containing two immunoglobulin-like domains, FcγRI has an a chain having an extracellular region containing three immunoglobulin-like domains, as a constituting component, and the a chain is involved in the IgG binding activity. In addition, FcγRI and FcγRIII have a γchain or ζ (chain as a constituting component which has a signal transduction function in association with the a chain (Annu. Rev. Immunol., 18, 709 (2000), Annu. Rev. Immunol., 19, 275 (2001)). FcγRIV has been described by Bruhns et al., Clin. Invest. Med., (Canada) 27:3D (2004).

To assess ADCC activity of an anti-CoV S glycoprotein antibody of interest, an in vitro ADCC assay can be used, such as that described in U.S. Pat. No. 5,500,362 or 5,821,337. The assay may also be performed using a commercially available kit, e.g. CytoTox 96® (Promega). Useful effector cells for such assays include, but are not limited to peripheral blood mononuclear cells (PBMC), Natural Killer (NK) cells, and NK cell lines. NK cell lines expressing a transgenic Fc receptor (e.g. CD16) and associated signaling polypeptide (e.g. FCεRI-γ) may also serve as effector cells (see, e.g. WO 2006/023148 A2 to Campbell). For example, the ability of any particular antibody to mediate lysis of the target cell by complement activation and/or ADCC can be assayed. The cells of interest are grown and labeled in vitro; the antibody is added to the cell culture in combination with immune cells which may be activated by the antigen antibody complexes; i.e., effector cells involved in the ADCC response. The antibody can also be tested for complement activation. In either case, cytolysis of the target cells is detected by the release of label from the lysed cells. The extent of target cell lysis may also be determined by detecting the release of cytoplasmic proteins (e.g. LDH) into the supernatant. In fact, antibodies can be screened using the patient's own serum as a source of complement and/or immune cells. The antibodies that are capable of mediating human ADCC in the in vitro test can then be used therapeutically in that particular patient. ADCC activity of the molecule of interest may also be assessed in vivo, e.g., in an animal model such as that disclosed in Clynes et al., Proc. Natl. Acad. Sci. (USA) 95:652-656 (1998). Moreover, techniques for modulating (i.e., increasing or decreasing) the level of ADCC, and optionally CDC activity, of an antibody are well-known in the art. See, e.g., U.S. Pat. No. 6,194,551. Antibodies of the present invention may be capable or may have been modified to have the ability of inducing ADCC and/or CDC. Assays to determine ADCC function can be practiced using human effector cells to assess human ADCC function. Such assays may also include those intended to screen for antibodies that induce, mediate, enhance, block cell death by necrotic and/or apoptotic mechanisms. Such methods including assays utilizing viable dyes, methods of detecting and analyzing caspases, and assays measuring DNA breaks can be used to assess the apoptotic activity of cells cultured in vitro with an anti-CoV S glycoprotein antibody of interest.

For example, Annexin V or TdT-mediated dUTP nick-end labeling (TUNEL) assays can be carried out as described in Decker et al., Blood (USA) 103:2718-2725 (2004) to detect apoptotic activity. The TUNEL assay involves culturing the cell of interest with fluorescein-labeled dUTP for incorporation into DNA strand breaks. The cells are then processed for analysis by flow cytometry. The Annexin V assay detects the appearance of phosphatidylserine (PS) on the outside of the plasma membrane of apoptotic cells using a fluorescein-conjugated Annexin V that specifically recognizes the exposed PS molecules. Concurrently, a viable dye such as propidium iodide can be used to exclude late apoptotic cells. The cells are stained with the labeled Annexin V and are analyzed by flow cytometry.

### Neutralizing Antibodies

In embodiments, the anti-CoV S glycoprotein antibodies described herein are neutralizing antibodies. In embodiments, the anti-CoV S glycoprotein antibodies neutralize a SARS-CoV-2 virus or variant thereof.

### Anti-CoV S Glycoprotein Antibody Conjugates

According to certain aspects of the invention, compounds may be conjugated to anti-CoV S glycoprotein antibodies for use in compositions and methods of the invention. In certain embodiments, these conjugates can be generated as fusion proteins.

Covalent modifications of anti-CoV S glycoprotein antibodies are included within the scope of this invention. They may be made by chemical synthesis or by enzymatic or chemical cleavage of the antibody, if applicable. Other types of covalent modifications of anti-CoV S glycoprotein antibodies are introduced into the molecule by reacting targeted amino acid residues of the antibody with an organic derivatizing agent that is capable of reacting with selected side chains or the N- or C-terminal residues.

Cysteinyl residues most commonly are reacted with a-haloacetates (and corresponding amines), such as chloroacetic acid or chloroacetamide, to give carboxymethyl or carboxyamidomethyl derivatives. Similarly, iodo-reagents may also be used. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction can be performed in 0.1 M sodium cacodylate at pH 6.0.

Lysyl and amino-terminal residues are reacted with succinic or other carboxylic acid anhydrides. Derivatization with these agents has the effect of reversing the charge of the lysinyl residues. Other suitable reagents for derivatizing α-amino-containing residues and/or ε-amino-containing residues include imidoesters such as methyl picolinimidate, pyridoxal phosphate, pyridoxal, chloroborohydride, trinitrobenzenesulfonic acid, 0-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginyl residues generally requires that the reaction be performed in alkaline conditions because of the high pKa of the guanidine functional group. Furthermore, these reagents may react with the ε-amino groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form 0-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimidc or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to the antibody. These procedures are advantageous in that they do not require production of the antibody in a host cell that has glycosylation capabilities for N- or 0-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published 11 Sep. 1987, and in Aplin and Wriston, CRC Crit. Rev. Biochem., pp. 259-306 (1981).

### Pharmaceutical Compositions

The invention also relates to compositions comprising anti-CoV S glycoprotein antibodies and methods of using the aforementioned compositions for the treatment of COVID-19 in human subjects.

The present invention relates to pharmaceutical compositions comprising anti-CoV S glycoprotein antibodies of the IgG1 or IgG3 human isotype. The present invention also relates to pharmaceutical compositions comprising anti-CoV S glycoprotein antibodies of the IgG2 or IgG4 human isotype that may mediate human ADCC. In certain embodiments, the present invention also relates to pharmaceutical compositions comprising monoclonal human, humanized, or chimerized anti-CoV S glycoprotein antibodies that can be produced by means known in the art.

In other particular embodiments, anti-CoV S glycoprotein antibodies may mediate ADCC, complement-dependent cellular cytoxicity, or apoptosis.

### Antibody Half-Life

In embodiments, the half-life of anti-CoV S glycoprotein antibodies described herein is about 1 hour to about 60 days. For example, the half-life of an anti-CoV S glycoprotein antibody is up to about 1 hour, up to about 2 hours, up to about 3 hours, up to about 4 hours, up to about 5 hours, up to about 6 hours, up to about 7 hours, up to about 8 hours, up to about 9 hours, up to about 10 hours, up to about 11 hours, up to about 12 hours, up to about 13 hours, up to about 14 hours, up to about 15 hours, up to about 16 hours, up to about 17 hours, up to about 18 hours, up to about 19 hours, up to about 20 hours, up to about 21 hours, up to about 22 hours, up to about 23 hours, up to about 24 hours, up to about 2 days, up to about 3 days, up to about 4 days, up to about 5 days, up to about 6 days, up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, up to about 20 days, up to about 21 days, up to about 22 days, up to about 23 days, up to about 24 days, up to about 25 days, up to about 26 days, up to about 27 days, up to about 28 days, up to about 29 days, up to about 30 days, up to about 31 days, up to about 32 days, up to about 33 days, up to about 34 days, up to about 35 days, up to about 36 days, up to about 37 days, up to about 38 days, up to about 39 days, up to about 40 days, up to about 41 days, up to about 42 days, up to about 43 days, up to about 44 days, up to about 45 days, up to about 46 days, up to about 47 days, up to about 48 days, up to about 49 days, up to about 50 days, up to about 51 days, up to about 52 days, up to about 53 days, up to about 54 days, up to about 55 days, up to about 56 days, up to about 57 days, up to about 58 days, up to about 59 days, or up to about 60 days. In certain embodiments, the half-lives of antibodies of compositions and methods of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375; and International Publication Nos. WO 98/23289 and WO 97/3461.

The serum circulation of anti-CoV S glycoprotein antibodies in vivo may also be prolonged by attaching inert polymer molecules such as high molecular weight polyethyleneglycol (PEG) to the antibodies with or without a multifunctional linker either through site-specific conjugation of the PEG to the N or C-terminus of the antibodies or via epsilon-amino groups present on lysyl residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

Further, the antibodies of compositions and methods of the invention can be conjugated to albumin in order to make the antibody more stable in vivo or have a longer half-life in vivo. The techniques are well known in the art, see, e.g., International Publication Nos. WO 93/15199, WO 93/15200, and WO 01/77137; and European Patent No. EP 413, 622, all of which are incorporated herein by reference.

### Pharmaceutical Formulations, Administration, and Dosing

Pharmaceutical formulations of the invention contain as the active ingredient anti-CoV S glycoprotein antibodies. The formulations contain naked antibody, immunoconjugate, or fusion protein in an amount effective for producing the desired response in a unit of weight or volume suitable for administration to a human patient, and are preferably sterile.

An anti-CoV S glycoprotein antibody composition may be formulated with a pharmaceutically acceptable carrier. The term “pharmaceutically acceptable” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, boric, formic, malonic, succinic, and the like. Also, pharmaceutically acceptable salts can be prepared as alkaline metal or alkaline earth salts, such as sodium, potassium or calcium salts. The term “carrier” denotes an organic or inorganic ingredient, natural or synthetic, with which the active ingredient is combined to facilitate the application. The components of the pharmaceutical compositions also are capable of being co-mingled with the antibodies of the present invention, and with each other, in a manner such that there is no interaction which would substantially impair the desired pharmaceutical efficacy.

According to certain aspects of the invention, anti-CoV S glycoprotein antibodies compositions can be prepared for storage by mixing the antibody or immunoconjugate having the desired degree of purity with optional physiologically acceptable carriers, excipients or stabilizers (Remington's Pharmaceutical Sciences, 16th edition, Osol, A. Ed. (1999)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS™ or polyethylene glycol (PEG).

Anti-CoV S glycoprotein antibodies compositions also may contain, optionally, suitable preservatives, such as: benzalkonium chloride; chlorobutanol; parabens and thimerosal.

Anti-CoV S glycoprotein antibodies compositions may conveniently be presented in unit dosage form and may be prepared by any of the methods well-known in the art of pharmacy. All methods include the step of bringing the active agent into association with a carrier which constitutes one or more accessory ingredients. In general, anti-CoV S glycoprotein antibodies compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of anti-CoV S glycoprotein antibodies, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administration can be found in Remington's Pharmaceutical Sciences, Mack Publishing Co., Easton, PA.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomcs, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration are typically sterile. This is readily accomplished by filtration through sterile filtration membranes.

Sustained-release preparations may be prepared. Suitable examples of sustained-release preparations include semipermeable matrices of solid hydrophobic polymers containing anti-CoV S glycoprotein antibodies, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devized for stabilization depending on the mechanism involved. For example, if the aggregation mechanism is discovered to be intermolecular S—S bond formation through thio-disulfide interchange, stabilization may be achieved by modifying sulthydryl residues, lyophilizing from acidic solutions, controlling moisture content, using appropriate additives, and developing specific polymer matrix compositions. In certain embodiments, the pharmaceutically acceptable carriers used in compositions of the invention do not affect human ADCC or CDC.

Anti-CoV S glycoprotein antibodies disclosed herein may also be formulated as immunoliposomes. A “liposome” is a small vesicle composed of various types of lipids, phospholipids and/or surfactant which is useful for delivery of a drug (such as anti-CoV S glycoprotein antibodies disclosed herein) to a human. The components of the liposome are commonly arranged in a bilayer formation, similar to the lipid arrangement of biological membranes. Liposomes containing antibodies of the invention are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. The antibody of the present invention can be conjugated to the liposomes as described in Martin et al., J. Biol. Chem., 257:286-288 (1982) via a disulfide interchange reaction. A therapeutic agent can also be contained within the liposome. See, Gabizon et al., J. National Cancer Inst., (19)1484 (1989).

In certain embodiments, apharmaceutical composition of the invention is stable at 4° C. In certain embodiments, a pharmaceutical composition of the invention is stable at room temperature.

Administration of compositions of the invention to a human patient can be by any route, including but not limited to intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intraplural, intracerebral, intra-arterial, intraperitoneal, oral, intralymphatic, intranasal, rectal or vaginal administration, by perfusion through a regional catheter, or by direct intralesional injection. In one embodiment, compositions of the invention are administered by intravenous push or intravenous infusion given over defined period (e.g., 0.5 to 2 hours). Compositions of the invention can be delivered by peristaltic means or in the form of a depot, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

In embodiments, the dose of a composition comprising an anti-CoV S glycoprotein antibody is measured in units of mg/kg of patient body weight. In other embodiments, the dose of a composition comprising anti-CoV S glycoprotein antibodies is measured in units of mg/kg of patient lean body weight (i.e., body weight minus body fat content). In yet other embodiments, the dose of a composition comprising anti-CoV S glycoprotein antibodies is measured in units of mg/m² of patient body surface area. In yet other embodiments, the dose of a composition comprising anti-CoV S glycoprotein antibodies is measured in units of mg per dose administered to a patient. Any measurement of dose can be used in conjunction with compositions and methods of the invention and dosage units can be converted by means standard in the art.

Those skilled in the art will appreciate that dosages can be selected based on a number of factors including the age, sex, species and condition of the subject. For example, effective amounts of compositions of the invention may be extrapolated from dose-response curves derived in vitro test systems or from animal model (e.g., the cotton rat or monkey) test systems. Models and methods for evaluation of the effects of antibodies are known in the art (Wooldridge et al., Blood, 89(8): 2994-2998 (1997)), incorporated by reference herein in its entirety).

Examples of dosing regimens that can be used in methods of the invention include, but are not limited to, daily, three times weekly (intermittent), weekly, every 14 days, every month, every 6-8 weeks, every 2 months, every 6 months, or every year.

In embodiments, the dose of anti-CoV S glycoprotein antibody ranges from 10 mg to about 2 g. For example, the dose of anti-CoV S glycoprotein antibody may be about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 1.05 g, about 1.1 g, about 1.15 g, about 1.2 g, about 1.25 g, about 1.3 g, about 1.35 g, about 1.4 g, about 1.45 g, about 1.5 g, about 1.55 g, about 1.6 g, about 1.65 g, about 1.7 g, about 1.75 g, about 1.8 g, about 1.85 g, about 1.9 g, about 1.95 g, about 2 g, or any range or subrange therebetween.

In embodiments, the present disclosure provides methods for treating a subject infected with a SARS-CoV-2 virus or variant thereof, comprising administering a composition comprising the anti-CoV S glycoprotein antibodies described herein. In embodiments, the SARS-CoV-2 variant thereof has a PANGO lineage selected from the group consisting of B.1.1.529; BA.1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1.

### Toxicity Testing

The tolerance, toxicity and/or efficacy of the compositions and/or treatment regimens of the present invention can be determined by standard pharmaceutical procedures in cell cultures or experimental animals, e.g., for determining the LD50 (the dose lethal to 50% of the population), the ED50 (the dose therapeutically effective in 50% of the population), and IC50 (the dose effective to achieve a 50% inhibition

Data obtained from the cell culture assays and animal studies can be used in formulating a range of dosages of the compositions and/or treatment regimens for use in humans. The dosage of such agents may lie within a range of circulating concentrations that include the ED50 with little or no toxicity. The dosage may vary within this range depending upon the dosage form employed and the route of administration utilized. For any therapy used in methods of the invention, a therapeutically effective dose can be estimated by appropriate animal models. Depending on the species of the animal model, the dose can be scaled for human use according to art-accepted formulas, for example, as provided by Freireich et al., Quantitative comparison of toxicity of anticancer agents in mouse, rat, monkey, dog, and human, Cancer Chemotherapy Reports, NCI 1966 40:219-244. Data obtained from cell culture assays can be useful for predicting potential toxicity. Animal studies can be used to formulate a specific dose to achieve a circulating plasma concentration range that includes the IC50 (i.e., the concentration of the test compound that achieves a half-maximal inhibition of symptoms) as determined in cell culture. Such information can be used to more accurately determine useful doses in humans. Plasma drug levels may be measured, for example, by high performance liquid chromatography, ELISA, or by cell based assays.

### EXAMPLES

### Example 1: Discovery of Antibodies 239.12; 322.3; 425.6; and 35.13

Hybridoma technology was utilized to generate five antibodies that bind to SARS-CoV-2 S polypeptides from SARS-CoV-2 viruses.

Mice were injected with the CoV S glycoproteins BV2373, BV2438, BV2465, or BV2540. BV2373, BV2438, BV2465, and BV2540 comprise the amino acid sequences of SEQ TD NOS: 35, 36, 37, and 119 respectively. The amino acid sequences of BV2373, BV2438, BV2465, and BV2540 are provided in Table A.

TABLE A
Amino Acid Sequences of BV2373, BV2438, and BV2465

| CoV S | | SEQ |
|---|---|---|
| glycoprotein | Amino Acid Sequence | ID NO: |
| BV2373 | QCVNLTTRTQLPPAYTNSFTRGVYYPDKVFRSSVLH | 35 |
| | STQDLFLPFFSNVTWFHAIHVSGTNGTKRFDNPVLPF | |
| | NDGVYFASTEKSNIIRGWIFGTTLDSKTQSLLIVNNA | |
| | TNVVIKVCEFQFCNDPFLGVYYHKNNKSWMESEFR | |
| | VYSSANNCTFEYVSQPFLMDLEGKQGNFKNLREFVF | |
| | KNIDGYFKIYSKHTPINLVRDLPQGFSALEPLVDLPIG | |
| | INITRFQTLLALHRSYLTPGDSSSGWTAGAAAYYVG | |
| | YLQPRTFLLKYNENGTITDAVDCALDPLSETKCTLK | |
| | SFTVEKGIYQTSNFRVQPTESIVRFPNITNLCPFGEVF | |
| | NATRFASVYAWNRKRISNCVADYSVLYNSASFSTFK | |
| | CYGVSPTKLNDLCFTNVYADSFVIRGDEVRQIAPGQ | |
| | TGKIADYNYKLPDDFTGCVIAWNSNNLDSKVGGNY | |
| | NYLYRLFRKSNLKPFERDISTEIYQAGSTPCNGVEGF | |
| | NCYFPLQSYGFQPTNGVGYQPYRVVVLSFELLHAPA | |
| | TVCGPKKSTNLVKNKCVNFNFNGLTGTGVLTESNK | |
| | KFLPFQQFGRDIADTTDAVRDPQTLEILDITPCSFGG | |
| | VSVITPGTNTSNQVAVLYQDVNCTEVPVAIHADQLT | |
| | PTWRVYSTGSNVFQTRAGCLIGAEHVNNSYECDIPI | |
| | GAGICASYQTQTNSPQQAQSVASQSIIAYTMSLGAE | |
| | NSVAYSNNSIAIPTNFTISVTTEILPVSMTKTSVDCTM | |
| | YICGDSTECSNLLLQYGSFCTQLNRALTGIAVEQDK | |
| | NTQEVFAQVKQIYKTPPIKDFGGFNFSQILPDPSKPSK | |
| | RSFIEDLLFNKVTLADAGFIKQYGDCLGDIAARDLIC | |
| | AQKFNGLTVLPPLLTDEMIAQYTSALLAGTITSGWT | |
| | FGAGAALQIPFAMQMAYRENGIGVTQNVLYENQKL | |
| | IANQFNSAIGKIQDSLSSTASALGKLQDVVNQNAQA | |
| | LNTLVKQLSSNFGAISSVLNDILSRLDPPEAEVQIDRL | |
| | ITGRLQSLQTYVTQQLIRAAEIRASANLAATKMSEC | |
| | VLGQSKRVDFCGKGYHLMSFPQSAPHGVVFLHVTY | |
| | VPAQEKNFTTAPAICHDGKAHFPREGVFVSNGTHWF | |
| | VTQRNFYEPQIITTDNTFVSGNCDVVIGIVNNTVYDP | |
| | LQPELDSFKEELDKYFKNHTSPDVDLGDISGINASVV | |
| | NIQKEIDRLNEVAKNLNESLIDLQELGKYEQYIKWP | |
| | WYIWLGFIAGLIAIVMVTIMLCCMTSCCSCLKGCCS | |
| | CGSCCKFDEDDSEPVLKGVKLHYT | |
| BV2438 | QCVNLTTRTQLPPAYTNSFTRGVYYPDKVFRSSVLH | 36 |
| | STQDLFLPFFSNVTWFHAIHVSGTNGTKRFANPVLPF | |
| | NDGVYFASTEKSNIIRGWIFGTTLDSKTQSLLIVNNA | |
| | TNVVIKVCEFQFCNDPFLGVYYHKNNKSWMESEFR | |
| | VYSSANNCTFEYVSQPFLMDLEGKQGNFKNLREFVF | |
| | KNIDGYFKIYSKHTPINLVRGLPQGFSALEPLVDLPIG | |
| | INITRFQTLHALHRSYLTPGDSSSGWTAGAAAYYVG | |
| | YLQPRTFLLKYNENGTITDAVDCALDPLSETKCTLK | |
| | SFTVEKGIYQTSNFRVQPTESIVRFPNITNLCPFGEVF | |
| | NATRFASVYAWNRKRISNCVADYSVLYNSASFSTFK | |
| | CYGVSPTKLNDLCFTNVYADSFVIRGDEVRQIAPGQ | |
| | TGNIADYNYKLPDDFTGCVIAWNSNNLDSKVGGNY | |
| | NYLYRLFRKSNLKPFERDISTEIYQAGSTPCNGVKGF | |
| | NCYFPLQSYGFQPTYGVGYQPYRVVVLSFELLHAPA | |
| | TVCGPKKSTNLVKNKCVNFNFNGLTGTGVLTESNK | |
| | KFLPFQQFGRDIADTTDAVRDPQTLEILDITPCSFGG | |
| | VSVITPGTNTSNQVAVLYQGVNCTEVPVAIHADQLT | |
| | PTWRVYSTGSNVFQTRAGCLIGAEHVNNSYECDIPI | |
| | GAGICASYQTQTNSPQQAQSVASQSIIAYTMSLGVE | |
| | NSVAYSNNSIAIPTNFTISVTTEILPVSMTKTSVDCTM | |
| | YICGDSTECSNLLLQYGSFCTQLNRALTGIAVEQDK | |
| | NTQEVFAQVKQIYKTPPIKDFGGNFSQILPDPSKPSK | |
| | RSFIEDLLFNKVTLADAGFIKQYGDCLGDIAARDLIC | |
| | AQKFNGLTVLPPLLTDEMIAQYTSALLAGTITSGWT | |
| | FGAGAALQIPFAMQMAYRENGIGVTQNVLYENQKL | |
| | IANQFNSAIGKIQDSLSSTASALGKLQDVVNQNAQA | |
| | LNTLVKQLSSNFGAISSVLNDILSRLDPPEAEVQIDRL | |
| | ITGRLQSLQTYVTQQLIRAAEIRASANLAATKMSEC | |
| | VLGQSKRVDFCGKGYHLMSFPQSAPHGVVFLHVTY | |
| | VPAQEKNFTTAPAICHDGKAHFPREGVFVSNGTHWF | |
| | VTQRNFYEPQIITTDNTFVSGNCDVVIGIVNNTVYDP | |
| | LQPELDSFKEELDKYFKNHTSPDVDLGDISGINASVV | |
| | NIQKEIDRLNEVAKNLNESLIDLQELGKYEQYIKWP | |
| | WYIWLGFIAGLIAIVMVTIMLCCMTSCCSCLKGCCS | |
| | CGSCCKFDEDDSEPVLKGVKLHYT | |
| BV2465 | QCVNLRTRTQLPPAYTNSFTRGVYYPDKVFRSSVLH | 37 |
| | STQDLFLPFFSNVTWFHAIHVSGTNGTKRFDNPVLPF | |
| | NDGVYFASTEKSNIIRGWIFGTTLDSKTQSLLIVNNA | |
| | TNVVIKVCEFQFCNDPFLDVYYHKNNKSWMESGVY | |
| | SSANNCTFEYVSQPFLMDLEGKQGNFKNLREFVFKN | |
| | IDGYFKIYSKHTPINLVRDLPQGFSALEPLVDLPIGINI | |
| | TRFQTLLALHRSYLTPGDSSSGWTAGAAAYYVGYL | |
| | QPRTFLLKYNENGTITDAVDCALDPLSETKCTLKSFT | |
| | VEKGIYQTSNFRVQPTESIVRFPNITNLCPFGEVENAT | |
| | RFASVYAWNRKRISNCVADYSVLYNSASFSTFKCYG | |
| | VSPTKLNDLCFTNVYADSFVIRGDEVRQIAPGQTGKI | |
| | ADYNYKLPDDFTGCVIAWNSNNLDSKVGGNYNYR | |
| | YRLFRKSNLKPFERDISTEIYQAGSKPCNGVEGENCY | |
| | FPLQSYGFQPTNGVGYQPYRVVVLSFELLHAPATVC | |
| | GPKKSTNLVKNKCVNFNFNGLTGTGVLTESNKKFLP | |
| | FQQFGRDIADTTDAVRDPQTLEILDITPCSFGGVSVIT | |
| | PGTNTSNQVAVLYQGVNCTEVPVAIHADQLTPTWR | |
| | VYSTGSNVFQTRAGCLIGAEHVNNSYECDIPIGAGIC | |
| | ASYQTQTNSRQQAQSVASQSIIAYTMSLGAENSVAY | |
| | SNNSIAIPTNFTISVTTEILPVSMTKTSVDCTMYICGD | |
| | STECSNLLLQYGSFCTQLNRALTGIAVEQDKNTQEV | |
| | FAQVKQIYKTPPIKDFGGFNFSQILPDPSKPSKRSFIE | |
| | DLLFNKVTLADAGFIKQYGDCLGDIAARDLICAQKF | |
| | NGLTVLPPLLTDEMIAQYTSALLAGTITSGWTFGAG | |
| | AALQIPFAMQMAYRENGIGVTQNVLYENQKLIANQ | |
| | FNSAIGKIQDSLSSTASALGKLQNVVNQNAQALNTL | |
| | VKQLSSNFGAISSVLNDILSRLDPPEAEVQIDRLITGR | |
| | LQSLQTYVTQQLIRAAEIRASANLAATKMSECVLGQ | |
| | SKRVDFCGKGYHLMSFPQSAPHGVVFLHVTYVPAQ | |
| | EKNFTTAPAICHDGKAHFPREGVFVSNGTHWFVTQR | |
| | NFYEPQIITTDNTFVSGNCDVVIGIVNNTVYDPLQPE | |
| | LDSFKEELDKYFKNHTSPDVDLGDISGINASVVNIQK | |
| | EIDRLNEVAKNLNESLIDLQELGKYEQYIKWPWYIW | |
| | LGFIAGLIAIVMVTIMLCCMTSCCSCLKGCCSCGSCC | |
| | KFDEDDSEPVLKGVKLHYT | |
| BV2540 | QCVNLITRTQSYTNSFTRGVYYPDKVFRSSVLHSTQ | 119 |
| | DLFLPFFSNVTWFHAISGTNGTKRFDNPVLPFNDGV | |
| | YFASTEKSNIIRGWIFGTTLDSKTQSLLIVNNATNVVI | |
| | KVCEFQFCNDPFLDVYYHKNNKSWMESEFRVYSSA | |
| | NNCTFEYVSQPFLMDLEGKQGNFKNLREFVFKNIDG | |
| | YFKIYSKHTPINLGRDLPQGFSALEPLVDLPIGINITRF | |
| | QTLLALHRSYLTPGDSSSGWTAGAAAYYVGYLQPR | |
| | TFLLKYNENGTITDAVDCALDPLSETKCTLKSFTVEK | |
| | GIYQTSNFRVQPTESIVRFPNITNLCPFDEVENATRFA | |
| | SVYAWNRKRISNCVADYSVLYNFAPFFAFKCYGVSP | |
| | TKLNDLCFTNVYADSFVIRGNEVSQIAPGQTGNIAD | |
| | YNYKLPDDFTGCVIAWNSNKLDSKVGGNYNYRYRL | |
| | FRKSNLKPFERDISTEIYQAGNKPCNGVAGVNCYFPL | |
| | QSYGFRPTYGVGHQPYRVVVLSFELLHAPATVCGPK | |
| | KSTNLVKNKCVNFNFNGLTGTGVLTESNKKFLPFQQ | |
| | FGRDIADTTDAVRDPQTLEILDITPCSFGGVSVITPGT | |
| | NTSNQVAVLYQGVNCTEVPVAIHADQLTPTWRVYS | |
| | TGSNVFQTRAGCLIGAEYVNNSYECDIPIGAGICASY | |
| | QTQTKSHQQAQSVASQSIIAYTMSLGAENSVAYSNN | |
| | SIAIPTNFTISVTTEILPVSMTKTSVDCTMYICGDSTEC | |
| | SNLLLQYGSFCTQLKRALTGIAVEQDKNTQEVFAQV | |
| | KQIYKTPPIKYFGGFNFSQILPDPSKPSKRSFIEDLLFN | |
| | KVTLADAGFIKQYGDCLGDIAARDLICAQKFNGLTV | |
| | LPPLLTDEMIAQYTSALLAGTITSGWTFGAGAALQIP | |
| | FAMQMAYRFNGIGVTQNVLYENQKLIANQFNSAIG | |
| | KIQDSLSSTASALGKLQDVVNHNAQALNTLVKQLSS | |
| | KFGAISSVLNDILSRLDPPEAEVQIDRLITGRLQSLQT | |
| | YVTQQLIRAAEIRASANLAATKMSECVLGQSKRVDF | |
| | CGKGYHLMSFPQSAPHGVVFLHVTYVPAQEKNFTT | |
| | APAICHDGKAHFPREGVFVSNGTHWFVTQRNFYEP | |
| | QIITTDNTFVSGNCDVVIGIVNNTVYDPLQPELDSFK | |
| | EELDKYFKNHTSPDVDLGDISGINASVVNIQKEIDRL | |
| | NEVAKNLNESLIDLQELGKYEQYIKWPWYIWLGFIA | |
| | GLIAIVMVTIMLCCMTSCCSCLKGCCSCGSCCKFDE | |
| | DDSEPVLKGVKLHYT | |

The antibodies 239.12 and 322.3 were identified from hybridomas produced from mice that were immunized with BV2373. The antibody 425.6 was identified from a hybridoma produced by mice that were immunized with BV2438. The antibody 35.13 was identified from a hybridoma produced by mice immunized with BV2465. The antibody 35.13 was identified from a hybridoma produced by mice immunized with BV2540.

The V_{H} and VL sequences of 239.12, 322.3, 425.6, 35.13, and 199.9 are identified in Table B.

TABLE B
VH and VL Sequences of 239.12, 322.3, 425.6, 35.13, and 199.9

| Antibody Name | VL | VH |
|---|---|---|
| 425.6 | QIVLTQSPAIMSVSPGEKV | VQLKQSGPSLVQPSQSLSI |
| | TMTCSASSTMSFMYWYQ | TCTVSGFSLTSYGIHWVR |
| | QKPGSSPRLLIYDTSNLAS | QSPGKGLEWLGVIWRGG |
| | GVPVRFSGSGSGTSYSLTI | STDYNAAFMSRLTITKDN |
| | SRVEAEDAATYYCQQWN | SKSQVFFKMNSLQADDT |
| | TYPLTFGAGTKLELKR | AIYYCATHTEGDDVDYW |
| | (SEQ ID NO: 1) | GQGTSVTVSS (SEQ ID |
| | | NO: 5) |
| 35.13 | EIQMTQSPSSMSASLGDRI | QVQLQQSGAELGKPGAS |
| | TITCQATQDIVKNLNWYQ | VKLSCRASGYTFTRYYIY |
| | QKPGKPPSFLIYHATELAK | WVKQRPGQGLEWIGEINP |
| | GVPSRFSGSGSGSDYSLTI | SNGDTHFNEKFKTKATLT |
| | SNLESEDFADYYCLQFYD | VDKSSSSAHIHLTSLTSED |
| | FPFTFGAGTKLELKR (SEQ | SAVYYCSIHAYSFDFWGQ |
| | ID NO: 2) | GTTLTVSS (SEQ ID NO: 6) |
| 239.12 | DIQMTQSPASLSASVGET | EVKLEESGPVLVKPSQSLS |
| | VTITCGASENIYGALNWY | LTCTVTGYSITSDYAWN |
| | QRKQGKSPQLLIYGATNL | WIRQFPGNKLEWMGYISN |
| | ADGMSSRFSGSGSGRQYS | SAGTSYNPSLKSRISITRD |
| | LKISSLHPDDVATYYCON | TSKNQFFLQLNSVTTEDT |
| | VLSTPYTFGGGTKLEIKR | ATYYCASGGWSSYYAMD |
| | (SEQ ID NO: 3) | YWGQGTSVTVSS (SEQ ID |
| | | NO: 7) |
| 322.3 | METDTLLLWVLLLWVPG | MGWSRIFLFLLSIIAGVHC |
| | STGNIVLTQSPASLAVSLG | RVQLQQSGPELVKPGASV |
| | QRATISCRASESVDSYGSS | TISCKASGYIFTSDYIHWV |
| | FMHWYQQKPGQPPKLLI | KQRPGQGPEWIGWIYPGN |
| | YLASNLESGVPARFSGSG | INTKYNEKFKGKATLTAD |
| | SRTDFTLTIDPVEADDAA | KSSSTAYMQLSSLTSEDS |
| | TYYCQQNNEDPRTFGGG | AVYFCARDDGNLYYYAM |
| | TKLEIK (SEQ ID NO: 4) | DYWGQGTSVTVSS (SEQ |
| | | ID NO: 8) |
| 199.9 | METDTLLLWVLLLWVPG | MGWSYIILFLVATATGVH |
| | STGNIVLTQSPASLAVSLG | SLVQLQQPGAELVKPGAS |
| | QRATISCRASESVDTYGN | VKLSCKASGYTFTSYFMY |
| | SFMHWYQQKPGQP | WVRQRPGQGLEWIGEINP |
| | PKLLIYLASNLESGVPARF | SNGGNDFNEKFKSKATLT |
| | SGSGSRTDFTLTIDPVEAD | VDKSSSTAYMQLSSLTSE |
| | DAATYYCQQNNEDPLTF | DSAVYYCTRSGDYGYDG |
| | GAGTKLELK (SEQ ID NO: | FAYWGQGTLVTVSA |
| | 74) | (SEQ ID NO: 75) |

### Example 2: Characterization of Binding of Antibodies 239.12; 322.3; 425.6; 35.13, and 199.9 to SARS-CoV-2 S Glycoproteins

Purpose: The ability of antibodies 239.12, 322.3, 425.6, 35.13, and 199.9 to bind to CoV S glycoproteins derived from the parent SARS-CoV-2 virus (SARS-CoV-2 virus having a SARS-CoV-2 S glycoprotein of SEQ TD NO: 9) and multiple SARS-CoV-2 variants was probed. The ability of the aforementioned antibodies to inhibit the interaction between hACE2 and the SARS-CoV-2 S glycoprotein was also probed. Finally, the ability of the aforementioned antibodies to neutralize live SARS-CoV-2 viruses and pseudoviruses was probed.

Methods—recombinant CoV S glycoprotein production: Genes encoding CoV S glycoproteins were codon optimized for expression in Spodoptera frugiperda (Sf9) cells and synthetically produced from the full-length CoV S glycoprotein gene sequences. The CoV S glycoproteins contained the inactive furin cleavage site QQAQ (SEQ ID NO: 144) and two proline point mutations were introduced at K986P and V987P, wherein the amino acids are numbered according to SEQ ID NO: 10. Table J contains the amino acid sequences of the SARS-CoV-2 S glycoproteins used herein.

Methods—Antibody Production: Antibodies were generated according to the methods described in Example 1.

Methods—Biolayer Inferometry: Binding kinetics of CoV S glycoproteins to brMAbs (i.e., 239.12; 322.3; 425.6; 35.13; and 199.9) captured on biosensors were performed using an Octet QK 384 instrument (ForteBio, Fremont, CA). Briefly, to measure binding to a CoV S glycoproteins (i.e., SARS-CoV-2 rS), respective brMAbs were coupled to anti-mouse Fc biosensors at 2 μg/mL for 600 seconds followed by a washing step where a baseline measurement was taken. The amino acid sequences of the CoV S glycoproteins are found in Table J. Then, association of the CoV S glycoprotein was measured for 600 seconds, followed by a 600-second dissociation step. Binding kinetics were analyzed using Octet software HT10.0. To measure binding kinetics of brMAbs to Spike RBD-His, the RBD-His (2 pg/mL) was coupled to Ni-NTA biosensors. After baseline measurement, association of the antibodies was measured for 600 seconds, followed by dissociation for 600 seconds. Binding kinetics were analyzed using Octet software HT10.0.

Methods ELISA—96-well microtiter were coated with 1.0 pg/mL of SARS-CoV-2 S proteins. After blocking non-specific binding, serial dilution of monoclonal antibodies were added and binding of antibodies were measured using horseradish peroxidase (HRP) conjugated anti-mouse. Substrate turnover was measured at OD 450 nm. EC50 values were calculated by 4-parameter curve fitting.

Methods—hACE2 Receptor Inhibition: The ability of the antibodies to block the interaction between the human angiotensin-converting enzyme 2 (hACE2) receptor and the CoV S glycoproteins were evaluated by ELISA. Briefly, 96-well plates were coated with 1.0 g/mL CoV S glycoproteins overnight at 4° C. Plates were washed with PBS-T and nonspecific binding was blocked with TBS Startblock blocking buffer. Sera or mAb solutions were serially diluted 2-fold starting with a 1:20 dilution and added to coated wells for 1 hour at room temperature. After washing, 30 ng/mL of histidine-tagged hACE2 was added to wells for 1 hour at room temperature. HRP-conjugated anti-histidine IgG was added and incubated for 1 hour followed by addition of TMB substrate. Plates were read at OD 450 nm with a SpectraMax Plus plate reader and data analyzed with SoftMax Pro software. The % Inhibition for each dilution for each sample was calculated using the following equation in the SoftMax Pro program: 100-[(MeanResults/ControlValue@PositiveControl)*100].

Serum dilution versus % Inhibition plot was generated and curve fitting was done by 4 parameter logistic (4PL) curve fitting to data. Serum antibody titer or antibody concentration at 50% binding inhibition (IC₅₀) of hACE2 to CoV S glycoproteins was determined in the SoftMax Pro program. Individual animal hACE2 receptor inhibiting titers, group geometric mean titers, and 95% CI were plotted using GraphPad Prism 7.05 software. For a titer below the assay LOD, a titer of <20 (starting dilution) was reported and a value of “10” assigned to the sample to calculate the group mean titer.

Methods—Live SARS-CoV-2 Neutralization Assay: Handling of live SARS-CoV-2 was performed in the select agent Animal Biosafety Level 3 facility at the University of Maryland, School of Medicine (Baltimore, MD). Vero/TMPRSS2 cells were maintained in complete media comprised of DMEM (Quality Biological), supplemented with 10% (v/v) fetal bovine serum (heat inactivated, Sigma-Aldrich), 1% (v/v) penicillin/streptomycin, and 1% (v/v) L-glutamine (2 mM final concentration, Gibco). Stock virus for the SARS-CoV-2 isolates were prepared in Vero/TMPRSS2 cells and sequence confirmed. Monoclonal antibodies were processed in duplicate for a final initial concentration of 10 μg/mL followed by 1:2 serial dilutions, resulting in a 12-dilution series with each well containing 100 μL. Lower sample concentrations were processed as necessary. All dilutions were performed in DMEM (Quality Biological), supplemented with 10% (v/v) fetal bovine serum (heat inactivated, Sigma), 1% (v/v) penicillin/streptomycin (Gemini Bio-products), and 1% (v/v) L-glutamine (2 mM final concentration, Gibco). Dilution plates were then transported into the BSL-3 laboratory and 100 μL of diluted SARS-CoV-2 inoculum was added to each well to result in a multiplicity of infection (MOI) of 0.01 upon transfer to titering plates. A non-treated, virus-only control and a mock infection control were included on every plate. The sample/virus mixture was then incubated at 37° C. (5.0% CO₂) for 1 h before transferring 100 μL to clear, 96-well titer plates with confluent Vero/TMPRSS2 cells. Titer plates were incubated at 37° C. (5.0% CO₂) for 48-72 h (depending on the variant), followed by visual CPE determination for each sample dilution. The first sample dilution to show CPE was reported as the minimum sample dilution required to neutralize >99% of the concentration of SARS-CoV-2 tested (Neut₉₉).

Methods—Pseudovirus Neutralization Assay: SARS-CoV-2 Pseudoviruses were generated using a lentivirus platform. Briefly, backbone and helper plasmids including the CoV S glycoproteins were obtained. Omicron variants in pcDNA3.1 were synthesized by GenScript using a gene encoding the CoV S glycoprotein sequence from the EPICoV database, followed by codon optimization and deletion of the cytoplasmic tail for Prototype (SARS-CoV-2 virus encoding a Spike glycoprotein of SEQ ID NO: 9) pseudovirus. HEK293T cells were seeded at 1×10⁶ cells/well in 6-well tissue culture plates and incubated at 37° C. overnight and transfected using LIPOFECTAMINE™ 3000 with a plasmid encoding lentiviral backbone, expressing a marker protein (luciferase or Zs green), a plasmid expressing a CoV S glycoprotein, and a plasmid expressing other HIV proteins for virion formation. Seventy-two hours after transfection, supernatants were collected and filtered through 0.45 μM filter to obtain pseudovirus stock. Aliquots of pseudovirus stock were stored at −80° C.

The pseudovirus neutralization assay was then performed using a HEK293T cell line stably expressing hACE2. Solutions containing the antibodies described herein were serially diluted two-fold in HEK293T cell culture media (DMEM+10% FBS+1% Penicillin+streptomycin+glutamine, without puromycin) and 50 μL was added to each well in 96-well tissue culture plate. Fifty microliters of SARS-CoV-2 Pseudovirus stock (corresponding to 3-7% GFP) was then added to each well, followed by incubation at 37° C. for one hour. Then, 2.5×10⁴ cells HEK293T/hACE2 cells in 100 μL of HEK293T medium containing puromycin were added to the wells, followed by incubation for 72 hours at 37° C. After incubation, medium was removed carefully using a pipette and 50 μL trypsin was added to dislodge cells. Manual agitation using a pipette was utilized to dislodge cells and 4% of paraformaldehyde prepared in PBS was added to each well. Virus replication was determined by measuring the fluorescence at 488-510 nm with a Guava flow cytometer and InCyte software (Luminex). Data were analyzed and neutralization curves were generated in GraphPad Prism for each sample, 50% Neutralization Titers (EC50) were calculated by 4-parameter curve fitting.

Results—Biolayer Inferometry: Table C shows the binding kinetics of antibodies 239.12, 322.3, 425.6, and 35.13 to the RBD of a CoV S glycoprotein related to a CoV S glycoprotein from the SARS-CoV-2 omicron strain.

TABLE C
Binding of 239.12, 322.3, 425.6, and 35.13 to the RBD of a
CoV S glycoprotein related to a CoV S glycoprotein
from the SARS-CoV-2 omicron strain.

| | Rate | Rate | |
|---|---|---|---|
| | constant of | constant of | |
| | association | dissociation | |
| Antibody Name | (1/Ms) | (1/s) | K_{D} (M) |
| 239.12 | No binding | | |
| 322.3 | 1.38E+05 | 1.27E+07 | 1.69E−12 |
| 425.6 | 2.72E+05 | 1.23E+03 | 4.89E−09 |
| 35.13 | 1.42E+05 | 1.77E+07 | 1.93E−12 |

Table D shows the binding kinetic parameters for each antibody evaluated. As Table D shows, each antibody bound to multiple CoV S glycoproteins. Specifically, 239.12 bound to CoV S glycoproteins related to the SARS-CoV-2 parent strain, the gamma strain, delta strain, and alpha strain. 322.3 binds to CoV S glycoproteins related to the SARS-CoV-2 parent strain, the gamma strain, beta strain, delta strain, alpha strain, and multiple omicron strains. 35.13 binds to CoV S glycoproteins related to the SARS-CoV-2 parent strain, the gamma strain, beta strain, delta strain, alpha strain, and multiple omicron strains. 425.6 binds to CoV S glycoproteins related to the SARS-CoV-2 parent strain, the gamma strain, beta strain, delta strain, alpha strain, and multiple omicron strains.

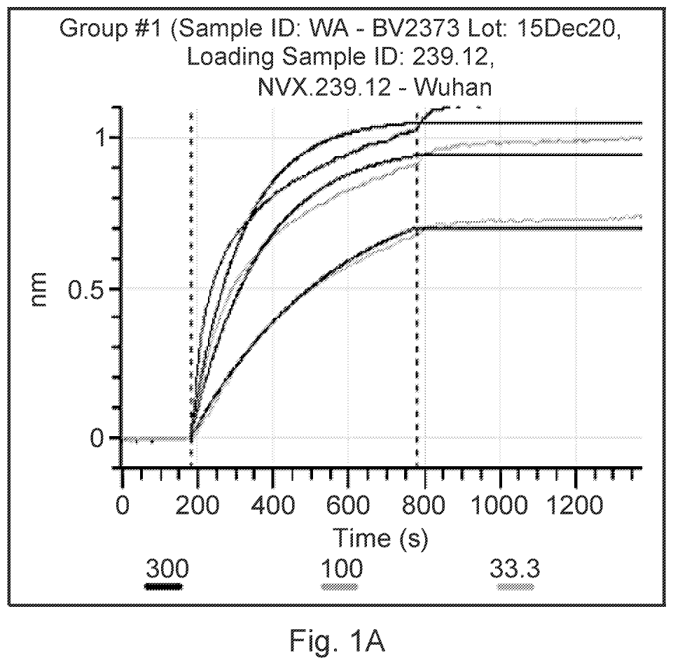

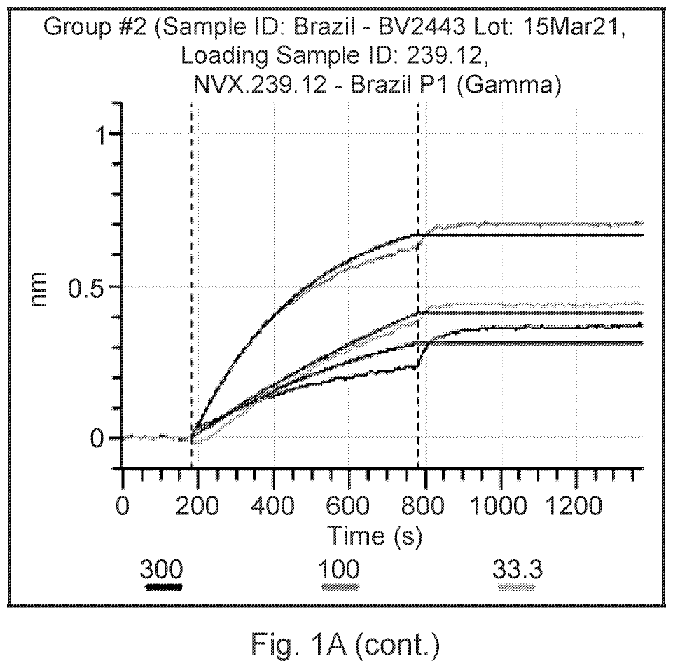

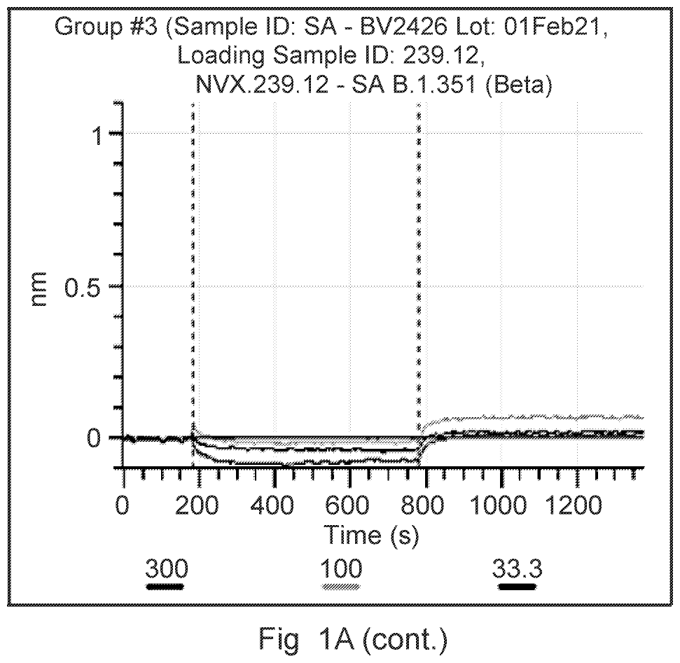

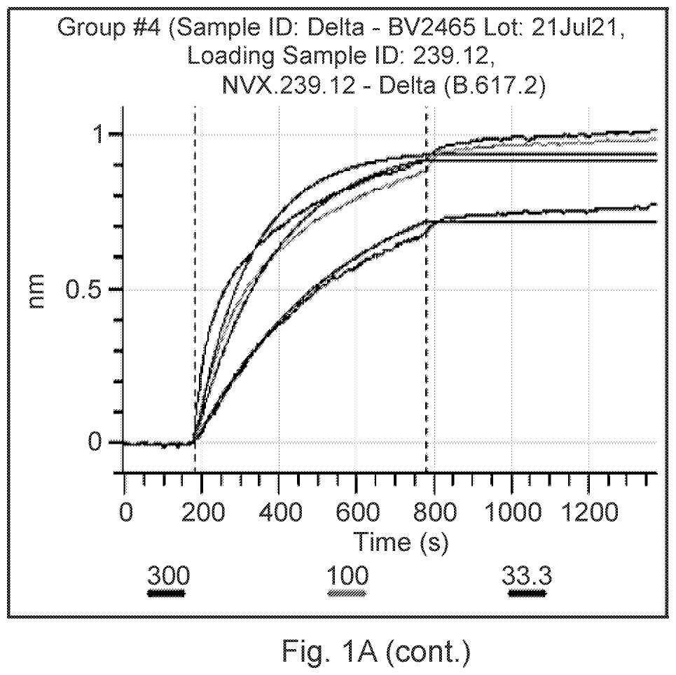

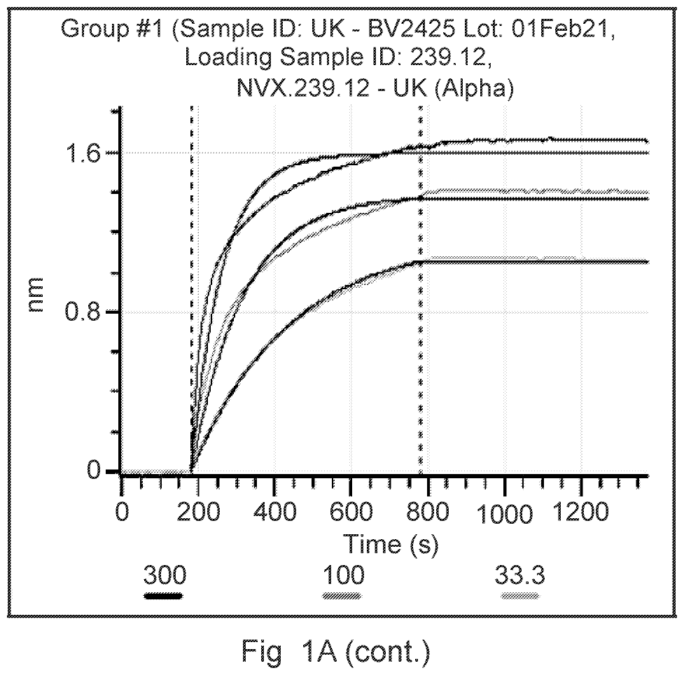

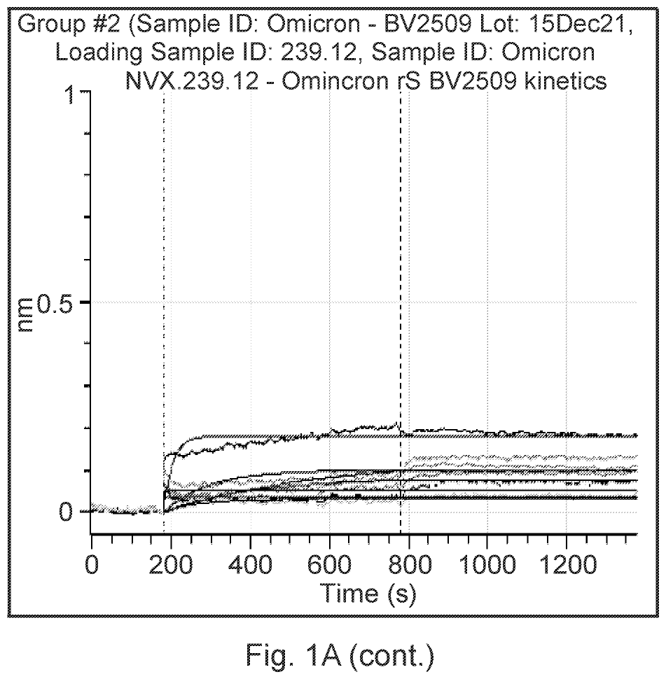

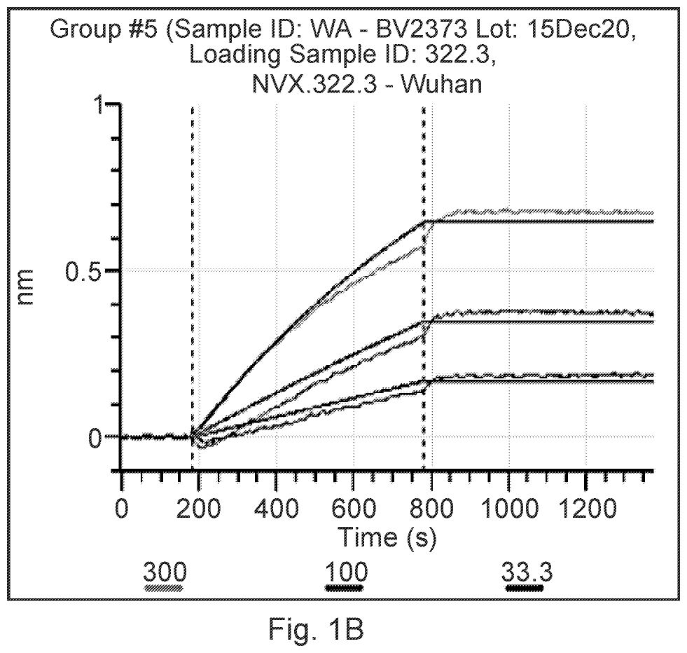

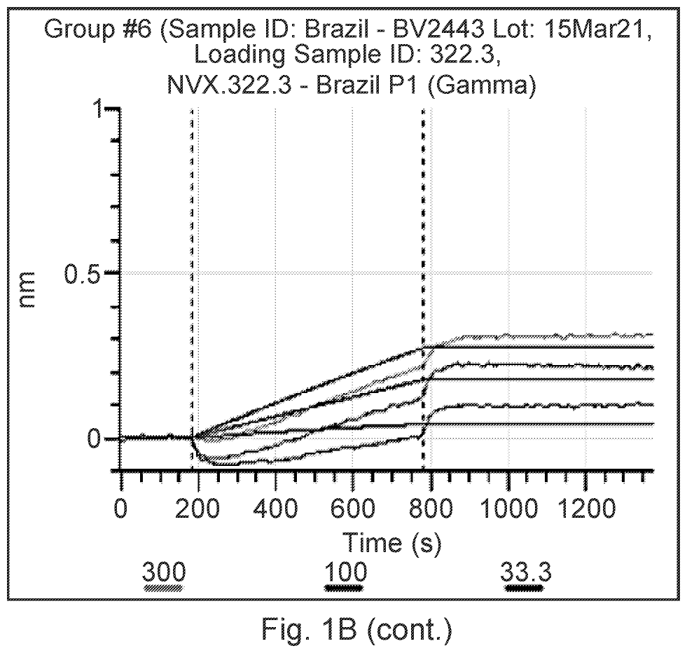

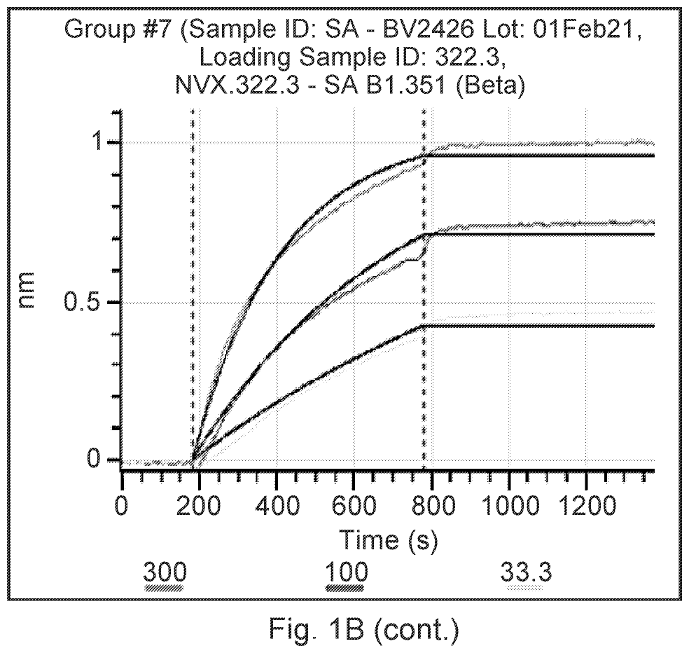

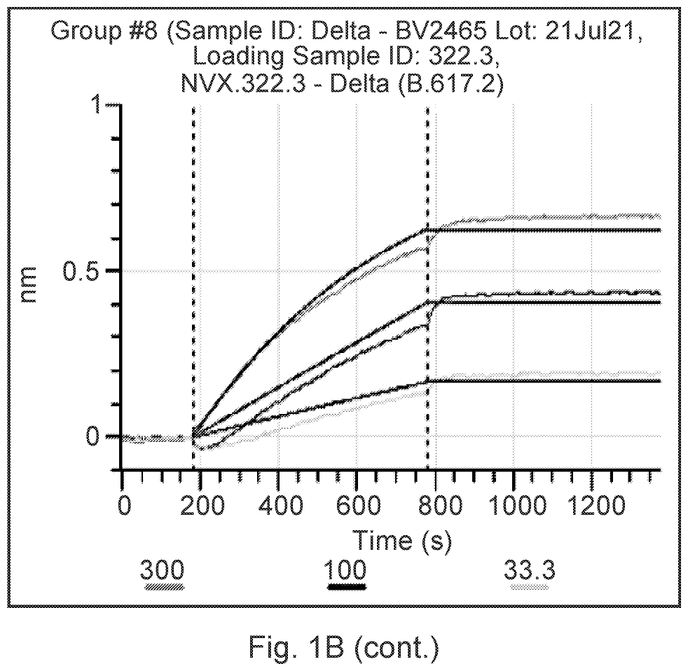

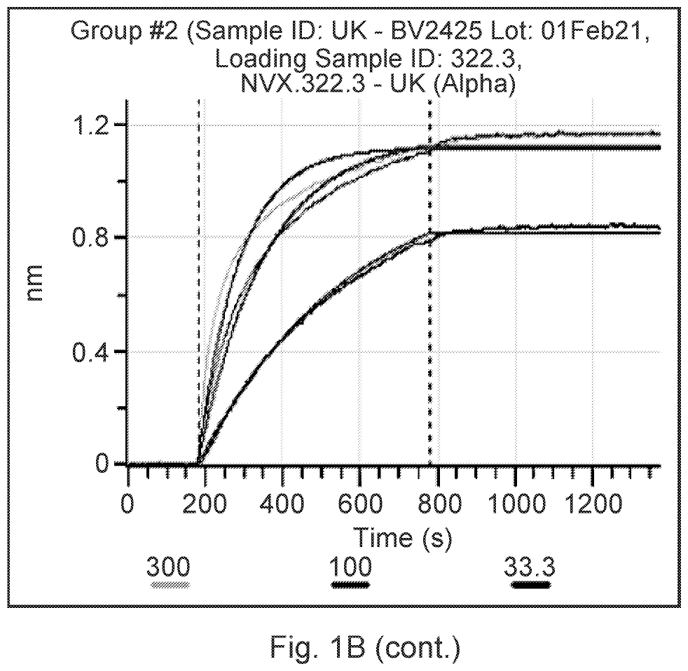

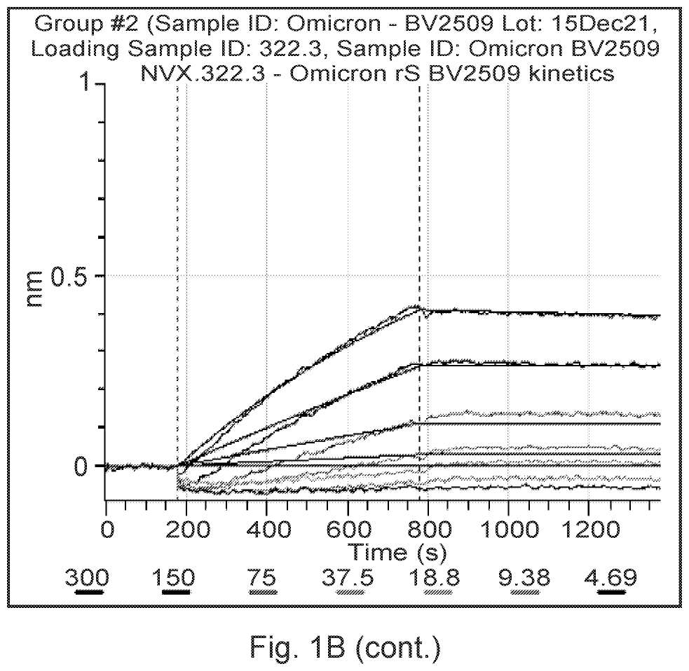

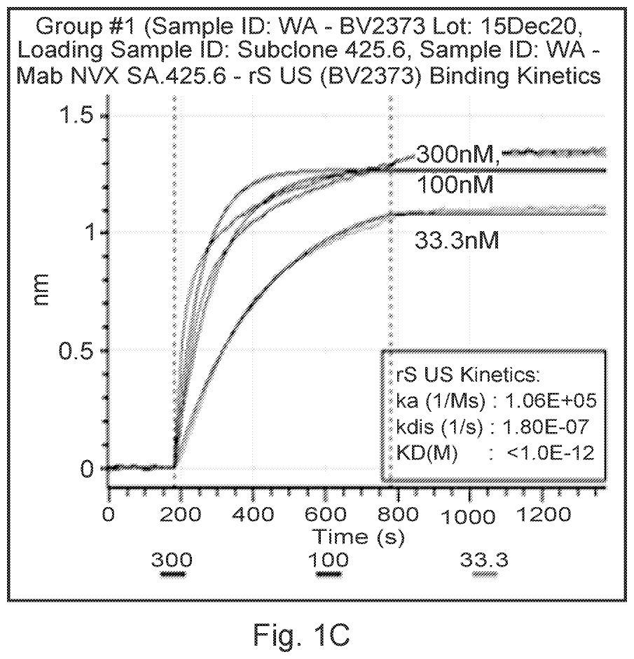

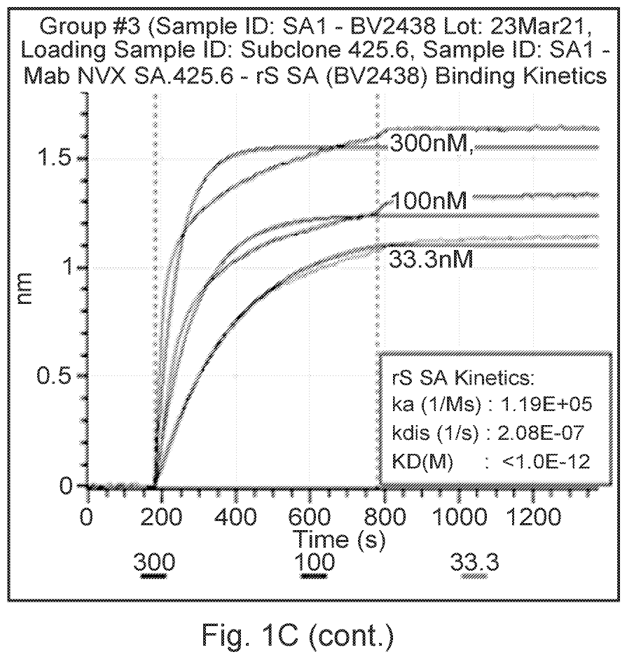

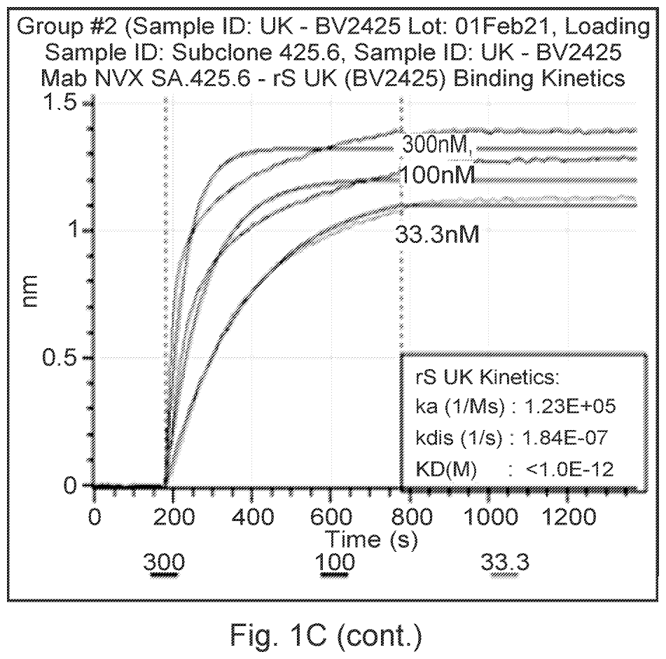

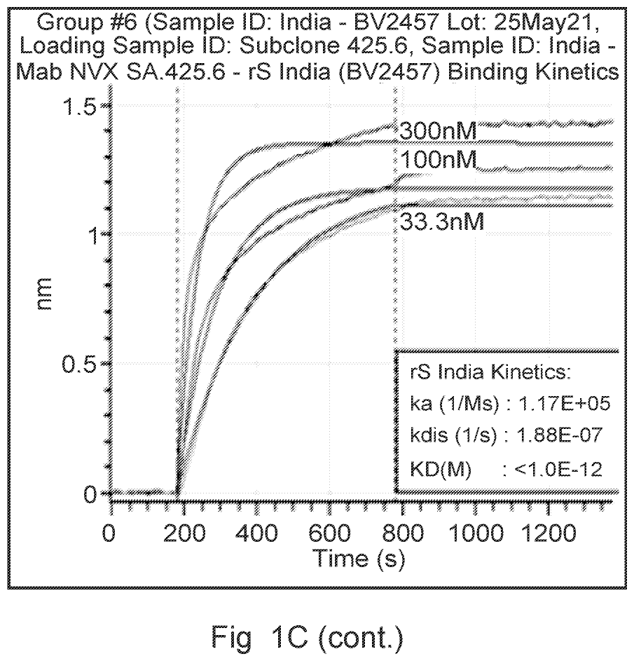

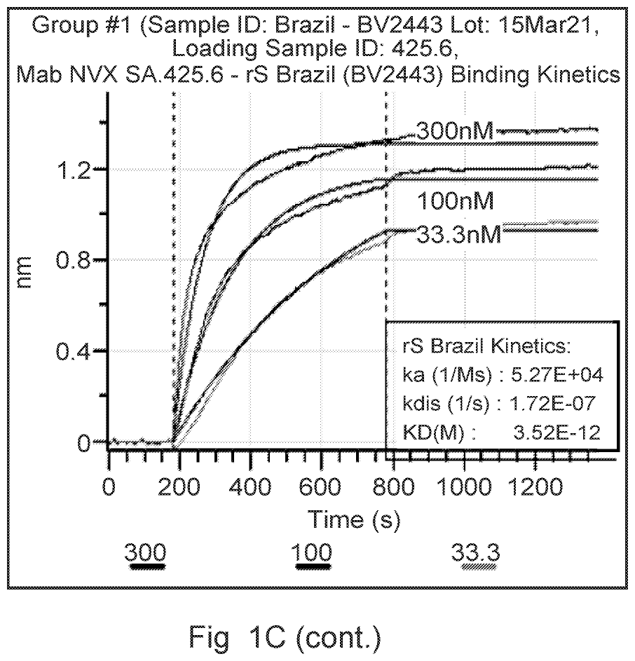

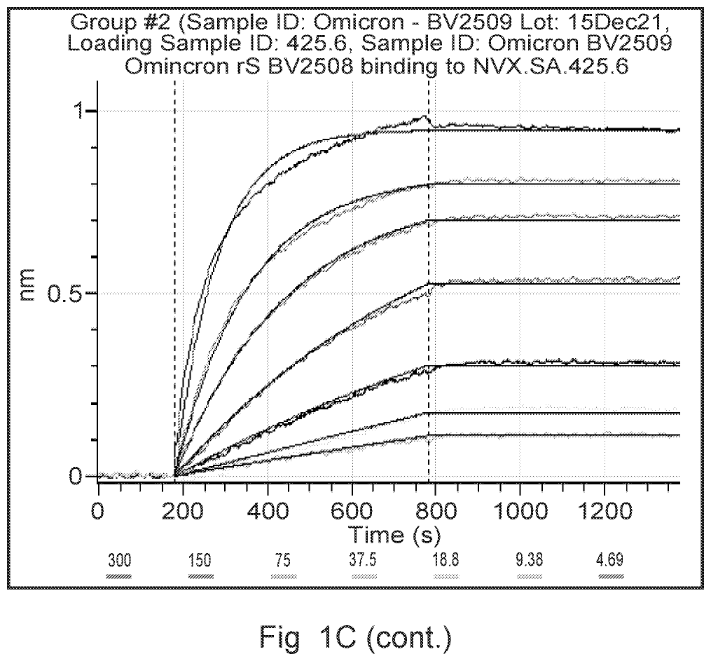

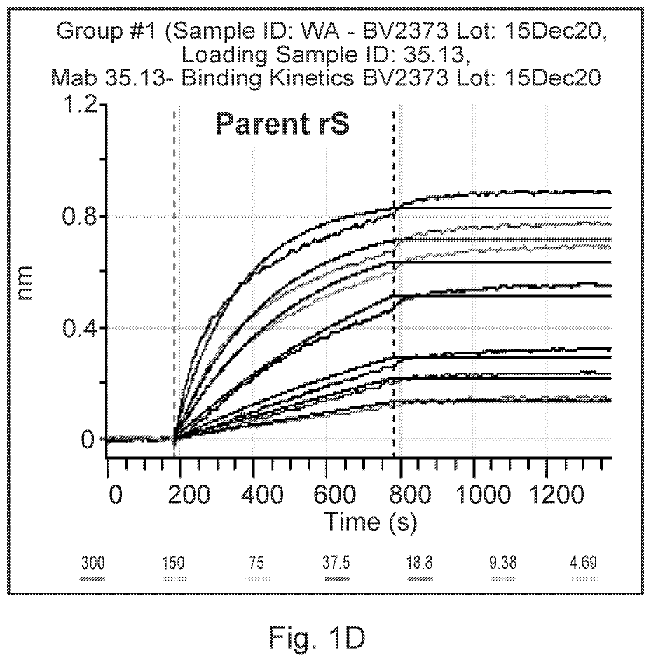

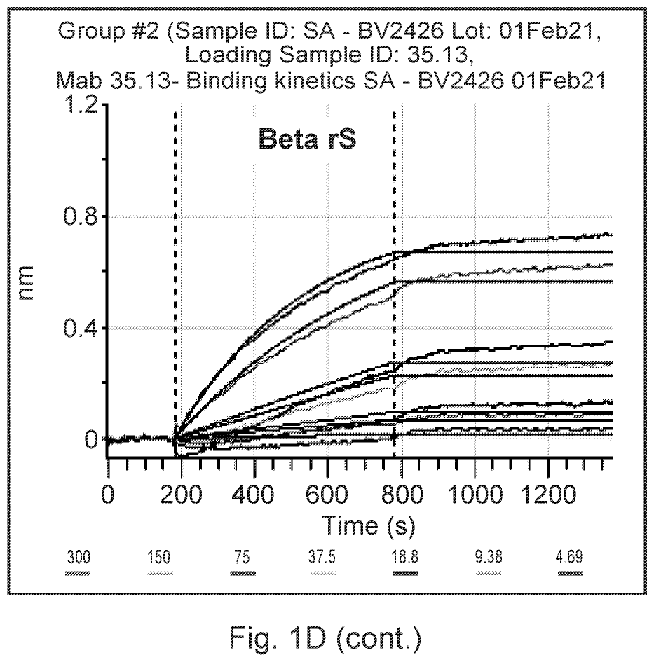

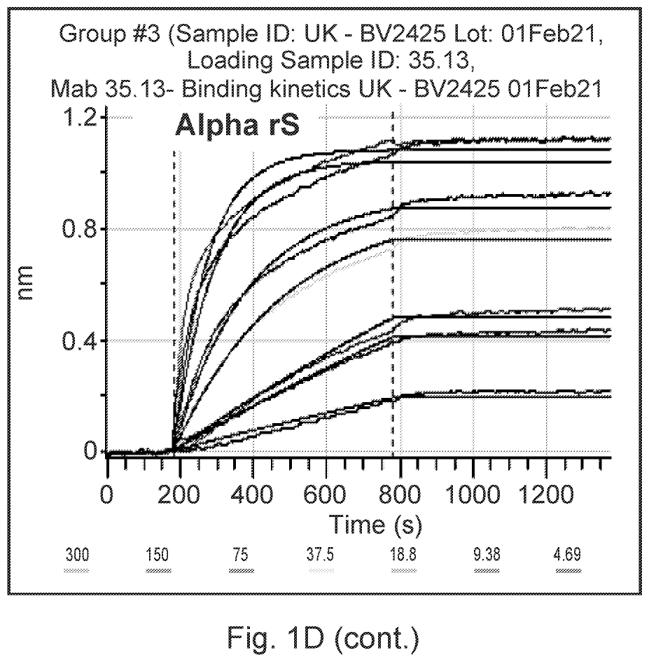

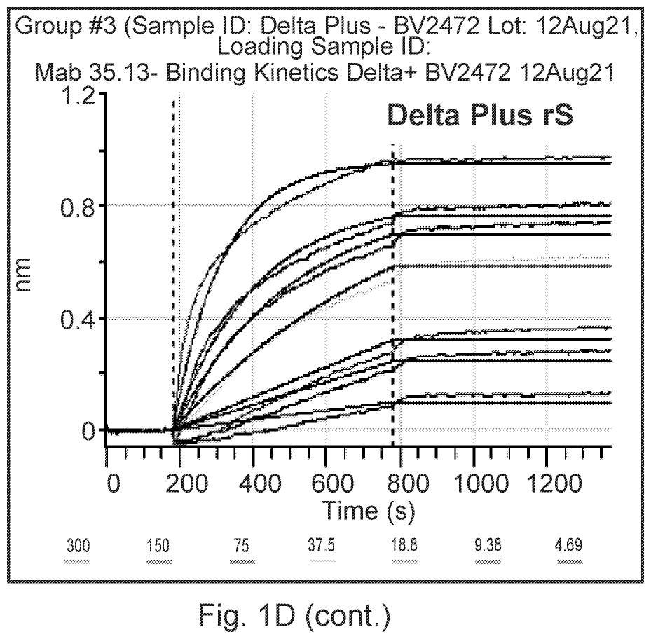

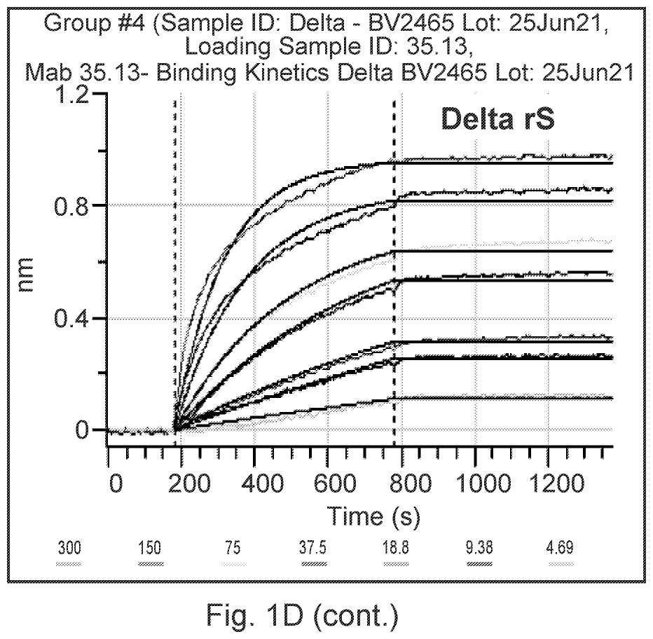

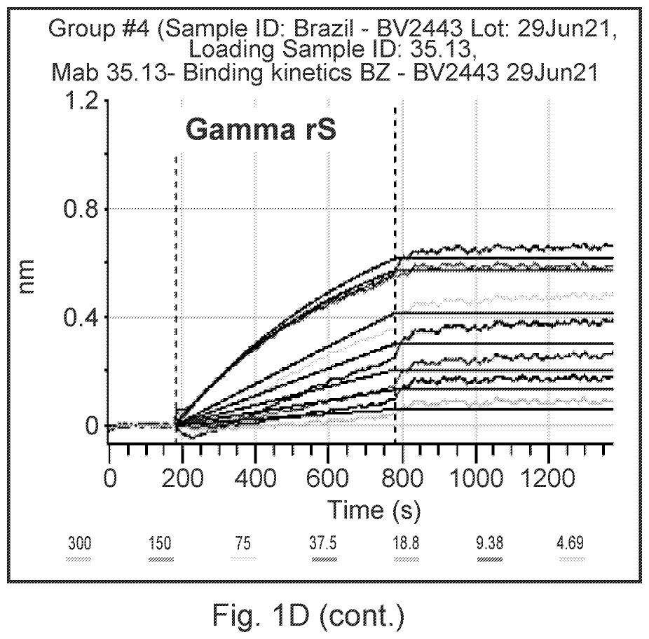

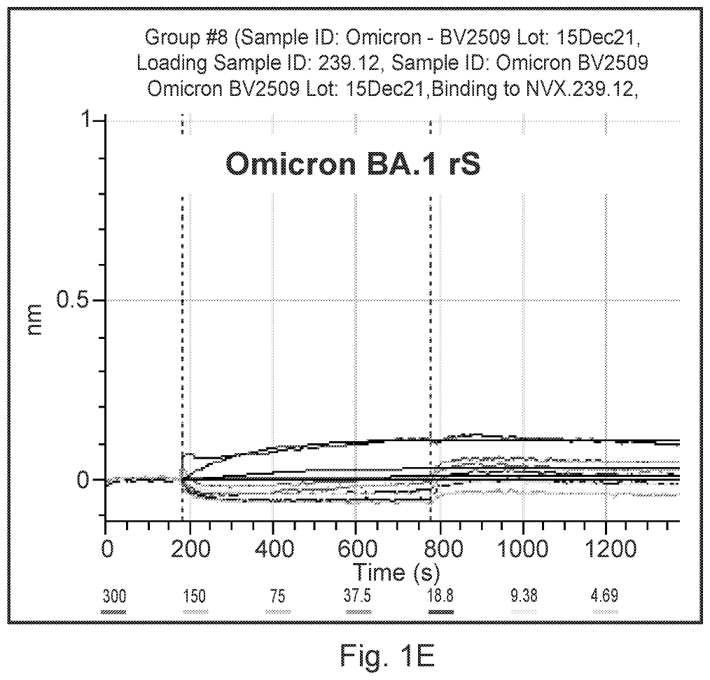

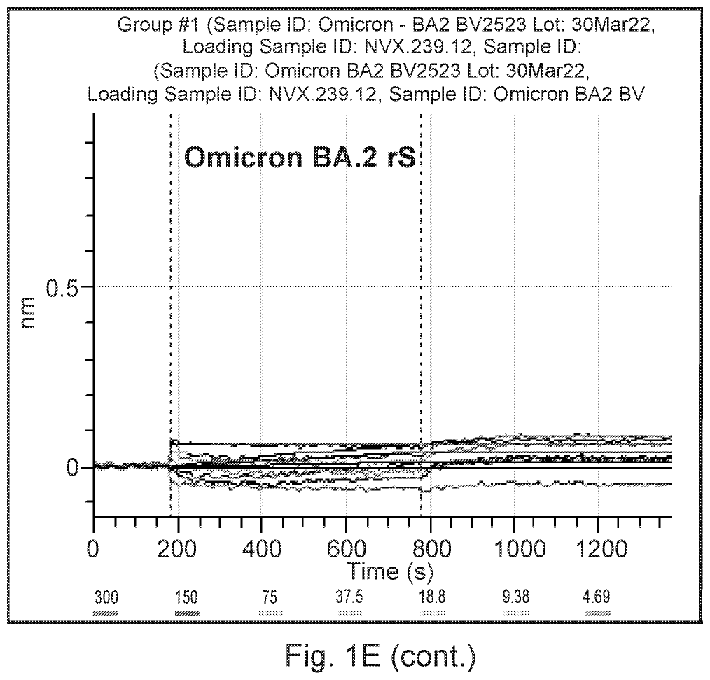

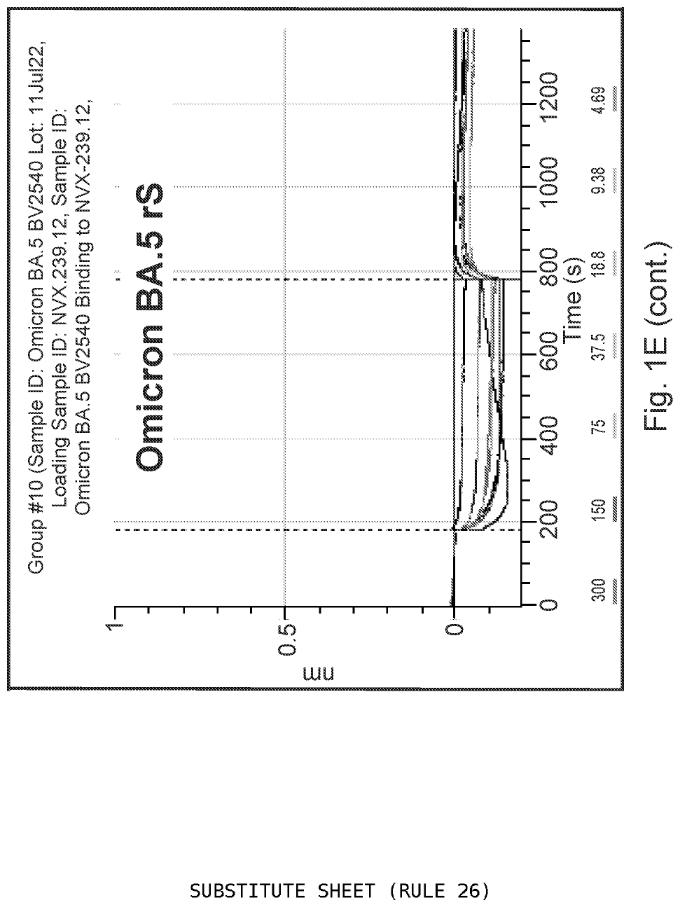

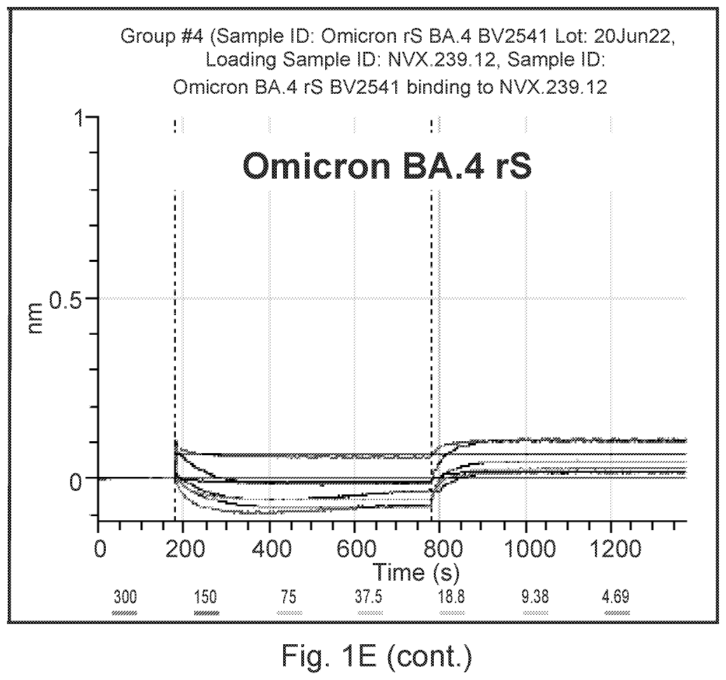

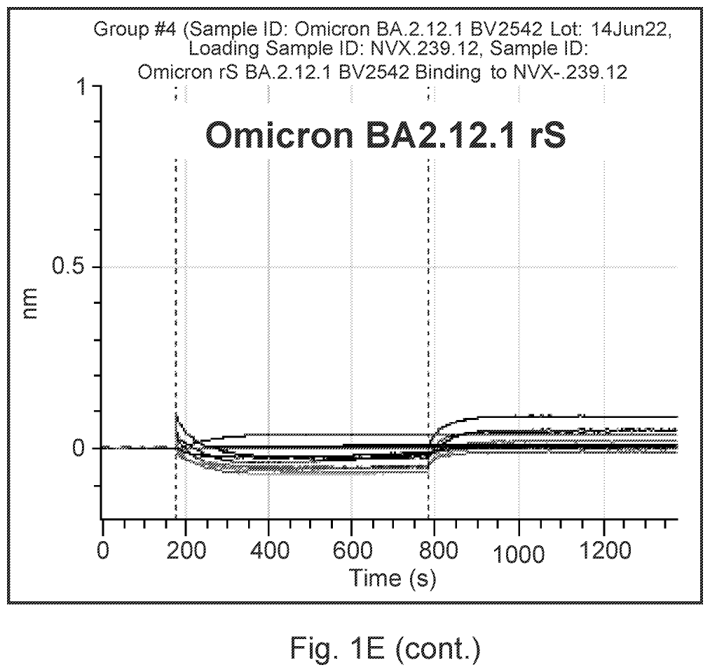

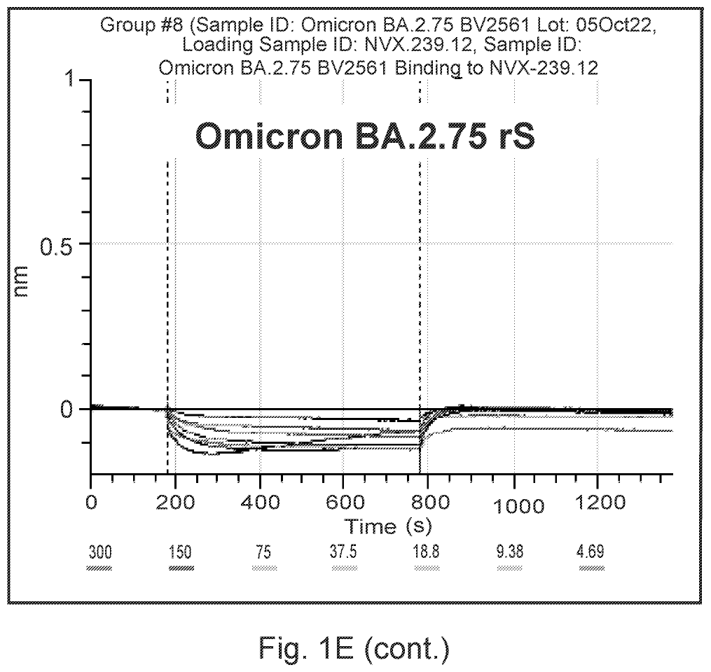

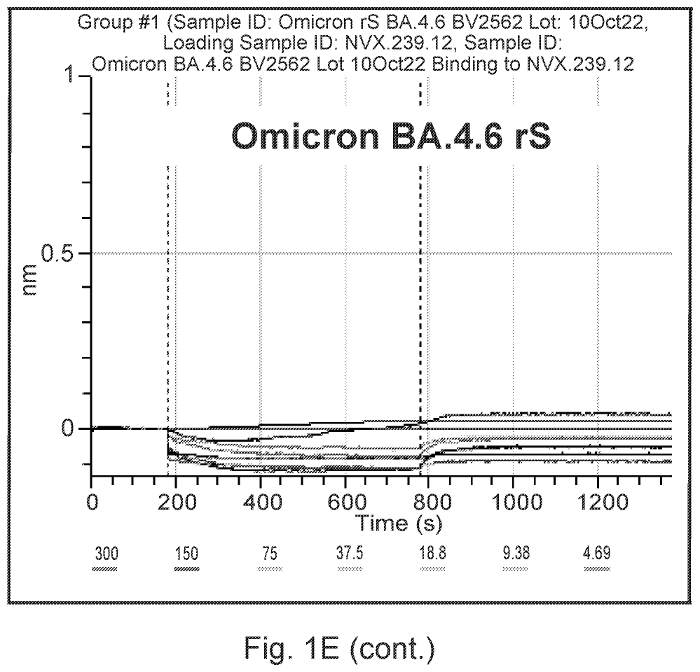

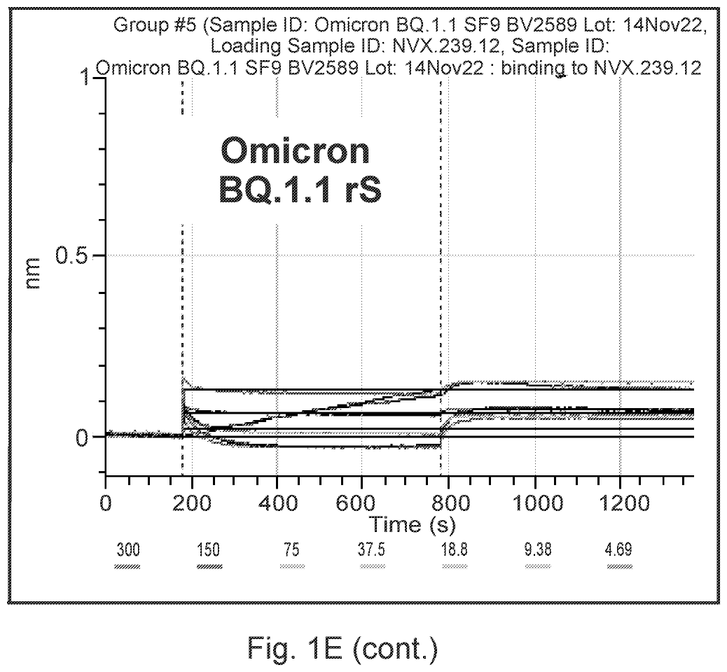

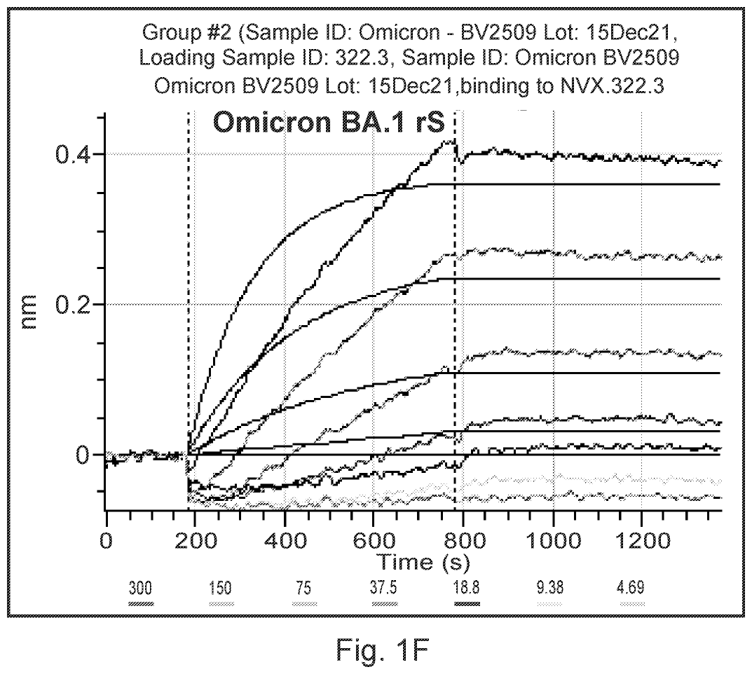

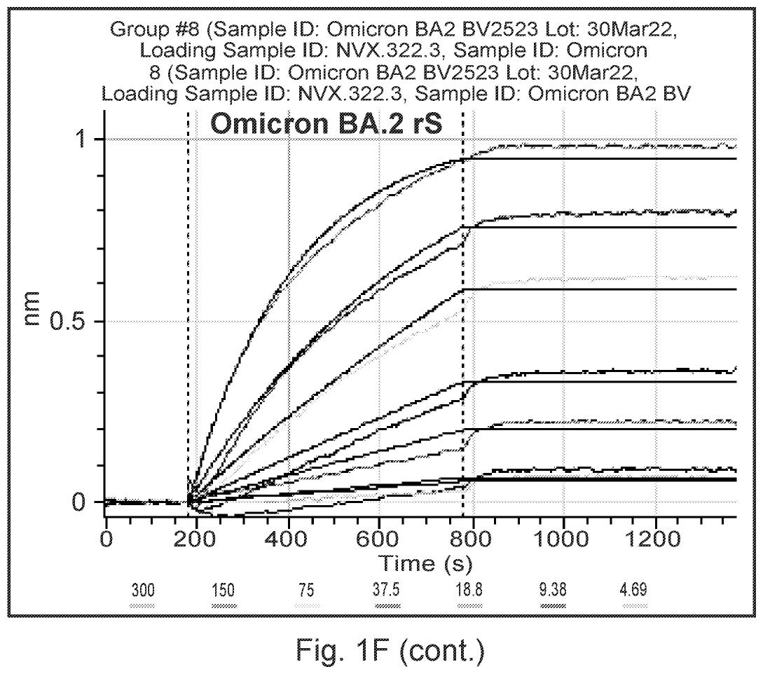

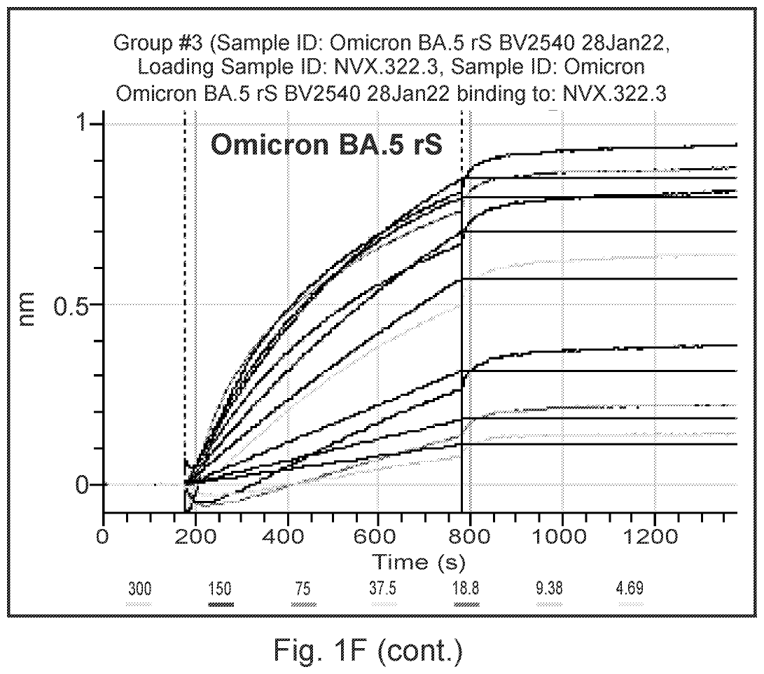

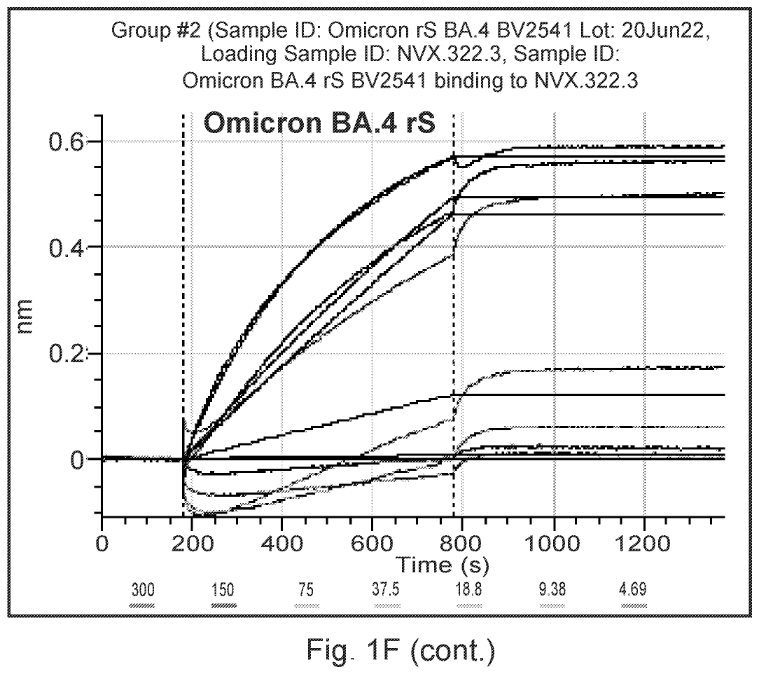

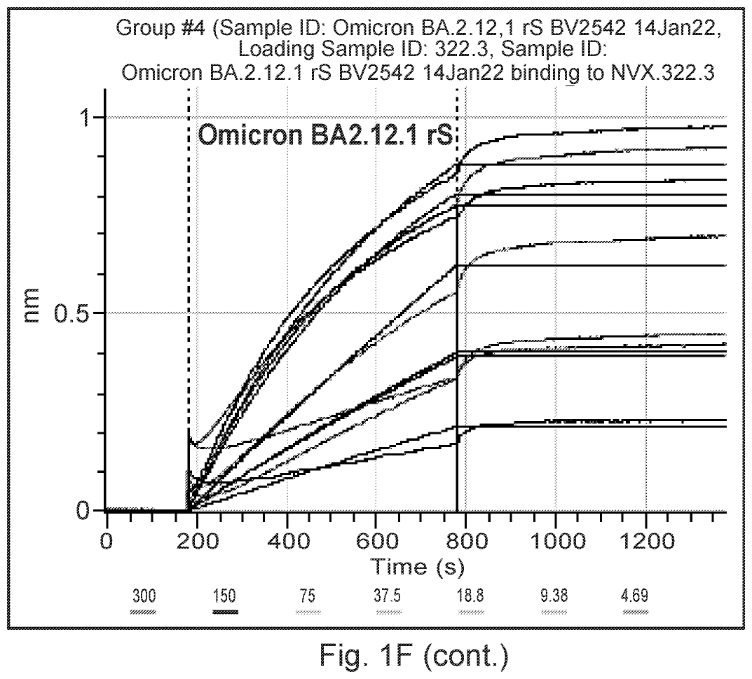

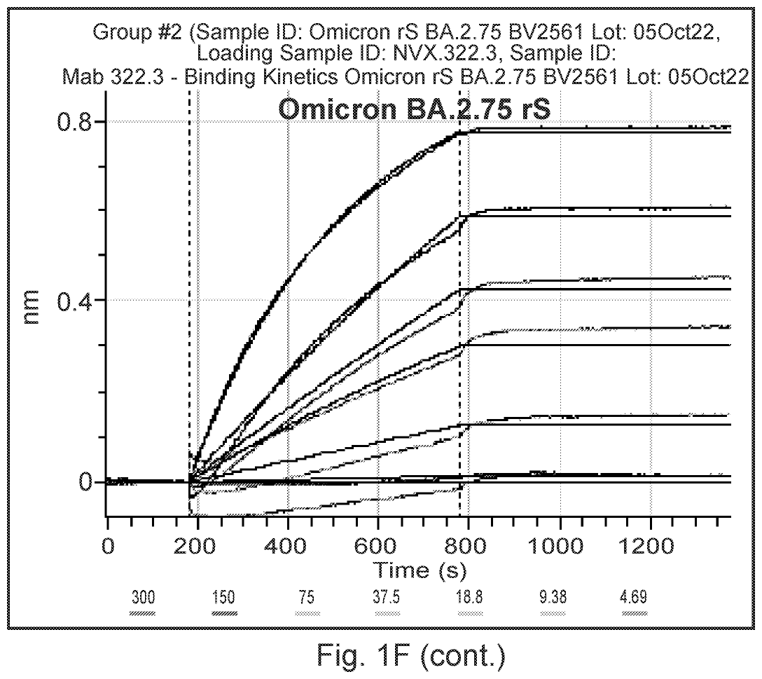

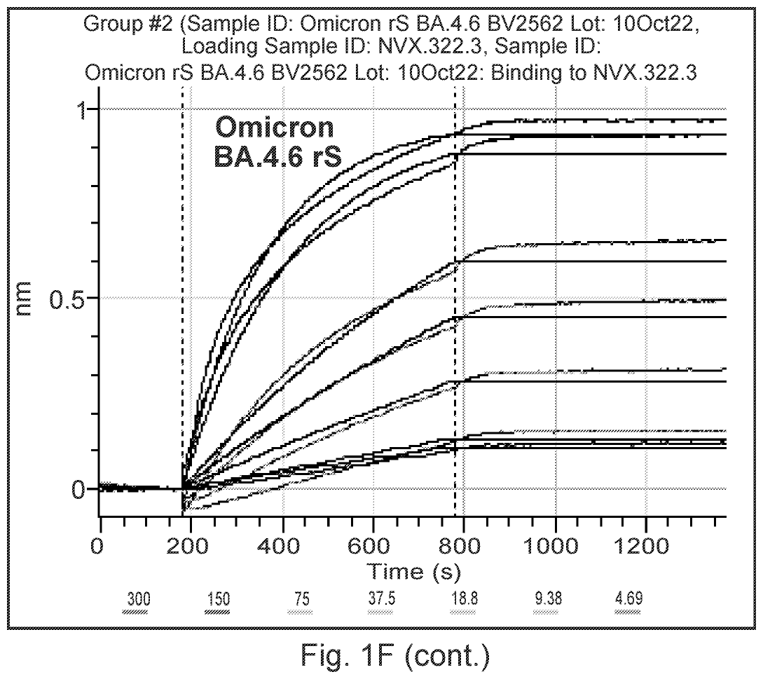

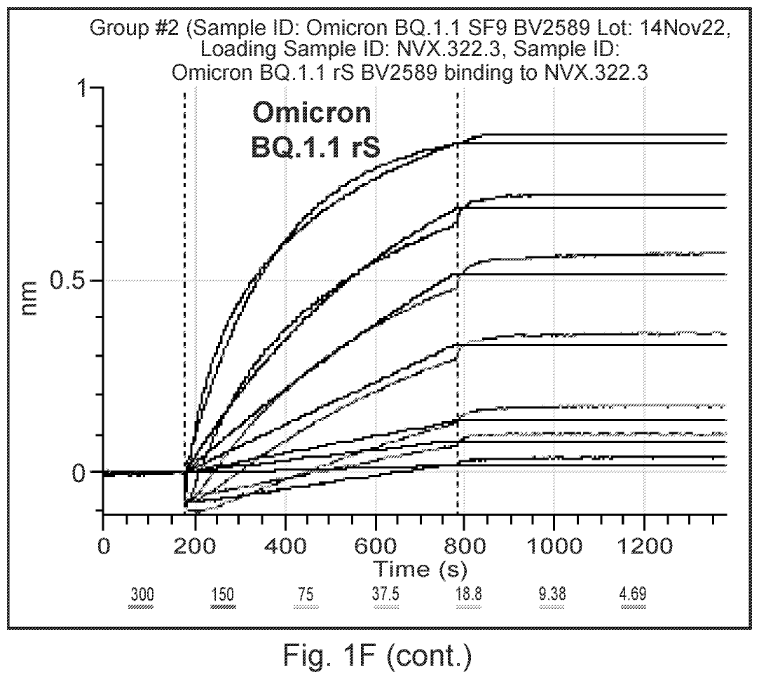

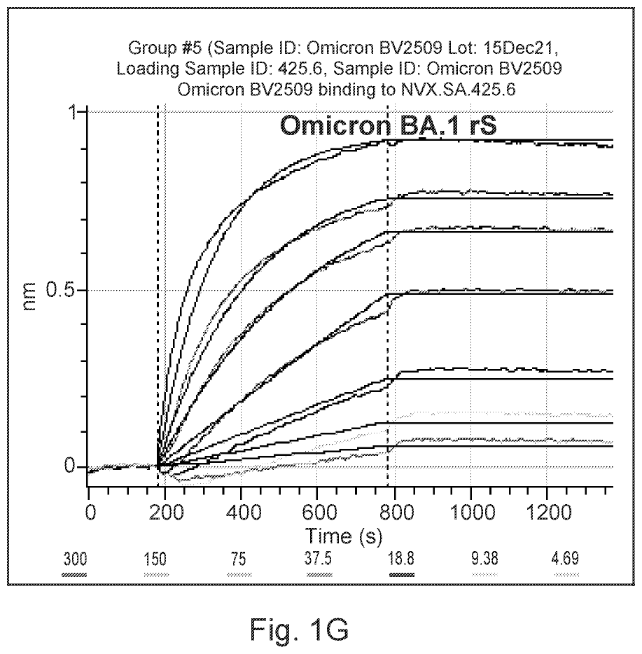

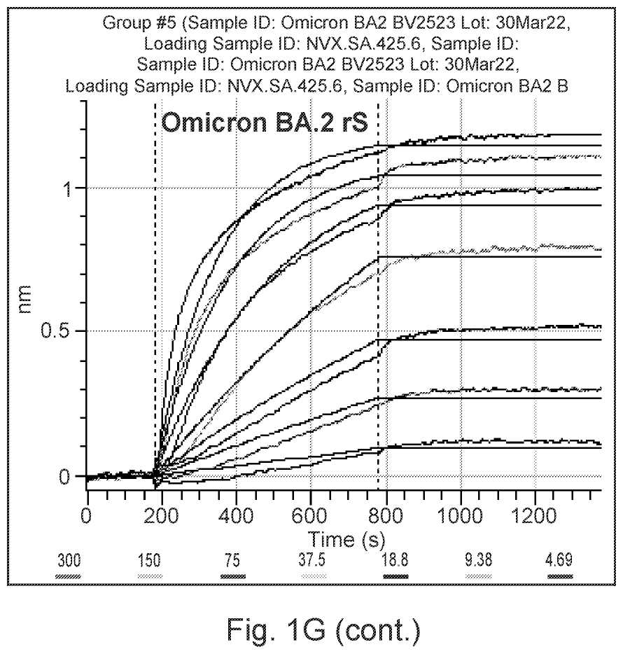

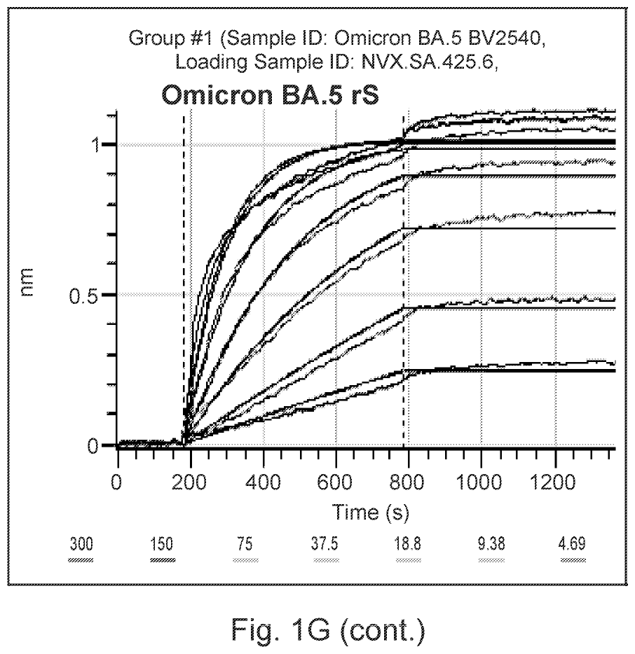

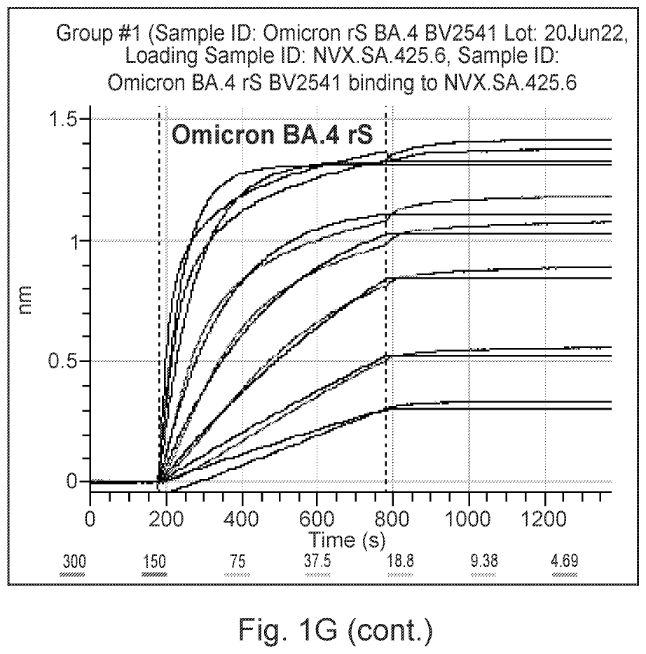

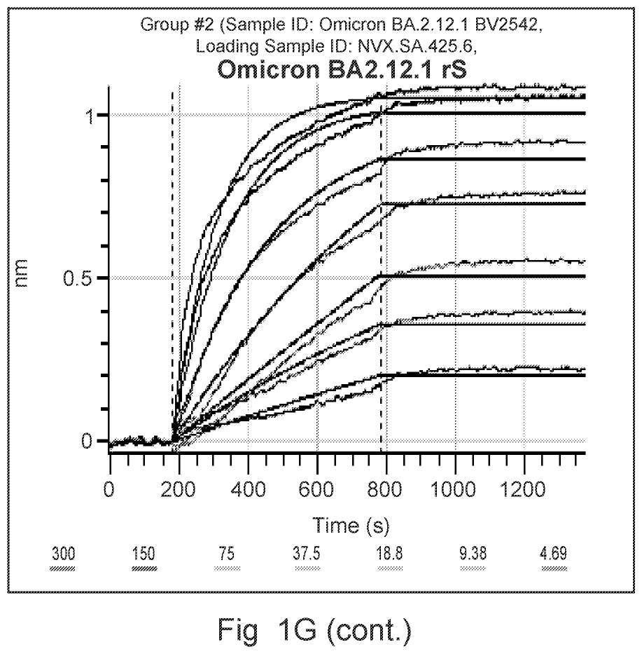

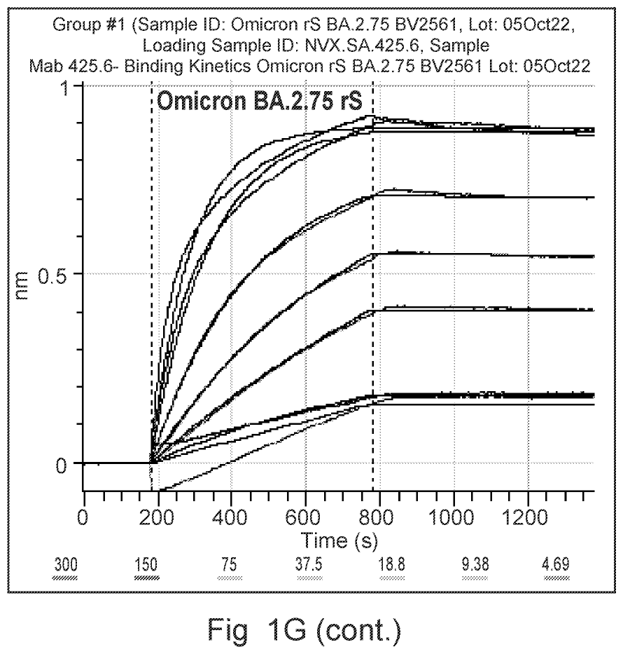

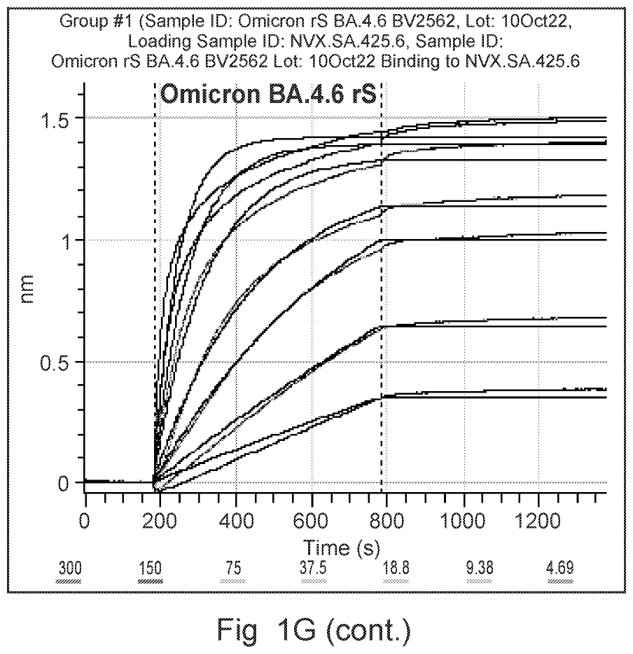

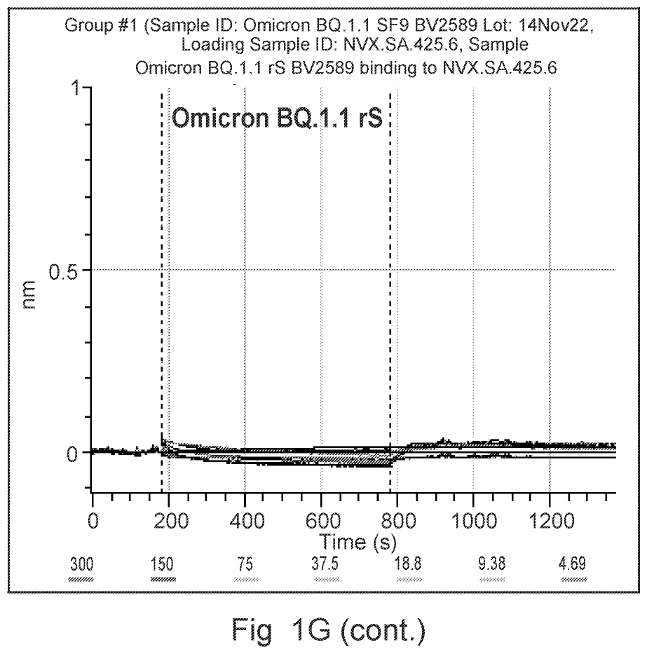

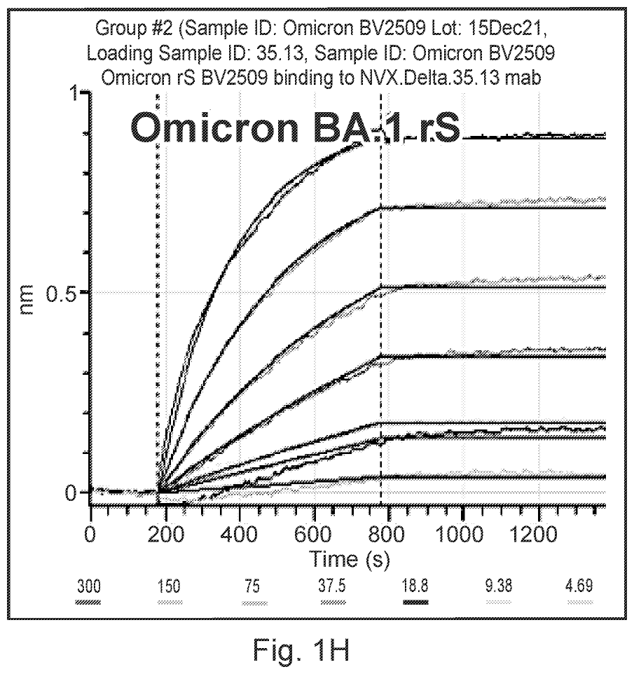

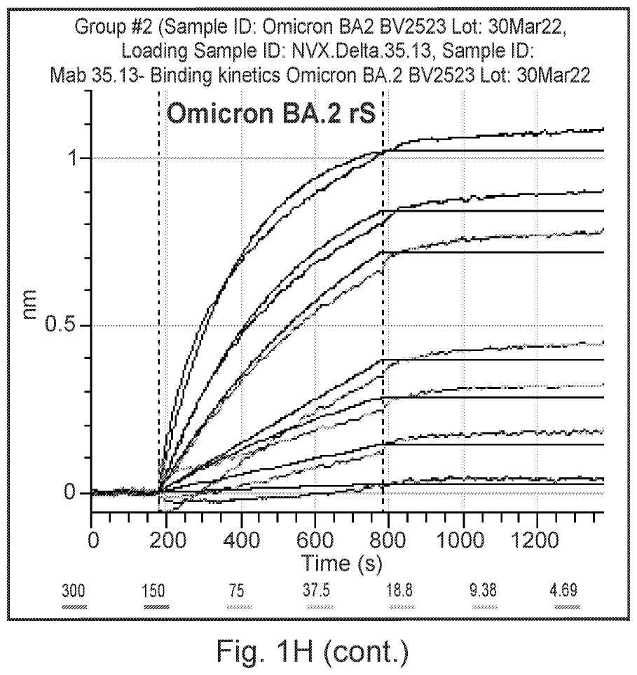

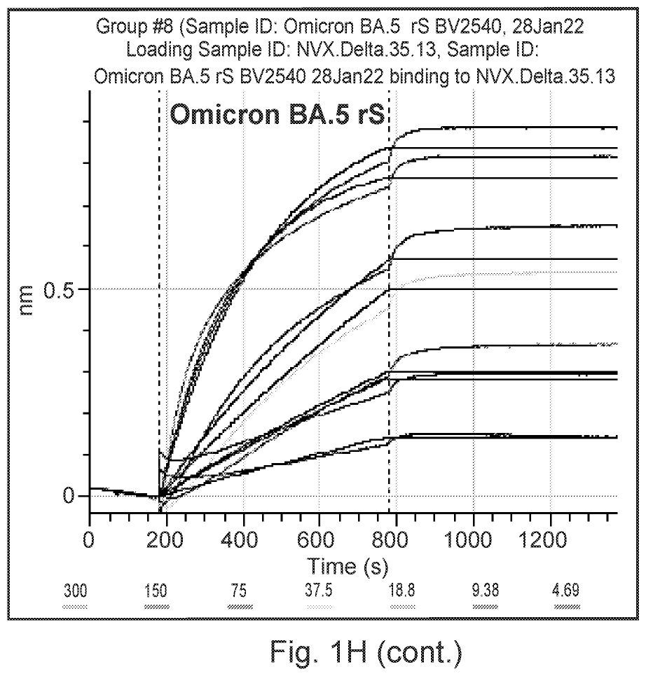

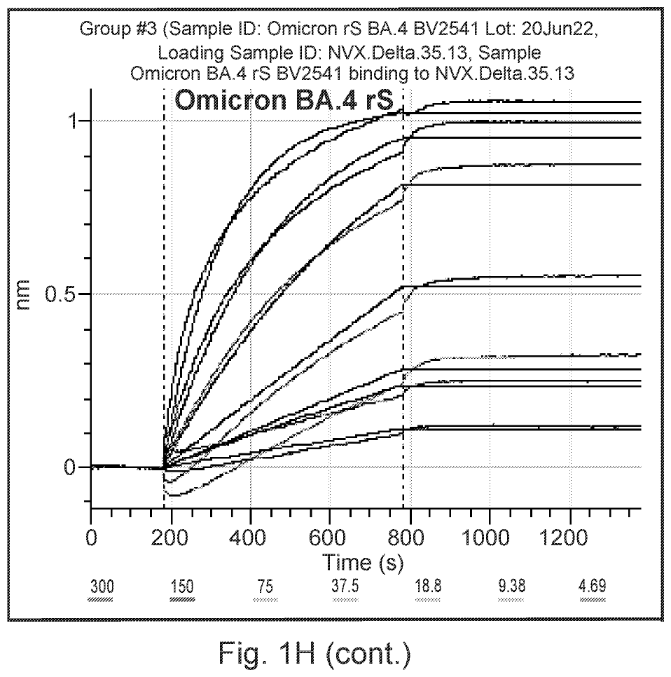

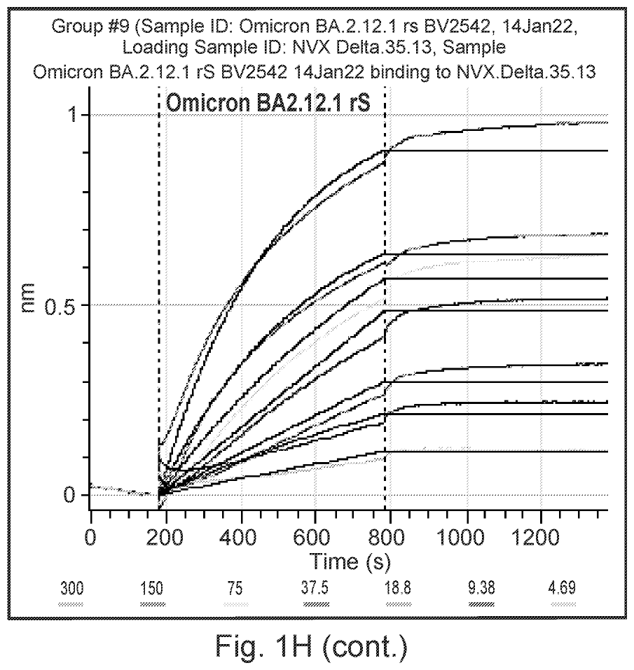

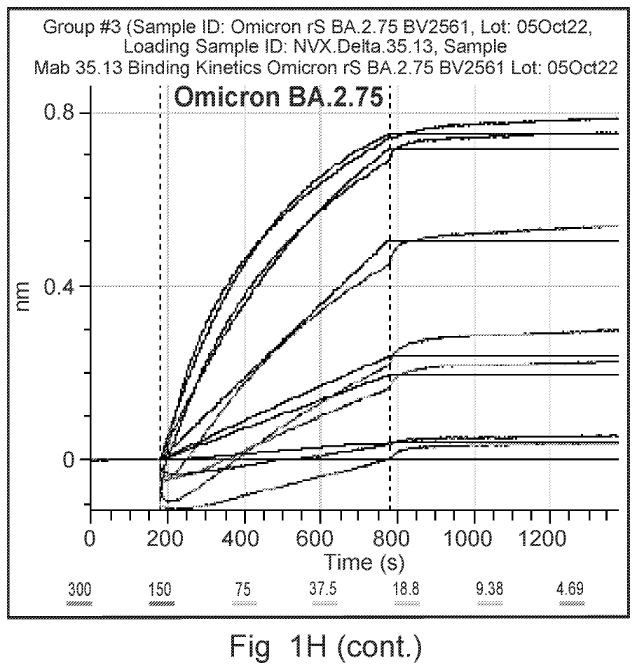

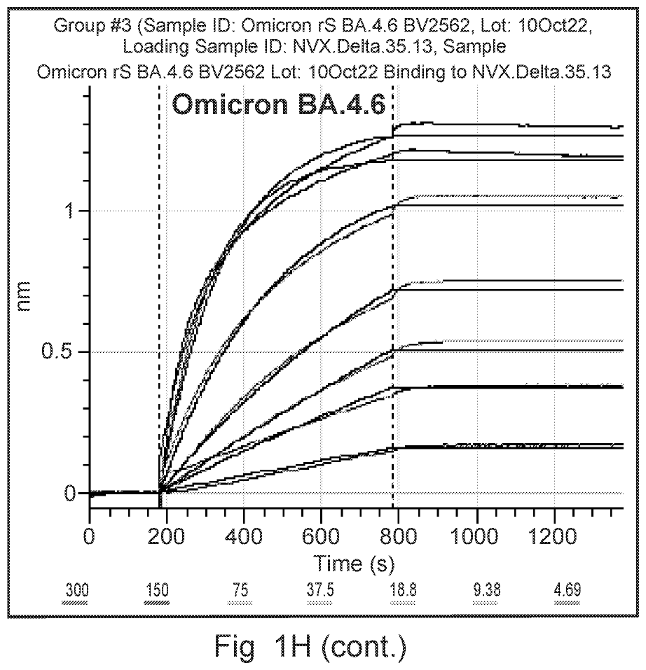

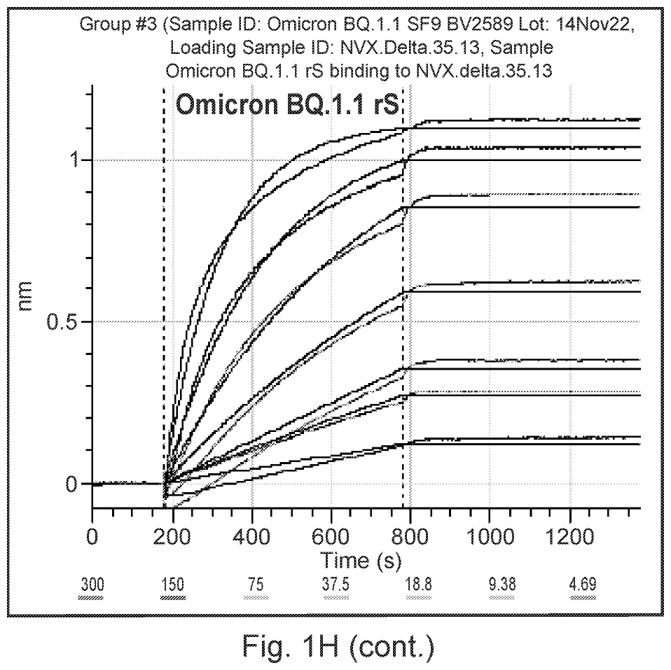

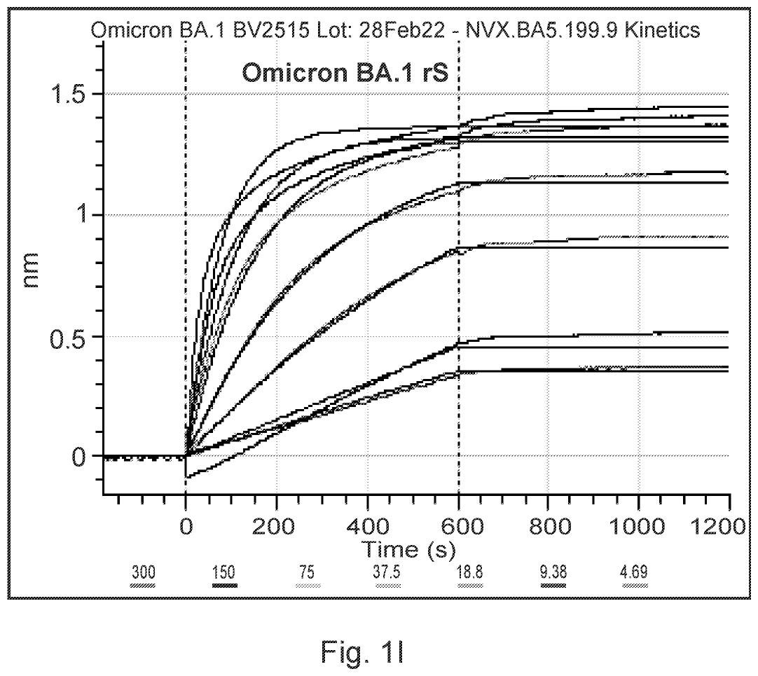

FIGS. 1A-1D shows binding curves of 239.12 (FIG. 1A), 322.3 (FIG. 1B), 425.6 (FIG. 1C), and 35.13 (FIG. 1D) to the CoV S glycoproteins to CoV S glycoproteins related to the SARS-CoV-2 parent strain (SEQ ID NO: 35), the SARS-CoV-2 gamma strain (SEQ ID NO: 38), the SARS-CoV-2 beta strain (SEQ ID NO: 36), the SARS-CoV-2 delta strain (SEQ ID NO: 37), the SARS-CoV-2 alpha strain (SEQ ID NO: 39), and the SARS-CoV-2 omicron strain (SEQ ID NO: 42).

FIGS. 1E-1I shows binding curves of 239.12 (FIG. 1E), 322.3 (FIG. 1F), 425.6 (FIG. 1G), 35.13 (FIG. 1H), and 199.9 (FIG. 1I) to various CoV S glycoproteins related to the SARS-CoV-2 S omicron strain.

TABLE D
Binding Kinetics of Antibodies 239.12, 322.3, 35.13, and 425.6 to CoV S glycoproteins

| | | 239.12 | | | 322.3 | | | 35.13 | | |
|---|---|---|---|---|---|---|---|---|---|---|
| | rS | ka | kd | KD | ka | kd | KD | ka | kd | KD |
| BV # | Variant | (a/Ms) | (1/dis) | (M) | (a/Ms) | (1/dis) | (M) | (a/Ms) | (1/dis) | (M) |
| BV2 | Prototype | 5.29E+04 | 1.79E−07 | 3.81E−12 | 2.72E+03 | 1.29E−07 | 5.71E−11 | 3.3E+04 | 1.67E−07 | 5.29E−12 |
| 373 | | | | | | | | | | |
| (SEQ ID | | | | | | | | | | |
| NO: 35) | | | | | | | | | | |
| BV2 | Alpha | 7.28E+04 | 1.62E−07 | 2.26E−12 | 5.57E+04 | 1.54E−07 | 7.87E−11 | 5.65E+04 | 1.43E−07 | 3.22E−12 |
| 425 | | | | | | | | | | |
| (SEQ ID | | | | | | | | | | |
| NO: 39) | | | | | | | | | | |
| BV2 | Beta | No | No | No | 2.01E+04 | 1.24E−07 | 6.48E−12 | 1.05E+04 | 1.54E−07 | 1.52E−11 |
| 438 | | binding | binding | binding | | | | | | |
| (SEQ ID | | | | | | | | | | |
| NO: 101) | | | | | | | | | | |
| BV2 | Gamma | 2.94E+04 | 1.25E−07 | 4.31E−12 | 1.18E+03 | 1.26E−07 | 1.07E−10 | 9.09E+03 | 1.70E−07 | 1.98E−11 |
| 443 | | | | | | | | | | |
| (SEQ ID | | | | | | | | | | |
| NO: 107) | | | | | | | | | | |
| BV2 | Delta | 4.97E+04 | 1.40E−07 | 3.45E−12 | 3.69E+03 | 1.63E−07 | 7.87E−11 | 4.19E+04 | 1.42E−07 | 3.73E−12 |
| 465 | | | | | | | | | | |
| (SEQ ID | | | | | | | | | | |
| NO: 113) | | | | | | | | | | |
| BV2 | BA.1 | No | No | No | 2.54E+03 | 1.43E−07 | 8.74E−11 | 1.55E+04 | 1.52E−07 | 9.82E−12 |
| 509 | | binding | binding | binding | | | | | | |
| (SEQ ID | | | | | | | | | | |
| NO: 42) | | | | | | | | | | |
| BV2 | BA.2 | No | No | No | 7.45E+03 | 1.78E−07 | 4.05E−11 | 1.91E+04 | 1.61E−07 | 8.43E−12 |
| 523 | | binding | binding | binding | | | | | | |
| (SEQ ID | | | | | | | | | | |
| NO: 118) | | | | | | | | | | |
| BV2 | BA.5 | No | No | No | 1.11E+04 | 1.46E−07 | 1.32E−11 | 1.76E+04 | 1.21E−07 | 7.23E−12 |
| 540 | | binding | binding | binding | | | | | | |
| (SEQ ID | | | | | | | | | | |
| NO: 119) | | | | | | | | | | |
| BV2 | BA.4 | No | No | No | 5.56E+03 | <1.0E−07 | <1.0E−12 | 2.27E+04 | <1.0E−07 | <1.0E−12 |
| 541 | | binding | binding | binding | | | | | | |
| (SEQ ID | | | | | | | | | | |
| NO: 120) | | | | | | | | | | |
| BV2 | BA.2.12.1 | No | No | No | 2.69E+03 | 1.22E−07 | 4.15E−11 | 1.50E+04 | 1.14E−07 | 7.87E−12 |
| 542 | | binding | binding | binding | | | | | | |
| (SEQ ID | | | | | | | | | | |
| NO: 122) | | | | | | | | | | |
| BV2 | BA.2.75 | No | No | No | 5.51E+03 | <1.0E−07 | 4.47E−12 | 8.30E+03 | <1.0E−07 | 6.87E−12 |
| 561 | | binding | binding | binding | | | | | | |
| (SEQ ID | | | | | | | | | | |
| NO: 145) | | | | | | | | | | |
| BV2 | BA.4.6 | No | No | No | 2.10E+04 | <1.0E−07 | <1.0E−12 | 3.85E+04 | <1.0E−07 | <1.0E−12 |
| 562 | | binding | binding | binding | | | | | | |
| (SEQ ID | | | | | | | | | | |
| NO: 146) | | | | | | | | | | |
| BV2 | BQ.1.1 | No | No | No | 1.27E+04 | <1.0E−07 | 1.63E−12 | 2.69E+04 | <1.0E−07 | <1.0E−12 |
| 589 | | binding | binding | binding | | | | | | |
| (SEQ ID | | | | | | | | | | |
| NO: 133) | | | | | | | | | | |

| | | 425.6 | | | 199.9 | | |
|---|---|---|---|---|---|---|---|
| | rS | ka | kd | KD | ka | kd | KD |
| BV # | Variant | (a/Ms) | (1/dis) | (M) | (a/Ms) | (1/dis) | (M) |
| BV2 | Prototype | 1.06E+05 | 1.80E−07 | <1.0E−12 | No | No | No |
| 373 | | | | | binding | binding | binding |
| (SEQ ID | | | | | | | |
| NO: 35) | | | | | | | |
| BV2 | Alpha | 1.23E+05 | 1.84E−07 | <1.0E−12 | * | * | * |
| 425 | | | | | | | |
| (SEQ ID | | | | | | | |
| NO: 39) | | | | | | | |
| BV2 | Beta | 1.19E+05 | 2.08E−07 | <1.0E−12 | * | * | * |
| 438 | | | | | | | |
| (SEQ ID | | | | | | | |
| NO: 101) | | | | | | | |
| BV2 | Gamma | 5.27E+04 | 1.72E−07 | <1.0E−12 | * | * | * |
| 443 | | | | | | | |
| (SEQ ID | | | | | | | |
| NO: 107) | | | | | | | |
| BV2 | Delta | 1.17E+05 | 1.88E−07 | <1.0E−12 | * | * | * |
| 465 | | | | | | | |
| (SEQ ID | | | | | | | |
| NO: 113) | | | | | | | |
| BV2 | BA.1 | 4.13E+04 | 1.54E−07 | 3.91E−12 | 8.04E+04 | <1.0E−07 | <1.0E−12 |
| 509 | | | | | | | |
| (SEQ ID | | | | | | | |
| NO: 42) | | | | | | | |
| BV2 | BA.2 | 3.82E+04 | 1.37E−07 | 3.69E−12 | 5.93E+04 | <1.0E−07 | <1.0E−12 |
| 523 | | | | | | | |
| (SEQ ID | | | | | | | |
| NO: 118) | | | | | | | |
| BV2 | BA.5 | 8.59E+04 | 1.50E−07 | 1.76E−12 | 7.44E+04 | <1.0E−07 | <1.0E−12 |
| 540 | | | | | | | |
| (SEQ ID | | | | | | | |
| NO: 119) | | | | | | | |
| BV2 | BA.4 | 6.29E+04 | <1.0E−07 | <1.0E−12 | * | * | * |
| 541 | | | | | | | |
| (SEQ ID | | | | | | | |
| NO: 120) | | | | | | | |
| BV2 | BA.2.12.1 | 4.02E+04 | 1.51E−07 | 3.81E−12 | 6.00E+04 | <1.0E−07 | <1.0E−12 |
| 542 | | | | | | | |
| (SEQ ID | | | | | | | |
| NO: 122) | | | | | | | |
| BV2 | BA.2.75 | 4.69E+04 | <1.0E−07 | <1.0E−12 | 5.09E+04 | <1.0E−07 | <1.0E−12 |
| 561 | | | | | | | |
| (SEQ ID | | | | | | | |
| NO: 145) | | | | | | | |
| BV2 | BA.4.6 | 9.83E+04 | <1.0E−07 | <1.0E−12 | 8.42E+04 | <1.0E−07 | <1.0E−12 |
| 562 | | | | | | | |
| (SEQ ID | | | | | | | |
| NO: 146) | | | | | | | |
| BV2 | BQ.1.1 | No | No | No | 7.65E+04 | <1.0E−07 | <1.0E−12 |
| 589 | | binding | binding | binding | | | |
| (SEQ ID | | | | | | | |
| NO: 133) | | | | | | | |

* Not evaluated

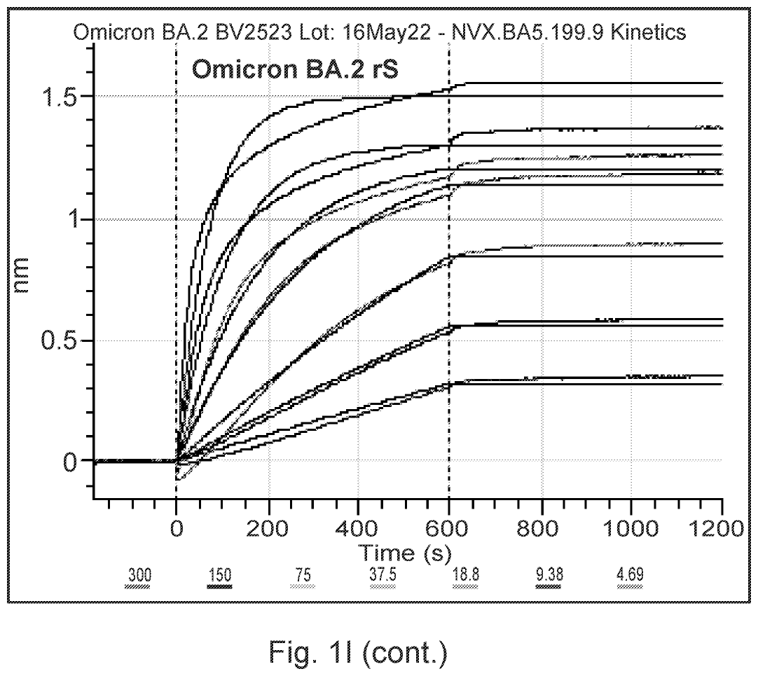

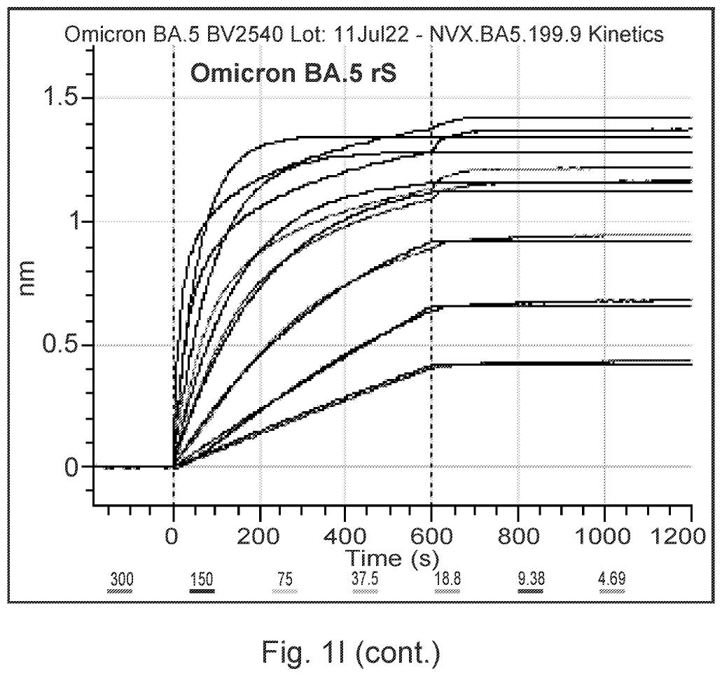

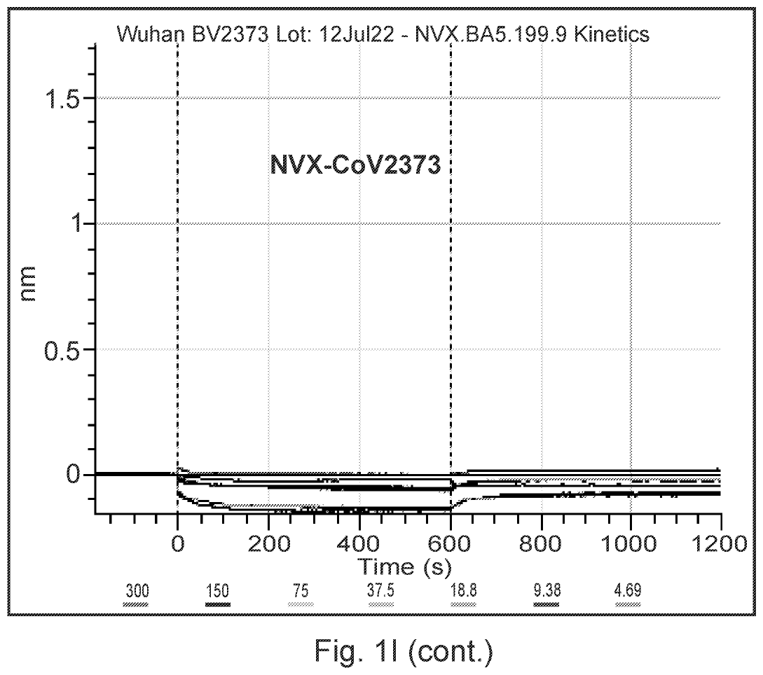

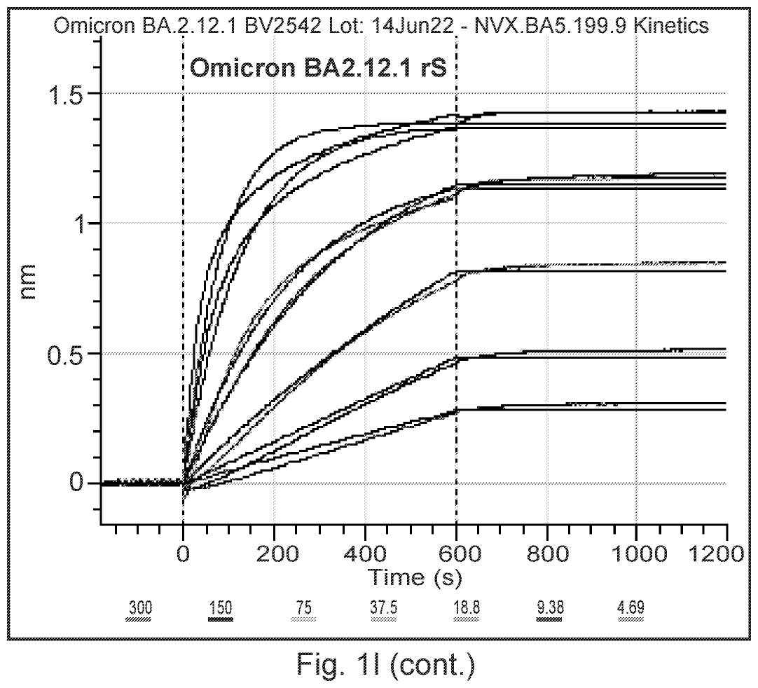

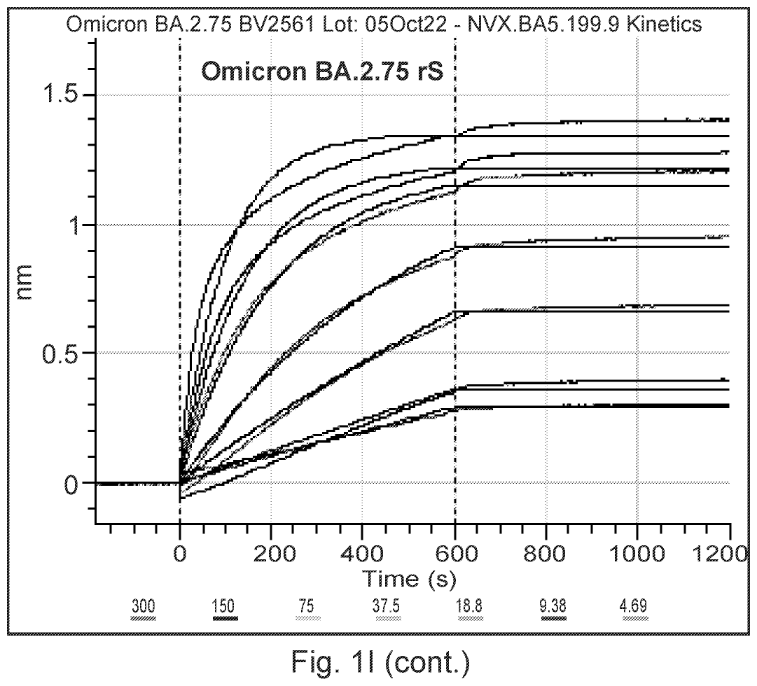

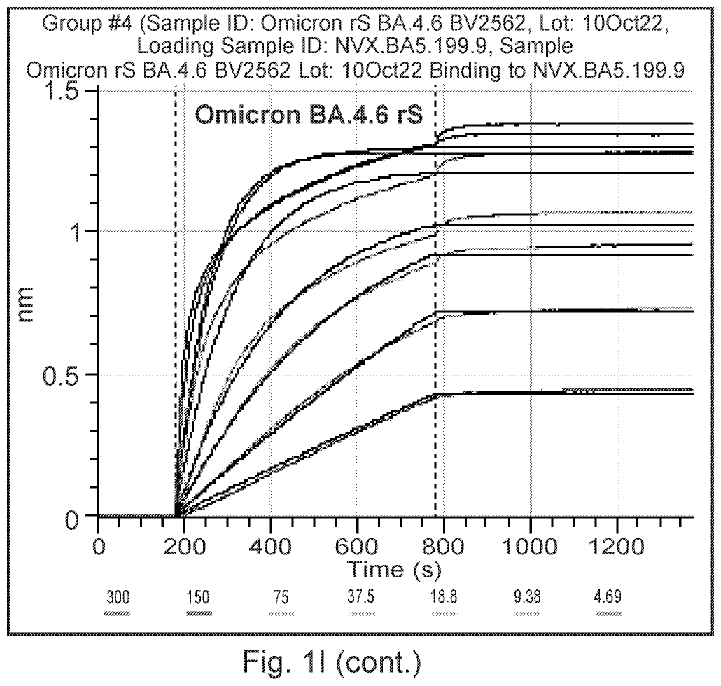

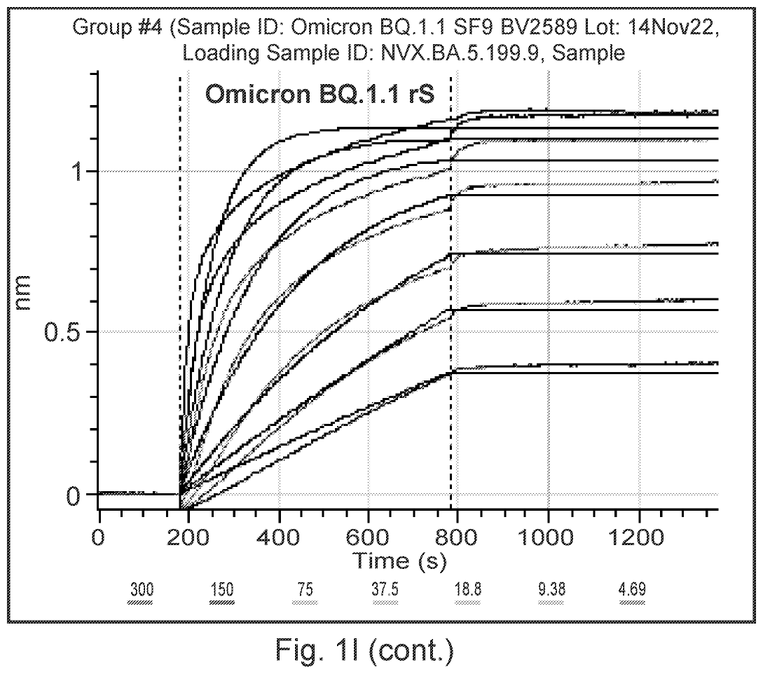

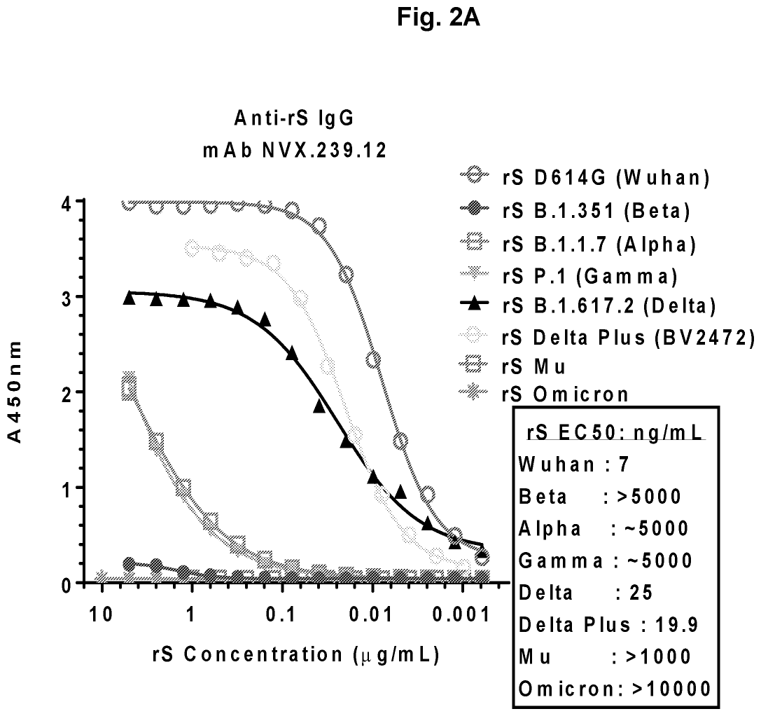

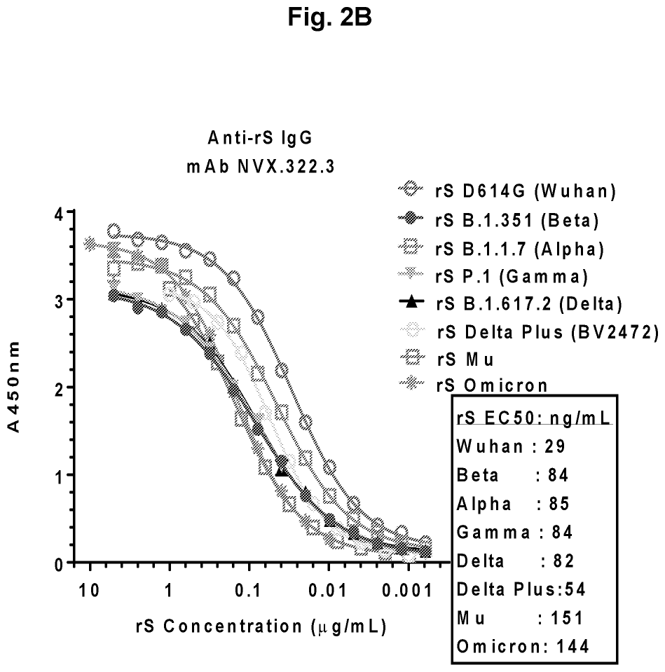

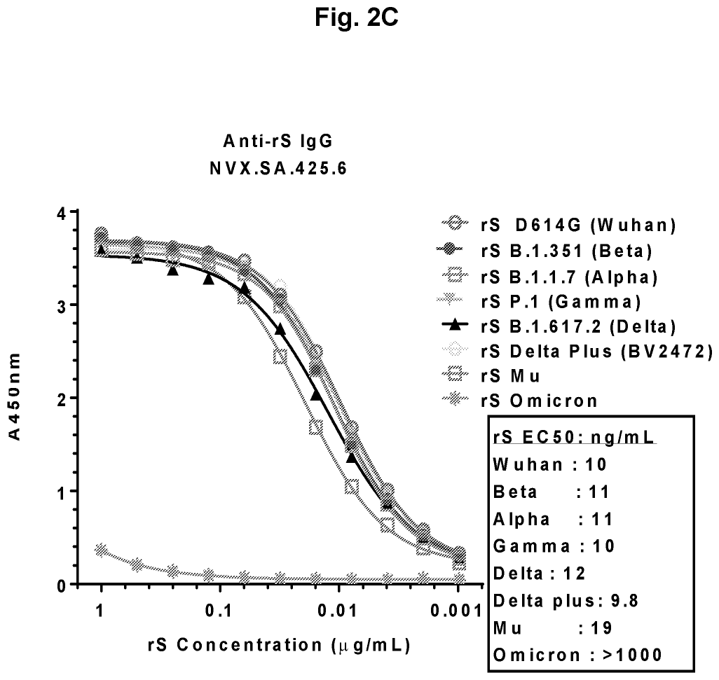

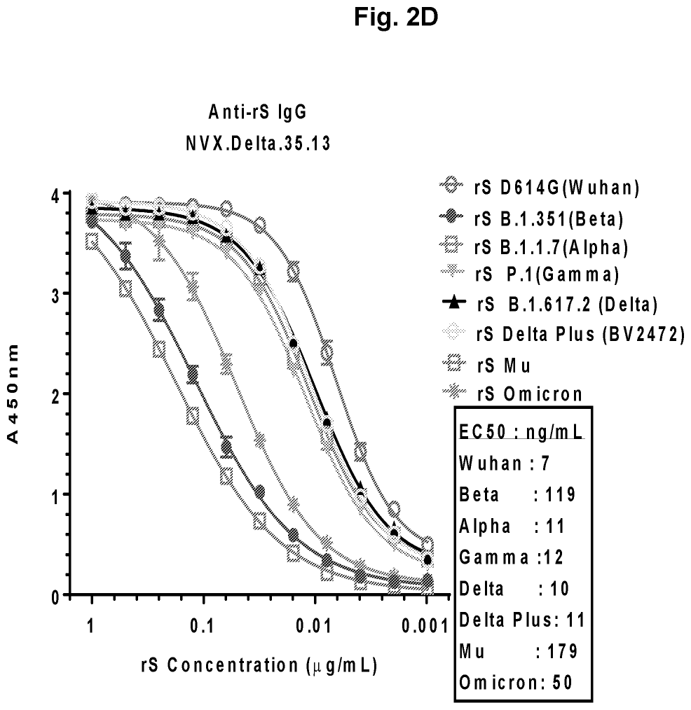

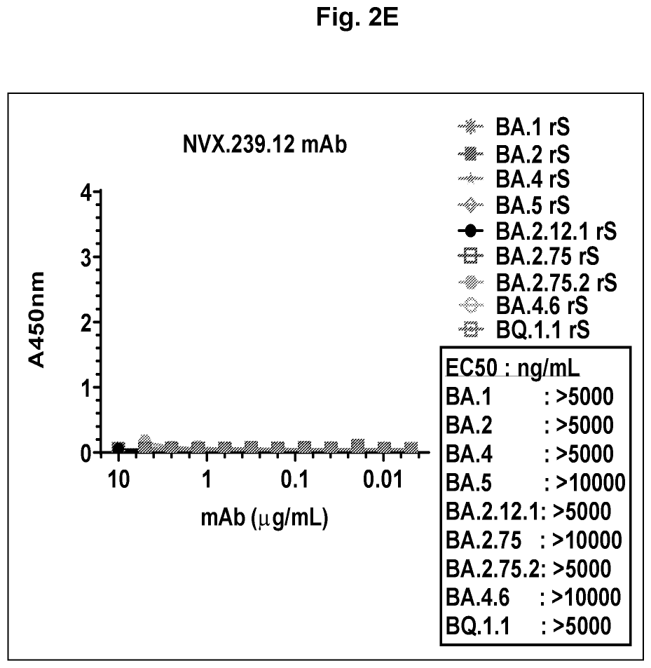

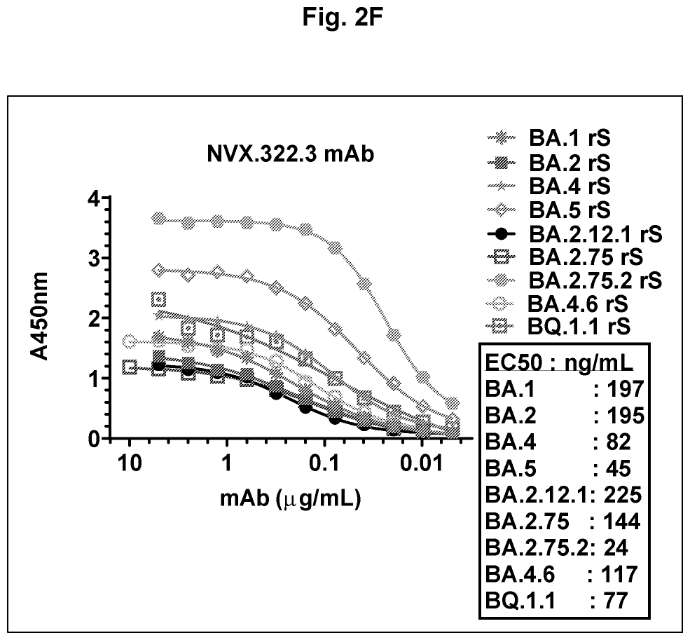

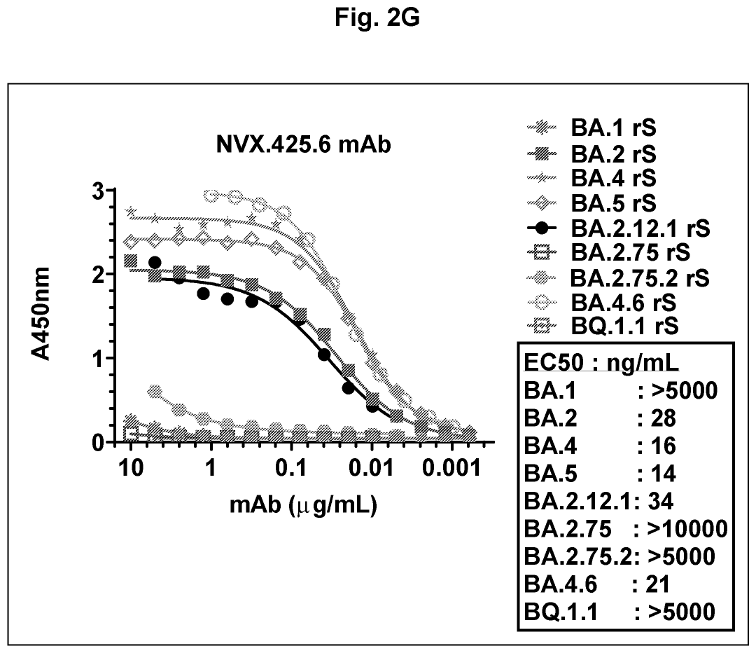

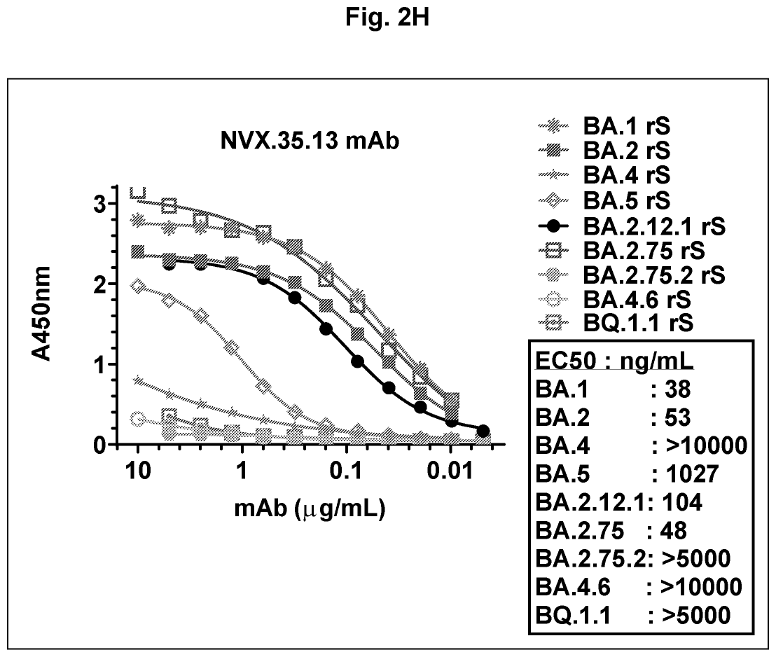

Results—ELISA: FIGS. 2A-2D show the EC50 of binding of 239.12 (FIG. 2A), 322.3 (FIG. 2B), 425.6 (FIG. 2C), 35.13 (FIG. 2D) to various CoV S glycoproteins. FIGS. 2E-2I show the EC50 of binding of of 239.12 (FIG. 2E), 322.3 (FIG. 2F), 425.6 (FIG. 2G), 35.13 (FIG. 2I), and 199.9 (FIG. 2I)to various CoV S glycoproteins related to the SARS-CoV-2 S omicron strain. Table E shows EC50s for binding of 35.13 to CoV S glycoproteins. Table F1 show the EC50s (ng/mL) of antibody binding (239.12, 322.3, 426.7, and 35.13) to CoV S glycoproteins derived from Omicron strains. Table F2 shows the EC50s (ng/mL) for binding of 199.9 to CoV S glycoproteins.

TABLE E
EC50 (ng/ml) of binding between Omicron related
CoV S glycoproteins and Antibodies

| | EC50 |
|---|---|
| SARS-CoV-2 S protein | (ng/mL) |
| Wuhan | 7 |
| Beta-B.1.351 | 119 |
| Alpha B.1.1.7 | 11 |
| Gamma P.1 | 12 |
| Delta B.1.617.2 | 10 |
| Delta Plus | 11 |
| Mu | 179 |
| Omicron | 50 |

TABLE F1
EC50 (ng/ml) of binding between Omicron related
CoV S glycoproteins and Antibodies

| SARS-CoV-2 | Anti-rS ELISA (EC50: ng/mL) | | | |
|---|---|---|---|---|
| Variant rS | 239.12 | 322.3 | 425.6 | 35.13 |
| BA.1 | >5000 | 197 | >5000 | 38 |
| BA.2 | >5000 | 195 | 28 | 53 |
| BA.4 | >5000 | 82 | 16 | >10000 |
| BA.5 | >10000 | 45 | 14 | 1027 |
| BA.2.12.1 | >5000 | 225 | 34 | 104 |
| BA.2.75 | >10000 | 144 | >10000 | 48 |
| BA.2.75.2 | >5000 | 24 | >5000 | >5000 |
| BA.4.6 | >10000 | 117 | 21 | >10000 |
| BQ.1.1 | >5000 | 77 | >5000 | >5000 |
| BQ.1 | >5000 | 85 | >5000 | >5000 |
| XBB | >5000 | 111 | >5000 | >5000 |

TABLE F2
EC50 (ng/ml) of binding between SARS-CoV-2 S
polypeptides and 199.9

| Strain CoV S glycoprotein is | Anti-rS ELISA |
|---|---|
| Related To | (EC50: ng/mL) |
| Parent | >5000 |
| Alpha | n.d. |
| Beta | n.d. |
| Gamma | n.d. |
| Delta | >5000 |
| BA.1 | ~5000 |
| BA.2 | ~5000 |
| BA.4 | 16 |
| BA.5 | 20 |
| BA.2.12.1 | >10000 |
| BA.2.75 | >10000 |
| BA.2.75.2 | 1 |
| BA.4.6 | 21 |
| BQ.1.1 | 16 |
| XBB | 3867 |

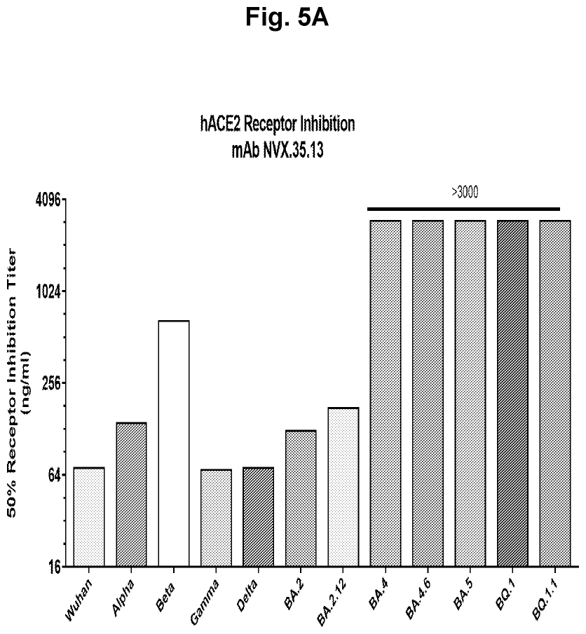

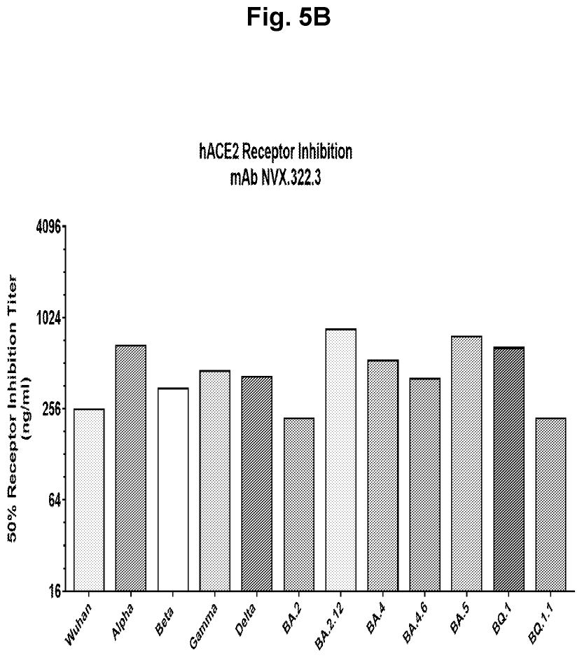

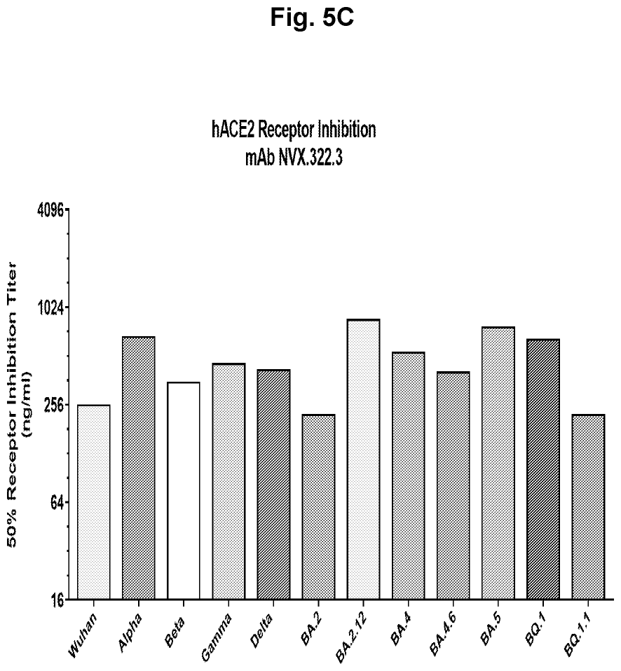

Results—hACE2 Receptor Inhibition: Table G shows the concentration of 35.13 which inhibits 50% of the interaction between hACE2 and the CoV S glycoprotein (IC50). Table H1 shows the concentrations of antibodies which inhibit 50% of the interaction between hACE2 and an omicron-related CoV S glycoprotein (IC50). Table H2 shows the concentrations of 199.9 which inhibit 50% of the interaction between hACE2 and a CoV S glycoprotein (IC50). FIGS. 5A-5C show the hACE2 receptor inhibition of 35.13 (FIG. 5A), 425.6 (FIG. 5B), and 322.3 (FIG. 5C).

TABLE G
IC50 of 35.13 and 425.6 for interaction between hACE2
and CoV S glycoproteins

| | 35.13 | 425.6 |
|---|---|---|
| | IC50 for interaction | IC50 for interaction |
| | between hACE2 and | between hACE2 and |
| CoV S | SARS-CoV-2 S protein | SARS-CoV-2 S protein |
| glycoproteins | (ng/mL) | (ng/mL) |
| Wuhan | 74 | 64 |
| Beta-B.1.351 | 922 | 48 |
| Alpha B.1.1.7 | 142 | 100 |
| Gamma P.1 | 70 | 48 |
| Kappa B.1.617.1 | * | 43 |
| Delta B.1.617.2 | 98 | 69 |
| Delta Plus | 65 | * |
| Mu | 982 | * |
| Omicron | 102 | >3000 |

* Not determined

TABLE H1
IC50 of Antibodies for interaction between hACE2 and
Omicron Related CoV S glycoproteins

| Strain CoV S | | | | |
|---|---|---|---|---|
| glycoprotein is | 50% Receptor Inhibition (IC50: ng/mL) | | | |
| Derived From | 239.12 | 322.3 | 425.6 | 35.13 |
| BA.1 | >3000 | 493 | >3000 | 97 |
| BA.2 | >3000 | 224 | 32 | 126 |
| BA.4 | >3000 | 543 | 37 | >5000 |
| BA.5 | >5000 | 779 | 28 | >3000 |
| BA.2.12.1 | >3000 | 866 | 28 | 177 |
| BA.2.75 | >5000 | >5000 | >5000 | 169 |
| BA.2.75.2 | >3000 | 184 | >3000 | >3000 |
| BA.4.6 | >5000 | 411 | 71 | >5000 |
| BQ.1.1 | >3000 | 265 | >3000 | >3000 |
| BQ.1 | >3000 | 656 | >3000 | >3000 |
| XBB | >3000 | 130 | >3000 | >3000 |

TABLE H2
IC50 of 199.9 for interaction between hACE2 and
Omicron Related CoV S glycoproteins

| | 50% Receptor Inhibition |
|---|---|
| CoV S glycoproteins | (IC50: ng/mL) |
| Parent | >3000 |
| Alpha | n.d. |
| Beta | n.d. |
| Gamma | n.d. |
| Delta | >3000 |
| BA.1 | 2106 |
| BA.2 | 676 |
| BA.4 | 38 |
| BA.5 | 28 |
| BA.2.12.1 | 2114 |
| BA.2.75 | 411 |
| BA.2.75.2 | 648 |
| BA.4.6 | 56 |
| BQ.1.1 | 32 |
| XBB | 3778 |

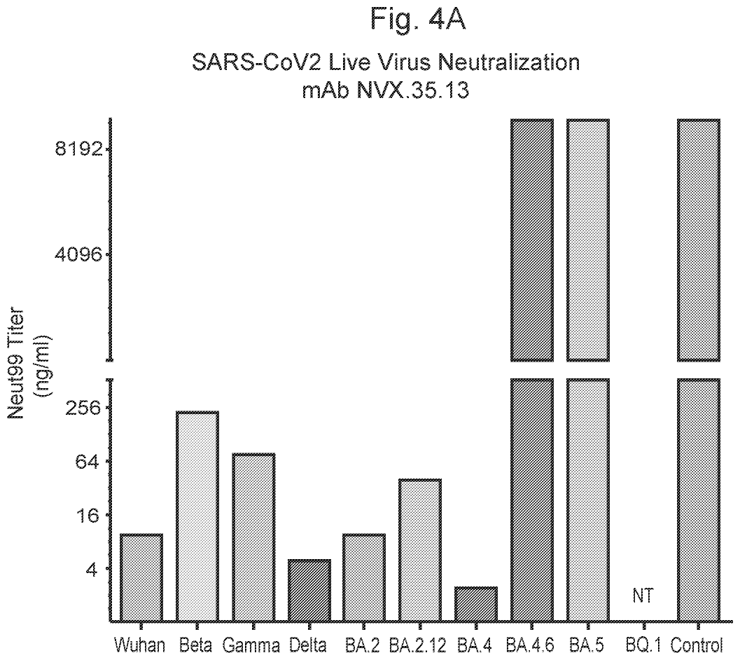

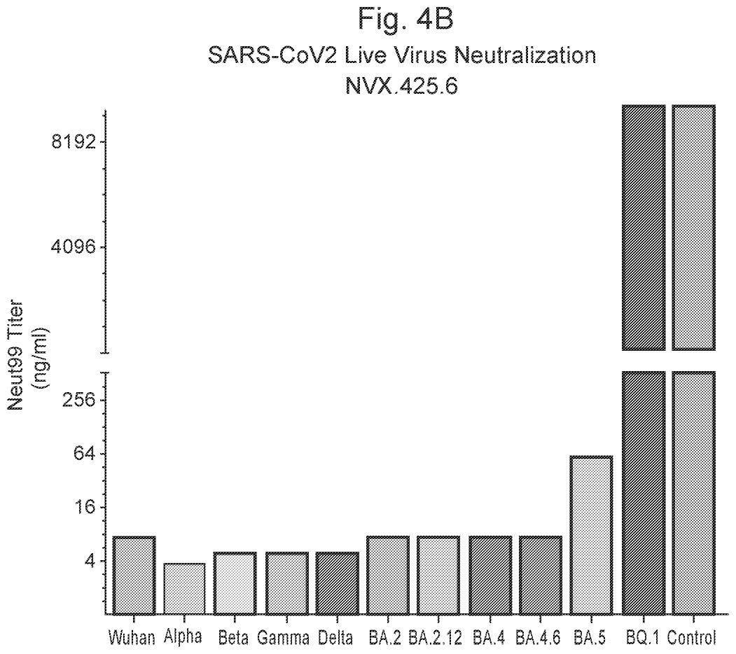

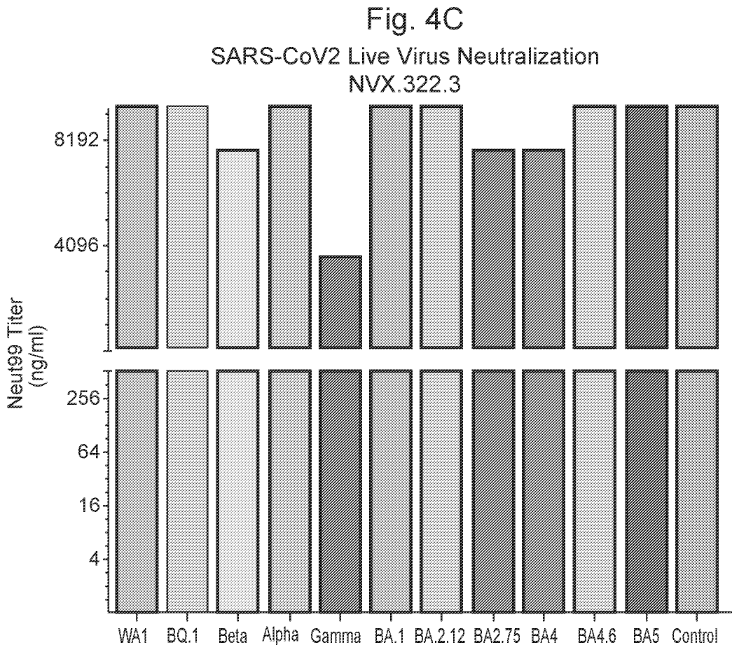

Results—Live SARS-CoV-2 Neutralization Assay: SARS-CoV-2 Neutralization Assay: 35.13 neutralized the parental SARS-CoV-2 as well as variant strains up to Omicron BA.4. 35.13 did not neutralize BA.4.6 or BA.5 in this assay. 425.6 exhibited potent neutralization activity against all variants tested except against Omicron BQ.1.1. FIGS. 4A-4C shows the minimum sample dilution of 35.13 (FIG. 4A), 425.6 (FIG. 4B), and 322.3 (FIG. 4C) required to neutralize greater than 99% of the concentration of SARS-CoV-2 tested (Neut99).

Table I shows the the concentration at which 50% of a SARS-CoV-2 virus is neutralized by 425.6. “N.D.” means the data point has not yet been collected.

TABLE I
IC50 of 425.6 for interaction between hACE2 and CoV S glycoprotein
and Concentration at which 50% of SARS-CoV-2 is Neutralized

| | 425.6 | 35.13 |
|---|---|---|
| | 50% SARS-COV-2 | 50% SARS-COV-2 |
| CoV S glycoprotein | Neutralization (ng/ml) | Neutralization (ng/mL) |
| Wuhan-WA | 24.5 | N.D. |
| Beta B.1.351 | 5.31 | N.D. |
| Alpha B.1.1.7 | 2.96 | N.D. |
| Gamma P.1 | 7.22 | N.D. |
| Kappa B.1.617.1 | 3.0 | N.D. |
| Delta B.1.617.2 | N.D. | N.D. |
| Omicron BA.1 | 210.51 | 7.32 |
| Omicron BA.2 | 2.38 | 7.32 |
| Omicron BA.4 | 1.23 | 2.44 |
| Omicron BA.5 | 4.27 | N.D. |
| Omicron BA.2.12.1 | 2.27 | 2.44 |

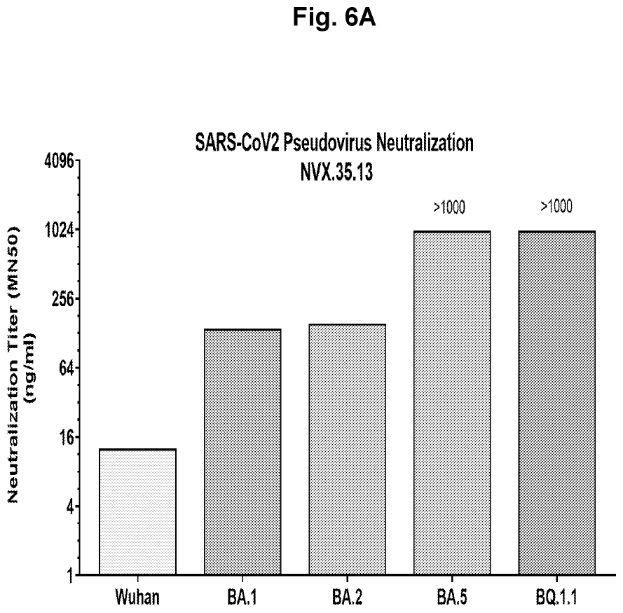

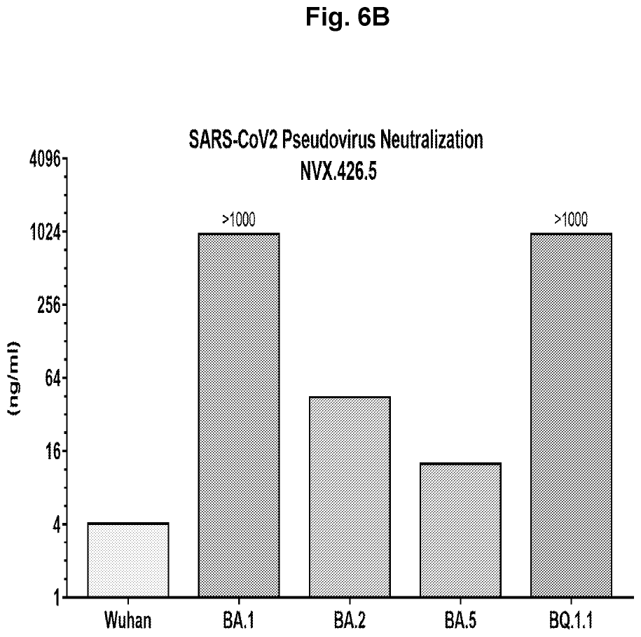

Results—Pseudovirus Neutralization: FIGS. 6A-6B show pseudovirus neutralization by antibodies 35.13 (FIG. 6A) and 425.6 (FIG. 6B).

TABLE J
CoV S glycoproteins polypeptides used to evaluate binding kinetics

| CoV S | | SEQ ID |
|---|---|---|
| glycoproteins | Amino Acid Sequence | NO: |
| BV2373 | QCVNLTTRTQLPPAYTNSFTRGVYYPDKVFRSSVLHS | 35 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2438 | QCVNLTTRTQLPPAYTNSFTRGVYYPDKVFRSSVLHS | 36 |
| (also called | TQDLFLPFFSNVTWFHAIHVSGTNGTKRFANPVLPFN | |
| BV2426) | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRGLPQGFSALEPLVDLPIGINIT | |
| | RFQTLHALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGNIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGVENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGNFSQILPDPSKPSKRSFIEDLLFNKVTL | |
| | ADAGFIKQYGDCLGDIAARDLICAQKFNGLTVLPPLL | |
| | TDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2465 | QCVNLRTRTQLPPAYTNSFTRGVYYPDKVFRSSVLHS | 37 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLDVYYHKNNKSWMESGVYSS | |
| | ANNCTFEYVSQPFLMDLEGKQGNFKNLREFVFKNID | |
| | GYFKIYSKHTPINLVRDLPQGFSALEPLVDLPIGINITR | |
| | FQTLLALHRSYLTPGDSSSGWTAGAAAYYVGYLQPR | |
| | TFLLKYNENGTITDAVDCALDPLSETKCTLKSFTVEK | |
| | GIYQTSNFRVQPTESIVRFPNITNLCPFGEVENATRFAS | |
| | VYAWNRKRISNCVADYSVLYNSASFSTFKCYGVSPT | |
| | KLNDLCFTNVYADSFVIRGDEVRQIAPGQTGKIADYN | |
| | YKLPDDFTGCVIAWNSNNLDSKVGGNYNYRYRLFR | |
| | KSNLKPFERDISTEIYQAGSKPCNGVEGENCYFPLQSY | |
| | GFQPTNGVGYQPYRVVVLSFELLHAPATVCGPKKST | |
| | NLVKNKCVNFNFNGLTGTGVLTESNKKFLPFQQFGR | |
| | DIADTTDAVRDPQTLEILDITPCSFGGVSVITPGTNTSN | |
| | QVAVLYQGVNCTEVPVAIHADQLTPTWRVYSTGSN | |
| | VFQTRAGCLIGAEHVNNSYECDIPIGAGICASYQTQT | |
| | NSRQQAQSVASQSIIAYTMSLGAENSVAYSNNSIAIPT | |
| | NFTISVTTEILPVSMTKTSVDCTMYICGDSTECSNLLL | |
| | QYGSFCTQLNRALTGIAVEQDKNTQEVFAQVKQIYK | |
| | TPPIKDFGGFNFSQILPDPSKPSKRSFIEDLLFNKVTLA | |
| | DAGFIKQYGDCLGDIAARDLICAQKFNGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQNVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2443 | QCVNFTNRTQLPSAYTNSFTRGVYYPDKVFRSSVLHS | 38 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNYPFLGVYYHKNNKSWMESEFRVYS | |
| | SANNCTFEYVSQPFLMDLEGKQGNFKNLSEFVFKNID | |
| | GYFKIYSKHTPINLVRDLPQGFSALEPLVDLPIGINITR | |
| | FQTLLALHRSYLTPGDSSSGWTAGAAAYYVGYLQPR | |
| | TFLLKYNENGTITDAVDCALDPLSETKCTLKSFTVEK | |
| | GIYQTSNFRVQPTESIVRFPNITNLCPFGEVENATRFAS | |
| | VYAWNRKRISNCVADYSVLYNSASFSTFKCYGVSPT | |
| | KLNDLCFTNVYADSFVIRGDEVRQIAPGQTGTIADYN | |
| | YKLPDDFTGCVIAWNSNNLDSKVGGNYNYLYRLFR | |
| | KSNLKPFERDISTEIYQAGSTPCNGVKGFNCYFPLQSY | |
| | GFQPTYGVGYQPYRVVVLSFELLHAPATVCGPKKST | |
| | NLVKNKCVNFNFNGLTGTGVLTESNKKFLPFQQFGR | |
| | DIADTTDAVRDPQTLEILDITPCSFGGVSVITPGTNTSN | |
| | QVAVLYQGVNCTEVPVAIHADQLTPTWRVYSTGSN | |
| | VFQTRAGCLIGAEYVNNSYECDIPIGAGICASYQTQT | |
| | NSPQQAQSVASQSIIAYTMSLGAENSVAYSNNSIAIPT | |
| | NFTISVTTEILPVSMTKTSVDCTMYICGDSTECSNLLL | |
| | QYGSFCTQLNRALTGIAVEQDKNTQEVFAQVKQIYK | |
| | TPPIKDFGGFNFSQILPDPSKPSKRSFIEDLLFNKVTLA | |
| | DAGFIKQYGDCLGDIAARDLICAQKFNGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQDVVNQNAQALNTLVKQLSSNFGAISS | |
| | VLNDILSRLDPPEAEVQIDRLITGRLQSLQTYVTQQLI | |
| | RAAEIRASANLAAIKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASFVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2425 | QCVNLTTRTQLPPAYTNSFTRGVYYPDKVFRSSVLHS | 39 |
| | TQDLFLPFFSNVTWFHAISGTNGTKRFDNPVLPFNDG | |
| | VYFASTEKSNIIRGWIFGTTLDSKTQSLLIVNNATNVV | |
| | IKVCEFQFCNDPFLGVYHKNNKSWMESEFRVYSSAN | |
| | NCTFEYVSQPFLMDLEGKQGNFKNLREFVFKNIDGY | |
| | FKIYSKHTPINLVRDLPQGFSALEPLVDLPIGINITRFQ | |
| | TLLALHRSYLTPGDSSSGWTAGAAAYYVGYLQPRTF | |
| | LLKYNENGTITDAVDCALDPLSETKCTLKSFTVEKGI | |
| | YQTSNFRVQPTESIVRFPNITNLCPFGEVENATRFASV | |
| | YAWNRKRISNCVADYSVLYNSASFSTFKCYGVSPTK | |
| | LNDLCFTNVYADSFVIRGDEVRQIAPGQTGKIADYNY | |
| | KLPDDFTGCVIAWNSNNLDSKVGGNYNYLYRLFRKS | |
| | NLKPFERDISTEIYQAGSTPCNGVEGENCYFPLQSYGF | |
| | QPTYGVGYQPYRVVVLSFELLHAPATVCGPKKSTNL | |
| | VKNKCVNFNFNGLTGTGVLTESNKKFLPFQQFGRDI | |
| | DDTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEHVNNSYECDIPIGAGICASYQTQTNS | |
| | HQQAQSVASQSIIAYTMSLGAENSVAYSNNSIAIPINF | |
| | TISVTTEILPVSMTKTSVDCTMYICGDSTECSNLLLQY | |
| | GSFCTQLNRALTGIAVEQDKNTQEVFAQVKQIYKTPP | |
| | IKDFGGFNFSQILPDPSKPSKRSFIEDLLFNKVTLADA | |
| | GFIKQYGDCLGDIAARDLICAQKENGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNQNAQALNTLVKQLSSNFGAISSVLN | |
| | DILARLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTHNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2472 | QCVNLRTRTQLPPAYTNSFTRGVYYPDKVFRSSVLHS | 40 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLDVYYHKNNKSWMESGVYSS | |
| | ANNCTFEYVSQPFLMDLEGKQGNFKNLREFVFKNID | |
| | GYFKIYSKHTPINLVRDLPQGFSALEPLVDLPIGINITR | |
| | FQTLLALHRSYLTPGDSSSGITAGAAAYYVGYLQPRT | |
| | FLLKYNENGTITDAVDCALDPLSETKCTLKSFTVEKG | |
| | IYQTSNFRVQPTESIVRFPNITNLCPFGEVFNATRFASV | |
| | YAWNRKRISNCVADYSVLYNSASFSTFKCYGVSPTK | |
| | LNDLCFTNVYADSFVIRGDEVRQIAPGQTGNIADYNY | |
| | KLPDDFTGCVIAWNSNNLDSKVGGNYNYRYRLFRKS | |
| | NLKPFERDISTEIYQAGSKPCNGVEGENCYFPLQSYGF | |
| | QPTNGVGYQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEHVNNSYECDIPIGAGICASYQTQTNS | |
| | RQQAQSVASQSIIAYTMSLGAENSVAYSNNSIAIPTNF | |
| | TISVTTEILPVSMTKTSVDCTMYICGDSTECSNLLLQY | |
| | GSFCTQLNRALTGIAVEQDKNTQEVFAQVKQIYKTPP | |
| | IKDFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQNVVNQNAQALNTLVKQLSSNFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2508 | QCVNLTTRTQLPPAYTNSFTRGVYYPDKVFRSSVLHS | 41 |
| | TQDLFLPFFSNVTWFHVIHVSGTNGTKRFDNPVLPFN | |
| | DGVYFASIEKSNIIRGWIFGTTLDSKTQSLLIVNNATN | |
| | VVIKVCEFQFCNDPFLDVYYHKNNKSWMESEFRVYS | |
| | SANNCTFEYVSQPFLMDLEGKQGNFKNLREFVFKNI | |
| | DGYFKIYSKHTPINIVRDLPQGFSALEPLVDLPIGINIT | |
| | RFQTLLALHRSYLPGDSSSGWTAGAAAYYVGYLQPR | |
| | TFLLKYNENGTITDAVDCALDPLSETKCTLKSFTVEK | |
| | GIYQTSNFRVQPTESIVRFPNITNLCPFDEVENATRFAS | |
| | VYAWNRKRISNCVADYSVLYNLAPFFTFKCYGVSPT | |
| | KLNDLCFTNVYADSFVIRGDEVRQIAPGQTGNIADYN | |
| | YKLPDDFTGCVIAWNSNKLDSKVSGNYNYLYRLFRK | |
| | SNLKPFERDISTEIYQAGNKPCNGVAGENCYFPLRSY | |
| | SFRPTYGVGHQPYRVVVLSFELLHAPATVCGPKKST | |
| | NLVKNKCVNFNFNGLKGTGVLTESNKKFLPFQQFGR | |
| | DIADTTDAVRDPQTLEILDITPCSFGGVSVITPGTNTSN | |
| | QVAVLYQGVNCTEVPVAIHADQLTPTWRVYSTGSN | |
| | VFQTRAGCLIGAEYVNNSYECDIPIGAGICASYQTQT | |
| | KSHQQAQSVASQSIIAYTMSLGAENSVAYSNNSIAIPT | |
| | NFTISVTTEILPVSMTKTSVDCTMYICGDSTECSNLLL | |
| | QYGSFCTQLKRALTGIAVEQDKNTQEVFAQVKQIYK | |
| | TPPIKYFGGFNFSQILPDPSKPSKRSFIEDLLFNKVTLA | |
| | DAGFIKQYGDCLGDIAARDLICAQKFKGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQDVVNHNAQALNTLVKQLSSKFGAISS | |
| | VLNDIFSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKELDKYFKNHT | |
| | SPDVDLGDISGINASVVNIQKEIDRLNEVAKNLNESLI | |
| | DLQELGKYEQYIKWPWYIWLGFIAGLIAIVMVTIMLC | |
| | CMTSCCSCLKGCCSCGSCCKFDEDDSEPVLKGVKLH | |
| | YT | |
| BV2509 | QCVNLTTRTQLPPAYTNSFTRGVYYPDKVFRSSVLHS | 42 |
| | TQDLFLPFFSNVTWFHVISGTNGTKRFDNPVLPFNDG | |
| | VYFASIEKSNIIRGWIFGTTLDSKTQSLLIVNNATNVVI | |
| | KVCEFQFCNDPFLDHKNNKSWMESEFRVYSSANNCT | |
| | FEYVSQPFLMDLEGKQGNFKNLREFVFKNIDGYFKIY | |
| | SKHTPIIVREPEDLPQGFSALEPLVDLPIGINITRFQTLL | |
| | ALHRSYLTPGDSSSGWTAGAAAYYVGYLQPRTFLLK | |
| | YNENGTITDAVDCALDPLSETKCTLKSFTVEKGIYQT | |
| | SNFRVQPTESIVRFPNITNLCPFDEVFNATRFASVYAW | |
| | NRKRISNCVADYSVLYNLAPFFTFKCYGVSPTKLNDL | |
| | CFTNVYADSFVIRGDEVRQIAPGQTGNIADYNYKLPD | |
| | DFTGCVIAWNSNKLDSKVSGNYNYLYRLFRKSNLKP | |
| | FERDISTEIYQAGNKPCNGVAGENCYFPLRSYSFRPTY | |
| | GVGHQPYRVVVLSFELLHAPATVCGPKKSTNLVKNK | |
| | CVNFNFNGLKGTGVLTESNKKFLPFQQFGRDIADTTD | |
| | AVRDPQTLEILDITPCSFGGVSVITPGTNTSNQVAVLY | |
| | QGVNCTEVPVAIHADQLTPTWRVYSTGSNVFQTRAG | |
| | CLIGAEYVNNSYECDIPIGAGICASYQTQTKSHQQAQ | |
| | SVASQSIIAYTMSLGAENSVAYSNNSIAIPTNFTISVTT | |
| | EILPVSMTKTSVDCTMYICGDSTECSNLLLQYGSFCT | |
| | QLKRALTGIAVEQDKNTQEVFAQVKQIYKTPPIKYFG | |
| | GFNFSQILPDPSKPSKRSFIEDLLFNKVTLADAGFIKQ | |
| | YGDCLGDIAARDLICAQKFKGLTVLPPLLTDEMIAQY | |
| | TSALLAGTITSGWTFGAGAALQIPFAMQMAYRENGI | |
| | GVTQNVLYENQKLIANQFNSAIGKIQDSLSSTASALG | |
| | KLQDVVNHNAQALNTLVKQLSSKFGAISSVLNDIFSR | |
| | LDPPEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BV2480 | QCVNLTTRTQLPPAYTNSFTRGVYYPDKVFRSSVLHS | 43 |
| | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLGVSNHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATKF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSHQQAQSVASQSIIAYTMSLGAENSVAYSNNSIA | |
| | IPTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQNVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| | QCVNLTTRTQLPPAYTNSFTRGVYYPDKVFRSSVLHS | 72 |
| BV2481 | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLGVTSNHKNNKSWMESEFRVY | |
| | SSANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVENATKF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSHQQAQSVASQSIIAYTMSLGAENSVAYSNNSIA | |
| | IPTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQNVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2457 | QCVNLTTRTQLPPAYTNSFTRGVYYPDKVFRSSVLHS | 73 |
| | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLDVYYHKNNKSWMKSEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYRYRL | |
| | FRKSNLKPFERDISTEIYQAGSTPCNGVQGFNCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSRQQAQSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAHEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2364 | ITNLCPFGEVENATRFASVYAWNRKRISNCVADYSVL | 90 |
| | YNSASFSTFKCYGVSPTKLNDLCFTNVYADSFVIRGD | |
| | EVRQIAPGQTGKIADYNYKLPDDFTGCVIAWNSNNL | |
| | DSKVGGNYNYLYRLFRKSNLKPFERDISTEIYQAGST | |
| | PCNGVEGENCYFPLQSYGFQPTNGVGYQPYRVVVLS | |
| | FELLHAPATVCGPKKSTNLVKNKCVNFNFNGLTGTG | |
| | VLTESNKKFLPFQQFGRDIADTTDAVRDPQTLEILDIT | |
| | PCSFGGVSVITPGTNTSNQVAVLYQDVNCTEVPVAIH | |
| | ADQLTPTWRVYSTGSNVFQTRAGCLIGAEHVNNSYE | |
| | CDIPIGAGICASYQTQTNSPRRARSVASQSIIAYTMSL | |
| | GAENSVAYSNNSIAIPTNFTISVTTEILPVSMTKTSVDC | |
| | TMYICGDSTECSNLLLQYGSFCTQLNRALTGIAVEQD | |
| | KNTQEVFAQVKQIYKTPPIKDFGGFNFSQILPDPSKPS | |
| | KRSFIEDLLFNKVTLADAGFIKQYGDCLGDIAARDLIC | |
| | AQKFNGLTVLPPLLTDEMIAQYTSALLAGTITSGWTF | |
| | GAGAALQIPFAMQMAYRENGIGVTQNVLYENQKLIA | |
| | NQFNSAIGKIQDSLSSTASALGKLQDVVNQNAQALN | |
| | TLVKQLSSNFGAISSVLNDILSRLDKVEAEVQIDRLIT | |
| | GRLQSLQTYVTQQLIRAAEIRASANLAATKMSECVL | |
| | GQSKRVDFCGKGYHLMSFPQSAPHGVVFLHVTYVP | |
| | AQEKNFTTAPAICHDGKAHFPREGVFVSNGTHWFVT | |
| | QRNFYEPQIITTDNTFVSGNCDVVIGIVNNTVYDPLQP | |
| | ELDSFKEELDKYFKNHTSPDVDLGDISGINASVVNIQ | |
| | KEIDRLNEVAKNLNESLIDLQELGKYEQYIKWPWYI | |
| | WLGFIAGLIAIVMVTIMLCCMTSCCSCLKGCCSCGSC | |
| | CKFDEDDSEPVLKGVKLHYT | |
| BV2365 | QCVNLTTRTQLPPAYTNSFTRGVYYPDKVFRSSVLHS | 91 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDKVEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2361/ | QCVNLTTRTQLPPAYTNSFTRGVYYPDKVFRS | 92 |
| BV2366 | SVLHSTQDLFLPFFSNVTWFHAIHVSGTNGTKRFDNP | |
| | VLPFNDGVYFASTEKSNIIRGWIFGTTLDSKTQSLLIV | |
| | NNATNVVIKVCEFQFCNDPFLGVYYHKNNKSWMES | |
| | EFRVYSSANNCTFEYVSQPFLMDLEGKQGNFKNLRE | |
| | FVFKNIDGYFKIYSKHTPINLVRDLPQGFSALEPLVDL | |
| | PIGINITRFQTLLALHRSYLTPGDSSSGWTAGAAAYY | |
| | VGYLQPRTFLLKYNENGTITDAVDCALDPLSETKCTL | |
| | KSFTVEKGIYQTSNFRVQPTESIVRFPNITNLCPFGEVF | |
| | NATRFASVYAWNRKRISNCVADYSVLYNSASFSTFK | |
| | CYGVSPTKLNDLCFTNVYADSFVIRGDEVRQIAPGQT | |
| | GKIADYNYKLPDDFTGCVIAWNSNNLDSKVGGNYN | |
| | YLYRLFRKSNLKPFERDISTEIYQAGSTPCNGVEGENC | |
| | YFPLQSYGFQPTNGVGYQPYRVVVLSFELLHAPATV | |
| | CGPKKSTNLVKNKCVNFNFNGLTGTGVLTESNKKFL | |
| | PFQQFGRDIADTTDAVRDPQTLEILDITPCSFGGVSVIT | |
| | PGTNTSNQVAVLYQDVNCTEVPVAIHADQLTPTWRV | |
| | YSTGSNVFQTRAGCLIGAEHVNNSYECDIPIGAGICAS | |
| | YQTQTNSPRRARSVASQSIIAYTMSLGAENSVAYSNN | |
| | SIAIPTNFTISVTTEILPVSMTKTSVDCTMYICGDSTEC | |
| | SNLLLQYGSFCTQLNRALTGIAVEQDKNTQEVFAQV | |
| | KQIYKTPPIKDFGGFNFSQILPDPSKPSKRSFIEDLLEN | |
| | KVTLADAGFIKQYGDCLGDIAARDLICAQKFNGLTV | |
| | LPPLLTDEMIAQYTSALLAGTITSGWTFGAGAALQIPF | |
| | AMQMAYRFNGIGVTQNVLYENQKLIANQFNSAIGKI | |
| | QDSLSSTASALGKLQDVVNQNAQALNTLVKQLSSNF | |
| | GAISSVLNDILSRLDKVEAEVQIDRLITGRLQSLQTYV | |
| | TQQLIRAAEIRASANLAATKMSECVLGQSKRVDFCG | |
| | KGYHLMSFPQSAPHGVVFLHVTYVPAQEKNFTTAPA | |
| | ICHDGKAHFPREGVFVSNGTHWFVTQRNFYEPQIITT | |
| | DNTFVSGNCDVVIGIVNNTVYDPLQPELDSFKEELDK | |
| | YFKNHTSPDVDLGDISGINASVVNIQKEIDRLNEVAK | |
| | NLNESLIDLQELGKYEQYIKWPWYIWLGFIAGLIAIV | |
| | MVTIMLCCMTSCCSCLKGCCSCGSCCKFDEDDSEPV | |
| | LKGVKLHYT | |
| BV2367 | QCVNLTTRTQLPPAYTNSFTRGVYYPDKVFRSSVLHS | 93 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPQQAQSVALGAENSVAYSNNSIAIPTNFTISVTT | |
| | EILPVSMTKTSVDCTMYICGDSTECSNLLLQYGSFCT | |
| | QLNRALTGIAVEQDKNTQEVFAQVKQIYKTPPIKDFG | |
| | GFNFSQILPDPSKPSKRSFIEDLLFNKVTLADAGFIKQ | |
| | YGDCLGDIAARDLICAQKFNGLTVLPPLLTDEMIAQY | |
| | TSALLAGTITSGWTFGAGAALQIPFAMQMAYRENGI | |
| | GVTQNVLYENQKLIANQFNSAIGKIQDSLSSTASALG | |
| | KLQDVVNQNAQALNTLVKQLSSNFGAISSVLNDILSR | |
| | LDKVEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BV2368 | QCVNLTTRTQLPPAYTNSFTRGVYYPDKVFRSSVLHS | 94 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGAENSVAYSNNSIAI | |
| | EILPVSMTKTSVDCTMYICGDSTECSNLLLQYGSFCT | |
| | QLNRALTGIAVEQDKNTQEVFAQVKQIYKTPPIKDFG | |
| | GFNFSQILPDPSKPSKRSFIEDLLFNKVTLADAGFIKQ | |
| | YGDCLGDIAARDLICAQKFNGLTVLPPLLTDEMIAQY | |
| | TSALLAGTITSGWTFGAGAALQIPFAMQMAYRENGI | |
| | GVTQNVLYENQKLIANQFNSAIGKIQDSLSSTASALG | |
| | KLQDVVNQNAQALNTLVKQLSSNFGAISSVLNDILSR | |
| | LDKVEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BV2369 | QCVNLTTRTQLPPAYTNSFTRGVYYPDKVFRSSVLHS | 95 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVENATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIADAGFIKQY | |
| | GDCLGDIAARDLICAQKFNGLTVLPPLLTDEMIAQYT | |
| | SALLAGTITSGWTFGAGAALQIPFAMQMAYRENGIG | |
| | VTQNVLYENQKLIANQFNSAIGKIQDSLSSTASALGK | |
| | LQDVVNQNAQALNTLVKQLSSNFGAISSVLNDILSRL | |
| | DKVEAEVQIDRLITGRLQSLQTYVTQQLIRAAEIRASA | |
| | NLAATKMSECVLGQSKRVDFCGKGYHLMSFPQSAP | |
| | HGVVFLHVTYVPAQEKNFTTAPAICHDGKAHFPREG | |
| | VFVSNGTHWFVTQRNFYEPQIITTDNTFVSGNCDVVI | |
| | GIVNNTVYDPLQPELDSFKEELDKYFKNHTSPDVDLG | |
| | DISGINASVVNIQKEIDRLNEVAKNLNESLIDLQELGK | |
| | YEQYIKWPWYIWLGFIAGLIAIVMVTIMLCCMTSCCS | |
| | CLKGCCSCGSCCKFDEDDSEPVLKGVKLHYT | |
| BV2374 | QCVNLTTRTQLPPAYTNSFTRGVYYPDKVFRSSVLHS | 96 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPRRARSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLENKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2374 | QCVNLTTRTQLPPAYTNSFTRGVYYPDKVFRSSVLHS | 97 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDKVEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYTHHHHHH | |
| BV2384 | QCVNLTTRTQLPPAYTNSFTRGVYYPDKVFRSSVLHS | 98 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQDVNCTEVPVAIHADQLTPTWRVYSTG | |
| | SNVFQTRAGCLIGAEHVNNSYECDIPIGAGICASYQT | |
| | QTNSPGSASSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2425 | QCVNLTTRTQLPPAYTNSFTRGVYYPDKVFRSSVLHS | 99 |
| | TQDLFLPFFSNVTWFHAISGTNGTKRFDNPVLPFNDG | |
| | VYFASTEKSNIIRGWIFGTTLDSKTQSLLIVNNATNVV | |
| | IKVCEFQFCNDPFLGVYHKNNKSWMESEFRVYSSAN | |
| | NCTFEYVSQPFLMDLEGKQGNFKNLREFVFKNIDGY | |
| | FKIYSKHTPINLVRDLPQGFSALEPLVDLPIGINITRFQ | |
| | TLLALHRSYLTPGDSSSGWTAGAAAYYVGYLQPRTF | |
| | LLKYNENGTITDAVDCALDPLSETKCTLKSFTVEKGI | |
| | YQTSNFRVQPTESIVRFPNITNLCPFGEVENATRFASV | |
| | YAWNRKRISNCVADYSVLYNSASFSTFKCYGVSPTK | |
| | LNDLCFTNVYADSFVIRGDEVRQIAPGQTGKIADYNY | |
| | KLPDDFTGCVIAWNSNNLDSKVGGNYNYLYRLFRKS | |
| | NLKPFERDISTEIYQAGSTPCNGVEGENCYFPLQSYGF | |
| | QPTYGVGYQPYRVVVLSFELLHAPATVCGPKKSTNL | |
| | VKNKCVNFNFNGLTGTGVLTESNKKFLPFQQFGRDI | |
| | DDTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEHVNNSYECDIPIGAGICASYQTQTNS | |
| | HQQAQSVASQSIIAYTMSLGAENSVAYSNNSIAIPINF | |
| | TISVTTEILPVSMTKTSVDCTMYICGDSTECSNLLLQY | |
| | GSFCTQLNRALTGIAVEQDKNTQEVFAQVKQIYKTPP | |
| | IKDFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNQNAQALNTLVKQLSSNFGAISSVLN | |
| | DILARLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTHNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2426 | QCVNLTTRTQLPPAYTNSFTRGVYYPDKVFRSSVLHS | 100 |
| | TQDLFLPFFSNVTWFHAIHVSGTNGTKRFANPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRGLPQGFSALEPLVDLPIGINIT | |
| | RFQTLHALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGNIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGVENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2438 | QCVNLTTRTQLPPAYTNSFTRGVYYPDKVFRSSVLHS | 101 |
| | TQDLFLPFFSNVTWFHAIHVSGTNGTKRFANPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRGLPQGFSALEPLVDLPIGINIT | |
| | RFQTLHALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGNIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGVENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGNFSQILPDPSKPSKRSFIEDLLFNKVTL | |
| | ADAGFIKQYGDCLGDIAARDLICAQKFNGLTVLPPLL | |
| | TDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2423 | QCVNLTTRTQLPPAYTNSFTRGVYYPDKVFRSSVLHS | 102 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGFNCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPGGSVASQSIIAYTMSLGAENSVAYSNNSIAIPT | |
| | NFTISVTTEILPVSMTKTSVDCTMYICGDSTECSNLLL | |
| | QYGSFCTQLNRALTGIAVEQDKNTQEVFAQVKQIYK | |
| | TPPIKDFGGFNFSQILPDPSKPSKRSFIEDLLFNKVTLA | |
| | DAGFIKQYGDCLGDIAARDLICAQKENGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2425-2 | QCVNLTTRTQLPPAYTNSFTRGVYYPDKVFRSSVLHS | 103 |
| | TQDLFLPFFSNVTWFHAISGTNGTKRFDNPVLPENDG | |
| | VYFASTEKSNIIRGWIFGTTLDSKTQSLLIVNNATNVV | |
| | IKVCEFQFCNDPFLGVYHKNNKSWMESEFRVYSSAN | |
| | NCTFEYVSQPFLMDLEGKQGNFKNLREFVFKNIDGY | |
| | FKIYSKHTPINLVRDLPQGFSALEPLVDLPIGINITRFQ | |
| | TLLALHRSYLTPGDSSSGWTAGAAAYYVGYLQPRTF | |
| | LLKYNENGTITDAVDCALDPLSETKCTLKSFTVEKGI | |
| | YQTSNFRVQPTESIVRFPNITNLCPFGEVFNATRFASV | |
| | YAWNRKRISNCVADYSVLYNSASFSTFKCYGVSPTK | |
| | LNDLCFTNVYADSFVIRGDEVRQIAPGQTGKIADYNY | |
| | KLPDDFTGCVIAWNSNNLDSKVGGNYNYLYRLFRKS | |
| | NLKPFERDISTEIYQAGSTPCNGVEGENCYFPLQSYGF | |
| | QPTYGVGYQPYRVVVLSFELLHAPATVCGPKKSTNL | |
| | VKNKCVNFNFNGLTGTGVLTESNKKFLPFQQFGRDI | |
| | DDTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEHVNNSYECDIPIGAGICASYQTQTNS | |
| | HGGSVASQSIIAYTMSLGAENSVAYSNNSIAIPINFTIS | |
| | VTTEILPVSMTKTSVDCTMYICGDSTECSNLLLQYGS | |
| | FCTQLNRALTGIAVEQDKNTQEVFAQVKQIYKTPPIK | |
| | DFGGFNFSQILPDPSKPSKRSFIEDLLFNKVTLADAGFI | |
| | KQYGDCLGDIAARDLICAQKFNGLTVLPPLLTDEMIA | |
| | QYTSALLAGTITSGWTFGAGAALQIPFAMQMAYRFN | |
| | GIGVTQNVLYENQKLIANQFNSAIGKIQDSLSSTASAL | |
| | GKLQDVVNQNAQALNTLVKQLSSNFGAISSVLNDIL | |
| | ARLDPPEAEVQIDRLITGRLQSLQTYVTQQLIRAAEIR | |
| | ASANLAATKMSECVLGQSKRVDFCGKGYHLMSFPQ | |
| | SAPHGVVFLHVTYVPAQEKNFTTAPAICHDGKAHFP | |
| | REGVFVSNGTHWFVTQRNFYEPQIITTHNTFVSGNCD | |
| | VVIGIVNNTVYDPLQPELDSFKEELDKYFKNHTSPDV | |
| | DLGDISGINASVVNIQKEIDRLNEVAKNLNESLIDLQE | |
| | LGKYEQYIKWPWYIWLGFIAGLIAIVMVTIMLCCMTS | |
| | CCSCLKGCCSCGSCCKFDEDDSEPVLKGVKLHYT | |
| BV2439 | QCVNLTTRTQLPPAYTNSFTRGVYYPDKVFRSSVLHS | 104 |
| | TQDLFLPFFSNVTWFHAIHVSGTNGTKRFANPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRGLPQGFSALEPLVDLPIGINIT | |
| | RFQTLHALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGNIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPGGSVASQSIIAYTMSLGVENSVAYSNNSIAIPT | |
| | NFTISVTTEILPVSMTKTSVDCTMYICGDSTECSNLLL | |
| | QYGSFCTQLNRALTGIAVEQDKNTQEVFAQVKQIYK | |
| | TPPIKDFGGNFSQILPDPSKPSKRSFIEDLLENKVTLAD | |
| | AGFIKQYGDCLGDIAARDLICAQKFNGLTVLPPLLTD | |
| | EMIAQYTSALLAGTITSGWTFGAGAALQIPFAMQMA | |
| | YRFNGIGVTQNVLYENQKLIANQFNSAIGKIQDSLSST | |
| | ASALGKLQDVVNQNAQALNTLVKQLSSNFGAISSVL | |
| | NDILSRLDPPEAEVQIDRLITGRLQSLQTYVTQQLIRA | |
| | AEIRASANLAATKMSECVLGQSKRVDFCGKGYHLMS | |
| | FPQSAPHGVVFLHVTYVPAQEKNFTTAPAICHDGKA | |
| | HFPREGVFVSNGTHWFVTQRNFYEPQIITTDNTFVSG | |
| | NCDVVIGIVNNTVYDPLQPELDSFKEELDKYFKNHTS | |
| | PDVDLGDISGINASVVNIQKEIDRLNEVAKNLNESLID | |
| | LQELGKYEQYIKWPWYIWLGFIAGLIAIVMVTIMLCC | |
| | MTSCCSCLKGCCSCGSCCKFDEDDSEPVLKGVKLHY | |
| | T | |
| BV2441 | QCVNFTTRTQLPPAYTNSFTRGVYYPDKVFRSSVLHS | 105 |
| | TQDLFLPFFSNVTWFHAIHVSGTNGTKRFANPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRGLPQGFSALEPLVDLPIGINIT | |
| | RFQTLHRSYLTPGDSSSGWTAGAAAYYVGYLQPRTF | |
| | LLKYNENGTITDAVDCALDPLSETKCTLKSFTVEKGI | |
| | YQTSNFRVQPTESIVRFPNITNLCPFGEVENATRFASV | |
| | YAWNRKRISNCVADYSVLYNSASFSTFKCYGVSPTK | |
| | LNDLCFTNVYADSFVIRGDEVRQIAPGQTGNIADYNY | |
| | KLPDDFTGCVIAWNSNNLDSKVGGNYNYLYRLFRKS | |
| | NLKPFERDISTEIYQAGSTPCNGVKGFNCYFPLQSYGF | |
| | QPTYGVGYQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEHVNNSYECDIPIGAGICASYQTQTNSP | |
| | QQAQSVASQSIIAYTMSLGVENSVAYSNNSIAIPTNFT | |
| | ISVTTEILPVSMTKTSVDCTMYICGDSTECSNLLLQYG | |
| | SFCTQLNRALTGIAVEQDKNTQEVFAQVKQIYKTPPI | |
| | KDFGGNFSQILPDPSKPSKRSFIEDLLFNKVTLADAGF | |
| | IKQYGDCLGDIAARDLICAQKFNGLTVLPPLLTDEMI | |
| | AQYTSALLAGTITSGWTFGAGAALQIPFAMQMAYRF | |
| | NGIGVTQNVLYENQKLIANQFNSAIGKIQDSLSSTASA | |
| | LGKLQDVVNQNAQALNTLVKQLSSNFGAISSVLNDIL | |
| | SRLDPPEAEVQIDRLITGRLQSLQTYVTQQLIRAAEIR | |
| | ASANLAATKMSECVLGQSKRVDFCGKGYHLMSFPQ | |
| | SAPHGVVFLHVTYVPAQEKNFTTAPAICHDGKAHFP | |
| | REGVFVSNGTHWFVTQRNFYEPQIITTDNTFVSGNCD | |
| | VVIGIVNNTVYDPLQPELDSFKEELDKYFKNHTSPDV | |
| | DLGDISGINASVVNIQKEIDRLNEVAKNLNESLIDLQE | |
| | LGKYEQYIKWPWYIWLGFIAGLIAIVMVTIMLCCMTS | |
| | CCSCLKGCCSCGSCCKFDEDDSEPVLKGVKLHYT | |
| BV2442 | QCVNFTTRTQLPPAYTNSFTRGVYYPDKVFRSSVLHS | 106 |
| | TQDLFLPFFSNVTWFHAIHVSGTNGTKRFANPVLPFN | |
| | DGVYFASTEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRGLPQGFSALEPLVDLPIGINIT | |
| | RFQTLHRSYLTPGDSSSGWTAGAAAYYVGYLQPRTF | |
| | LLKYNENGTITDAVDCALDPLSETKCTLKSFTVEKGI | |
| | YQTSNFRVQPTESIVRFPNITNLCPFGEVENATRFASV | |
| | YAWNRKRISNCVADYSVLYNSASFSTFKCYGVSPTK | |
| | LNDLCFTNVYADSFVIRGDEVRQIAPGQTGNIADYNY | |
| | KLPDDFTGCVIAWNSNNLDSKVGGNYNYLYRLFRKS | |
| | NLKPFERDISTEIYQAGSTPCNGVKGFNCYFPLQSYGF | |
| | QPTYGVGYQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEHVNNSYECDIPIGAGICASYQTQTNSP | |
| | GGSVASQSIIAYTMSLGVENSVAYSNNSIAIPTNFTISV | |
| | TTEILPVSMTKTSVDCTMYICGDSTECSNLLLQYGSF | |
| | CTQLNRALTGIAVEQDKNTQEVFAQVKQIYKTPPIKD | |
| | FGGNFSQILPDPSKPSKRSFIEDLLFNKVTLADAGFIK | |
| | QYGDCLGDIAARDLICAQKFNGLTVLPPLLTDEMIAQ | |
| | YTSALLAGTITSGWTFGAGAALQIPFAMQMAYRENG | |
| | IGVTQNVLYENQKLIANQFNSAIGKIQDSLSSTASALG | |
| | KLQDVVNQNAQALNTLVKQLSSNFGAISSVLNDILSR | |
| | LDPPEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BV2443 | QCVNFTNRTQLPSAYTNSFTRGVYYPDKVFRSSVLHS | 107 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNYPFLGVYYHKNNKSWMESEFRVYS | |
| | SANNCTFEYVSQPFLMDLEGKQGNFKNLSEFVFKNID | |
| | GYFKIYSKHTPINLVRDLPQGFSALEPLVDLPIGINITR | |
| | FQTLLALHRSYLTPGDSSSGWTAGAAAYYVGYLQPR | |
| | TFLLKYNENGTITDAVDCALDPLSETKCTLKSFTVEK | |
| | GIYQTSNFRVQPTESIVRFPNITNLCPFGEVENATRFAS | |
| | VYAWNRKRISNCVADYSVLYNSASFSTFKCYGVSPT | |
| | KLNDLCFTNVYADSFVIRGDEVRQIAPGQTGTIADYN | |
| | YKLPDDFTGCVIAWNSNNLDSKVGGNYNYLYRLFR | |
| | KSNLKPFERDISTEIYQAGSTPCNGVKGFNCYFPLQSY | |
| | GFQPTYGVGYQPYRVVVLSFELLHAPATVCGPKKST | |
| | NLVKNKCVNFNFNGLTGTGVLTESNKKFLPFQQFGR | |
| | DIADTTDAVRDPQTLEILDITPCSFGGVSVITPGTNTSN | |
| | QVAVLYQGVNCTEVPVAIHADQLTPTWRVYSTGSN | |
| | VFQTRAGCLIGAEYVNNSYECDIPIGAGICASYQTQT | |
| | NSPQQAQSVASQSIIAYTMSLGAENSVAYSNNSIAIPT | |
| | NFTISVTTEILPVSMTKTSVDCTMYICGDSTECSNLLL | |
| | QYGSFCTQLNRALTGIAVEQDKNTQEVFAQVKQIYK | |
| | TPPIKDFGGFNFSQILPDPSKPSKRSFIEDLLFNKVTLA | |
| | DAGFIKQYGDCLGDIAARDLICAQKFNGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQDVVNQNAQALNTLVKQLSSNFGAISS | |
| | VLNDILSRLDPPEAEVQIDRLITGRLQSLQTYVTQQLI | |
| | RAAEIRASANLAAIKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASFVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2448 | QCVNLTTRTQLPPAYTNSFTRGVYYPDKVFRSSVLHS | 108 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLGVYYHKNNKSCMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYRYRL | |
| | FRKSNLKPFERDISTEIYQAGSTPCNGVEGFNCYFPLQ | |
| | PYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV1.526NY- | QCVNLTTRTQLPPAYTNSFTRGVYYPDKVFRSSVLHS | 109 |
| 1 | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGGSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGVENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV1.526NY- | QCVNLTTRTQLPPAYTNSFTRGVYYPDKVFRSSVLHS | 110 |
| 2 | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLGVYYHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGGSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGNTPCNGVEGENCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSPQQAQSVASQSIIAYTMSLGVENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLENKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2508 | QCVNLTTRTQLPPAYTNSFTRGVYYPDKVFRSSVLHS | 111 |
| | TQDLFLPFFSNVTWFHVIHVSGTNGTKRFDNPVLPFN | |
| | DGVYFASIEKSNIIRGWIFGTTLDSKTQSLLIVNNATN | |
| | VVIKVCEFQFCNDPFLDVYYHKNNKSWMESEFRVYS | |
| | SANNCTFEYVSQPFLMDLEGKQGNFKNLREFVFKNI | |
| | DGYFKIYSKHTPINIVRDLPQGFSALEPLVDLPIGINIT | |
| | RFQTLLALHRSYLPGDSSSGWTAGAAAYYVGYLQPR | |
| | TFLLKYNENGTITDAVDCALDPLSETKCTLKSFTVEK | |
| | GIYQTSNFRVQPTESIVRFPNITNLCPFDEVFNATRFAS | |
| | VYAWNRKRISNCVADYSVLYNLAPFFTFKCYGVSPT | |
| | KLNDLCFTNVYADSFVIRGDEVRQIAPGQTGNIADYN | |
| | YKLPDDFTGCVIAWNSNKLDSKVSGNYNYLYRLFRK | |
| | SNLKPFERDISTEIYQAGNKPCNGVAGENCYFPLRSY | |
| | SFRPTYGVGHQPYRVVVLSFELLHAPATVCGPKKST | |
| | NLVKNKCVNFNFNGLKGTGVLTESNKKFLPFQQFGR | |
| | DIADTTDAVRDPQTLEILDITPCSFGGVSVITPGTNTSN | |
| | QVAVLYQGVNCTEVPVAIHADQLTPTWRVYSTGSN | |
| | VFQTRAGCLIGAEYVNNSYECDIPIGAGICASYQTQT | |
| | KSHQQAQSVASQSIIAYTMSLGAENSVAYSNNSIAIPT | |
| | NFTISVTTEILPVSMTKTSVDCTMYICGDSTECSNLLL | |
| | QYGSFCTQLKRALTGIAVEQDKNTQEVFAQVKQIYK | |
| | TPPIKYFGGFNFSQILPDPSKPSKRSFIEDLLFNKVTLA | |
| | DAGFIKQYGDCLGDIAARDLICAQKFKGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQDVVNHNAQALNTLVKQLSSKFGAISS | |
| | VLNDIFSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKELDKYFKNHT | |
| | SPDVDLGDISGINASVVNIQKEIDRLNEVAKNLNESLI | |
| | DLQELGKYEQYIKWPWYIWLGFIAGLIAIVMVTIMLC | |
| | CMTSCCSCLKGCCSCGSCCKFDEDDSEPVLKGVKLH | |
| | YT | |
| BV2509 | QCVNLTTRTQLPPAYTNSFTRGVYYPDKVFRSSVLHS | 112 |
| | TQDLFLPFFSNVTWFHVISGTNGTKRFDNPVLPFNDG | |
| | VYFASIEKSNIIRGWIFGTTLDSKTQSLLIVNNATNVVI | |
| | KVCEFQFCNDPFLDHKNNKSWMESEFRVYSSANNCT | |
| | FEYVSQPFLMDLEGKQGNFKNLREFVFKNIDGYFKIY | |
| | SKHTPIIVREPEDLPQGFSALEPLVDLPIGINITRFQTLL | |
| | ALHRSYLTPGDSSSGWTAGAAAYYVGYLQPRTFLLK | |
| | YNENGTITDAVDCALDPLSETKCTLKSFTVEKGIYQT | |
| | SNFRVQPTESIVRFPNITNLCPFDEVFNATRFASVYAW | |
| | NRKRISNCVADYSVLYNLAPFFTFKCYGVSPTKLNDL | |
| | CFTNVYADSFVIRGDEVRQIAPGQTGNIADYNYKLPD | |
| | DFTGCVIAWNSNKLDSKVSGNYNYLYRLFRKSNLKP | |
| | FERDISTEIYQAGNKPCNGVAGENCYFPLRSYSFRPTY | |
| | GVGHQPYRVVVLSFELLHAPATVCGPKKSTNLVKNK | |
| | CVNFNFNGLKGTGVLTESNKKFLPFQQFGRDIADTTD | |
| | AVRDPQTLEILDITPCSFGGVSVITPGTNTSNQVAVLY | |
| | QGVNCTEVPVAIHADQLTPTWRVYSTGSNVFQTRAG | |
| | CLIGAEYVNNSYECDIPIGAGICASYQTQTKSHQQAQ | |
| | SVASQSIIAYTMSLGAENSVAYSNNSIAIPTNFTISVTT | |
| | EILPVSMTKTSVDCTMYICGDSTECSNLLLQYGSFCT | |
| | QLKRALTGIAVEQDKNTQEVFAQVKQIYKTPPIKYFG | |
| | GFNFSQILPDPSKPSKRSFIEDLLFNKVTLADAGFIKQ | |
| | YGDCLGDIAARDLICAQKFKGLTVLPPLLTDEMIAQY | |
| | TSALLAGTITSGWTFGAGAALQIPFAMQMAYRENGI | |
| | GVTQNVLYENQKLIANQFNSAIGKIQDSLSSTASALG | |
| | KLQDVVNHNAQALNTLVKQLSSKFGAISSVLNDIFSR | |
| | LDPPEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BV2465 | QCVNLRTRTQLPPAYTNSFTRGVYYPDKVFRSSVLHS | 113 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLDVYYHKNNKSWMESGVYSS | |
| | ANNCTFEYVSQPFLMDLEGKQGNFKNLREFVFKNID | |
| | GYFKIYSKHTPINLVRDLPQGFSALEPLVDLPIGINITR | |
| | FQTLLALHRSYLTPGDSSSGWTAGAAAYYVGYLQPR | |
| | TFLLKYNENGTITDAVDCALDPLSETKCTLKSFTVEK | |
| | GIYQTSNFRVQPTESIVRFPNITNLCPFGEVENATRFAS | |
| | VYAWNRKRISNCVADYSVLYNSASFSTFKCYGVSPT | |
| | KLNDLCFTNVYADSFVIRGDEVRQIAPGQTGKIADYN | |
| | YKLPDDFTGCVIAWNSNNLDSKVGGNYNYRYRLFR | |
| | KSNLKPFERDISTEIYQAGSKPCNGVEGENCYFPLQSY | |
| | GFQPTNGVGYQPYRVVVLSFELLHAPATVCGPKKST | |
| | NLVKNKCVNFNFNGLTGTGVLTESNKKFLPFQQFGR | |
| | DIADTTDAVRDPQTLEILDITPCSFGGVSVITPGTNTSN | |
| | QVAVLYQGVNCTEVPVAIHADQLTPTWRVYSTGSN | |
| | VFQTRAGCLIGAEHVNNSYECDIPIGAGICASYQTQT | |
| | NSRQQAQSVASQSIIAYTMSLGAENSVAYSNNSIAIPT | |
| | NFTISVTTEILPVSMTKTSVDCTMYICGDSTECSNLLL | |
| | QYGSFCTQLNRALTGIAVEQDKNTQEVFAQVKQIYK | |
| | TPPIKDFGGFNFSQILPDPSKPSKRSFIEDLLFNKVTLA | |
| | DAGFIKQYGDCLGDIAARDLICAQKFNGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQNVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2457 | QCVNLTTRTQLPPAYTNSFTRGVYYPDKVFRSSVLHS | 114 |
| | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLDVYYHKNNKSWMKSEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATRF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYRYRL | |
| | FRKSNLKPFERDISTEIYQAGSTPCNGVQGFNCYFPLQ | |
| | SYGFQPTNGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSRQQAQSVASQSIIAYTMSLGAENSVAYSNNSIAI | |
| | PTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQDVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAHEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2472 | QCVNLRTRTQLPPAYTNSFTRGVYYPDKVFRSSVLHS | 115 |
| | TQDLFLPFFSNVTWFHAIHVSGTNGTKRFDNPVLPFN | |
| | DGVYFASTEKSNIIRGWIFGTTLDSKTQSLLIVNNATN | |
| | VVIKVCEFQFCNDPFLDVYYHKNNKSWMESGVYSS | |
| | ANNCTFEYVSQPFLMDLEGKQGNFKNLREFVFKNID | |
| | GYFKIYSKHTPINLVRDLPQGFSALEPLVDLPIGINITR | |
| | FQTLLALHRSYLTPGDSSSGITAGAAAYYVGYLQPRT | |
| | FLLKYNENGTITDAVDCALDPLSETKCTLKSFTVEKG | |
| | IYQTSNFRVQPTESIVRFPNITNLCPFGEVENATRFASV | |
| | YAWNRKRISNCVADYSVLYNSASFSTFKCYGVSPTK | |
| | LNDLCFTNVYADSFVIRGDEVRQIAPGQTGNIADYNY | |
| | KLPDDFTGCVIAWNSNNLDSKVGGNYNYRYRLFRKS | |
| | NLKPFERDISTEIYQAGSKPCNGVEGFNCYFPLQSYGF | |
| | QPTNGVGYQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEHVNNSYECDIPIGAGICASYQTQTNS | |
| | RQQAQSVASQSIIAYTMSLGAENSVAYSNNSIAIPTNF | |
| | TISVTTEILPVSMTKTSVDCTMYICGDSTECSNLLLQY | |
| | GSFCTQLNRALTGIAVEQDKNTQEVFAQVKQIYKTPP | |
| | IKDFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQNVVNQNAQALNTLVKQLSSNFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2480 | QCVNLTTRTQLPPAYTNSFTRGVYYPDKVFRSSVLHS | 116 |
| | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLGVSNHKNNKSWMESEFRVYS | |
| | SANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATKF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSHQQAQSVASQSIIAYTMSLGAENSVAYSNNSIA | |
| | IPTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQNVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2481 | QCVNLTTRTQLPPAYTNSFTRGVYYPDKVFRSSVLHS | 117 |
| | TQDLFLPFFSNVTWFHAIHVSGTNGTKRFDNPVLPFN | |
| | DGVYFASIEKSNIIRGWIFGTTLDSKTQSLLIVNNATN | |
| | VVIKVCEFQFCNDPFLGVTSNHKNNKSWMESEFRVY | |
| | SSANNCTFEYVSQPFLMDLEGKQGNFKNLREFVFKNI | |
| | DGYFKIYSKHTPINLVRDLPQGFSALEPLVDLPIGINIT | |
| | RFQTLLALHRSYLTPGDSSSGWTAGAAAYYVGYLQP | |
| | RTFLLKYNENGTITDAVDCALDPLSETKCTLKSFTVE | |
| | KGIYQTSNFRVQPTESIVRFPNITNLCPFGEVFNATKF | |
| | ASVYAWNRKRISNCVADYSVLYNSASFSTFKCYGVS | |
| | PTKLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVKGFNCYFPLQ | |
| | SYGFQPTYGVGYQPYRVVVLSFELLHAPATVCGPKK | |
| | STNLVKNKCVNFNFNGLTGTGVLTESNKKFLPFQQF | |
| | GRDIADTTDAVRDPQTLEILDITPCSFGGVSVITPGTN | |
| | TSNQVAVLYQGVNCTEVPVAIHADQLTPTWRVYSTG | |
| | SNVFQTRAGCLIGAEHVNNSYECDIPIGAGICASYQT | |
| | QTNSHQQAQSVASQSIIAYTMSLGAENSVAYSNNSIA | |
| | IPTNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNKVT | |
| | LADAGFIKQYGDCLGDIAARDLICAQKFNGLTVLPPL | |
| | LTDEMIAQYTSALLAGTITSGWTFGAGAALQIPFAMQ | |
| | MAYRFNGIGVTQNVLYENQKLIANQFNSAIGKIQDSL | |
| | SSTASALGKLQNVVNQNAQALNTLVKQLSSNFGAISS | |
| | VLNDILSRLDPPEAEVQIDRLITGRLQSLQTYVTQQLI | |
| | RAAEIRASANLAATKMSECVLGQSKRVDFCGKGYHL | |
| | MSFPQSAPHGVVFLHVTYVPAQEKNFTTAPAICHDG | |
| | KAHFPREGVFVSNGTHWFVTQRNFYEPQIITTDNTFV | |
| | SGNCDVVIGIVNNTVYDPLQPELDSFKEELDKYFKNH | |
| | TSPDVDLGDISGINASVVNIQKEIDRLNEVAKNLNESL | |
| | IDLQELGKYEQYIKWPWYIWLGFIAGLIAIVMVTIML | |
| | CCMTSCCSCLKGCCSCGSCCKFDEDDSEPVLKGVKL | |
| | HYT | |
| BV2523 | QCVNLITRTQSYTNSFTRGVYYPDKVFRSSVLHSTQD | 118 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHKNNKSWMESEFRVYSSA | |
| | NNCTFEYVSQPFLMDLEGKQGNFKNLREFVFKNIDG | |
| | YFKIYSKHTPINLGRDLPQGFSALEPLVDLPIGINITRF | |
| | QTLLALHRSYLTPGDSSSGWTAGAAAYYVGYLQPRT | |
| | FLLKYNENGTITDAVDCALDPLSETKCTLKSFTVEKG | |
| | IYQTSNFRVQPTESIVRFPNITNLCPFDEVFNATRFASV | |
| | YAWNRKRISNCVADYSVLYNFAPFFAFKCYGVSPTK | |
| | LNDLCFTNVYADSFVIRGNEVSQIAPGQTGNIADYNY | |
| | KLPDDFTGCVIAWNSNKLDSKVGGNYNYLYRLFRKS | |
| | NLKPFERDISTEIYQAGNKPCNGVAGENCYFPLRSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| | QCVNLITRTQSYTNSFTRGVYYPDKVFRSSVLHSTQD | 119 |
| BV2540 | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATRFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNKLDSKVGGNYNYRYRLFRKSN | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2541 | QCVNLITRTQSYTNSFTRGVYYPDKVFRSSVLHSTQD | 120 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATRFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNNLDSKVGGNYNYRYRLFRKSN | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTVSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2515 | QCVNLTTRTQLPPAYTNSFTRGVYYPDKVFRSSVLHS | 121 |
| | TQDLFLPFFSNVTWFHVISGTNGTKRFDNPVLPENDG | |
| | VYFASIEKSNIIRGWIFGTTLDSKTQSLLIVNNATNVVI | |
| | KVCEFQFCNDPFLDHKNNKSWMESEFRVYSSANNCT | |
| | FEYVSQPFLMDLEGKQGNFKNLREFVFKNIDGYFKIY | |
| | SKHTPIIVREPEDLPQGFSALEPLVDLPIGINITRFQTLL | |
| | ALHRSYLTPGDSSSGWTAGAAAYYVGYLQPRTFLLK | |
| | YNENGTITDAVDCALDPLSETKCTLKSFTVEKGIYQT | |
| | SNFRVQPTESIVRFPNITNLCPFDEVFNATRFASVYAW | |
| | NRKRISNCVADYSVLYNSAPFFTFKCYGVSPTKLNDL | |
| | CFTNVYADSFVIRGDEVRQIAPGQTGNIADYNYKLPD | |
| | DFTGCVIAWNSNKLDSKVSGNYNYLYRLFRKSNLKP | |
| | FERDISTEIYQAGNKPCNGVAGFNCYFPLRSYSFRPTY | |
| | GVGHQPYRVVVLSFELLHAPATVCGPKKSTNLVKNK | |
| | CVNFNFNGLKGTGVLTESNKKFLPFQQFGRDIADTTD | |
| | AVRDPQTLEILDITPCSFGGVSVITPGTNTSNQVAVLY | |
| | QGVNCTEVPVAIHADQLTPTWRVYSTGSNVFQTRAG | |
| | CLIGAEYVNNSYECDIPIGAGICASYQTQTKSHQQAQ | |
| | SVASQSIIAYTMSLGAENSVAYSNNSIAIPTNFTISVTT | |
| | EILPVSMTKTSVDCTMYICGDSTECSNLLLQYGSFCT | |
| | QLKRALTGIAVEQDKNTQEVFAQVKQIYKTPPIKYFG | |
| | GFNFSQILPDPSKPSKRSFIEDLLFNKVTLADAGFIKQ | |
| | YGDCLGDIAARDLICAQKFKGLTVLPPLLTDEMIAQY | |
| | TSALLAGTITSGWTFGAGAALQIPFAMQMAYRENGI | |
| | GVTQNVLYENQKLIANQFNSAIGKIQDSLSSTASALG | |
| | KLQDVVNHNAQALNTLVKQLSSKFGAISSVLNDIFSR | |
| | LDPPEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BV2542 | QCVNLITRTQSYTNSFTRGVYYPDKVFRSSVLHSTQD | 122 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHKNNKSWMESEFRVYSSA | |
| | NNCTFEYVSQPFLMDLEGKQGNFKNLREFVFKNIDG | |
| | YFKIYSKHTPINLGRDLPQGFSALEPLVDLPIGINITRF | |
| | QTLLALHRSYLTPGDSSSGWTAGAAAYYVGYLQPRT | |
| | FLLKYNENGTITDAVDCALDPLSETKCTLKSFTVEKG | |
| | IYQTSNFRVQPTESIVRFPNITNLCPFDEVFNATRFASV | |
| | YAWNRKRISNCVADYSVLYNFAPFFAFKCYGVSPTK | |
| | LNDLCFTNVYADSFVIRGNEVRQIAPGQTGNIADYNY | |
| | KLPDDFTGCVIAWNSNKLDSKVGGNYNYQYRLFRKS | |
| | NLKPFERDISTEIYQAGNKPCNGVAGENCYFPLRSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENLVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2515-1 | QCVNLTTRTQLPPAYTNSFTRGVYYPDKVFRSSVLHS | 123 |
| | TQDLFLPFFSNVTWFHVISGTNGTKRFDNPVLPFNDG | |
| | VYFASIEKSNIIRGWIFGTTLDSKTQSLLIVNNATNVVI | |
| | KVCEFQFCNDPFLDHKNNKSWMESEFRVYSSANNCT | |
| | FEYVSQPFLMDLEGKQGNFKNLREFVFKNIDGYFKIY | |
| | SKHTPIIVREPEDLPQGFSALEPLVDLPIGINITRFQTLL | |
| | ALHRSYLTPGDSSSGWTAGAAAYYVGYLQPRTFLLK | |
| | YNENGTITDAVDCALDPLSETKCTLKSFTVEKGIYQT | |
| | SNFRVQPTESIVRFPNITNLCPFDEVFNATRFASVYAW | |
| | NRKRISNCVADYSVLYNLAPFFTFKCYGVSPTKLNDL | |
| | CFTNVYADSFVIRGDEVRQIAPGQTGNIADYNYKLPD | |
| | DFTGCVIAWNSNKLDSKVSGNYNYLYRLFRKSNLKP | |
| | FERDISTEIYQAGNKPCNGVAGFNCYFPLRSYSFRPTY | |
| | GVGHQPYRVVVLSFELLHAPATVCGPKKSTNLVKNK | |
| | CVNFNFNGLKGTGVLTESNKKFLPFQQFGRDIADTTD | |
| | AVRDPQTLEILDITPCSFGGVSVITPGTNTSNQVAVLY | |
| | QGVNCTEVPVAIHADQLTPTWRVYSTGSNVFQTRAG | |
| | CLIGAEYVNNSYECDIPIGAGICASYQTQTKSHQQAQ | |
| | SVASQSIIAYTMSLGAENSVAYSNNSIAIPTNFTISVTT | |
| | EILPVSMTKTSVDCTMYICGDSTECSNLLLQYGSFCT | |
| | QLKRALTGIAVEQDKNTQEVFAQVKQIYKTPPIKYFG | |
| | GFNFSQILPDPSKPSKRSFIEDLLFNKVTLADAGFIKQ | |
| | YGDCLGDIAARDLICAQKFKGLTVLPPLLTDEMIAQY | |
| | TSALLAGTITSGWTFGAGAALQIPFAMQMAYRENGI | |
| | GVTQNVLYENQKLIANQFNSAIGKIQDSLSSTASALG | |
| | KLQDVVNHNAQALNTLVKQLSSKFGAISSVLNDIFSR | |
| | LDPPEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BV2523-1 | QCVNLITRTQSYTNSFTRGVYYPDKVFRSSVLHSTQD | 124 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHKNNKSWMESEFRVYSSA | |
| | NNCTFEYVSQPFLMDLEGKQGNFKNLREFVFKNIDG | |
| | YFKIYSKHTPINLGRDLPQGFSALEPLVDLPIGINITRF | |
| | QTLLALHRSYLTPGDSSSGWTAGAAAYYVGYLQPRT | |
| | FLLKYNENGTITDAVDCALDPLSETKCTLKSFTVEKG | |
| | IYQTSNFRVQPTESIVRFPNITNLCPFDEVFNATRFASV | |
| | YAWNRKRISNCVADYSVLYNFAPFFAFKCYGVSPTK | |
| | LNDLCFTNVYADSFVIRGNEVSQIAPGQTGNIADYNY | |
| | KLPDDFTGCVIAWNSNKLDSKVGGNYNYLYRLFRKS | |
| | NLKPFERDISTEIYQAGNKPCNGVAGENCYFPLRSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2540-1 | QCVNLITRTQSYTNSFTRGVYYPDKVFRSSVLHSTQD | 125 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATRFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNKLDSKVGGNYNYRYRLFRKSN | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2541-1 | QCVNLITRTQSYTNSFTRGVYYPDKVFRSSVLHSTQD | 126 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATRFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNNLDSKVGGNYNYRYRLFRKSN | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTVSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2549 | QCVNLITRTQSYTNSFTRGVYYPDKVFRSSVLHSTQD | 127 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHKNNKSWMESEFRVYSSA | |
| | NNCTFEYVSQPFLMDLEGKQGNFKNLREFVFKNIDG | |
| | YFKIYSKHTPINLGRDLPQGFSALEPLVDLPIGINITRF | |
| | QTLLALHRSYLTPGDSSSGWTAGAAAYYVGYLQPRT | |
| | FLLKYNENGTITDAVDCALDPLSETKCTLKSFTVEKG | |
| | IYQTSNFRVQPTESIVRFPNITNLCPFDEVFNATRFASV | |
| | YAWNRKRISNCVADYSVLYNFAPFFAFKCYGVSPTK | |
| | LNDLCFTNVYADSFVIRGNEVSQIAPGQTGNIADYNY | |
| | KLPDDFTGCVIAWNSNKLDSKVGGNYNYQYRLFRKS | |
| | NLKPFERDISTEIYQAGNKPCNGVAGENCYFPLRSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENLVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2542-2 | QCVNLITRTQSYTNSFTRGVYYPDKVFRSSVLHSTQD | 128 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHKNNKSWMESEFRVYSSA | |
| | NNCTFEYVSQPFLMDLEGKQGNFKNLREFVFKNIDG | |
| | YFKIYSKHTPINLGRDLPQGFSALEPLVDLPIGINITRF | |
| | QTLLALHRSYLTPGDSSSGWTAGAAAYYVGYLQPRT | |
| | FLLKYNENGTITDAVDCALDPLSETKCTLKSFTVEKG | |
| | IYQTSNFRVQPTESIVRFPNITNLCPFDEVFNATRFASV | |
| | YAWNRKRISNCVADYSVLYNFAPFFAFKCYGVSPTK | |
| | LNDLCFTNVYADSFVIRGNEVRQIAPGQTGNIADYNY | |
| | KLPDDFTGCVIAWNSNKLDSKVGGNYNYQYRLFRKS | |
| | NLKPFERDISTEIYQAGNKPCNGVAGENCYFPLRSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENLVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2562 | QCVNLITRTQSYTNSFTRGVYYPDKVFRSSVLHSTQD | 129 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATTFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNKLDSKVGGNYNYRYRLFRKSN | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNSSYECDIPIGAGICASYQTQTKSH | |
| | QQAQSVASQSIIAYTMSLGAENSVAYSNNSIAIPTNFT | |
| | ISVTTEILPVSMTKTSVDCTMYICGDSTECSNLLLQYG | |
| | SFCTQLKRALTGIAVEQDKNTQEVFAQVKQIYKTPPI | |
| | KYFGGFNFSQILPDPSKPSKRSFIEDLLFNKVTLADAG | |
| | FIKQYGDCLGDIAARDLICAQKFNGLTVLPPLLTDEMI | |
| | AQYTSALLAGTITSGWTFGAGAALQIPFAMQMAYRF | |
| | NGIGVTQNVLYENQKLIANQFNSAIGKIQDSLSSTASA | |
| | LGKLQDVVNHNAQALNTLVKQLSSKFGAISSVLNDIL | |
| | SRLDPPEAEVQIDRLITGRLQSLQTYVTQQLIRAAEIR | |
| | ASANLAATKMSECVLGQSKRVDFCGKGYHLMSFPQ | |
| | SAPHGVVFLHVTYVPAQEKNFTTAPAICHDGKAHFP | |
| | REGVFVSNGTHWFVTQRNFYEPQIITTDNTFVSGNCD | |
| | VVIGIVNNTVYDPLQPELDSFKEELDKYFKNHTSPDV | |
| | DLGDISGINASVVNIQKEIDRLNEVAKNLNESLIDLQE | |
| | LGKYEQYIKWPWYIWLGFIAGLIAIVMVTIMLCCMTS | |
| | CCSCLKGCCSCGSCCKFDEDDSEPVLKGVKLHYT | |
| BV2559 | QCVNLITRTQSYTNSFTRGVYYPDKVFRSSVLHSTQD | 130 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHENNKSRMESELRVYSSAN | |
| | NCTFEYVSQPFLMDLEGKQGNFKNLREFVFKNIDGY | |
| | FKIYSKHTPVNLGRDLPQGFSALEPLVDLPIGINITRFQ | |
| | TLLALHRSYLTPGDSSSSWTAGAAAYYVGYLQPRTF | |
| | LLKYNENGTITDAVDCALDPLSETKCTLKSFTVEKGI | |
| | YQTSNFRVQPTESIVRFPNITNLCPFHEVFNATRFASV | |
| | YAWNRKRISNCVADYSVLYNFAPFFAFKCYGVSPTK | |
| | LNDLCFTNVYADSFVIRGNEVSQIAPGQTGNIADYNY | |
| | KLPDDFTGCVIAWNSNKLDSKVSGNYNYLYRLFRKS | |
| | KLKPFERDISTEIYQAGNKPCNGVAGENCYFPLQSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2577 | QCVNLITRTQSYTNSFTRGVYYPDKVFRSSVLHSTQD | 131 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHENNKSRMESELRVYSSAN | |
| | NCTFEYVSQPFLMDLEGKQGNFKNLREFVFKNIDGY | |
| | FKIYSKHTPVNLGRDLPQGFSALEPLVDLPIGINITRFQ | |
| | TLLALHRSYLTPGDSSSSWTAGAAAYYVGYLQPRTF | |
| | LLKYNENGTITDAVDCALDPLSETKCTLKSFTVEKGI | |
| | YQTSNFRVQPTESIVRFPNITNLCPFHEVFNATTFASV | |
| | YAWNRKRISNCVADYSVLYNFAPFFAFKCYGVSPTK | |
| | LNDLCFTNVYADSFVIRGNEVSQIAPGQTGNIADYNY | |
| | KLPDDFTGCVIAWNSNKLDSKVSGNYNYRYRLFRKS | |
| | KLKPFERDISTEIYQAGNKPCNGVAGSNCYFPLQSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNISNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLENKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLINL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2578 | QCVNLITRTQSYTNSFTRGVYYPDKVFRSSVLHSTQD | 132 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATTFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNKLDSKVGGNYNYRYRLFRKSN | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2589 | QCVNLITRTQSYTNSFTRGVYYPDKVFRSSVLHSTQD | 133 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATTFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNKLDSTVGGNYNYRYRLFRKSK | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2590 | QCVNLITRTQSYTNSFTRGVYYPDKVFRSSVLHSTQD | 134 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYYHKNNKSWMESEFRVYSSANN | |
| | CTFEYVSQPFLMDLEGKQGNFKNLREFVFKNIDGYF | |
| | KIYSKHTPINLGRDLPQGFSALEPLVDLPIGINITRFQT | |
| | LLALHRSYLTPGDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFDEVFNATRFASVY | |
| | AWNRKRISNCVADYSVLYNFAPFFAFKCYGVSPTKL | |
| | NDLCFTNVYADSFVIRGNEVSQIAPGQTGNIADYNYK | |
| | LPDDFTGCVIAWNSNKLDSTVGGNYNYRYRLFRKSK | |
| | LKPFERDISTEIYQAGNKPCNGVAGVNCYFPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2592 | QCVNLITRTQSYTNSFTRGVYYPDKVFRSSVLHSTQD | 135 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYQKNNKSWMESEFRVYSSANNCT | |
| | FEYVSQPFLMDLEGKEGNFKNLREFVFKNIDGYFKIY | |
| | SKHTPINLERDLPQGFSALEPLVDLPIGINITRFQTLLA | |
| | LHRSYLTPGDSSSGWTAGAAAYYVGYLQPRTFLLKY | |
| | NENGTITDAVDCALDPLSETKCTLKSFTVEKGIYQTS | |
| | NFRVQPTESIVRFPNITNLCPFHEVFNATTFASVYAW | |
| | NRKRISNCVADYSVIYNFAPFFAFKCYGVSPTKLNDL | |
| | CFTNVYADSFVIRGNEVSQIAPGQTGNIADYNYKLPD | |
| | DFTGCVIAWNSNKLDSKPSGNYNYLYRLFRKSKLKP | |
| | FERDISTEIYQAGNKPCNGVAGSNCYSPLQSYGFRPT | |
| | YGVGHQPYRVVVLSFELLHAPATVCGPKKSTNLVKN | |
| | KCVNFNFNGLTGTGVLTESNKKFLPFQQFGRDIADTT | |
| | DAVRDPQTLEILDITPCSFGGVSVITPGTNTSNQVAVL | |
| | YQGVNCTEVPVAIHADQLTPTWRVYSTGSNVFQTRA | |
| | GCLIGAEYVNNSYECDIPIGAGICASYQTQTKSHQQA | |
| | QSVASQSIIAYTMSLGAENSVAYSNNSIAIPTNFTISVT | |
| | TEILPVSMTKTSVDCTMYICGDSTECSNLLLQYGSFC | |
| | TQLKRALTGIAVEQDKNTQEVFAQVKQIYKTPPIKYF | |
| | GGFNFSQILPDPSKPSKRSFIEDLLFNKVTLADAGFIK | |
| | QYGDCLGDIAARDLICAQKFNGLTVLPPLLTDEMIAQ | |
| | YTSALLAGTITSGWTFGAGAALQIPFAMQMAYRFNG | |
| | IGVTQNVLYENQKLIANQFNSAIGKIQDSLSSTASALG | |
| | KLQDVVNHNAQALNTLVKQLSSKFGAISSVLNDILSR | |
| | LDPPEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| XBB.1 | QCVNLITRTQSYTNSFTRGVYYPDKVFRSSVLHSTQD | 136 |
| | LFLPFFSNVTWFHAIHVSGTNGTKRFDNPALPFNDGV | |
| | YFASTEKSNIIRGWIFGTTLDSKTQSLLIVNNATNVVI | |
| | KVCEFQFCNDPFLDVYQKNNKSWMESEFRVYSSAN | |
| | NCTFEYVSQPFLMDLEGKEGNFKNLREFVFKNIDGYF | |
| | KIYSKHTPINLERDLPQGFSALEPLVDLPIGINITRFQT | |
| | LLALHRSYLTPVDSSSGWTAGAAAYYVGYLQPRTFL | |
| | LKYNENGTITDAVDCALDPLSETKCTLKSFTVEKGIY | |
| | QTSNFRVQPTESIVRFPNITNLCPFHEVFNATTFASVY | |
| | AWNRKRISNCVADYSVIYNFAPFFAFKCYGVSPTKL | |
| | NDLCFTNVYADSFVIRGNEVSQIAPGQTGNIADYNYK | |
| | LPDDFTGCVIAWNSNKLDSKPSGNYNYLYRLFRKSK | |
| | LKPFERDISTEIYQAGNKPCNGVAGSNCYSPLQSYGF | |
| | RPTYGVGHQPYRVVVLSFELLHAPATVCGPKKSTNL | |
| | VKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | |
| | T | |
| BV2591 | QCVNLITRTQSYTNSFTRGVYYPDKVFRSSVLHSTQD | 137 |
| | LFLPFFSNVTWFHAISGTNGTKRFDNPVLPFNDGVYF | |
| | ASTEKSNIIRGWIFGTTLDSKTQSLLIVNNATNVVIKV | |
| | CEFQFCNDPFLDVYHKNNKSWMESEFRVYSSANNCT | |
| | FEYVSQPFLMDLEGKQGNFKNLREFVFKNIDGYFKIY | |
| | SKHTPINLGRDLPQGFSALEPLVDLPIGINITRFQTLLA | |
| | LHRSYLTPGDSSSGWTAGAAAYYVGYLQPRTFLLKY | |
| | NENGTITDAVDCALDPLSETKCTLKSFTVEKGIYQTS | |
| | NFRVQPTESIVRFPNITNLCPFDEVFNATTFASVYAW | |
| | NRKRISNCVADYSVLYNFAPFFAFKCYGVSPTKLNDL | |
| | CFTNVYADSFVIRGNEVSQIAPGQTGNIADYNYKLPD | |
| | DFTGCVIAWNSNKLDSKVGGNYNYRYRLFRKSNLKP | |
| | FERDISTEIYQAGNKPCNGVAGVNCYFPLQSYGFRPT | |
| | YGVGHQPYRVVVLSFELLHAPATVCGPKKSTNLVKN | |
| | KCVNFNFNGLTGTGVLTESNKKFLPFQQFGRDIADTT | |
| | DAVRDPQTLEILDITPCSFGGVSVITPGTNTSNQVAVL | |
| | YQGVNCTEVPVAIHADQLTPTWRVYSTGSNVFQTRA | |
| | GCLIGAEYVNNSYECDIPIGAGICASYQTQTKSHQQA | |
| | QSVASQSIIAYTMSLGAENSVAYSNNSIAIPTNFTISVT | |
| | TEILPVSMTKTSVDCTMYICGDSTECSNLLLQYGSFC | |
| | TQLKRALTGIAVEQDKNTQEVFAQVKQIYKTPPIKYF | |
| | GGFNFSQILPDPSKPSKRSFIEDLLFNKVTLADAGFIK | |
| | QYGDCLGDIAARDLICAQKFNGLTVLPPLLTDEMIAQ | |
| | YTSALLAGTITSGWTFGAGAALQIPFAMQMAYRENG | |
| | IGVTQNVLYENQKLIANQFNSAIGKIQDSLSSTASALG | |
| | KLQDVVNHNAQALNTLVKQLSSKFGAISSVLNDILSR | |
| | LDPPEAEVQIDRLITGRLQSLQTYVTQQLIRAAEIRAS | |
| | ANLAATKMSECVLGQSKRVDFCGKGYHLMSFPQSA | |
| | PHGVVFLHVTYVPAQEKNFTTAPAICHDGKAHFPRE | |
| | GVFVSNGTHWFVTQRNFYEPQIITTDNTFVSGNCDVV | |
| | IGIVNNTVYDPLQPELDSFKEELDKYFKNHTSPDVDL | |
| | GDISGINASVVNIQKEIDRLNEVAKNLNESLIDLQELG | |
| | KYEQYIKWPWYIWLGFIAGLIAIVMVTIMLCCMTSCC | |
| | SCLKGCCSCGSCCKFDEDDSEPVLKGVKLHYT | |
| BN.1 | QCVNLITRTQSYTNSFTRGVYYPDKVFRSSVLHSTQD | 138 |
| | LFLPFFSNVTWFHAIHVSGTNGTKRFDNPVLPFNDGV | |
| | YFASTEKSNIIRGWIFGTTLDSKTQSLLIVNNATNVVI | |
| | KVCEFQFCNDPFLDVYYHXNNKSRMESELRVYSSAN | |
| | NCTFEYVSQPFLMDLEGKQGNFKNLREFVFKNIDGY | |
| | FKIYSKHTPVNLGRDLPQGFSALEPLVDLPIGINITRFQ | |
| | TLLALHRSYLTPGDSSSSWTAGAAAYYVGYLQPRTF | |
| | LLKYNENGTITDAVDCALDPLSETKCTLKSFTVEKGI | |
| | YQTSNFRVQPTESIVRFPNITNLCPFHEVFNATTFASV | |
| | YAWNRTRISNCVADYSVLYNFAPFFAFKCYGVSPTK | |
| | LNDLCFTNVYADSFVIRGNEVSQIAPGQTGNIADYNY | |
| | KLPDDFTGCVIAWNSNKLDSKVSGNYNYLYRLFRKS | |
| | KLKPFERDISTEIYQAGNKPCNGVAGENCYSPLQSYG | |
| | FRPTYGVGHQPYRVVVLSFELLHAPATVCGPKKSTN | |
| | LVKNKCVNFNFNGLTGTGVLTESNKKFLPFQQFGRDI | |
| | ADTTDAVRDPQTLEILDITPCSFGGVSVITPGTNTSNQ | |
| | VAVLYQGVNCTEVPVAIHADQLTPTWRVYSTGSNVF | |
| | QTRAGCLIGAEYVNNSYECDIPIGAGICASYQTQTKS | |
| | HQQAQSVASQSIIAYTMSLGAENSVAYSNNSIAIPTNF | |
| | TISVTTEILPVSMTKTSVDCTMYICGDSTECSNLLLQY | |
| | GSFCTQLKRALTGIAVEQDKNTQEVFAQVKQIYKTPP | |
| | IKYFGGFNFSQILPDPSKPSKRSFIEDLLFNKVTLADA | |
| | GFIKQYGDCLGDIAARDLICAQKFNGLTVLPPLLTDE | |
| | MIAQYTSALLAGTITSGWTFGAGAALQIPFAMQMAY | |
| | RFNGIGVTQNVLYENQKLIANQFNSAIGKIQDSLSSTA | |
| | SALGKLQDVVNHNAQALNTLVKQLSSKFGAISSVLN | |
| | DILSRLDPPEAEVQIDRLITGRLQSLQTYVTQQLIRAA | |
| | EIRASANLAATKMSECVLGQSKRVDFCGKGYHLMSF | |
| | PQSAPHGVVFLHVTYVPAQEKNFTTAPAICHDGKAH | |
| | FPREGVFVSNGTHWFVTQRNFYEPQIITTDNTFVSGN | |
| | CDVVIGIVNNTVYDPLQPELDSFKEELDKYFKNHTSP | |
| | DVDLGDISGINASVVNIQKEIDRLNEVAKNLNESLIDL | |
| | QELGKYEQYIKWPWYIWLGFIAGLIAIVMVTIMLCC | |
| | MTSCCSCLKGCCSCGSCCKFDEDDSEPVLKGVKLHY | 139 |
| | T | |
| Deltacron | MFVFLVLLPLVSSQCVNLTTRTQLPPAYTNSFTRGVY | |
| | YPDKVFRSSVLHSTQDLFLPFFSNVTWFHVISGTNGT | |
| | KRFDNPVLPFNDGVYFASIEKSNIIRGWIFGTTLDSKT | |
| | QSLLIVNNATNVVIKVCEFQFCNDPFLDHKNNKSWM | |
| | ESEFRVYSSANNCTFEYVSQPFLMDLEGKQGNFKNL | |
| | REFVFKNIDGYFKIYSKHTPIIVREPEDLPQGFSALEPL | |
| | VDLPIGINITRFQTLLALHRSYLTPGDSSSGWTAGAAA | |
| | YYVGYLQPRTFLLKYNENGTITDAVDCALDPLSETK | |
| | CTLKSFTVEKGIYQTSNFRVQPTESIVRFPNITNLCPFD | |
| | EVFNATRFASVYAWNRKRISNCVADYSVLYNLAPFF | |
| | TFKCYGVSPTKLNDLCFTNVYADSFVIRGDEVRQIAP | |
| | GQTGNIADYNYKLPDDFTGCVIAWNSNKLDSKVSGN | |
| | YNYLYRLFRKSNLKPFERDISTEIYQAGNKPCNGVAG | |
| | FNCYFPLRSYSFRPTYGVGHQPYRVVVLSFELLHAPA | |
| | TVCGPKKSTNLVKNKCVNFNFNGLKGTGVLTESNKK | |
| | FLPFQQFGRDIADTTDAVRDPQTLEILDITPCSFGGVS | |
| | VITPGTNTSNQVAVLYQGVNCTEVPVAIHADQLTPT | |
| | WRVYSTGSNVFQTRAGCLIGAEYVNNSYECDIPIGAG | |
| | ICASYQTQTKSHQQAQSVASQSIIAYTMSLGAENSVA | |
| | YSNNSIAIPTNFTISVTTEILPVSMTKTSVDCTMYICGD | |
| | STECSNLLLQYGSFCTQLKRALTGIAVEQDKNTQEVF | |
| | AQVKQIYKTPPIKYFGGFNFSQILPDPSKPSKRSFIEDL | |
| | LFNKVTLADAGFIKQYGDCLGDIAARDLICAQKFKGL | |
| | TVLPPLLTDEMIAQYTSALLAGTITSGWTFGAGAALQ | |
| | IPFAMQMAYRFNGIGVTQNVLYENQKLIANQFNSAIG | |
| | KIQDSLSSTASALGKLQDVVNHNAQALNTLVKQLSS | |
| | KFGAISSVLNDIFSRLDPPEAEVQIDRLITGRLQSLQTY | |
| | VTQQLIRAAEIRASANLAATKMSECVLGQSKRVDFC | |
| | GKGYHLMSFPQSAPHGVVFLHVTYVPAQEKNFTTAP | |
| | AICHDGKAHFPREGVFVSNGTHWFVTQRNFYEPQIIT | |
| | TDNTFVSGNCDVVIGIVNNTVYDPLQPELDSFKEELD | |
| | KYFKNHTSPDVDLGDISGINASVVNIQKEIDRLNEVA | |
| | KNLNESLIDLQELGKYEQYIKWPWYIWLGFIAGLIAI | |
| | VMVTIMLCCMTSCCSCLKGCCSCGSCCKFDEDDSEP | |
| | VLKGVKLHYT | |
| BV2561 | MFVFLVLLPLVSSQCVNLITRTQSYTNSFTRGVYYPD | 145 |
| | KVFRSSVLHSTQDLFLPFFSNVTWFHAIHVSGTNGTK | |
| | RFDNPVLPFNDGVYFASTEKSNIIRGWIFGTTLDSKTQ | |
| | SLLIVNNATNVVIKVCEFQFCNDPFLDVYYHENNKSR | |
| | MESELRVYSSANNCTFEYVSQPFLMDLEGKQGNFKN | |
| | LREFVFKNIDGYFKIYSKHTPVNLGRDLPQGFSALEPL | |
| | VDLPIGINITRFQTLLALHRSYLTPGDSSSSWTAGAAA | |
| | YYVGYLQPRTFLLKYNENGTITDAVDCALDPLSETK | |
| | CTLKSFTVEKGIYQTSNFRVQPTESIVRFPNITNLCPFH | |
| | EVFNATRFASVYAWNRKRISNCVADYSVLYNFAPFF | |
| | AFKCYGVSPTKLNDLCFTNVYADSFVIRGNEVSQIAP | |
| | GQTGNIADYNYKLPDDFTGCVIAWNSNKLDSKVSGN | |
| | YNYLYRLFRKSKLKPFERDISTEIYQAGNKPCNGVAG | |
| | FNCYFPLQSYGFRPTYGVGHQPYRVVVLSFELLHAP | |
| | ATVCGPKKSTNLVKNKCVNFNFNGLTGTGVLTESNK | |
| | KFLPFQQFGRDIADTTDAVRDPQTLEILDITPCSFGGV | |
| | SVITPGTNTSNQVAVLYQGVNCTEVPVAIHADQLTPT | |
| | WRVYSTGSNVFQTRAGCLIGAEYVNNSYECDIPIGAG | |
| | ICASYQTQTKSHQQAQSVASQSIIAYTMSLGAENSVA | |
| | YSNNSIAIPTNFTISVTTEILPVSMTKTSVDCTMYICGD | |
| | STECSNLLLQYGSFCTQLKRALTGIAVEQDKNTQEVF | |
| | AQVKQIYKTPPIKYFGGFNFSQILPDPSKPSKRSFIEDL | |
| | LFNKVTLADAGFIKQYGDCLGDIAARDLICAQKENGL | |
| | TVLPPLLTDEMIAQYTSALLAGTITSGWTFGAGAALQ | |
| | IPFAMQMAYRENGIGVTQNVLYENQKLIANQFNSAIG | |
| | KIQDSLSSTASALGKLQDVVNHNAQALNTLVKQLSS | |
| | KFGAISSVLNDILSRLDPPEAEVQIDRLITGRLQSLQTY | |
| | VTQQLIRAAEIRASANLAATKMSECVLGQSKRVDFC | |
| | GKGYHLMSFPQSAPHGVVFLHVTYVPAQEKNFTTAP | |
| | AICHDGKAHFPREGVFVSNGTHWFVTQRNFYEPQIIT | |
| | TDNTFVSGNCDVVIGIVNNTVYDPLQPELDSFKEELD | |
| | KYFKNHTSPDVDLGDISGINASVVNIQKEIDRLNEVA | |
| | KNLNESLIDLQELGKYEQYIKWPWYIWLGFIAGLIAI | |
| | VMVTIMLCCMTSCCSCLKGCCSCGSCCKFDEDDSEP | |
| | VLKGVKLHYT | |
| BV2562 | MFVFLVLLPLVSSQCVNLITRTQSYTNSFTRGVYYPD | 146 |
| | KVFRSSVLHSTQDLFLPFFSNVTWFHAISGTNGTKRF | |
| | DNPVLPFNDGVYFASTEKSNIIRGWIFGTTLDSKTQSL | |
| | LIVNNATNVVIKVCEFQFCNDPFLDVYYHKNNKSWM | |
| | ESEFRVYSSANNCTFEYVSQPFLMDLEGKQGNFKNL | |
| | REFVFKNIDGYFKIYSKHTPINLGRDLPQGFSALEPLV | |
| | DLPIGINITRFQTLLALHRSYLTPGDSSSGWTAGAAAY | |
| | YVGYLQPRTFLLKYNENGTITDAVDCALDPLSETKCT | |
| | LKSFTVEKGIYQTSNFRVQPTESIVRFPNITNLCPFDEV | |
| | FNATTFASVYAWNRKRISNCVADYSVLYNFAPFFAF | |
| | KCYGVSPTKLNDLCFTNVYADSFVIRGNEVSQIAPGQ | |
| | TGNIADYNYKLPDDFTGCVIAWNSNKLDSKVGGNY | |
| | NYRYRLFRKSNLKPFERDISTEIYQAGNKPCNGVAGV | |
| | NCYFPLQSYGFRPTYGVGHQPYRVVVLSFELLHAPA | |
| | TVCGPKKSTNLVKNKCVNFNFNGLTGTGVLTESNKK | |
| | FLPFQQFGRDIADTTDAVRDPQTLEILDITPCSFGGVS | |
| | VITPGTNTSNQVAVLYQGVNCTEVPVAIHADQLTPT | |
| | WRVYSTGSNVFQTRAGCLIGAEYVNSSYECDIPIGAG | |
| | ICASYQTQTKSHQQAQSVASQSIIAYTMSLGAENSVA | |
| | YSNNSIAIPTNFTISVTTEILPVSMTKTSVDCTMYICGD | |
| | STECSNLLLQYGSFCTQLKRALTGIAVEQDKNTQEVF | |
| | AQVKQIYKTPPIKYFGGFNFSQILPDPSKPSKRSFIEDL | |
| | LFNKVTLADAGFIKQYGDCLGDIAARDLICAQKENGL | |
| | TVLPPLLTDEMIAQYTSALLAGTITSGWTFGAGAALQ | |
| | IPFAMQMAYRFNGIGVTQNVLYENQKLIANQFNSAIG | |
| | KIQDSLSSTASALGKLQDVVNHNAQALNTLVKQLSS | |
| | KFGAISSVLNDILSRLDPPEAEVQIDRLITGRLQSLQTY | |
| | VTQQLIRAAEIRASANLAATKMSECVLGQSKRVDFC | |
| | GKGYHLMSFPQSAPHGVVFLHVTYVPAQEKNFTTAP | |
| | AICHDGKAHFPREGVFVSNGTHWFVTQRNFYEPQIIT | |
| | TDNTFVSGNCDVVIGIVNNTVYDPLQPELDSFKEELD | |
| | KYFKNHTSPDVDLGDISGINASVVNIQKEIDRLNEVA | |
| | KNLNESLIDLQELGKYEQYIKWPWYIWLGFIAGLIAI | |
| | VMVTIMLCCMTSCCSCLKGCCSCGSCCKFDEDDSEP | |
| | VLKGVKLHYT | |
| BV2577 | MFVFLVLLPLVSSQCVNLITRTQSYTNSFTRGVYYPD | 147 |
| | KVFRSSVLHSTQDLFLPFFSNVTWFHAIHVSGTNGTK | |
| | RFDNPVLPFNDGVYFASTEKSNIIRGWIFGTTLDSKTQ | |
| | SLLIVNNATNVVIKVCEFQFCNDPFLDVYYHENNKSR | |
| | MESELRVYSSANNCTFEYVSQPFLMDLEGKQGNFKN | |
| | LREFVFKNIDGYFKIYSKHTPVNLGRDLPQGFSALEPL | |
| | VDLPIGINITRFQTLLALHRSYLTPGDSSSSWTAGAAA | |
| | YYVGYLQPRTFLLKYNENGTITDAVDCALDPLSETK | |
| | CTLKSFTVEKGIYQTSNFRVQPTESIVRFPNITNLCPFH | |
| | EVENATTFASVYAWNRKRISNCVADYSVLYNFAPFF | |
| | AFKCYGVSPTKLNDLCFTNVYADSFVIRGNEVSQIAP | |
| | GQTGNIADYNYKLPDDFTGCVIAWNSNKLDSKVSGN | |
| | YNYRYRLFRKSKLKPFERDISTEIYQAGNKPCNGVAG | |
| | SNCYFPLQSYGFRPTYGVGHQPYRVVVLSFELLHAP | |
| | ATVCGPKKSTNLVKNKCVNFNFNGLTGTGVLTESNK | |
| | KFLPFQQFGRDIADTTDAVRDPQTLEILDITPCSFGGV | |
| | SVITPGTNISNQVAVLYQGVNCTEVPVAIHADQLTPT | |
| | WRVYSTGSNVFQTRAGCLIGAEYVNNSYECDIPIGAG | |
| | ICASYQTQTKSHQQAQSVASQSIIAYTMSLGAENSVA | |
| | YSNNSIAIPTNFTISVTTEILPVSMTKTSVDCTMYICGD | |
| | STECSNLLLQYGSFCTQLKRALTGIAVEQDKNTQEVF | |
| | AQVKQIYKTPPIKYFGGFNFSQILPDPSKPSKRSFIEDL | |
| | LFNKVTLADAGFIKQYGDCLGDIAARDLICAQKENGL | |
| | TVLPPLLTDEMIAQYTSALLAGTITSGWTFGAGAALQ | |
| | IPFAMQMAYRFNGIGVTQNVLYENQKLIANQFNSAIG | |
| | KIQDSLSSTASALGKLQDVVNHNAQALNTLVKQLSS | |
| | KFGAISSVLNDILSRLDPPEAEVQIDRLITGRLQSLQTY | |
| | VTQQLIRAAEIRASANLAATKMSECVLGQSKRVDFC | |
| | GKGYHLMSFPQSAPHGVVFLHVTYVPAQEKNFTTAP | |
| | AICHDGKAHFPREGVFVSNGTHWFVTQRNFYEPQIIT | |
| | TDNTFVSGNCDVVIGIVNNTVYDPLQPELDSFKEELD | |
| | KYFKNHTSPDVDLGDISGINASVVNIQKEIDRLNEVA | |
| | KNLNESLINLQELGKYEQYIKWPWYIWLGFIAGLIAI | |
| | VMVTIMLCCMTSCCSCLKGCCSCGSCCKFDEDDSEP | |
| | VLKGVKLHYT | |

### Example 3: Epitope Mapping of Antibodies 239.12; 322.3; 425.6; and 35.13

Purpose: Alanine scanning mutagenesis was performed to identify the epitopes of the 239.12, 322.3, 425.6, and 35.13 antibodies.

Methods: Shotgun Mutagenesis epitope mapping services were provided by Integral Molecular (Philadelphia, PA) using a SARS-CoV-2 (Wuhan-Hu-1 strain) S protein RBD shotgun mutagenesis mutation library, made using a full-length expression construct for S protein. One hundred and eighty four residues of the RBD were mutated individually to alanine, and alanine residues to serine. The mutant library was arrayed in 384-well microplates, transiently transfected into HEK293T cells, and allowed to express for 22 h. Cells were then incubated with antibodies at concentrations pre-determined using an independent binding titration curve on cells expressing wild type spike. Cells were fixed in 4% (v/v) paraformaldehyde (Electron Microscopy Sciences), and permeabilized with 0.2% (w/v) saponin (Sigma-Aldrich) in PBS plus calcium and magnesium (PBS++) before incubation with brMAbs diluted in PBS++, 10% normal goat serum (Sigma), and 0.1% saponin. Antibodies were detected using 3.75 μg/mL of Alexa-Fluor-488-conjugated secondary antibodies (Jackson ImmunoResearch Laboratories) in 10% normal goat serum with 0.1% saponin. Cells were washed three times with PBS++/0.1% saponin followed by two washes in PBS, and mean cellular fluorescence was detected using a high-throughput Intellicyte iQue flow cytometer (Sartorius). Antibody reactivity against each mutant S protein clone was calculated relative to wild-type S protein reactivity by subtracting the signal from mock-transfected controls and normalizing to the signal from wild-type S-transfected controls. Mutations within clones were identified as critical to the mAb epitope if they did not support reactivity of the test mAb but supported reactivity of other SARS-CoV-2 antibodies. This counter-screen strategy facilitates the exclusion of S protein mutants that are locally misfolded or have an expression defect. Amino acid sequences of Novavax recombinant spike (rS) proteins were aligned in Clustal Omega (ebi.ac.uk/Tools/msa/clustalo). Any point mutations from the ancestral sequence were noted, and critical amino acids for brMAb binding were highlighted. Residues involved in hACE2 binding and Class 1-4 antibody binding were noted as described by Barnes, C.O., Jette, C.A., Abernathy, M.E. et al. SARS-CoV-2 neutralizing antibody structures inform therapeutic strategies. Nature 588, 682-687 (2020). It is noted that Barnes' antibody classes were defined using human mAbs, and the mAbs described in this work were generated in mice.

Results: Table K shows binding reactivity of 322.3, 239.12, and 425.6 Fabs to a mutant SARS-CoV-2 S RBD compared to binding to the wild-type SARS-CoV-2 S RBD. Underlined amino acids were determined to be important for binding. The amino acids in Table K are numbered with respect to a SARS-CoV-2 S protein of SEQ ID NO: 10.

TABLE K
Binding Reactivity of Fabs to a Mutant SARS-CoV-2 S
RBD Compared to Wild-Type

| Mutation in SARS-CoV-2 | Binding Reactivity (% WT) | | |
|---|---|---|---|
| S protein | 322.3 | 239.12 | 425.6 |
| A352S | 65 (5) | 104.1 (19) | 1.5 (1) |
| K378A | 12.1 (1) | 105.9 (18) | 129.4 (21) |
| T385A | 8.4 (0) | 115.3 (14) | 131.5 (29) |
| L387A | 0.9 (0) | 43.9 (1) | 48.1 (8) |
| C391A | 32 (11) | 72.6 (13) | 33 (4) |
| K444A | 80.9 (2) | 71.8 (11) | −3.9 (0) |
| V445A | 87.6 (2) | 2.7 (3) | 12.3 (4) |
| G446A | 77 (0) | 94.7 (15) | 0.4 (3) |
| N448A | 50.4 (6) | 44.4 (5) | 18.3 (8) |
| R457A | 22.7 (1) | 29.9 (11) | −4.6 (2) |
| S459A | 80.5 (7) | 113.8 (1) | 0.2 (1) |
| G496A | 113.1 (31) | 11.3 (4) | 138.4 (44) |
| Q498A | 116 (16) | −3.9 (0) | 137 (14) |
| P499A | 48.4 (95) | 12.1 (1) | 34 (8) |
| T500A | 99.7 (6) | 11.4 (2) | 101.1 (6) |
| H519A | 93.6 (20) | 106.1 (22) | 3.6 (9) |
| C525A | 14 (0) | 69.9 (8) | 29.9 (1) |
| G526A | 39.5 (7) | 76.8 (21) | 87.3 (17) |

The critical amino acids (residues 476, 485, 486, 487, 489 of SEQ ID NO: 10) for binding of 35.13 Fab to SARS-CoV-2 glycoprotein are shown in Table L. Further structural data showed that amino acids 485, 486, 487, and 489 were particularly critical for binding of 35.13 to SARS-CoV-2 S glycoproteins. Additionally, structural data confirmed that amino acids 378 and 385 were critical for binding of 322.3 to SARS-CoV-2 S glycoproteins and that amino acids 444, 445, 446, and 448 were critical for binding of 425.6 to SARS-CoV-2 S glycoproteins.

TABLE L
Critical Residues for binding of 35.13 Fab to
SARS-CoV-2 glycoprotein

| Mutation in SARS-CoV-2 | Binding Reactivity |
|---|---|
| S protein | (% WT) of 35.13 |
| G476A | 12.8 (4) |
| G485A | 2.8 (1) |
| F486A | 3.6 (1) |
| N487A | 4.2 (0) |
| Y489A | 4.9 (10) |

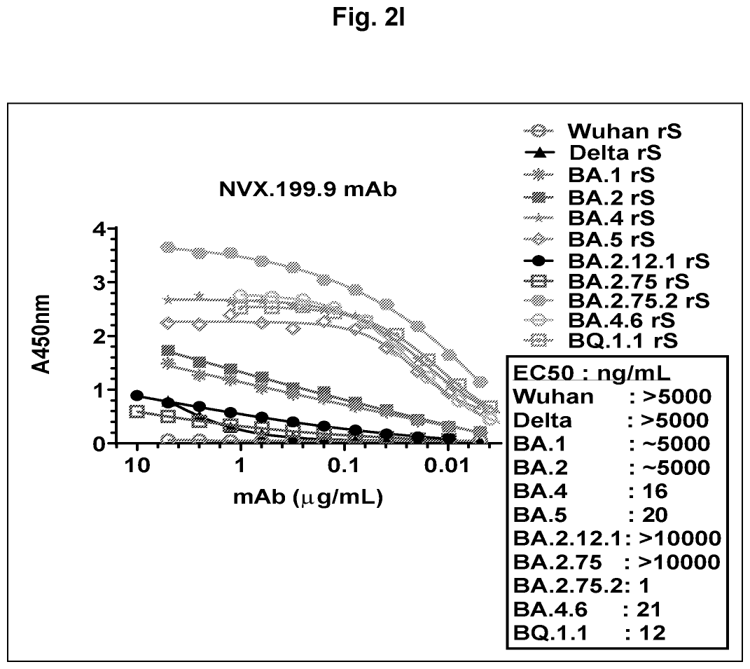

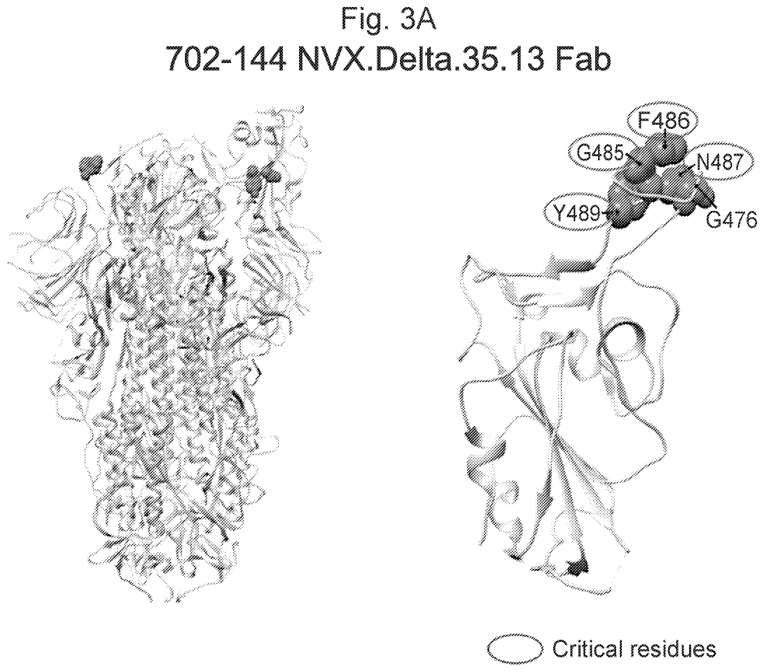

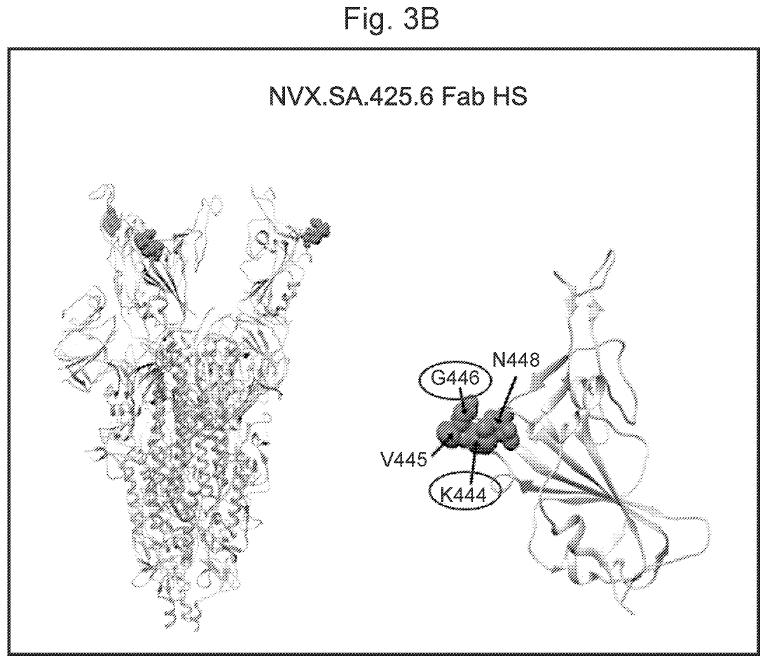

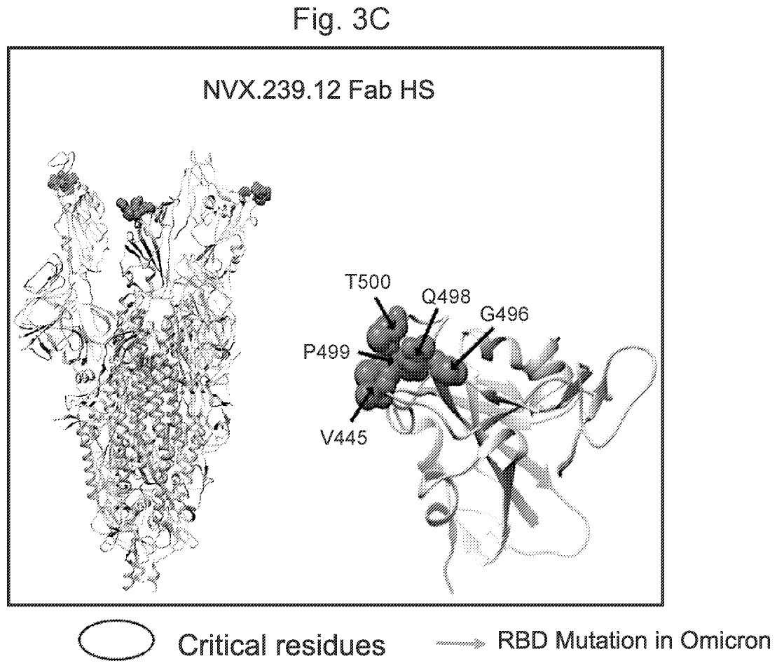

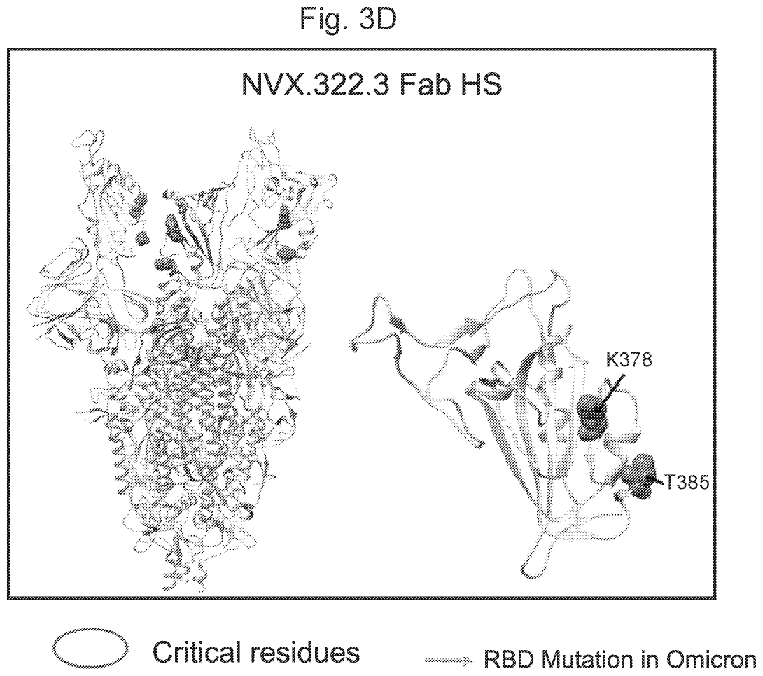

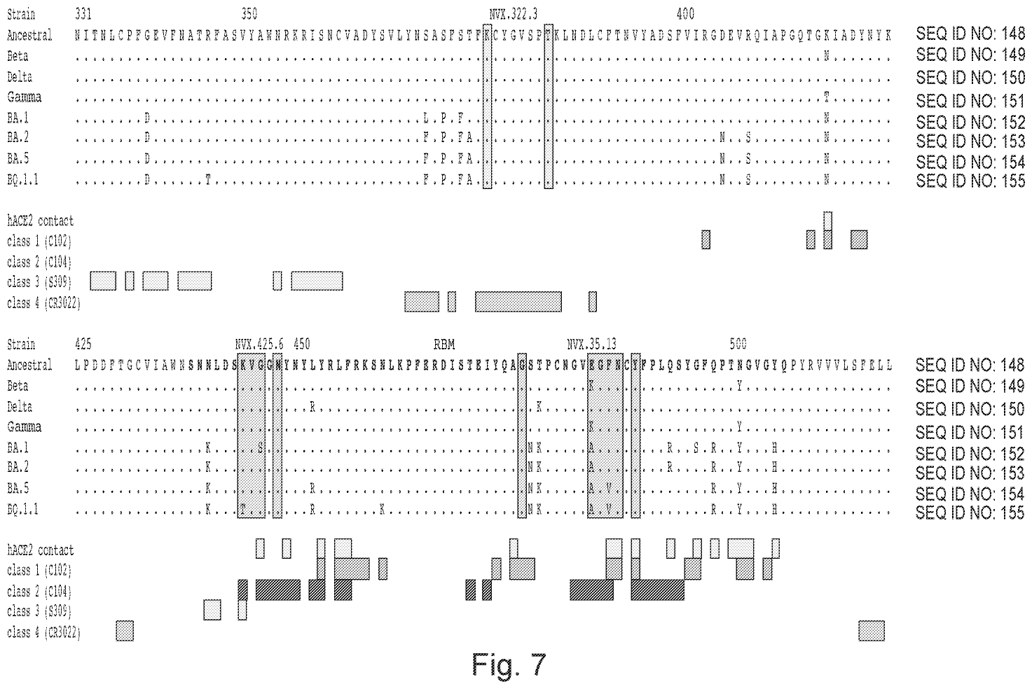

FIGS. 3A-3D show a crystal structure of a SARS-CoV-2 S glycoprotein (Protein Databank ID: 6XCN. Critical residues for binding the 35.13 (FIG. 3A), 425.6 (FIG. 3B), 239.12 (FIG. 3C), and 322.3 (FIG. 3D) Fabs are shown as spheres. The right structure in each figure shows the critical residues of the SARS-CoV-2 S receptor binding domain (RBD) for binding each Fab (Protein Databank ID: 6Z2M). FIG. 7 shows an alignment of the SARS-CoV-2 S glycoproteins from the ancestral, beta, delta, gamma, BA.1, BA.2, BA.5, and BQ.1.1 SARS-CoV-2 viruses. Critical amino acids (K378 and T385) for binding of the 322.3 to the SARS-CoV-2 S glycoproteins are boxed. Critical amino acids (K444, V445, G446, and N448) for binding of the 425.6 to the SARS-CoV-2 S glycoproteins are boxed. Critical amino acids (K444, V445, G446, and N448) for binding of the 425.6 to the SARS-CoV-2 S glycoproteins are boxed. The numbering of the critical amino acids is relative to a SARS-CoV-2 S glycoprotein of SEQ ID NO: 10.

### Numbered Embodiments

1. An antibody or fragment thereof that binds to a sudden acute respiratory syndrome coronavirus 2 (SARS-CoV-2) Spike (S) protein.

2. The antibody or fragment thereof of embodiment 1, wherein the light chain complementarity-determining region 1 (VL CDR1) is selected from the group consisting of SEQ ID NOS: 11-14; the light chain complementarity-determining region 2 (VL CDR2) is selected from the group consisting of SEQ ID NOS: 15-18; the light chain complementarity-determining region 3 (VL CDR3) is selected from the group consisting of SEQ ID NOS: 19-22; the heavy chain complementarity-determining region 1 (VH CDR1) is selected from the group consisting of SEQ ID NOS: 23-26; the heavy chain complementarity-determining region 2 (VH CDR2) is selected from the group consisting of SEQ ID NOS: 27-30; and the heavy chain complementarity-determining region 3 (VH CDR3) is selected from the group consisting of SEQ ID NOS: 31-34.

3. The antibody or fragment thereof of embodiment 1 or 2, comprising:
(i) a VH CDR1 according to SEQ ID NO: 23, a VH CDR2 according to SEQ ID NO: 27, and a VH CDR3 according to SEQ ID NO: 31; a VL CDR1 according to SEQ ID NO: 11, a VL CDR2 according to SEQ ID NO: 15; and a VL CDR3 according to SEQ ID NO: 19;
(ii) a VH CDR1 according to SEQ ID NO: 24; a VH CDR2 according to SEQ ID NO: 28; a VH CDR3 according to SEQ ID NO: 32; a VL CDR1 according to SEQ ID NO: 12; a VL CDR2 according to SEQ ID NO: 16; and a VL CDR3 according to SEQ ID NO: 20;
(iii) a VH CDR1 according to SEQ ID NO: 25; a VH CDR2 according to SEQ ID NO: 29; a VH CDR3 according to SEQ ID NO: 33; a VL CDR1 according to SEQ ID NO: 13; a VL CDR2 according to SEQ ID NO: 17; and a VL CDR3 according to SEQ ID NO: 21; or
(iv) a VH CDR1 according to SEQ ID NO: 26; a VH CDR2 according to SEQ ID NO: 30; a VH CDR3 according to SEQ ID NO: 34; a VL CDR1 according to SEQ ID NO: 14; a VL CDR2 according to SEQ ID NO: 18; and a VL CDR3 according to SEQ ID NO: 22.

4. The antibody or fragment thereof of any one of embodiments 1-3, wherein the amino acid sequence of the variable heavy (VH) domain comprises or consists of an amino acid sequence with at least 90%, at least 95%, at least 96%, at least 97%, at least 98%, at least 99%, or 100% identity to a polypeptide having the amino acid sequence of any one of SEQ ID NOS: 5-8.

5. The antibody or fragment thereof of any one of embodiments 1-4, wherein the amino acid sequence of the variable light (VL) domain comprises or consists of an amino acid sequence with at least 90%, at least 95%, at least 96%, at least 97%, at least 98%, at least 99%, or 100% identity to a polypeptide having the amino acid sequence of any one of SEQ ID NOS: 1-4.

6. The antibody or fragment thereof of any one of embodiments 1-5, wherein said antibody is selected from the group consisting of:
an antibody comprising (i) a VH comprising an amino acid sequence with at least 90%, at least 95%, at least 96%, at least 97%, at least 98%, at least 99%, or 100% identity to SEQ ID NO:5; and (ii) a VL comprising an amino acid sequence with at least 90%, at least 95%, at least 96%, at least 97%, at least 98%, at least 99%, or 100% identity to SEQ ID NO: 1;
an antibody comprising a (i) a VH comprising an amino acid sequence with at least 900%, at least 95%, at least 96%, at least 97%, at least 98%, at least 99%, or 100% identity to SEQ ID NO:6; and (ii) a VL comprising an amino acid sequence with at least 90%, at least 95%, at least 96%, at least 97%, at least 98%, at least 99%, or 100% identity to SEQ ID NO: 2;
an antibody comprising (i) a VH comprising an amino acid sequence with at least 90%, at least 95%, at least 96%, at least 97%, at least 98%, at least 99%, or 100% identity to SEQ ID NO:7; and (ii) a VL comprising an amino acid sequence with at least 90%, at least 95%, at least 96%, at least 97%, at least 98%, at least 99%, or 100% identity to SEQ ID NO: 3;and
an antibody comprising (i) a VH comprising an amino acid sequence with at least 90%, at least 95%, at least 96%, at least 97%, at least 98%, at least 99%, or 100% identity to SEQ ID NO: 4; and (ii) a VL comprising an amino acid sequence with at least 90%, at least 95%, at least 96%, at least 97%, at least 98%, at least 99%, or 100% identity to SEQ ID NO: 8.

7. The antibody or fragment thereof of any one of embodiments 1-6, wherein the antibody or fragment thereof is a monoclonal antibody, a Fab, F(ab′)2, Fab′, a scFv, or a single domain antibody (sdAb).

8. The antibody or fragment thereof of any one of embodiments 1-7, wherein the antibody comprises a human IgG1 or IgG4 domain.

9. The antibody or fragment thereof of any one of embodiments 1-8, wherein the antibody or fragment thereof has a dissociation constant (KD) for a SARS-CoV-2 S polypeptide or variant thereof of 50 nM or less, 10 nM or less, 1 nM or less, 0.5 nM or less, 0.1 nM or less, 0.05 nM or less, 0.01 nM or less, or 0.001 nM or less.

10. The antibody or fragment thereof of any one of embodiments 1-9, wherein the antibody or fragment thereof binds to one or more CoV S polypeptides with at least 90%, at least 91%, at least 92%, at least 93%, at least 94%, at least 95%, at least 96%, at least 97%, at least 98%, at least 99%, or 100% identity to a polypeptide according to SEQ ID NOS: 9, 10, 35-43, and 72-73.

11. An isolated nucleic acid molecule encoding the antibody or fragment thereof of any one of embodiments 1-10.

12. An expression vector comprising a nucleic acid segment encoding the antibody or fragment thereof of any one of embodiments 1-10.

13. A host cell comprising the expression vector of embodiment 12.

14. A pharmaceutical composition comprising the antibody of any one of embodiments 1-10 and a pharmaceutically-acceptable carrier.

15. A method of treating a subject in need thereof infected with a SARS-CoV-2 virus or variant thereof comprising administering to the subject an antibody or fragment thereof according to any one of embodiments 1-10 or the pharmaceutical composition of embodiment 14.

16. The method of embodiment 15, wherein the subject is aged 65 or older.

17. The method of embodiment 15, wherein the subject is immunocompromised.

18. The method of embodiment 15, wherein the subject is a pregnant female.

19. The method of embodiment 15, wherein the SARS-CoV-2 variant is selected from the group consisting of: B.1.1.7 SARS-CoV-2 strain; B.1.351 SARS-CoV-2 strain; P.1 SARS-CoV-2 strain; Cal.20C SARS-CoV-2 strain; B.1.617.2 SARS-CoV-2 strain; B.1.525 SARS-CoV-2 strain; B.1.526 SARS-CoV-2 strain; B.1.617.1 SARS-CoV-2 strain; C.37 SARS-CoV-2 strain; B.1.621 SARS-CoV-2 strain; and B.1.1.529 SARS-CoV-2 strain.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. The following patent documents are incorporated by reference herein in their entireties for all purposes: International Application No. PCT/US2022/080700, filed Nov. 30, 2022; International Publication No. 2021/154812; and International Publication No. 2022/203963.

1. An antibody or fragment thereof that binds to a sudden acute respiratory syndrome coronavirus 2 (CoV) Spike (S) glycoprotein, wherein the antibody or fragment thereof comprises:
(i) a variable light chain complementarity-determining region 1 (VL CDR1) with at least 80%, at least 85%, at least 90%, at least 95%, or 100% identity to a sequence selected from the group consisting of SEQ ID NOS: 11-14 and 76;
(ii) a variable light chain complementarity-determining region 2 (VL CDR2) with at least 80%, at least 85%, at least 90%, at least 95%, or 100% identity to a sequence selected from the group consisting of SEQ ID NOS: 15-18 and 77;
(iii) a variable light chain complementarity-determining region 3 (VL CDR3) with at least 80%, at least 85%, at least 90%, at least 95%, or 100% identity to a sequence selected from the group consisting of SEQ ID NOS: 19-22 and 78;
(iv) a variable heavy chain complementarity-determining region 1 (VH CDR1) with at least 80%, at least 85%, at least 90%, at least 95%, or 100% identity to a sequence selected from the group consisting of SEQ ID NOS: 23-26 and 79;
(v) a variable heavy chain complementarity-determining region 2 (VH CDR2) with at least 80%, at least 85%, at least 90%, at least 95%, or 100% identity to a sequence selected from the group consisting of SEQ ID NOS: 27-30 and 80; and
(vi) a variable heavy chain complementarity-determining region 3 (VH CDR3) with at least 80%, at least 85%, at least 90%, at least 95%, or 100% identity to a sequence selected from the group consisting of SEQ ID NOS: 31-34 and 81.

2. An antibody or fragment thereof that binds to a sudden acute respiratory syndrome coronavirus 2 (SARS-CoV-2) Spike (S) protein, wherein the antibody or fragment thereof comprises:
(i) a variable heavy (VH) domain comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NOS: 5-8 and 75; and
(ii) a variable light (VL) domain comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1-4 and 74.

3. The antibody or fragment thereof of claim 1, wherein the antibody or fragment thereof comprises:
(i) a variable heavy (VH) domain comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NOS: 5-8 and 75; and
(ii) a variable light (VL) domain comprising an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1-4 and 74.

4. The antibody or fragment thereof of claim 2, wherein the antibody or fragment thereof comprises:
(i) a variable light chain complementarity-determining region 1 (VL CDR1) with at least 80%, at least 85%, at least 90%, at least 95%, or 100% identity to a sequence selected from the group consisting of SEQ ID NOS: 11-14 and 76;
(ii) a variable light chain complementarity-determining region 2 (VL CDR2) with at least 80%, at least 85%, at least 90%, at least 95%, or 100% identity to a sequence selected from the group consisting of SEQ ID NOS: 15-18 and 77;
(iii) a variable light chain complementarity-determining region 3 (VL CDR3) with at least 80%, at least 85%, at least 90%, at least 95%, or 100% identity to a sequence selected from the group consisting of SEQ ID NOS: 19-22 and 78;
(iv) a variable heavy chain complementarity-determining region 1 (VH CDR1) with at least 80%, at least 85%, at least 90%, at least 95%, or 100% identity to a sequence selected from the group consisting of SEQ ID NOS: 23-26 and 79;
(v) a variable heavy chain complementarity-determining region 2 (VH CDR2) with at least 80%, at least 85%, at least 90%, at least 95%, or 100% identity to a sequence selected from the group consisting of SEQ ID NOS: 27-30 and 80; and
(vi) a variable heavy chain complementarity-determining region 3 (VH CDR3) with at least 80%, at least 85%, at least 90%, at least 95%, or 100% identity to a sequence selected from the group consisting of SEQ ID NOS: 31-34 and 81.

5. The antibody or fragment thereof of claim 4, wherein the antibody or fragment thereof is selected from the group consisting of: a VH CDR1 according to SEQ ID NOS: 23-26, 79, a V_{H} CDR2 according to SEQ ID NOS: 27-30, 80, a V_{H} CDR3 according to SEQ ID NOS: 31-34, 81, a VL CDR1 according to SEQ ID NOS: 11-14,76, a VL CDR2 according to SEQ ID NOS: 15-18,77, and a VL CDR3 according to SEQ ID NOS: 19-22, 78.

6-9. (canceled)

10. The antibody or fragment thereof of claim 4, wherein the antibody or fragment thereof is selected from the group consisting of:
(i) a VH comprising the amino acid sequence of SEQ ID NOS:5-8, 75; and
(ii) a VL comprising the amino acid sequence of SEQ ID NOS: 1-4, 74.

11-14. (canceled)

15. The antibody or fragment thereof of claim 4, wherein the antibody or fragment thereof is a monoclonal antibody, a Fab, F(ab′)₂, Fab′, a scFv, or a single domain antibody (sdAb).

16. The antibody or fragment thereof of claim 4, wherein the antibody comprises a human IgG1 or IgG4 domain.

17. The antibody or fragment thereof of claim 4, wherein the antibody or fragment thereof has an equilibrium dissociation constant (K_{D}) for a CoV S glycoprotein or variant thereof of 50 nM or less, 10 nM or less, 1 nM or less, 0.5 nM or less, 0.1 nM or less, 0.05 nM or less, 0.01 nM or less, or 0.001 nM or less.

18. The antibody or fragment thereof of claim 4, wherein the antibody or fragment thereof binds to a CoV S glycoprotein or variant thereof with an equilibrium dissociation constant (Kd) of less than 1.0×10⁻⁹ moles per liter (M), less than 1.0×10⁻¹⁰ M, less than 1.0×10⁻¹¹ M, or less than 1.0×10⁻¹² M.

19. The antibody or fragment thereof of claim 4, wherein the antibody or fragment thereof binds to one or more CoV S polypeptides with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide according to any one of SEQ ID NOS: 9, 10, 35-43, 72-73, 90-139, and 145-147.

20-21. (canceled)

22. The antibody or fragment thereof of claim 4, wherein the antibody or fragment thereof binds to an epitope on a CoV S glycoprotein, wherein the epitope comprises amino acids 476, 485, 486, 487, and 489 of the CoV S glycoprotein of SEQ ID NO: 10.

23. (canceled)

24. The antibody or fragment thereof of claim 4, wherein the antibody or fragment thereof binds to an epitope on a CoV S glycoprotein, wherein the epitope comprises amino acids 378 and 385 of the CoV S glycoprotein of SEQ ID NO: 10.

25. The antibody or fragment thereof of claim 4, wherein the antibody or fragment thereof binds to an epitope on a CoV S glycoprotein, wherein the epitope comprises amino acids 444, 445, 446, and 448 of the CoV S glycoprotein of SEQ ID NO: 10.

26-28. (canceled)

29. A pharmaceutical composition, comprising an antibody or fragment thereof of claim 4 and a pharmaceutically-acceptable carrier.

30-36. (canceled)


SEQUENCE LISTING (155 sequences)

SEQ ID NO: 1 - protein, 107 aa, synthetic construct
QIVLTQSPAIMSVSPGEKVTMTCSASSTMSFMYWYQQKPGSSPRLLIYDTSNLASGVPVRFSGSGSGTSYSLTISRVEAEDAATYYCQQWNTYPLTFGAGTKLELKR

SEQ ID NO: 2 - protein, 108 aa, synthetic construct
EIQMTQSPSSMSASLGDRITITCQATQDIVKNLNWYQQKPGKPPSFLIYHATELAKGVPSRFSGSGSGSDYSLTISNLESEDFADYYCLQFYDFPFTFGAGTKLELKR

SEQ ID NO: 3 - protein, 108 aa, synthetic construct
DIQMTQSPASLSASVGETVTITCGASENIYGALNWYQRKQGKSPQLLIYGATNLADGMSSRFSGSGSGRQYSLKISSLHPDDVATYYCQNVLSTPYTFGGGTKLEIKR

SEQ ID NO: 4 - protein, 131 aa, synthetic construct
METDTLLLWVLLLWVPGSTGNIVLTQSPASLAVSLGQRATISCRASESVDSYGSSFMHWYQQKPGQPPKLLIYLASNLESGVPARFSGSGSRTDFTLTIDPVEADDAATYYCQQNNEDPRTFGGGTKLEIK

SEQ ID NO: 5 - protein, 116 aa, synthetic construct
VQLKQSGPSLVQPSQSLSITCTVSGFSLTSYGIHWVRQSPGKGLEWLGVIWRGGSTDYNAAFMSRLTITKDNSKSQVFFKMNSLQADDTAIYYCATHTEGDDVDYWGQGTSVTVSS

SEQ ID NO: 6 - protein, 116 aa, synthetic construct
QVQLQQSGAELGKPGASVKLSCRASGYTFTRYYIYWVKQRPGQGLEWIGEINPSNGDTHFNEKFKTKATLTVDKSSSSAHIHLTSLTSEDSAVYYCSIHAYSFDFWGQGTTLTVSS

SEQ ID NO: 7 - protein, 120 aa, synthetic construct
EVKLEESGPVLVKPSQSLSLTCTVTGYSITSDYAWNWIRQFPGNKLEWMGYISNSAGTSYNPSLKSRISITRDTSKNQFFLQLNSVTTEDTATYYCASGGWSSYYAMDYWGQGTSVTVSS

SEQ ID NO: 8 - protein, 140 aa, synthetic construct
MGWSRIFLFLLSIIAGVHCRVQLQQSGPELVKPGASVTISCKASGYIFTSDYIHWVKQRPGQGPEWIGWIYPGNINTKYNEKFKGKATLTADKSSSTAYMQLSSLTSEDSAVYFCARDDGNLYYYAMDYWGQGTSVTVSS

SEQ ID NO: 9 - protein, 1260 aa, Severe acute respiratory syndrome-related coronavirus
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 10 aa, synthetic construct
SASSTMSFMY

SEQ ID NO: 12 - protein, 11 aa, synthetic construct
QATQDIVKNLN

SEQ ID NO: 13 - protein, 11 aa, synthetic construct
GASENIYGALN

SEQ ID NO: 14 - protein, 15 aa, synthetic construct
RASESVDSYGSSFMH

SEQ ID NO: 15 - protein, 7 aa, synthetic construct
DTSNLAS

SEQ ID NO: 16 - protein, 7 aa, synthetic construct
HATELAK

SEQ ID NO: 17 - protein, 7 aa, synthetic construct
GATNLAD

SEQ ID NO: 18 - protein, 7 aa, synthetic construct
LASNLES

SEQ ID NO: 19 - protein, 9 aa, synthetic construct
QQWNTYPLT

SEQ ID NO: 20 - protein, 9 aa, synthetic construct
LQFYDFPFT

SEQ ID NO: 21 - protein, 9 aa, synthetic construct
QNVLSTPYT

SEQ ID NO: 22 - protein, 9 aa, synthetic construct
QQNNEDPRT

SEQ ID NO: 23 - protein, 5 aa, synthetic construct
SYGIH

SEQ ID NO: 24 - protein, 5 aa, synthetic construct
RYYIY

SEQ ID NO: 25 - protein, 6 aa, synthetic construct
SDYAWN

SEQ ID NO: 26 - protein, 5 aa, synthetic construct
SDYIH

SEQ ID NO: 27 - protein, 16 aa, synthetic construct
VIWRGGSTDYNAAFMS

SEQ ID NO: 28 - protein, 17 aa, synthetic construct
EINPSNGDTHFNEKFKT

SEQ ID NO: 29 - protein, 16 aa, synthetic construct
YISNSAGTSYNPSLKS

SEQ ID NO: 30 - protein, 17 aa, synthetic construct
WIYPGNINTKYNEKFKG

SEQ ID NO: 31 - protein, 9 aa, synthetic construct
HTEGDDVDY

SEQ ID NO: 32 - protein, 7 aa, synthetic construct
HAYSFDF

SEQ ID NO: 33 - protein, 11 aa, synthetic construct
GGWSSYYAMDY

SEQ ID NO: 34 - protein, 12 aa, synthetic construct
DDGNLYYYAMDY

SEQ ID NO: 35 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 36 - protein, 1259 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - protein, 1258 aa, synthetic construct
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - protein, 1260 aa, synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - protein, 1257 aa, synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - protein, 1258 aa, synthetic construct
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGITAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 41 - protein, 1258 aa, synthetic construct
QCVNLTTRTQLPPAYTNSFTRGVYYPDKVFRSSVLHSTQDLFLPFFSNVTWFHVIHVSGTNGTKRFDNPVLPFNDGVYFASIEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINIVRDLPQGFSALEPLVDLPIGINITRFQTLLALHRSYLPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 42 - protein, 1257 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 43 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVSNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 12 aa, synthetic construct
MSVSPGEKVTMT

SEQ ID NO: 45 - protein, 12 aa, synthetic construct
MSASLGDRITIT

SEQ ID NO: 46 - protein, 19 aa, synthetic construct
MTQSPASLSASVGETVTIT

SEQ ID NO: 47 - protein, 22 aa, synthetic construct
NIVLTQSPASLAVSLGQRATIS

SEQ ID NO: 48 - protein, 10 aa, synthetic construct
QKPGSSPRLL

SEQ ID NO: 49 - protein, 10 aa, synthetic construct
QKPGKPPSFL

SEQ ID NO: 50 - protein, 10 aa, synthetic construct
RKQGKSPQLL

SEQ ID NO: 51 - protein, 10 aa, synthetic construct
QKPGQPPKLL

SEQ ID NO: 52 - protein, 31 aa, synthetic construct
GVPVRFSGSGSGTSYSLTISRVEAEDAATYY

SEQ ID NO: 53 - protein, 31 aa, synthetic construct
GVPSRFSGSGSGSDYSLTISNLESEDFADYY

SEQ ID NO: 54 - protein, 31 aa, synthetic construct
GMSSRFSGSGSGRQYSLKISSLHPDDVATYY

SEQ ID NO: 55 - protein, 31 aa, synthetic construct
GVPARFSGSGSRTDFTLTIDPVEADDAATYY

SEQ ID NO: 56 - protein, 7 aa, synthetic construct
TKLELKR

SEQ ID NO: 57 - protein, 7 aa, synthetic construct
TKLELKR

SEQ ID NO: 58 - protein, 7 aa, synthetic construct
TKLEIKR

SEQ ID NO: 59 - protein, 6 aa, synthetic construct
TKLEIK

SEQ ID NO: 60 - protein, 19 aa, synthetic construct
QLKQSGPSLVQPSQSLSIT

SEQ ID NO: 61 - protein, 20 aa, synthetic construct
VQLQQSGAELGKPGASVKLS

SEQ ID NO: 62 - protein, 20 aa, synthetic construct
VKLEESGPVLVKPSQSLSLT

SEQ ID NO: 63 - protein, 21 aa, synthetic construct
RVQLQQSGPELVKPGASVTIS

SEQ ID NO: 64 - protein, 7 aa, synthetic construct
RQSPGKG

SEQ ID NO: 65 - protein, 7 aa, synthetic construct
KQRPGQG

SEQ ID NO: 66 - protein, 7 aa, synthetic construct
RQFPGNK

SEQ ID NO: 67 - protein, 7 aa, synthetic construct
KQRPGQG

SEQ ID NO: 68 - protein, 7 aa, synthetic construct
TSVTVSS

SEQ ID NO: 69 - protein, 7 aa, synthetic construct
TTLTVSS

SEQ ID NO: 70 - protein, 7 aa, synthetic construct
TSVTVSS

SEQ ID NO: 71 - protein, 7 aa, synthetic construct
TSVTVSS

SEQ ID NO: 72 - protein, 1261 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVTSNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 73 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 74 - protein, 131 aa, synthetic construct
METDTLLLWVLLLWVPGSTGNIVLTQSPASLAVSLGQRATISCRASESVDTYGNSFMHWYQQKPGQPPKLLIYLASNLESGVPARFSGSGSRTDFTLTIDPVEADDAATYYCQQNNEDPLTFGAGTKLELK

SEQ ID NO: 75 - protein, 139 aa, synthetic construct
MGWSYIILFLVATATGVHSLVQLQQPGAELVKPGASVKLSCKASGYTFTSYFMYWVRQRPGQGLEWIGEINPSNGGNDFNEKFKSKATLTVDKSSSTAYMQLSSLTSEDSAVYYCTRSGDYGYDGFAYWGQGTLVTVSA

SEQ ID NO: 76 - protein, 15 aa, synthetic construct
RASESVDTYGNSFMH

SEQ ID NO: 77 - protein, 7 aa, synthetic construct
LASNLES

SEQ ID NO: 78 - protein, 9 aa, synthetic construct
QQNNEDPLT

SEQ ID NO: 79 - protein, 9 aa, synthetic construct
YTFTSYFMY

SEQ ID NO: 80 - protein, 17 aa, synthetic construct
EINPSNGGNDFNEKFKS

SEQ ID NO: 81 - protein, 10 aa, synthetic construct
GDYGYDGFAY

SEQ ID NO: 82 - protein, 39 aa, synthetic construct
DTLLLWVLLLWVPGSTGNIVLTQSPASLAVSLGQRATIS

SEQ ID NO: 83 - protein, 10 aa, synthetic construct
QKPGQPPKLL

SEQ ID NO: 84 - protein, 31 aa, synthetic construct; identical to SEQ ID NO: 55

SEQ ID NO: 85 - protein, 6 aa, synthetic construct
TKLELK

SEQ ID NO: 86 - protein, 21 aa, synthetic construct
LVQLQQPGAELVKPGASVKLS

SEQ ID NO: 87 - protein, 7 aa, synthetic construct
RQRPGQG

SEQ ID NO: 88 - protein, 26 aa, synthetic construct
LTVDKSSSTAYMQLSSLTSEDSAVYY

SEQ ID NO: 89 - protein, 6 aa, synthetic construct
TLVTVS

SEQ ID NO: 90 - protein, 942 aa, synthetic construct
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 91 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 92 - protein, 1260 aa, synthetic construct; identical to SEQ ID NO: 9

SEQ ID NO: 93 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVALGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 94 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 95 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 96 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 97 - protein, 1266 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTHHHHHH

SEQ ID NO: 98 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 99 - protein, 1257 aa, synthetic construct; identical to SEQ ID NO: 39

SEQ ID NO: 100 - protein, 1260 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 101 - protein, 1259 aa, synthetic construct; identical to SEQ ID NO: 36

SEQ ID NO: 102 - protein, 1258 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 103 - protein, 1255 aa, synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGGSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 104 - protein, 1257 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 105 - protein, 1256 aa, synthetic construct
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 106 - protein, 1254 aa, synthetic construct
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 107 - protein, 1260 aa, synthetic construct; identical to SEQ ID NO: 38

SEQ ID NO: 108 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 109 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 110 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 111 - protein, 1258 aa, synthetic construct; identical to SEQ ID NO: 41

SEQ ID NO: 112 - protein, 1257 aa, synthetic construct; identical to SEQ ID NO: 42

SEQ ID NO: 113 - protein, 1258 aa, synthetic construct; identical to SEQ ID NO: 37

SEQ ID NO: 114 - protein, 1260 aa, synthetic construct; identical to SEQ ID NO: 73

SEQ ID NO: 115 - protein, 1258 aa, synthetic construct; identical to SEQ ID NO: 40

SEQ ID NO: 116 - protein, 1260 aa, synthetic construct; identical to SEQ ID NO: 43

SEQ ID NO: 117 - protein, 1261 aa, synthetic construct; identical to SEQ ID NO: 72

SEQ ID NO: 118 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 119 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 120 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNNLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTVSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 121 - protein, 1257 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 122 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVRQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 123 - protein, 1257 aa, synthetic construct; identical to SEQ ID NO: 42

SEQ ID NO: 124 - protein, 1257 aa, synthetic construct; identical to SEQ ID NO: 118

SEQ ID NO: 125 - protein, 1255 aa, synthetic construct; identical to SEQ ID NO: 119

SEQ ID NO: 126 - protein, 1255 aa, synthetic construct; identical to SEQ ID NO: 120

SEQ ID NO: 127 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 128 - protein, 1257 aa, synthetic construct; identical to SEQ ID NO: 122

SEQ ID NO: 129 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 130 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 131 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNISNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 132 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 133 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 134 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 135 - protein, 1254 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 136 - protein, 1256 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 137 - protein, 1254 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 138 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHXNNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 139 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 140 - protein, 29 aa, synthetic construct
RLTITKDNSKSQVFFKMNSLQADDTAIYY

SEQ ID NO: 141 - protein, 29 aa, synthetic construct
KATLTVDKSSSSAHIHLTSLTSEDSAVYY

SEQ ID NO: 142 - protein, 29 aa, synthetic construct
RISITRDTSKNQFFLQLNSVTTEDTATYY

SEQ ID NO: 143 - protein, 29 aa, synthetic construct
KATLTADKSSSTAYMQLSSLTSEDSAVYF

SEQ ID NO: 144 - protein, 4 aa, synthetic construct
QQAQ

SEQ ID NO: 145 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 146 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 147 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNISNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 148 - protein, 188 aa, Severe acute respiratory syndrome-related coronavirus
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELL

SEQ ID NO: 149 - protein, 188 aa, Severe acute respiratory syndrome-related coronavirus
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELL

SEQ ID NO: 150 - protein, 188 aa, Severe acute respiratory syndrome-related coronavirus
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELL

SEQ ID NO: 151 - protein, 188 aa, Severe acute respiratory syndrome-related coronavirus
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELL

SEQ ID NO: 152 - protein, 188 aa, Severe acute respiratory syndrome-related coronavirus
NITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELL

SEQ ID NO: 153 - protein, 188 aa, Severe acute respiratory syndrome-related coronavirus
NITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELL

SEQ ID NO: 154 - protein, 188 aa, Severe acute respiratory syndrome-related coronavirus
NITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELL

SEQ ID NO: 155 - protein, 188 aa, Severe acute respiratory syndrome-related coronavirus
NITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELL